(19) United States
(12) Patent Application Publication
(10) Pub. No.: US 2026/0174801 A1
(43) Pub. Date: Jun. 25, 2026

(54) FROZEN ACTIVATED PLATELET COMPOSITIONS AND COLLECTIONS, AND METHODS OF MAKING AND USING THE SAME

(71) Applicant: Cellphire, Inc., Rockville, MD (US)

(72) Inventors: Michael Alexander MATHEWS, Arlington, VA (US); Stephen Edward AMOS, Buckeystown, MD (US); Benjamin J. KUHN, Arlington, VA (US); Glen Michael FITZPATRICK, North Potomac, MD (US); Rafael JORDA, Merignac (FR); Keith Andrew MOSKOWITZ, Westfield, IN (US)

(73) Assignee: Cellphire, Inc., Rockville, MD (US)

(21) Appl. No.: 19/402,962

(22) Filed: Nov. 26, 2025

Related U.S. Application Data
(60) Provisional application No. 63/726,137, filed on Nov. 27, 2024.
(60) Provisional application No. 63/810,669, filed on May 22, 2025.
(60) Provisional application No. 63/862,559, filed on Aug. 12, 2025.
(60) Provisional application No. 63/891,967, filed on Oct. 1, 2025.
(60) Provisional application No. 63/905,297, filed on Oct. 24, 2025.

(51) Int. Cl. A61K 35/19; A61P 7/04; C12N 5/00

(52) U.S. Cl. CPC A61K 35/19; A61P 7/04; C12N 5/526; C12N 5/545

(57) ABSTRACT

The present disclosure provides collections of cryo-vessels and compositions comprising cryopreserved platelets, for example frozen activated platelets, comprising a population of platelet particles in a cryopreservation medium in a frozen state. Such collections and compositions can include cryopreserved platelets having a biomolecule profile indicative of more than 1 platelet donor and can have various additional disclosed properties. Also provided herein, in some aspects are processes for preparing a batch of cryopreserved platelet compositions, and methods for reducing bleeding in a subject comprising administering cryopreserved platelets provided herein. In some aspects, provided herein are processes for preparing cryopreserved platelets from a platelet pool of more than 1 donor to provide collections of cryopreserved platelets with improved vial to vial and lot to lot consistency for example, in DMSO concentration. Also provided herein is a collection, a composition, and a process of preparing a batch of cryopreserved platelets in cryo-vessels that upon thawing and storing at room temperature for at least 8 hours have the property of exhibiting a pH of greater than 6.2. Furthermore, provided herein are compositions comprising frozen platelets that when stored at a temperature in a range of −10° C. to −30° C., for at least 1 month are capable of yielding a platelet count of at least 1.0×10¹¹/35 ml of the composition and other disclosed properties.

### CROSS-REFERENCE TO RELATED APPLICATIONS

This application claims priority to U.S. Provisional Application Ser. No. 63/905,297, filed on Oct. 24, 2025, U.S. Provisional Application Ser. No. 63/891,967, filed on Oct. 1, 2025, U.S. Provisional Application Ser. No. 63/862,559, filed on Aug. 12, 2025, U.S. Provisional Application Ser. No. 63/810,669, filed on May 22, 2025, and U.S. Provisional Application Ser. No. 63/726,137, filed on Nov. 27, 2024. Each of the above-mentioned patent applications is incorporated herein by reference in its entirety.

### STATEMENT OF GOVERNMENT INTEREST

The invention was made with government support under Contract No. W81XWH20C0030 awarded by the Defense Health Agency (DHA) of the U.S. Department of Defense. The government has certain rights in the invention.

### TECHNICAL FIELD

The present disclosure relates generally to blood products, and, more particularly, to cryopreserved platelet and cryopreserved platelet compositions, including process for preparing same.

### BACKGROUND

Platelets in a liquid stored apheresis form are used to treat blood-related issues, such as hemorrhages and thrombocytopenia. Typically, the platelets used in blood-related treatments have a shelf life of five days. This limits the availability of platelets for blood-related treatments. The short shelf life can also render 10-20% of available platelet collections unusable, causing about 200 million dollars to be lost annually. There are currently no commercially available cryopreserved platelets available for human transfusion.

Cryopreserved platelets (CPP) that are described in the field mostly relate to single donor processes, meaning that one apheresis unit is processed into one cryopreserved platelet unit. Apheresis platelet units, and single donor cryopreserved platelet units, have an inherent donor to donor variability since they are both single donor products. This variability includes multiple parameters, not to mention the variation in the percentage of cryoprotectant, such as dimethyl sulfoxide (DMSO), total number of platelets, and platelet concentration in the single donor products. Furthermore, even for CPPs made from pooled platelets, have too much lot-to-lot or batch-to-batch variability in these parameters. Accordingly, there is a long-felt need in the art to overcome this donor-to-donor and lot-to-lot or batch-to-batch variability in cryopreserved platelets, or cryopreserved platelet compositions.

It is known that stringent freezing temperature conditions are required for storing cryopreserved platelets, for example, ultra-freezers are required to maintain temperatures at ≤−65° C. in order to have a functional platelet product for applications such as in a battlefield, or in a hospital or other patient treatment center. However, depending on geographical locations, such battlefields or hospitals may not be equipped with ultra-freezers due to the size of the freezer unit the cost of one or especially more such freezer units, and/or the lack of an adequate electrical power supply, thereby, making it difficult to store the cryopreserved platelets at such temperatures. Accordingly, there is a long-felt need in the art to have a cryopreserved platelet composition that is effective at controlling bleeding, is readily manufacturable without highly specialized equipment and is capable of being stored in standard −20° C. freezers for months or even years.

The risk of uncontrolled bleeding in a subject during a surgical intervention, or in a post-surgery phase presents a serious health risk because there remains a need for effective therapies for reducing or controlling, and/or treating bleeding in a subject, and not to mention in a subject who has one or more indications that can cause the subject to bleed in an uncontrolled manner. For example, there remains a need for improved treatments for decreasing bleeding in a subject who is undergoing invasive surgery, such as a cardiopulmonary bypass surgery.

### SUMMARY

To overcome the above-mentioned and additional problems in the art, the present disclosure provides aspects and embodiments that include frozen platelets, frozen activated platelets, frozen platelet derivatives, cryopreserved platelets, and/or cryopreserved platelet derivatives. In some aspects and embodiments, pooled cryopreserved platelets (CPP), or frozen activated platelets are provided that can be aliquoted into multiple doses of a final product from a starting material that typically includes multiple platelet units. Improved processes of the present disclosure create a final product, for example, pooled cryopreserved platelets, or frozen activated platelets that exhibits decreased lot to lot variability, when compared to the donor-to-donor variability of current standard of care products (single donor CPP or apheresis platelet units). The novel process aspects and embodiments herein allow for increased in-process controls (IPCs) and reduces variability in the processing compared to single donor CPP processes. The processes provided herein facilitate increased control of the excipients and freezing volume compared to single donor process and prior CPP processes. This increased control reduces variation of the DMSO content, dosage volume, and total cell number that is administered to a patient, which increases the safety profile of a CPP product provided herein. The ability to create multiple doses, such as multiple cryo-vessels of a batch of CPPs from a pool of platelet units allows for quality control testing of the final product for release of lots which is not achievable for single donor CPP.

Further, in some aspects, provided herein is a process for preparing a cryopreserved platelet composition that can be stored at a temperature higher than −65° C., for example, at a temperature in the range of −10° C. to −30° C. for a time period of at least 1 month, 3 months, 12 months, or until the cryopreserved platelets are required for treating a subject in need thereof. Also provided is a frozen platelet composition, which is capable of being stored at a temperature higher than −65° C., for example at a temperature in the range of −10° C. to −30° C. for a time period of at least 1 month, 3 months, 12 months, or until the cryopreserved platelets are required for treating a subject in need thereof.

Accordingly, provided herein in one aspect is a process for preparing a batch of a cryopreserved platelet composition, comprising:
a) forming a concentrated pooled platelet resuspension (CPR) with a weight or volume based on the number of platelet units (PU) to form the CPR
b) adding a cryoprotectant to the CPR to obtain a CPR having the cryoprotectant;
c) distributing the CPR having DMSO among more than 1 cryo-vessel from a collection of cryo-vessels; and
d) freezing the collection of cryo-vessels to prepare the batch of the cryopreserved platelet composition. In illustrative embodiments, the forming the CPR is done with a weight based on the number of PU. In illustrative embodiments, the cryoprotectant comprises dimethyl sulfoxide (DMSO), and the concentration of DMSO in the CPR having DMSO is in the range of 4% to 8%. In illustrative embodiments, the distributing is done to obtain 1 PU equivalent weight of the CPR having DMSO in each cryo-vessel. Typically, the freezing is initiated within 3 hours after adding the cryoprotectant, for example, DMSO.

Accordingly, provided herein in one aspect is a process for preparing a cryopreserved platelet composition comprising cryopreserved platelets, said process comprising:
i) freezing a population of platelets in a cryopreservation medium at a temperature of equal to or less than −50° C. to form an initial frozen platelet composition;
ii) subjecting the initial frozen platelet composition to a temperature of equal to or more than −30° C. but less than 0° C., or less than −1° C.; and
iii) storing the initial frozen platelet composition at the temperature of equal to or more than −30° C. but less than 0° C., or less than −1° C., to form the cryopreserved platelet composition comprising the cryopreserved platelets.

Accordingly, provided herein in one aspect is a process for preparing a cryopreserved platelet composition comprising cryopreserved platelets, said process comprising:
i) freezing a population of platelets in a cryopreservation medium at a temperature of equal to or less than −50° C. to form an initial frozen platelet composition; and
ii) storing the initial frozen platelet composition at a temperature of equal to or more than −30° C. but less than 0° C., or less than −1° C., for at least 1 month to form the cryopreserved platelet composition.

Accordingly, provided herein in one aspect is a process for preparing a batch of a cryopreserved platelets, comprising:
a) pooling at least 2 platelet units into one vessel and at least another platelet unit into another vessel, wherein the platelet units are from more than one donor;
b) centrifuging each vessel to obtain a supernatant comprising plasma, and a pellet comprising platelets;
c) resuspending the pellet in each vessel to form a resuspension wherein the resuspension has a target weight determined by the number of units pooled or provided in the vessel;
d) pooling the resuspension from each vessel to form a pooled resuspension in a pooled resuspension vessel;
e) adding a cryoprotectant to the pooled resuspension vessel having the pooled resuspension to obtain a pooled resuspension having the cryoprotectant;
f) distributing the pooled resuspension having the cryoprotectant from the pooled resuspension vessel among a number of cryo-vessels; and
g) freezing the pooled resuspension having the cryoprotectant in the cryo-vessels, to form the batch of cryopreserved platelets. The target weight in non-limiting examples, can be in the range of 15.0 to 30.0 g, 15.0 to 29.0 g, 15.0 to 28.5 g, or in illustrative embodiments 15.9 g to 27.9 g times the number of units pooled or provided in the vessel. In illustrative embodiments, the cryoprotectant is or comprises DMSO.

Accordingly, provided herein in one aspect is a collection of cryo-vessels,
wherein each cryo-vessel in the collection comprises a population of cryopreserved platelets,
wherein the population of the cryopreserved platelets in each cryo-vessel have a set of biomolecule profiles indicative of more than 1 platelet donor,
wherein the cryopreserved platelets in each cryo-vessel of a batch of cryo-vessels of the collection have an indistinguishable set of biomolecular profiles comprising a phosphatidylserine positivity, when measured using lactadherin binding of at least 30%,
wherein each batch of the collection has a different set of biomolecular profiles than any other batch in the collection,
wherein the collection comprises a plurality of at least 2 batches of cryo-vessels,
wherein the population of cryopreserved platelets in the collection, upon thawing have a capacity to generate thrombin in an in vitro thrombin generation assay, and
wherein the population of cryopreserved platelets in the collection have the property of exhibiting a pH of greater than 6.0, in illustrative embodiments, greater than 6.2. In illustrative embodiments, the cryopreserved platelets in the collection have the property of exhibiting a pH of greater than 6.2 upon thawing and storing at a temperature in the range of 15° C. to 30° C. for 6 hours to 24 hours.

Accordingly, provided herein in one aspect is a composition, or a frozen composition comprising frozen platelets in a cryopreservation medium in a frozen state,
wherein the composition comprises frozen platelets displaying a CD62 positivity of at least 30%, and/or a phosphatidylserine positivity, when measured using lactadherin binding of at least 50%,
wherein the frozen platelets have a set of biomolecule profiles indicative of more than 1 platelet donor,
wherein the composition comprising frozen platelets, upon thawing has a capacity to generate thrombin in an in vitro thrombin generation assay, and
wherein the composition comprising frozen platelets has a property of exhibiting a pH of greater than 6.0, in illustrative embodiments, greater than 6.2. In illustrative embodiments, the composition comprising frozen platelets in a cryopreservation medium in a frozen state, have the property of exhibiting a pH of greater than 6.2 upon thawing and storing at a temperature in the range of 15° C. to 30° C. for 6 hours to 24 hours.

Accordingly, provided herein in one aspect is a collection of cryo-vessels, wherein each cryo-vessel in the collection comprises cryopreserved platelets,
wherein the cryopreserved platelets in each cryo-vessel have a set of biomolecule profiles indicative of more than 1 platelet donor,
wherein the cryopreserved platelets in each cryo-vessel of a batch of cryo-vessels of the collection have an indistinguishable set of biomolecular profiles comprising a CD62 positivity of at least 30%, and/or a phosphatidylserine positivity, when measured using lactadherin of at least 50%,
wherein each batch of the collection has a different set of biomolecular profiles than any other batch in the collection,
wherein the collection comprises a plurality of at least 2 batches of cryo-vessels,
wherein the cryopreserved platelets in the collection, upon thawing have a capacity to generate thrombin in an in vitro thrombin generation assay, and
wherein the cryopreserved platelets in the collection, upon thawing have less than 10×10⁶ CD61-positive microparticles/μl.

Accordingly, provided herein in one aspect is a cryopreserved platelet composition comprising frozen activated platelets comprising a population of platelet particles in a cryopreservation medium in a frozen state in a cryo-vessel,
wherein the frozen activated platelets display a CD62 positivity of at least 60%, and/or a phosphatidylserine positivity, when measured using lactadherin of at least 50%,
wherein the frozen activated platelets have a set of biomolecule profiles indicative of more than 1 platelet donor,
wherein the frozen activated platelets, upon thawing has a capacity to generate thrombin in an in vitro thrombin generation assay, and
wherein the frozen activated platelets, upon thawing do not comprise more than 10×10⁶ CD61-positive microparticles/μl.

Accordingly, provided herein in one aspect is a collection of cryo-vessels comprising cryopreserved platelets, wherein the cryopreserved platelets in each cryo-vessel have a biomolecule profile indicative of more than 1 platelet donor, and wherein the concentration of the cryoprotectant, in illustrative embodiments DMSO, in the cryopreserved platelets of a first cryo-vessel is within 15%, 12%, 10%, 9%, 7%, 5%, 3%, 2%, 1%, or 0.5% of the concentration of cryoprotectant, in illustrative embodiments DMSO, in the cryopreserved platelets of a second cryo-vessel. In some embodiments, the collection comprises at least 2, 5, 10, 15, 20, or more cryo-vessels from 1 or in illustrative embodiments, 2, 3, 4, 5 or more batches, wherein a batch of cryo-vessels has an identical set of biomolecule profiles, and wherein each batch of the collection has a different set of biomolecule profiles than any other batch in the collection. In some embodiments, exactly or at least 3, 4, 5, 6, 7, 8, 9, 10, 11, 12, or more cryo-vessels are in each batch.

Accordingly, provided herein in one aspect is a cryopreserved platelet composition comprising cryopreserved platelets, wherein the cryopreserved platelets are stored at about −20° C. to −60° C. for a time period of at least 1 month.

Accordingly, provided herein in one aspect is a composition comprising frozen platelets in a cryopreservation medium in a frozen state, wherein the composition is capable of yielding one or more of the following recited properties after storage for at least 1 month, 2, 3, 4, 5, or 6 months, upon thawing:
a) is in a liquid state without requiring the addition of a liquid to achieve such liquid state;
b) exhibits a platelet count of at least 1.0×10¹¹/35 ml of the composition;
c) yields a single peak that corresponds to a compromised membrane peak in a membrane integrity assay;
d) exhibits a CD61-positive-microparticle content of less than 50% of the CD61 positive particles, and/or a phosphatidylserine positivity, when measured using lactadherin of at least 50%, in the composition, and
e) generates thrombin in an in vitro thrombin generation assay.

Provided herein in one aspect is a method for reducing/decreasing, or treating bleeding in a subject, comprising:
thawing a cryo-vessel of cryopreserved platelets from the batch of cryopreserved platelets obtained from any of the aspects or embodiments herein, a cryopreserved platelet composition obtained from any of the aspects or embodiments herein, a cryo-vessel from a collection of cryo-vessels comprising cryopreserved platelets of any of the aspects or embodiments herein, or a composition of any of the aspects or embodiments herein, to form a thawed composition, and
administering the thawed composition comprising a dose, a first dose, or an effective amount of the cryopreserved platelets to the subject. In illustrative embodiments, the subject is undergoing surgery, or has undergone surgery.

Further details regarding aspects and embodiments of the present disclosure are provided throughout this patent application. Sections and section headers are for ease of reading and are not intended to limit combinations of disclosure, such as methods, compositions, and kits or functional elements therein across sections. Further details regarding aspects and embodiments of the present disclosure are provided throughout this patent application. Sections and section headers are for ease of reading and are not intended to limit combinations of disclosure, such as methods, compositions, or other functional elements therein across sections.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. As the color drawings are being filed electronically via EFS-Web, only one set of the drawings is submitted.

FIG. 1A, FIG. 1B and FIG. 1C are non-limiting flowcharts of steps of exemplary processes for preparing a batch or a lot of cryo-vessels comprising cryopreserved platelets.

FIG. 1D is a schematic of a tubing tree that can be used as per one of the aspects of process herein.

FIG. 2A shows a non-limiting flow chart of an exemplary process for preparing a cryopreserved platelet composition that is capable of storing at a temperature in the range of −10° C. to −30° C.

FIG. 2B shows a non-limiting flow chart of an exemplary process for preparing a cryopreserved platelet composition that integrates the exemplary process of FIG. 1A and FIG. 2A.

FIG. 3 displays the target % DMSO that can be achieved using the calculation provided in the single donor process of Vitalant for preparing cryopreserved platelets.

FIG. 4A shows a freezing volume distribution of 255 units prepared by the single donor process of Vitalant for preparing cryopreserved platelets.

FIG. 4B shows a freezing volume distribution for units with aggregates observed within 6 hours following thawing and resuspension for 32 units.

FIG. 5 shows a comparison of the correlation of APC volume to % DMSO of the single donor method for preparing cryopreserved platelets of Vitalant, and CPP pooled process as disclosed herein.

FIG. 6 shows the correlation between post-expression volume (resuspension volume), and % DMSO for the CPP pooled process as disclosed herein.

FIG. 7 shows the percentage recovery of platelets for the batches stored at −80° C. (single temperature cryopreserved-product) and −20° C. (transition temperature cryopreserved-product).

FIG. 8 shows the percentage aggregation of platelets for the batches stored at −80° C. (single temperature cryopreserved-product) and −20° C. (transition temperature cryopreserved-product), and comparison with apheresis platelets with arachidonic acid (AA), collagen, and thrombin receptor-activating peptide 6 (TRAP-6).

FIG. 9 shows the peak distribution of platelets for the batches stored at −80° C. (single temperature cryopreserved-product) depicted by “#” and −20° C. (transition temperature cryopreserved-product) depicted by “*”, and apheresis platelets depicted by “+”.

FIG. 10 shows the differences in attributes of platelet counts, platelet concentration, thrombin elastography (TEG), thrombin generation ability (TGA), platelet markers characterized using flow cytometry, and pH between single donor cryopreserved platelet product, and pooled multiple donor cryopreserved platelet product.

FIG. 11 shows a representative scatter plot (FSC-H on x-axis and APC-H on y-axis) distinctively showing two populations originating from cryopreserved platelets upon thawing: a first population comprising smaller CD61-positive microparticles marked with a polygon; and a second population comprising larger platelet-like particles.

FIG. 12 shows the difference between thrombin generation ability of cryopreserved platelets as disclosed herein (pooled CPP product), and room temperature platelets (RTP).

FIG. 13 shows the flow cytometry-based characterization, in terms of mean fluorescent intensity (MFI) of cryopreserved platelets as disclosed herein (pooled CPP product), and room temperature platelets (RTP) for various platelet-associated markers—GPVI, GPIaIIa (CD49), GPIbα (CD42b), vWF, thrombospondin, CD62P (P-Selectin), LAMP-3, phosphatidylserine (PS), fibrinogen, and GPIIIa (CD61).

FIG. 14 and FIG. 15 show the difference in the aggregation response in terms of platelet count, and aggregation %, respectively between the cryopreserved platelets as disclosed herein (pooled CPP product), and room temperature platelets (RTP).

FIG. 16A shows the difference in platelet concentration by platelet count per ul, between the RTP, cold stored platelets (CSP), and pooled CPP product.

FIG. 16B shows the difference in platelet total count by platelet count per unit, between RTP, CSP, and pooled CPP product.

FIG. 17 shows the difference in pH between RTP, CSP, and pooled CPP product.

FIG. 18A shows the difference in percent positivity for p-selectin (CD62P) and PS (Lactadherin), between RTP, CSP, and pooled CPP product.

FIG. 18B shows the difference in percent positivity for GPVI and GPIba (CD42b) between RTP, CSP, and pooled CPP product.

FIG. 18C shows the difference in percent positivity for VWF, thrombospondin, and fibrinogen, between RTP, CSP, and pooled CPP product.

FIG. 19A shows the difference in MFI for p-selectin (CD62P) and PS (Lactadherin), between RTP, CSP, and pooled CPP product.

FIG. 19B shows the difference in MFI for GPVI and GPIba (CD42b), between RTP, CSP, and pooled CPP product.

FIG. 19C shows the difference in MFI for VWF, thrombospondin, and fibrinogen, between RTP, CSP, and pooled CPP product.

FIG. 20A shows two sub-populations: a more activated sub-population, and a less activated sub-population in a pooled CPP product as disclosed herein upon thawing, when analyzed using flow cytometry.

FIG. 20B shows the difference in terms of median height in FSC, and SSC for the more activated sub-population, and the less activated sub-population.

FIG. 20C shows the difference in MFI for PS, CD41, TSP-1, GPVI, and CD42, for two sub-populations.

FIG. 21 shows the difference in TGPU, expressed as IU per 10⁶ particles, measured on a CLARIOstar^{plus} instrument, between RTP, CSP, and pooled CPP product.

FIG. 22A shows the thrombin generation per unit (TGPU), expressed as IU per 10⁶ particles, measured on a Thrombinoscope TGA instrument, for RTP, CSP, and pooled CPP product.

FIG. 22B shows the difference in thrombin peak height (nM) over time (minutes), for RTP, CSP, and pooled CPP product.

FIG. 22C shows the lag time, expressed in minutes, for RTP, CSP, and pooled CPP product.

FIG. 22D shows the time to peak, expressed in minutes, for RTP, CSP, and pooled CPP product.

FIG. 22E shows the velocity index, expressed as nM/minute, for RTP, CSP, and pooled CPP product.

FIG. 23A shows the optical density (O.D.) at 405 nm over time, expressed in seconds, for RTP day 4, CSP day 7, and pooled CPP product TO.

FIG. 23B shows the maximum slope, expressed as optical density (O.D.) per second, as a function of particle concentration (particles/μL) for RTP, CSP, and pooled CPP product.

FIG. 23C shows the time to maximum slope, in seconds, as a function of particle concentration (particles/μL), for RTP, CSP, and pooled CPP product.

FIG. 24A shows the R time, as a ratio of untreated sample, for untreated, RTP, CSP, and pooled CPP product.

FIG. 24B shows the angle, in degrees, for untreated, RTP, CSP, and pooled CPP product.

FIG. 24C shows the max amplitude (MA), in mm, for untreated, RTP, CSP, and pooled CPP product.

FIG. 25A shows the max aggregation, as a percentage, in response to collagen, for RTP, CSP, and pooled CPP product.

FIG. 25B shows the max aggregation, as a percentage, in response to ADP, for RTP, CSP, and pooled CPP product.

FIG. 25C shows the max aggregation, as a percentage, in response to AA, for RTP, CSP, and pooled CPP product.

FIG. 25D shows the max aggregation, as a percentage, in response to TRAP-6, for RTP, CSP, and pooled CPP product.

FIG. 25E shows the max aggregation, as a percentage, in response to thrombin, for RTP, CSP, and pooled CPP product.

FIG. 26A shows the max aggregation, as a percentage, in the presence of TRAP+ADP+epinephrine (epi), for RTP, CSP, and pooled CPP product.

FIG. 26B shows the max aggregation, as a percentage, in the presence of collagen+epi, for RTP, CSP, and pooled CPP product.

FIG. 26C shows the max aggregation, as a percentage, in the presence of AA+ADP, for RTP, CSP, and pooled CPP product.

FIG. 26D shows the max aggregation, as a percentage, in the presence of TRAP, for RTP, CSP, and pooled CPP product.

FIG. 26E shows the max aggregation, as a percentage, in the presence of collagen, for RTP, CSP, and pooled CPP product.

FIG. 27A shows the amount of ATP, in nmol, in response to TRAP+ADP+epi, for RTP, CSP, and pooled CPP product.

FIG. 27B shows the amount of ATP, in nmol, in response to collagen+epi, for RTP, CSP, and pooled CPP product.

FIG. 27C shows the amount of ATP, in nmol, in response to AA+ADP, for RTP, CSP, and pooled CPP product.

FIG. 27D shows the amount of ATP, in nmol, in response to TRAP, for RTP, CSP, and pooled CPP product.

FIG. 27E shows the amount of ATP, in nmol, in response to collagen, for RTP, CSP, and pooled CPP product.

FIG. 28A shows the percentage of PAC-1 positive platelets, at resting and stimulated conditions, for RTP, CSP, and pooled CPP product.

FIG. 28B shows the MFI of PAC-1 positive platelets, at resting and stimulated conditions, for RTP, CSP, and pooled CPP product.

FIG. 28C shows the MFI, within percent positivity gate, of PAC-1 positive platelets, at resting and stimulated conditions, for RTP, CSP, and pooled CPP product.

FIG. 29A shows the total platelet count per unit, for RTP (day 4 and 7), CSP (day 7 and 14), and pooled CPP product.

FIG. 29B shows the mean normalization value of total platelet count per unit, for RTP (day 4 and 7), CSP (day 7 and 14), and pooled CPP product.

FIG. 30A shows the pH, for RTP (day 4 and 7), CSP (day 7 and 14), and pooled CPP product.

FIG. 30B shows the mean normalized value of pH, for RTP (day 4 and 7), CSP (day 7 and 14), and pooled CPP product.

FIG. 31A shows the percentage of particles positive for lactadherin, for RTP (day 4 and 7), CSP (day 7 and 14), and pooled CPP product.

FIG. 31B shows the mean normalized values, of the percentage of particles positive for lactadherin, for RTP (day 4 and 7), CSP (day 7 and 14), and pooled CPP product.

FIG. 32A shows the thrombin generation per unit (TGPU), expressed as IU per 10⁶ particles, measured on a Thrombinoscope instrument, for RTP (Day 4 and Day 7), CSP (Day 7 and Day 14), and pooled CPP product.

FIG. 32B shows the mean normalized values, of thrombin generation per unit (TGPU), expressed as IU per 10⁶ particles, measured on a Thrombinoscope instrument, for RTP (Day 4 and Day 7), CSP (Day 7 and Day 14), and pooled CPP product.

FIG. 33 shows preliminary pooled CPP product water bath thawing temperature probe data for six units, for the average, minimum, and maximum temperatures over time (minutes) during thawing.

FIG. 34 shows the pH for six different platelet products as disclosed herein, GMP CPP (n=91), QC release platelets, pooled CPP product thawed using QuickThaw (5 min), pooled CPP product thawed using Sahara III (7.5 min), pooled CPP product thawed using ZipThaw (4-5 min), and 36-48 month pooled CPP product.

FIG. 35 shows the total platelet count (as x10¹¹ platelets) for six different platelet products as disclosed herein, GMP CPP (n=91), QC release platelets, pooled CPP product thawed using QuickThaw (5 min), pooled CPP product thawed using Sahara III (7.5 min), pooled CPP product thawed using ZipThaw (4-5 min), and 36-48 month pooled CPP product.

FIG. 36 shows the platelet concentration (platelets/nL) for six different platelet products as disclosed herein, GMP CPP (n=91), QC release platelets, pooled CPP product thawed using QuickThaw (5 min), pooled CPP product thawed using Sahara III (7.5 min), pooled CPP product thawed using ZipThaw (4-5 min), and 36-48 month pooled CPP product.

FIG. 37 shows the CD61+microparticle concentration (MPs/nL) for six different platelet products as disclosed herein, GMP CPP (n=91), QC release platelets, pooled CPP product thawed using QuickThaw (5 min), pooled CPP product thawed using Sahara III (7.5 min), pooled CPP product thawed using ZipThaw (4-5 min), and 36-48 month pooled CPP product.

FIG. 38 shows the percentage of particles positive for lactadherin for six different platelet products as disclosed herein, GMP CPP (n=91), QC release platelets, pooled CPP product thawed using QuickThaw (5 min), pooled CPP product thawed using Sahara III (7.5 min), pooled CPP product thawed using ZipThaw (4-5 min), and 36-48 month pooled CPP product.

FIG. 39 shows thrombin generation assay values, as IU per 10⁶ platelets, for six different platelet products as disclosed herein, GMP CPP (n=91), QC release platelets, pooled CPP product thawed using QuickThaw (5 min), pooled CPP product thawed using Sahara III (7.5 min), pooled CPP product thawed using ZipThaw (4-5 min), and 36-48 month pooled CPP product.

FIG. 40A shows the mean normalized value for Lactadherin binding positivity for pooled CPP, CSP, and RTP from different timepoints of storage.

FIG. 40B shows the mean normalized value for pH for pooled CPP, CSP, and RTP from different timepoints of storage.

FIG. 40C shows the mean normalized value for platelet count for pooled CPP, CSP, and RTP from different timepoints of storage.

FIG. 40 D shows the mean normalized value for thrombin generation for pooled CPP, CSP, and RTP from different timepoints of storage.

FIG. 40 E shows the mean normalized value for R time for pooled CPP, CSP, and RTP from different timepoints of storage.

FIG. 40 F shows the mean normalized value for maximum amplitude (MA) for pooled CPP, CSP, and RTP from different timepoints of storage.

FIG. 41A shows the output from the image analysis for determining the adhesion of thawed activated platelets herein to a collagen-coated channel.

FIG. 41B shows the output from image analysis for determining the adhesion of platelets from platelet rich plasma to a collagen-coated channel.

### DEFINITIONS

As used herein, a “composition comprising frozen platelets” can also be referred to as a “cryopreserved platelet composition”, “frozen platelet composition”, “CPP”, “cryopreserved platelets”, or “frozen platelets”.

As used herein, a “cryopreserved platelet composition” can also be referred to as “cryopreserved platelets”, “frozen platelets”, “CPP”, “frozen platelet composition”, or “composition comprising frozen platelets”.

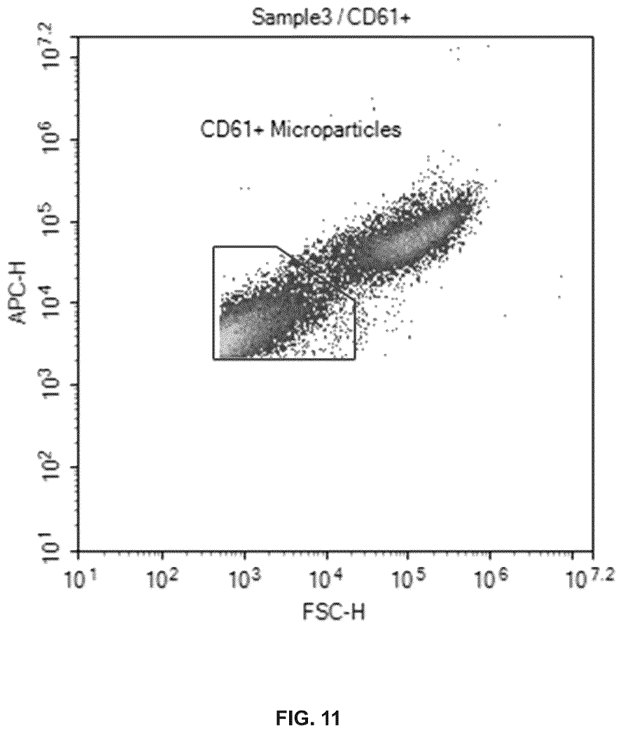

As used herein, “cryopreserved platelets”, can also be referred to as “cryopreserved platelet composition”, “frozen platelets”, “CPP”, “frozen platelet composition”, or “composition comprising frozen platelets”. Cryopreserved platelets are frozen platelet particles that when thawed are in a liquid state regardless of whether any liquid is added to the frozen platelet particles after thawing. Accordingly, cryopreserved platelets, or frozen platelets are not fresh platelets, liquid stored platelets, or apheresis platelets, and they are not freeze-dried platelet derivatives. During processing cryopreserved platelets are not dried. The term “cryopreserved platelets”, or “frozen platelets” does not imply any minimum length of time such platelets are present in a frozen state. However, cryopreserved platelets are typically stable for at least 1, 2, 3, 4, 5, 6, 9, or 12 months, and in illustrative embodiments are stable for at least 18, 24, 36, or 48 hours. Cryopreserved platelets are typically suspended in a cryoprotectant in a frozen state, until thawing before use. In some embodiments herein, cryopreserved platelets are stored for a period of at least 1, 2, 3, 4, 5, 6, 9, or 12 months at a temperature of −20° C. Cryopreserved platelet compositions when analyzed after thawing include two populations of particles that can be categorized broadly based on the size of particles obtained: thawed platelet particles and microparticles. Thus, upon thawing, cryopreserved platelet compositions provide a thawed composition that contains at least a first population of particles, also referred to herein as “thawed platelet particles” and a second population of particles referred to as “microparticles”. Thawed platelet particles are similar, or much more similar in size to in-dated stored platelets, or liquid stored platelets and are larger in size than microparticles. These populations in a cryopreserved platelet composition can be identified using techniques including, but not limited to flow cytometry. One of the representative images of a scatter plot obtained in flow cytometry-based studies distinctively showing the populations of microparticles and platelets is shown in FIG. 11. The microparticles, such as CD61-positive microparticles can be identified as the population of particles (shown inside the polygon) that are smaller in size as compared to the larger-sized population as per the forward scatter (FSC-H) (FIG. 11). It will be understood that additional populations of particles such as exosomes might be present, but if present, they will be even smaller than microparticles and they may not be detectable depending on the technology used for particle analysis.

As used herein, “CPP” can also be referred to as a “cryopreserved platelet composition”, “cryopreserved platelets”, “frozen platelets”, “frozen platelet composition”, or “composition comprising frozen platelets”.

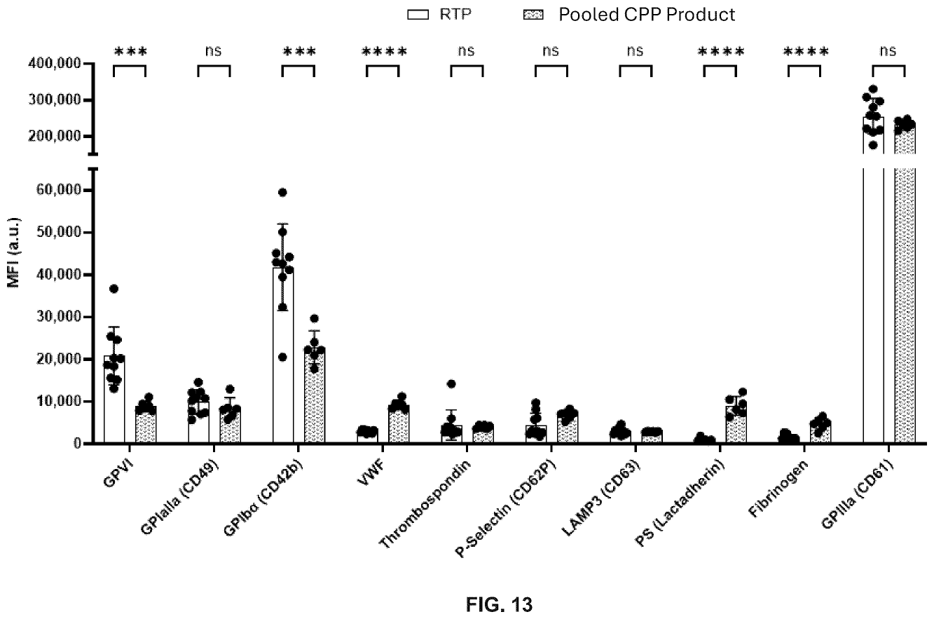

As used herein, “frozen activated platelets”, are cryopreserved platelets wherein the population of particles therein that have a particle size, for example, diameter of at least 0.3 μm, have a CD62 percent positivity of at least 50%, and a phosphatidylserine positivity of at least 50%, when measured using lactadherin binding. Both CD62 and phosphatidylserine positivity can be determined using a flow cytometer. Furthermore, the amount of CD62 and phosphatidylserine in frozen activated platelets is greater than the amount of these markers in apheresis platelets, liquid stored platelets, or room temperature platelets as illustrated in FIG. 13 herein.

As used herein, a “frozen platelet composition” can also be referred to as a “composition comprising frozen platelets”, “cryopreserved platelet composition”, “CPP”, “cryopreserved platelets”, or “frozen platelets”.

As used herein, “frozen platelets” can also be referred to as a “composition comprising frozen platelets”, “cryopreserved platelet composition”, “frozen platelet composition”, “CPP”, or “cryopreserved platelets”.

As used herein, “microparticles” are a population of particles within cryopreserved platelets that are smaller in size than the population of thawed platelet particles therein.

As used herein “platelet derivatives” in the context of cryopreserved platelets, or a composition comprising frozen platelets and/or platelet derivatives implies particles that, unlike fresh platelets, do not have intact cell membranes, for example, as demonstrated by a Calcein AM membrane integrity assay (See e.g., Example 8). Accordingly, in some embodiments, a composition provided herein comprises platelet derivatives, and such platelet derivatives are not freeze-dried platelet derivatives.

As used herein “thawed platelet particles” are a population of particles within cryopreserved platelets that are larger than the population of microparticles therein.

As used herein, “room temperature” refers to a temperature that is within the range of 20° C. to 25° C. including the values of the lower and upper limit.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 1 to 49, 1 to 25, 1.7 to 31.9, and so forth (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. When multiple low and multiple high values for ranges are given that overlap, a skilled artisan will recognize that a selected range will include a low value that is less than the high value.

As used herein, the symbol “<” means less than or in the context of temperatures, can mean below a recited temperature. As used herein, the symbol “/” in the context of temperatures, can mean to include a range of temperature, for example −20° C.+/−2° C. would mean a temperature from −18° C. to −22° C. As used herein, “about” or “consisting essentially of” mean±10% of the indicated range, value, or structure, unless otherwise indicated. As used herein, the terms “include” and “comprise” are open ended and are used synonymously. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

Although methods and materials similar or equivalent to those described herein can be used in the practice or testing of the present disclosure, suitable methods and materials are described below. All publications, patent applications, patents, and other references mentioned herein are incorporated by reference in their entireties. In case of conflict, the present specification, including explanations of terms, will control. In addition, the materials, methods, and examples are illustrative only and not intended to be limiting.

It is appreciated that certain features of aspects and embodiments herein, which are, for clarity, discussed in the context of separate embodiments, may also be provided in combination in a single embodiment. Conversely, various aspects and embodiments, which are, for brevity, discussed in the context of a single aspect or embodiment, may also be provided separately or in any suitable sub-combination. All combinations of aspects and embodiments are specifically embraced herein and are disclosed herein just as if each and every combination was individually and explicitly disclosed. In addition, all sub-combinations of the various aspects and embodiments and elements thereof are also specifically disclosed herein even if each and every such sub-combination is not individually and explicitly disclosed herein.

While the embodiments of the present disclosure are amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

It is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### DETAILED DESCRIPTION

The present disclosure addresses many long-felt needs and long-standing problems in the art, such as, but not limited to those mentioned in the Background section herein. To overcome the above-mentioned and additional problems in the art, the present disclosure provides aspects and embodiments that include frozen platelets, frozen platelet compositions comprising frozen platelets, frozen platelet derivatives, cryopreserved platelet compositions comprising cryopreserved platelets, cryopreserved platelets, and/or cryopreserved platelet derivatives. In illustrative aspects and embodiments, such compositions comprise pooled cryopreserved platelets, also referred to as pooled cryopreserved platelet products or pooled multiple donor cryopreserved platelets and are prepared by aliquoting or distributing a concentrated pooled platelet resuspension into multiple doses of a final product, thus from a starting material that typically includes multiple platelet units. Illustrative processes provided in the present disclosure create a final product that has unexpected properties such as increased stability after thawing and decreased microparticle content. For example, the composition comprising cryopreserved platelets herein upon thawing and storing for 6, 8, or 24 hours is capable of displaying a pH of above 6.0, typically, above 6.2. Additionally, illustrative compositions comprising cryopreserved platelets herein upon thawing have less than 10×10⁶ CD61-positive microparticles/μl of the composition. Furthermore, illustrative batches of cryopreserved platelets (CPP) made using processes provided herein, exhibit decreased lot to lot or batch-to-batch variability, when compared to the donor-to-donor variability of current standard of care products (e.g., single donor CPP or apheresis platelet units). Aspects and embodiments herein allow for increased in-process controls (IPCs) and reduce variability in the processing compared to single donor CPP processes. The processes provided herein facilitate increased control of the excipients and freezing volume compared to single donor process and prior CPP processes. This increased control reduces variation of the cryoprotectant (e.g., DMSO) content, dosage volume, and total cell/platelet number that is administered to a patient, which increases the safety profile of a CPP product provided herein.

The ability to create multiple doses, such as multiple cryo-vessels from the same batch of CPPs made from a pool of platelet units allows for quality control testing of the final product for release of lots, and provides samples for archiving that can be used for future testing and troubleshooting, which is not achievable for single donor CPP. Accordingly, CPP formed or prepared from platelets obtained from more than one platelet donor are herein referred to as pooled CPP product, or pooled multi-donor CPP product, whereas, CPP formed or prepared from platelets obtained from a single donor is referred to as single donor CPP. In some cases, single donor CPP can include 1 or more than 1 platelet unit from a single donor. Additionally, since multiple doses are created from a common pool, the process allows for direct comparisons for stability studies and provides archive samples that can be used for later analysis. Further, illustrative processes herein use approximately 90% less cryoprotectant, such as DMSO to achieve a batch or a lot of cryopreserved platelets as compared to current standard-of-care cryopreserved platelets. And illustrative processes herein provide improved batch-to-batch consistency of cryoprotectant concentrations, such as dimethyl sulfoxide (DMSO) concentrations, total number of platelets, and platelet concentration, than current standard-of-care CPP products. Furthermore, processes herein provide cryopreserved platelets in a cryo-vessel, such that the cryo-vessels contain 1 platelet unit equivalent of platelets or platelet particles in a lower volume as compared to the standard-of-care cryopreserved platelets, cold stored platelets, or room temperature platelets. For example, cryo-vessels comprising cryopreserved platelets or frozen activated platelets provided herein can comprise 1.5×10¹¹ to 5×10¹¹ platelets or platelet particles in a volume of 20 to 35 ml, as compared to a similar number of platelets in 200 to 300 ml of plasma or plasma additive solution in case of apheresis platelet units.

For example, the process of preparing a batch of cryopreserved platelets, and a collection of cryo-vessels comprising cryopreserved platelets as disclosed herein provide cryopreserved platelets that are homogenous within a batch and across multiple batches. The homogeneity observed can include but is not limited to the concentration of DMSO, platelet concentration, microparticle concentration (for example, CD61-positive microparticles), pH of the composition comprising cryopreserved platelets, volume of the composition comprising cryopreserved platelets, and total number of platelets in the composition comprising cryopreserved platelets. Such homogeneity is generally not observable in cryopreserved platelets produced from a single unit of platelets owing to donor-to-donor variability or prior processes for making CPPs even if there was a mention of the use of a pool of donors. In illustrative embodiments, the collection of cryo-vessels comprising cryopreserved platelets herein, or the process for preparing a batch of cryopreserved platelets herein provides cryo-vessels having consistent properties between the cryo-vessels across batches, so as to provide homogeneity across the batches. Therefore, in some cases, homogeneity enables quality testing of each batch using any cryo-vessel prepared in the batch. Also, the collection of cryo-vessels, or the process for preparing a batch of cryopreserved platelets herein is a highly scalable collection or process. For example, the process herein can be modified to create a batch having 3, 4, or 5 cryo-vessels on the low end to 100, 200, or 300 cryo-vessels on the high end, while maintaining the homogeneity of the composition across the batches. Further, since the composition in one cryo-vessel is typically identical to the composition in any other cryo-vessel in a batch, the process herein allows preparing a cGMP manufacturable batch of cryopreserved platelets. For example, the process herein allows preparing archivable samples of cryopreserved platelets. The process herein also readily facilitates stockpiling so that whenever there is a requirement of a hemostatic agent, such as platelets, compositions provided herein can be available.

Additionally, the composition comprising cryopreserved platelets herein, such as those having a biomolecular profile indicative of more than 1 platelet donor, or the composition comprising cryopreserved platelets obtained from a process herein, such as those prepared from platelet units from a plurality of donors, can have attributes or properties that are different as compared to the attributes of a single donor CPP. A non-limiting illustrative example of a single donor CPP is disclosed herein in Example 1. For example, cryopreserved platelets herein can be activated to a higher level, or are highly activated as compared to single donor CPP. In some cases, compositions comprising cryopreserved platelets herein, for example, upon thawing have a higher thrombin generation ability (TGA) as compared to standard of care single donor CPP. In some cases, compositions comprising cryopreserved platelets herein, for example, upon thawing have a higher thrombin generation ability (TGA) as compared to the TGA of the starting material such as, apheresis platelet units.

Accordingly, such processes, which also can be referred to as methods, in non-limiting examples, can include the following steps:
a) forming a concentrated pooled platelet resuspension (CPR) with a weight or volume based on the number of platelet units (PU) to form the CPR
b) adding dimethyl sulfoxide (DMSO) to the CPR to obtain a CPR having DMSO such that the concentration of DMSO is in the range of 4% to 8%;
c) distributing the CPR having DMSO among more than 1 cryo-vessel from a collection of cryo-vessels; and
d) freezing the collection of cryo-vessels to prepare the batch of cryopreserved platelets, wherein the distributing is done to obtain 1 PU equivalent weight of the CPR having DMSO in each cryo-vessel, and
wherein the freezing is initiated within 2 hours after adding the DMSO.

Furthermore, collections of cryo-vessels that have unique properties are provided herein. The above processes can be used to preparate such collections. Accordingly, in some examples provided herein is a collection of cryo-vessels, wherein each cryo-vessel in the collection comprises a composition comprising cryopreserved platelets,
wherein the cryopreserved platelets in each cryo-vessel have a set of biomolecule profiles indicative of more than 1 platelet donor,
wherein a batch of cryo-vessels has an indistinguishable set of biomolecular profiles comprising a
phosphatidylserine positivity, when measured using lactadherin binding of at least 40%, or 50%,
wherein each batch of the collection has a different set of biomolecular profiles than any other batch in the collection,
wherein, the collection comprises a plurality of at least 2 batches of cryo-vessels,
wherein each composition in the collection has the property of exhibiting a pH of greater than 6.0, upon thawing and storing at a temperature in the range of 20° C. to 35° C. for 6 hours to 24 hours, and
wherein each composition in the collection has a capacity to generate thrombin in an in vitro thrombin generation assay. In some examples, the pH of the cryopreserved platelets, or the frozen platelets, upon thawing and storing at the temperature for 6 hours, 8 hours, or 24 hours does not change by more than 0.2 pH unit. In some examples provided herein is a collection of cryo-vessels, wherein each cryo-vessel in the collection comprises a composition comprising cryopreserved platelets,
wherein the cryopreserved platelets in each cryo-vessel have a set of biomolecule profiles indicative of more than 1 platelet donor, wherein a batch of cryo-vessels has an indistinguishable set of biomolecular profiles, and wherein each batch of the collection has a different set of biomolecular profiles than any other batch in the collection,
wherein, the collection comprises a plurality of at least 2 batches of cryo-vessels, and
wherein all of the compositions in the collection have less than 10×10⁶/μl CD61-positive microparticles.

Further aspects provided herein address the long-felt need of storing cryopreserved platelets at a temperature higher than −65° C., for example in a −20° C. freezer. These aspects provide a process to form cryopreserved platelets, which in some illustrative embodiments are cryopreserved platelet derivatives, that includes a transition in freezing temperatures from an initial freezing temperature to a storage freezing temperature. Such processes can include an initial freezing step at a temperature (i.e., initial temperature) less than or equal to −50° C., for example −65° C. or −80° C., to form an initial frozen platelet composition, followed by storing the initial frozen platelet composition in a frozen state at a temperature (i.e., storage temperature) equal to or greater than −30° C., for example −20° C., to form a cryopreserved platelet composition. Surprisingly, it was found that the cryopreserved platelets formed by the process as disclosed herein have the property of being stable and retain hemostatic abilities when stored at higher temperatures as compared to that required for storing conventional cryopreserved platelets.

### Processes for Preparing Batches of Cryopreserved Platelets

Illustrative examples of processes for preparing a cryopreserved platelet preparation (CPP), cryopreserved platelets, or batches of CPP provide a number of advantages. For example, they reduce donor variability and lot-to-lot variability and provide a consistent manufacturing process for preparing a platelet-based product. Illustrative examples of processes provided herein, allow for the generation of uniform cryo-vessels, each comprising an equivalent dose derived from the pooled platelet source, and reduce the variability of the concentration of platelets, microparticles, and the cryoprotectant in each cryo-vessel comprising CPP. Furthermore, they reduce and in illustrative embodiments, eliminate elevated excursions of microparticles in some cryo-vessels, for example above 10×10⁶ microparticles per microliter.

A non-limiting example of a process for preparing a batch, or a multiple number of batches, of cryopreserved platelets can include the following steps:
a) forming a concentrated pooled platelet resuspension (CPR) with a volume, or in illustrative examples a weight based on the number of platelet units (PU) to form the CPR;
b) adding dimethyl sulfoxide (DMSO) to the CPR to obtain a CPR having DMSO, in illustrative embodiments such that the concentration of DMSO is in the range of 4% to 8%;
c) distributing the CPR having DMSO among more than 1 cryo-vessel from a collection of cryo-vessels; and
d) freezing the collection of cryo-vessels to prepare the batch of cryopreserved platelets. In illustrative examples, the distributing is done to obtain 1 PU equivalent weight of the CPR having DMSO in each cryo-vessel. In further illustrative examples, the freezing is initiated within 2 hours after adding the DMSO.

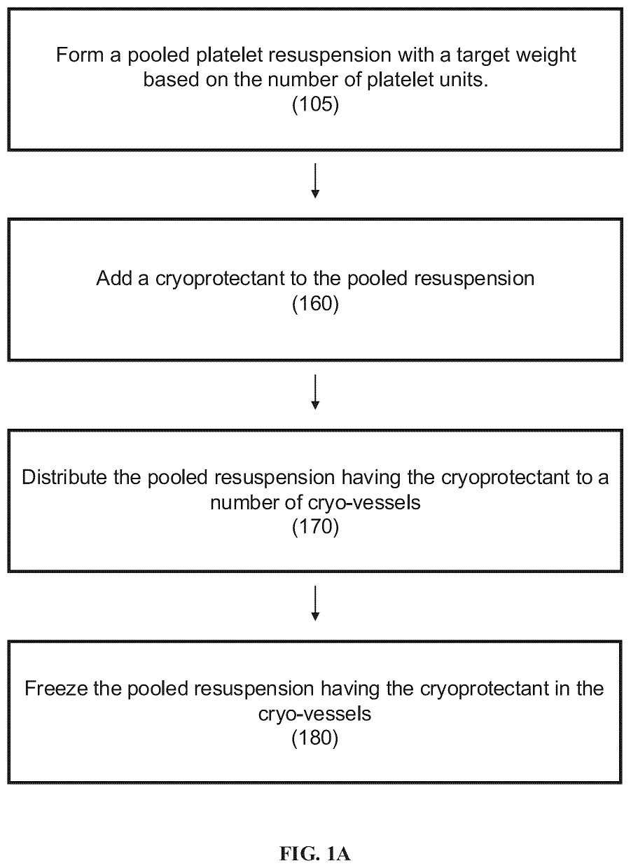

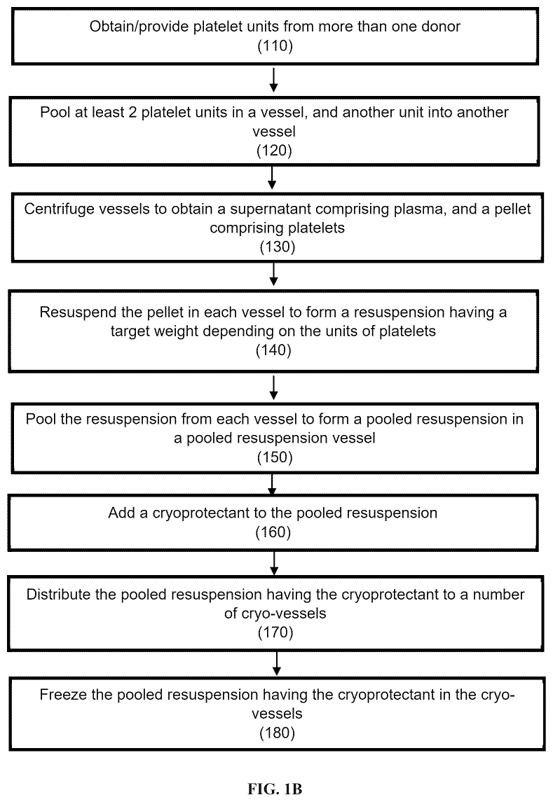

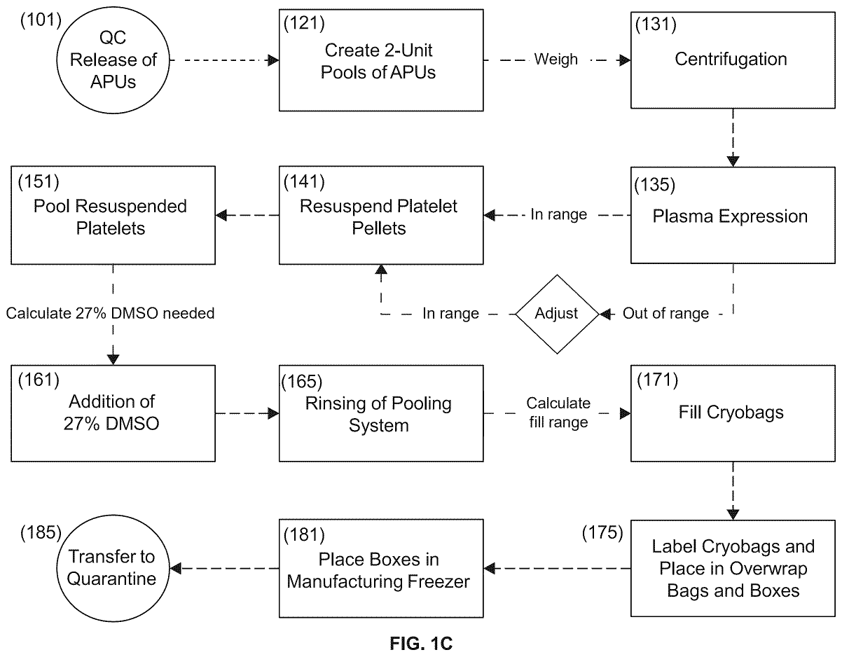

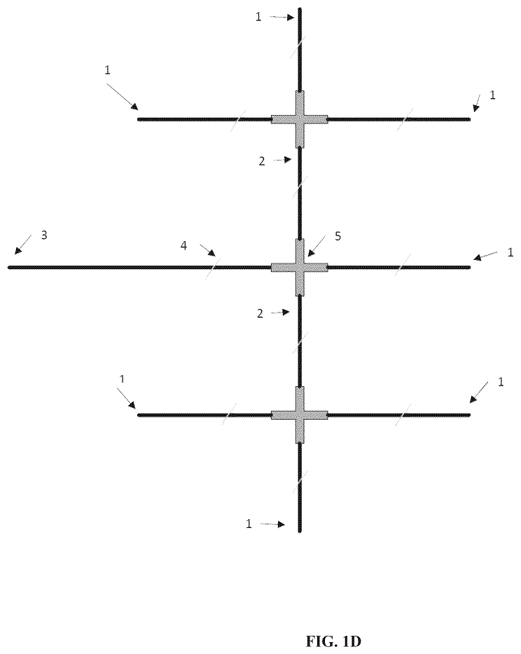

Accordingly, a non-limiting example of a process for preparing a batch of cryopreserved platelets is illustrated in FIG. 1A. The process of FIG. 1A can be employed in the manufacture of a collection of cryo-vessels that contain CPP that are intended to be delivered to humans, including product candidates such as CLPH 511 (Cellphire Inc., Gaithersburg, MD), which is currently in clinical development. In step 105, the pooled platelet resuspension, which can be referred to as a CPR since it is typically a concentrated resuspension, is typically formed after pooling platelet units from one, or typically more than one donor. Platelets in the pool can be concentrated using known methods such as centrifugation or tangential flow filtration (TFF). The concentrated pooled platelets are resuspended or retained in a concentrating device such as a TFF device, until a target volume, or in illustrative embodiments, a target weight is achieved to form the pooled platelet resuspension.

After the pooled platelet resuspension is formed, a cryoprotectant, in illustrative examples DMSO, is added to the pooled platelet resuspension. As a non-limiting example, the DMSO can be added to the CPR to a final concentration in the range of 4% to 8% DMSO. The pooled platelet resuspension in cryoprotectant is then distributed into a number of cryo-vessels. In illustrative examples each cryo-vessel comprises an amount of pooled platelet resuspension equivalent in weight to one PU (170). The cryo-vessels that contain the pooled platelet resuspension in DMSO are then frozen (180). In certain examples, the freezing is initiated within 3 hours, 2 hours, or in illustrative examples within 1 hour of the cryoprotectant (e.g., DMSO) to the pooled platelet resuspension.

In illustrative embodiments, the step of adding the cryoprotectant (160) can be performed using a tubing tree system. In illustrative embodiments, adding a cryoprotectant (160) is performed until a target weight of the cryoprotectant (e.g., DMSO) is achieved. In illustrative examples, the addition of the cryoprotectant (e.g., DMSO) (160) is performed using a tubing tree system. In illustrative examples, distributing the pooled resuspension (170) can be performed using a dosing tree system, which can be different from the tubing tree system, or the same tubing tree system can be washed and used as the dosing tree system.

Another example of a non-limiting process for preparing a batch of cryopreserved platelets is illustrated in FIG. 1B. Such a process includes the following steps:
a) obtaining or providing platelet units from more than one donor (110);
b) pooling at least 2 platelet units into one vessel and at least another platelet unit into another vessel (120), such that there will be more than one vessel at the end of the step;
c) centrifuging each vessel to obtain a supernatant comprising plasma, and a pellet comprising platelets (130);
d) resuspending the pellet in each vessel to form a resuspension wherein the resuspension has a target weight determined by the number of units pooled or provided in the vessel (140);
e) pooling the resuspension from each vessel to form a pooled resuspension in a pooled resuspension vessel (150);
f) adding a cryoprotectant to the pooled resuspension vessel having the pooled resuspension to obtain a pooled resuspension having the cryoprotectant (160);
g) distributing the pooled resuspension having the cryoprotectant from the pooled resuspension vessel among a number of cryo-vessels (170); and
h) freezing the pooled resuspension having the cryoprotectant in the cryo-vessels, to form the batch of cryopreserved platelets (180).

The initial step of this non-limiting process illustrated in FIG. 1B, involves obtaining platelet units from more than 1 donor (110). Such platelet units can be obtained, for example, from any organization that collects blood, such as, as non-limiting examples, a hospital or a blood bank. Typically, at least 3, 4, or 5 platelet units are provided and available for pooling (120). The platelet units can be from exactly or more than 2, 3, 4, or 5 donors. In this non-limiting example, the platelet units are pooled into one or more vessels, typically more than one vessel (120). The platelet units are typically pooled such that, for example, at least 2 platelet units are pooled into one vessel, and in certain examples, one or more other platelet units are pooled into one or more other vessels. The platelet units can be pooled to a target volume, or in illustrative examples, a target weight.

The pooled platelets can then be concentrated using known methods such as centrifugation (130) or tangential flow filtration (TFF). In illustrative examples, the vessels containing pooled platelets can undergo centrifugation to obtain a supernatant comprising plasma and a pellet comprising platelets. After the centrifugation in this example, typically some of the plasma supernatant is removed.

The step after concentration, in illustrative examples using centrifugation, typically includes resuspending the platelets to form a platelet resuspension (140). In this non-limiting example, the platelets are resuspended to achieve a target volume, or in illustrative examples, a target weight. Such target weight in this non-limiting examples depends on the number of platelet units that were concentrated in the vessel in the concentrating step. The target weight in non-limiting examples, can be in the range of 15.0 to 30.0 g, 15.0 to 29.0 g, 15.0 to 28.5 g, or in illustrative embodiments 15.9 g to 27.9 g times the number of units pooled or provided in the vessel.

Accordingly, in the step preceding the resuspending step (140), in illustrative examples supernatant is removed to achieve a target weight of the resuspension. Alternatively, the step before the resuspending step can include removing a part of the supernatant and adding a buffer composition before resuspending to the target weight.

After the platelet resuspension is formed, the platelet resuspension from more than one vessel can be pooled to form a pooled resuspension (150). Thus, a pool of pools of resuspended platelet units is formed. In illustrative embodiments, the platelet resuspensions from all the vessels that were created are pooled to form the pooled resuspension in the pooled resuspension vessel. In such illustrative embodiment, the pools of resuspension can typically be pooled into a single pooled resuspension vessel. To accommodate a higher number of platelet units in a single batch, the number of pooled resuspension vessels can be, in non-limiting examples, 1, 2, 3, or more vessels.

After the pooled platelet resuspension is created, the remaining steps of this example can be the same as those described for FIG. 1A. For example, a cryoprotectant, in illustrative examples DMSO, can be added to the pooled resuspension (160) to a target concentration of cryoprotectant. Typically, the target concentration of cryoprotectant can be in the range of 2% to 10%, in illustrative examples, such target concentration can be in the range of 4% to 8%. In illustrative embodiments, the steps of pooling the resuspension (150), and adding the cryoprotectant (160) can be performed using a tubing tree system.

The pooled resuspension having cryoprotectant is then distributed into a number of cryo-vessels (170). Distribution can be performed using a dosing tree system. Such distribution can be performed until a target fill-volume, or in illustrative examples, a target fill-weight is achieved. The target fill-weight or target fill-volume can depend on the number of platelet units processed. In a non-limiting example, if 12 platelet units are processed and equal to the number of cryo-vessels, in such non-limiting example 12, the target fill-weight can be determined by dividing the weight of the pooled resuspension, typically having cryoprotectant, by 12.

After such distribution, the cryo-vessels that contain the pooled resuspension having DMSO are frozen (180). Freezing can be performed by subjecting the cryo-vessels containing pooled resuspension having DMSO to a freezing environment set to achieve freezing, typically having a temperature at or below freezing, to form cryopreserved platelets. In non-limiting examples, the freezing can include subjecting the cryo-vessels to an initial temperature at or below freezing, followed by subjecting the cryo-vessels to another temperature. In non-limiting examples, the cryo-vessels can be subjected to an initial temperature in the range of −70° C. to −90° C. In non-limiting examples, the cryo-vessels can be subjected to an initial temperature, followed by another temperature in the range of 0° C. to −30° C.

The freezing step is initiated upon placement of the cryo-vessels into the freezing environment. Typically, the freezing is initiated within 4 hours, 3 hours, 2 hours, or in illustrative examples, within 1 hour of the addition of the cryoprotectant (e.g., DMSO) to the pooled resuspension. Additional details regarding steps 160, 170 and 180 are provided in the paragraphs immediately above. Further details regarding processes for preparing cryopreserved platelets or batches thereof are provided in other sections herein.

Another example of a non-limiting process for preparing a batch of cryopreserved platelets is illustrated in FIG. 1C. The Examples section herein provides a specific non-limiting example of a process according to such FIG. 1C. The initial step of a non-limiting process depicted in FIG. 1C involves quality control release testing of apheresis platelet units (APUs). Such APUs in this non-limiting example of FIG. 1C are obtained from more than 1 donor and pass initial quality control release testing (101). Quality control tested parameters for release of APUs can include for example, white blood cell count, age, red blood cell contamination, pH, total platelet count per unit, exposure to radiation, and packaging integrity. Such platelet units can be obtained, for example, from any organization that collects blood, such as, as non-limiting examples, a hospital or a blood bank, or as another non-limiting example can be drawn directly at the entity performing processes for preparing cryopreserved platelets, and compositions and batches of compositions thereof. It will be understood that the entity performing the process can be an organization that, as part of their activities independent of performing the process, collects blood from donors.

In this non-limiting example of FIG. 1C, at least 2, two-unit pools are created (121) by pooling 4 different APUs into 2 vessels, such that 2 APUs are in one vessel and 2 APUs are in the other vessel. There can be additional vessels having 2 APUs in this example. Then each vessel is weighed after the pool is created within the vessel. The vessel weight is determined and recorded and is then used in the plasma removal step (see below).

After the step of pooling the platelet units is performed (121) and the resulting vessels are weighed, as discussed above, the vessels are subjected to centrifugation to pellet the platelets therein (131). The centrifugation can be performed, in this non-limiting example, at a force of 1000 to 2000 G for 5 to 25 minutes. Thus, in this example the concentration step results in a supernatant comprising plasma and a pellet comprising platelets within the vessel.

Next, in the example depicted in FIG. 1C, some of the supernatant is removed from the pooled platelets in the vessels, also known as plasma expression (135), to achieve a weight that is within a target weight range based on the number of platelet units in the vessel. Typically, plasma is removed from the vessel and in this example, the weight of the vessel determined before centrifugation is used as an initial guide for the weight of supernatant to remove to hit the target weight. However, if the weight does not come in within the target weight range after this initial plasma removal based on the weight before centrifugation, additional supernatant can be added or removed from the pooled platelets in the vessels to adjust the weight until it is within the target weight range. The pooled platelets in each vessel, in this non-limiting example comprising the supernatant and platelet pellet, are then resuspended (141) to form a platelet resuspension and typically agitated until the platelet pellet is no longer visible.

After the platelet resuspension step, such resuspensions in each vessel can be pooled (151) to form a concentrated pooled platelet resuspension within a single vessel. As a non-limiting example, 6 platelet resuspensions in 6 different vessels of 2 APUs each, can be pooled to form 1 concentrated pooled platelet resuspension. It can be understood that the concentrated pooled platelet resuspension formed (151) can be considered to be a pool of pools of platelet units. In non-limiting examples, pooling the platelet resuspensions to form the concentrated pooled platelet resuspension can be performed using a pooling tree system.

In the next step in the exemplary process of FIG. 1C, DMSO is added to the concentrated pooled platelet resuspension (161) from a 27% DMSO stock solution, to form a concentrated pooled platelet resuspension having DMSO. Typically, the DMSO is added to achieve a target concentration in the range of 4% to 8% (e.g., 6%). The amount of DMSO from the stock solution to be added can be determined based on volume, but in illustrative examples, it is based on weight (e.g., using Equation 7 herein). Addition of the DMSO to the pooled platelet resuspension can be performed using the pooling tree system. The DMSO can be used to rinse the pooling tree system (165), in non-limiting examples, to remove any residual material that remains in such system.

The concentrated pooled platelet resuspension having DMSO can then be distributed to fill a number of cryobags (171), in a non-limiting example, 12 cryobags. The distribution can be performed to achieve a target fill weight. Typically, the target fill weight can be a range, such that the range includes a minimum and maximum. Such minimum and maximum can be determined based on the weight of the cryobag and the concentrated pooled platelet resuspension having DMSO. Such distribution into the cryobags can be performed using a tubing tree system. After distribution, the cryobags can be placed in one or more overwrap bags or boxes (175).

After the concentrated pooled platelet resuspension is distributed to a number of cryobags and one or more bags or boxes containing the cryobags, they are placed in a manufacturing freezer, or freezing environment (181). Typically, the freezer can be set to a temperature at or below freezing. In a non-limiting example, the freezer temperature can be set to a range between −60° C. to −90° C. (e.g., −80° C.). In non-limiting examples, the time elapsed from the addition of the DMSO (161) to the initiation of freezing (i.e., the moment the cryobags are placed in the freezer), can be less than 3 hours, 2 hours, in illustrative examples, less than 1 hour. After the step of placing the cryobags in the freezer is performed, the cryobags can be transferred to quarantine (185), in this non-limiting example, in an environment at or below freezing, for storage. Further details regarding processes for preparing cryopreserved platelets or batches thereof are provided in other sections herein.

### Collection of Cryo-Vessels Comprising Cryopreserved Platelets

Provided herein in some aspects, is a collection of cryo-vessels comprising cryopreserved platelets, wherein the cryopreserved platelets in each cryo-vessel have a biomolecule profile indicative of more than 1 platelet donor. The concentration of a cryoprotectant, such as DMSO in the cryopreserved platelets of a first cryo-vessel can be within 10%, 9%, 8%, 7%, 6%, 5%, 4%, 3%, 2%, or 1% of the concentration of the cryoprotectant, such as DMSO in the cryopreserved platelets of a second cryo-vessel. For example, the concentration of DMSO in the cryopreserved platelets of a first cryo-vessel can be in the range of 0.001-10%, 0.001-8%, 0.001-6%, 0.001-4%, 0.001-2%, or 0.001-1% of the concentration of DMSO in the cryopreserved platelets of a second cryo-vessel. Collection as provided herein can comprise a plurality of cryo-vessels comprising the cryopreserved platelets. Collection can be a collection of cryo-vessels in batches, for example, each batch can have at least 2, 3, 4, 5, 6, 7, 8, 9, 10, or more cryo-vessels, and a collection can have at least 2, 3, 4, 5, 6, 7, 8, 9, 10, 11, 12, 15, 20, 50, or more number of batches. A plurality of cryo-vessels or cryo-containers having the cryopreserved platelets herein, can be referred to as a “batch” or a “lot”. Typically, the cryopreserved platelets in each cryo-vessel have a set of biomolecule profiles indicative of more than 1 platelet donor and a batch of cryo-vessels has an identical set of biomolecule profiles. In order to clarify, the population of cryopreserved platelets from different cryo-vessels obtained from the same pool of platelets from multiple donors typically will have the same biomolecule profile amongst them. The cryo-vessels thus obtained from the same pool of platelets can also be referred to as the cryo-vessels from the same batch. Whereas the cryopreserved platelets obtained from the pool of platelets that is different than another pool of platelets can be referred to as belonging to a different batch. The set of biomolecule profiles indictive of more than 1 platelet donor in a batch can be different as compared to the set of biomolecule profiles of another batch. Thus, CPPs of a first lot typically have a biomolecule profile a first set of donors, and CPPs of a second lot typically have a biomolecule profile of a second set donors, wherein the second set of donors is not identical to the first set of donors. A skilled artisan will understand that there are numerous characteristics that can be used to differentiate CPPs from a first set of donors from CPPs from a second set of donors. Accordingly, a collection of cryo-vessels having CPPs can have a plurality of batches, such that, within one batch the set of biomolecule profiles indicative of more than 1 platelet donor is identical across the cryo-vessels of that batch, and is different from the cryo-vessels of other batches in the collection. Typically, a collection of cryo-vessels as provided herein is homogenous across batches and within batches and has significantly less donor-to-donor variation. Some non-limiting parameters to assess homogeneity can be concentration of cryoprotectant, platelet concentration, total number of platelets, pH, thrombin generation ability (IU), and CD61-positive microparticle concentration. In some embodiments the collections comprise frozen or cryopreserved platelets and/or platelet derivatives having one or more of the recited properties provided herein.

A collection of cryo-vessels comprising cryopreserved platelets herein, in some embodiments, can include platelet concentrations in manner wherein the concentration of platelets in the cryopreserved platelets in a cryo-vessel within a batch or across batches varies within 30%, 25%, 20%, 15%, 12%, 10%, 9%, 8%, 7%, 6%, 5%, or 4%. For example, the concentration of platelets in the cryopreserved platelets in a cryo-vessel within a batch or across batches varies in the range of 0.5-30%, 1-30%, 2-30%, 3-30%, 0.5-25%, 0.5-20%, 0.5-15%, 0.5-10%, 0.5-7%, or 0.5-5%. For example, the concentration of platelets in the cryopreserved platelets in a cryo-vessel across at least 5 batches varies within 20%, 15%, 12%, 10%, 9%, 8%, 7%, 6%, 5%, or 4%. For example, the concentration of platelets in the cryopreserved platelets in a cryo-vessel across at least 5 batches varies in the range of 0.5-30%, 1-30%, 2-30%, 3-30%, 0.5-25%, 0.5-20%, 0.5-15%, 0.5-10%, 0.5-7%, or 0.5-5%. For example, the concentration of platelets in the cryopreserved platelets in a cryo-vessel across at least 10 batches varies within 20%, 15%, 12%, 10%, 9%, 8%, 7%, 6%, 5%, or 4%. For example, the concentration of platelets in the cryopreserved platelets in a cryo-vessel across at least 10 batches varies in the range of 0.5-30%, 1-30%, 2-30%, 3-30%, 0.5-25%, 0.5-20%, 0.5-15%, 0.5-10%, 0.5-7%, or 0.5-5%. In some embodiments, the concentration of platelets in the cryopreserved platelets in a cryo-vessel across at least 5 batches has a mean intra-batch coefficient of variance within 20%, 15%, 12%, 10%, 9%, 8%, 7%, 6%, 5%, or 4%. For example, the concentration of platelets in the cryopreserved platelets in a cryo-vessel across at least 5 batches has a mean intra-batch coefficient of variance within 0.5-30%, 1-30%, 2-30%, 3-30%, 0.5-25%, 0.5-20%, 0.5-15%, 0.5-10%, 0.5-7%, or 0.5-5%. In some embodiments, the concentration of platelets in the cryopreserved platelets in a cryo-vessel within a batch has a coefficient of variance within 20%, 15%, 12%, 10%, 9%, 8%, 7%, 6%, 5%, or 4%. For example, the concentration of platelets in the cryopreserved platelets in a cryo-vessel across within a batch has a coefficient of variance within 0.5-30%, 1-30%, 2-30%, 3-30%, 0.5-25%, 0.5-20%, 0.5-15%, 0.5-10%, 0.5-7%, or 0.5-5%.

A collection of cryo-vessels comprising cryopreserved platelets herein, in some embodiments, can include total number of platelets in a manner wherein the total number of platelets in the cryopreserved platelets in a cryo-vessel within a batch or across batches varies within 30%, 25%, 20%, 15%, 12%, 10%, 9%, 8%, 7%, 6%, 5%, or 4%. For example, the total number of platelets in the cryopreserved platelets in a cryo-vessel within a batch or across batches varies in the range of 0.5-30%, 1-30%, 2-30%, 3-30%, 0.5-25%, 0.5-20%, 0.5-15%, 0.5-10%, 0.5-7%, or 0.5-5%. For example, the total number of platelets in the cryopreserved platelets in a cryo-vessel across at least 5 batches varies within 20%, 15%, 12%, 10%, 9%, 8%, 7%, 6%, 5%, or 4%. For example, the total number of platelets in the cryopreserved platelets in a cryo-vessel across at least 5 batches varies in the range of 0.5-30%, 1-30%, 2-30%, 3-30%, 0.5-25%, 0.5-20%, 0.5-15%, 0.5-10%, 0.5-7%, or 0.5-5%. For example, the concentration of platelets in the cryopreserved platelets in a cryo-vessel across at least 10 batches varies within 20%, 15%, 12%, 10%, 9%, 8%, 7%, 6%, 5%, or 4%. For example, the concentration of platelets in the cryopreserved platelets in a cryo-vessel across at least 10 batches varies in the range of 0.5-30%, 1-30%, 2-30%, 3-30%, 0.5-25%, 0.5-20%, 0.5-15%, 0.5-10%, 0.5-7%, or 0.5-5%. In some embodiments, the concentration of platelets in the cryopreserved platelets in a cryo-vessel across at least 5 batches has a mean intra-batch coefficient of variance within 20%, 15%, 12%, 10%, 9%, 8%, 7%, 6%, 5%, or 4%. For example, the concentration of platelets in the cryopreserved platelets in a cryo-vessel across at least 5 batches has a mean intra-batch coefficient of variance within 0.5-30%, 1-30%, 2-30%, 3-30%, 0.5-25%, 0.5-20%, 0.5-15%, 0.5-10%, 0.5-7%, or 0.5-5%. In some embodiments, the concentration of platelets in the cryopreserved platelets in a cryo-vessel within a batch has a coefficient of variance within 20%, 15%, 12%, 10%, 9%, 8%, 7%, 6%, 5%, or 4%. For example, the concentration of platelets in the cryopreserved platelets in a cryo-vessel across within a batch has a coefficient of variance within 0.5-30%, 1-30%, 2-30%, 3-30%, 0.5-25%, 0.5-20%, 0.5-15%, 0.5-10%, 0.5-7%, or 0.5-5%.

A collection of cryo-vessels comprising cryopreserved platelets herein, in some embodiments, can include a homogeneity in pH of cryo-vessels in a manner wherein the pH of the cryopreserved platelets in a cryo-vessel within a batch or across batches varies within 5%, 4%, 3%, 2%, 1%, 0.9%, or 0.75%. For example, the pH of the cryopreserved platelets in a cryo-vessel within a batch or across batches varies in the range of 0.001-5%, 0.001-4%, 0.001-3%, 0.001-2%, 0.001-1%, 0.01-1%, 0.05-1%, or 0.5-1%. In some embodiments, the pH of the cryopreserved platelets in a cryo-vessel across at least 5 batches has a mean intra batch coefficient of variance within 10%, 7%, 5%, 4%, 3%, 2%, or 1%. For example, the pH of the cryopreserved platelets in a cryo-vessel across at least 5 batches has a mean intra batch coefficient of variance in the range of 0.001-5%, 0.001-4%, 0.001-3%, 0.001-2%, 0.001-1%, 0.01-1%, 0.05-1%, or 0.5-1%. In some embodiments, the pH of the cryopreserved platelets in a cryo-vessel across at least 5 batches has a coefficient of variance within 10%, 7%, 5%, 4%, 3%, 2%, or 1%. For example, the pH of the cryopreserved platelets in a cryo-vessel across at least 5 batches has a coefficient of variance in the range of 0.001-5%, 0.001-4%, 0.001-3%, 0.001-2%, 0.001-1%, 0.01-1%, 0.05-1%, or 0.5-1%.

A collection of cryo-vessels comprising cryopreserved platelets herein, in some embodiments, can include a homogeneity in the concentration of CD61-positive microparticles, such that the concentration of CD61-positive microparticles in the cryopreserved platelets in a cryo-vessel within a batch or across batches varies within 20%, 15%, 12%, 10%, 9%, 8%, or 7%. For example, the concentration of CD61-positive microparticles in the cryopreserved platelets in a cryo-vessel within a batch or across batches vanes in the range of 1-20%, 1-15%, 1-12%, 1-10%, 3-20%, 3-15%, 3-12%, 3-10%, or 5-10%. In some embodiments, the concentration of CD61-positive microparticles in the cryopreserved platelets in a cryo-vessel across at least 5 batches has a mean intra batch coefficient of variance in the range of 1-20%, 1-15%, 1-12%, 1-10%, 3-20%, 3-15%, 3-12%, 3-10%, or 5-10%. For example, the concentration of CD61-positive microparticles in the cryopreserved platelets in a cryo-vessel across at least 5 batches has a coefficient of variance in the range of 1-20%, 1-15%, 1-12%, 1-10%, 3-20%, 3-15%, 3-12%, 3-10%, or 5-10%. For example, the concentration of CD61-positive microparticles in the cryopreserved platelets in a cryo-vessel across at least 5 batches has a coefficient of variance within 25%, 20%, 15%, 10%, or 8%.

A collection of cryo-vessels comprising cryopreserved platelets herein, in some embodiments, can include a homogeneity in the thrombin generation ability of the cryopreserved platelets, such that a measure of thrombin generation per 10⁶ platelets across batches or within a batch varies within 20%, 15%, 12%, 10%, 9%, 8%, 7%, 6%, 5%, 4%, 3%, or 2%. A skilled artisan would understand that a measure of thrombin generation can be any appropriate units based on the assay used, for example, thrombin generation assay can be performed to determine the thrombin generation ability in terms of IU per 10⁶ platelets. An illustrative and non-limiting example of a method for assessing thrombin generation is shown in Example 4. For example, a measure of thrombin generation per 10⁶ platelets across batches or within a batch varies in the range of 0.5-20%, 0.5-15%, 0.5-12%, 0.5-10%, 0.5-8%, or 0.5-5%. For example, a measure of thrombin generation per 10⁶ platelets across batches has a mean intra batch coefficient of variance within 20%, 15%, 12%, 10%, 9%, 8%, 7%, 6%, 5%, 4%, 3%, or 2%. In some embodiments, a measure of thrombin generation per 10⁶ platelets across batches has a mean intra batch coefficient in the range of 0.5-20%, 0.5-15%, 0.5-12%, 0.5-10%, 0.5-8%, or 0.5-5%. For example, a measure of thrombin generation per 10⁶ platelets across batches has a coefficient of variance within 20%, 15%, 12%, 10%, 9%, 8%, 7%, 6%, 5%, 4%, or 3%. For example, a measure of thrombin generation per 10⁶ platelets across batches has a coefficient of variance in the range of 0.5-20%, 0.5-15%, 0.5-12%, 0.5-10%, 0.5-8%, or 0.5-5%.

Cryo-vessels having a composition comprising cryopreserved platelets or frozen activated platelets, for example, CPPs herein in illustrative embodiments are prepared using platelets that are pooled from a plurality of donors (e.g. pooled platelets). Thus, cryo-vessels or cryo-vials comprising CPPs herein, and processes for preparing and using the same, in illustrative embodiments include a population of CPPs that have a biomolecule profile indicative of more than 1 platelet donor. A skilled artisan will understand that there are various molecular tests that can be used to confirm that CPPs were prepared from a plurality of donors.

Cryo-vessels having frozen activated platelets, or cryopreserved platelets herein that comprise a population of platelet particles having a biomolecule profile indicative of more than 1 platelet donor, in illustrative embodiments comprise the equivalent of 1 unit of frozen activated platelets or cryopreserved platelets. The volume, weight, and/or number of platelet particles in 1 unit of frozen activated platelets in each cryo-vessel of a collection herein, or a batch of cryopreserved platelet compositions obtained by a process as disclosed herein can be based upon the platelet units obtained from a plurality of donors that were used for pooling. In some cases, 1 unit of frozen activated platelets or cryopreserved platelets can comprise at least 1×10¹¹, 1.5×10¹¹, or 1.7×10¹¹ platelets or platelet particles. In some cases, 1 unit of frozen activated platelets or cryopreserved platelets can comprise platelets or platelet particles in the range of 1×10¹¹ to 5×10¹¹, 1.5×10¹¹ to 5×10¹¹, or 1.7×10¹¹ to 5×10¹¹. Some aspects and embodiments herein, include the equivalent of 1 platelet unit per cryo-vessel of pooled cryopreserved platelets, for example in cryopreserved platelet compositions, or a collections of cryopreserved platelets in cryo-vessels. An equivalent platelet unit can be based upon the total number of platelet units, such that if some number (e.g., 5) platelet units are pooled, and then used to prepare that number (e.g., 5) cryo-vessels comprising frozen activated platelets using a process for making cryopreserved platelets disclosed herein, then each of the cryo-vessels thus obtained would have the equivalent of 1 platelet unit. Accordingly, the exact number of platelets in each cryo-vessel can depend upon the total number of platelets that were pooled.

In some embodiments, the biomolecule profile indicative of more than 1 platelet donor is a protein profile. For example, the biomolecule profile can be the amino acid sequence of one or more proteins, for example one or more proteins that are present in or associated with the CPPs in a lot of cryo-vials produced from a pool of donors. Such profile can include, for example, 3 or more amino acid sequences of a target protein from a single gene. These amino acid sequences can be those of polymorphs of the protein. It will be understood that wherein a single donor has one or two amino acid sequences of this protein, for example depending on whether they are homozygous or heterozygous, the presence of 3 or more amino acid sequences for this protein in the CPPs can be indicative of more than 1 donor. Furthermore, if a set of donors whose platelets were pooled to make CPPs of a first lot is not identical to the donors use to make CPPs of a second lot, then the set of amino acid sequence variants/alleles/versions of the target protein(s) in the first lot can be different than the set of amino acid sequence variants/alleles/versions of the target protein(s) in the second lot. For example, if 5 donors are used to make a first pool of platelets used to make a first lot, and 5 different donors are used to make a second pool of platelets for a second lot, for a target protein, there could be up to 10 different alleles/variants/versions in each pool for a protein originally expressed from a single gene, and at least 1 allele/variant/version of the target protein could be unique to each lot versus the other lot.

In some embodiments that rely more on quantitative information, the biomolecule profile indicative of more than 1 platelet donor is the presence of two or more alleles/variants/versions/amino acid sequences of at least a first protein from at least a first gene that are significantly different than 50% in frequency within the composition. A 50% frequency would be expected, for example, if such composition was from a single donor that was heterozygous for alleles at the first gene.

In a similar manner to the discussion above regarding a target protein(s), a biomolecule profile indicative of more than 1 platelet donor can be detected and/or quantified by detecting and/or quantifying nucleic acids that are present in or associated with the CPPs. Such detecting can use techniques such as, but not limiting to, PCR, typically, quantitative reverse transcription polymerase chain reaction (qRT-PCR). qRT-PCR is considered as one of the techniques available for quantifying RNA, such as mRNA in a sample. The RNA to be identified can be RNA specific for an individual donating the platelets, or can be used to identify platelets donated by a single donor. In illustrative embodiments, such an RNA molecule can be detected and/or quantified from a platelet sample for establishing that platelets have been donated by more than 1 individual.

In some cases, a biomolecule profile indicative of more than 1 platelet donor can be detected by analyzing Human Leukocyte Antigen (HLA) on the platelets in compositions and collections comprising such compositions, comprising cryopreserved platelets, disclosed herein. In humans, HLA genes are part of the Major Histocompatibility Complex (MHC) on chromosome 6 and are polymorphic, and each individual inherits one set of HLA genes from each parent, resulting in a maximum of two alleles per locus. Therefore, the composition can be analyzed for the HLA genes, and based on the number of alleles, in illustrative embodiments 3 or more alleles, it can be established whether the platelet composition arose from a plurality of platelet donors. Platelets typically express HLA Class-I antigens, such as HLA-A, HLA-B, and HLA-C. HLA-A, and HLA-B are abundantly expressed as compared to HLA-C, which sometimes remain undetectable. Typically, in the case of a single-donor cryopreserved platelet product, there will be no more than two alleles at each HLA locus. However, in case of a cryopreserved platelet product prepared from platelets from more than 1 donor, there will be more than two alleles at a given HLA locus. For example, in case of cryopreserved platelets, or CPP as disclosed herein that have a biomolecule profile indicative of more than 1 platelet donor, or the CPP prepared in accordance with the process as exemplified in Example 2, there would be more than two alleles at a given HLA locus. A skilled artisan can employ known techniques to analyze the HLA locus, for example, PCR methods based on sequence-specific primers (SSP), or sequence-specific oligonucleotides (SSO). Other techniques can include next generation sequencing (NGS) based HLA typing that has an advantage of providing high resolution. In some cases, a biomolecule profile can be determined by analyzing other MHC alleles apart from HLA, for example, using non-classical MHC Class I genes, such as HLA-E, HLA-F, and HLA-G. In some cases, the biomolecule profile can include MHC-linked minor histocompatibility antigens (MiHAs), MHC-encoded complement genes, for example, C4A, C4B, and Factor B. In some cases, the biomolecule profile can include MHC-linked cytokine genes, for example, tumor necrosis factor-α (TNF-α), and lymphotoxin-α (LTA).

In some embodiments, a collection of cryo-vessels herein can comprise frozen platelets in a cryopreservation medium in a frozen state, wherein the composition is capable of yielding one or more of the recited properties herein, for example one or more of the following properties after storage for at least 1 month, 2, 3, 4, 5, 6, 8, 10, or 12 months, in an illustrative embodiments at a temperature in a range of −10° C. to −30° C., or −20° C. +/−5° C., upon thawing:
a) is in a liquid state without the addition of a liquid;
b) exhibits a platelet count of at least 1.0×10¹¹/35 ml of the composition;
c) yields a single peak that corresponds to a compromised membrane peak in a membrane integrity assay;
d) exhibits a CD61-positive-microparticle content of less than 50% of the CD61 positive particles in the composition, and
e) generates thrombin in an in vitro thrombin generation assay. In some embodiments, a composition is capable of yielding two or more, three or more, four, or all of the properties. In illustrative embodiments, a composition is capable of yielding all of the properties. In some embodiments, a composition is capable of yielding properties a), b), and d). In some embodiments, a composition is capable of yielding properties a), b), d), and e).

### Stability, and Attributes of the Cryopreserved Platelets

In some embodiments of aspects that include process for preparing a batch of cryopreserved platelets, a process for preparing a cryopreserved platelet composition including a transition in freezing temperatures from an initial freezing temperature to a storage freezing temperature, and/or cryopreserved platelets, a cryopreserved platelet composition, a composition comprising frozen platelets, a collection or a batch comprising cryopreserved platelets, the cryopreserved platelets, or the frozen platelets herein can be stable when stored frozen at −80° C., −70° C., −60° C., −50° C., −40° C., −30° C., −20° C., or higher. In some embodiments, cryopreserved platelets, or frozen platelets, as disclosed herein, or cryopreserved platelets formed by a process as disclosed herein, in illustrative embodiments, cryopreserved platelets formed by a process that comprises freezing platelets in a cryopreservation medium, or freezing a pooled resuspension having a cryoprotectant as disclosed herein, at a temperature of less than or equal to −50° C., −55° C., −60° C., in illustrative embodiments, less than or equal to −65° C., −70° C., −75° C., or −80° C., to form an initial frozen platelet composition, and a second step comprising storing the initial frozen platelet composition at a temperature higher than or equal to −40° C., −35° C., −30° C., −25° C., in illustrative embodiments, higher than or equal to −20° C., −15° C., or −10° C., but less than 0° C., are stable when stored at a temperature higher than or equal to −40° C., −35° C., −30° C., −25° C., −15° C., or −10° C., but less than 0° C. Stability of the cryopreserved platelets present in a cryo-vessel as provided in a collection of cryo-vessels herein, can be assessed by a non-limiting list of parameters including visual inspection of cracks, tears, breaks of the cryo-vessel, such as a cryo-bag, visual inspection of aggregate free swirling of the cryopreserved platelets in the cryo-vessel, such as a cryo-bag, platelet counts per cryo-vessel, and pH of the cryopreserved platelets in the cryo-vessel. In illustrative embodiments, the stability, or the functional stability of the cryopreserved platelets, or the frozen platelets after thawing can be assessed by the pH of the platelets after thawing or post-thawing, or the composition comprising platelets after thawing or post-thawing. For example, in some non-limiting embodiments, cryopreserved platelets herein are stable when stored at a specific temperature, for example, at about −20° C., or higher when the cryopreserved platelets swirl without the presence of any aggregation on a visual inspection. For example, when stored at −20° C. +/−10° C., −20° C. +/−8° C., −20° C. +/−5° C., or −20° C. +/−2° C. the cryopreserved platelets swirl without the presence of any aggregation on a visual inspection. For example, in some non-limiting embodiments, cryopreserved platelets herein are stable when stored at a specific temperature, for example, at about −20° C., or higher, for example, stored at −20° C. +/−5° C. the pH of the cryopreserved platelets, typically upon thawing is equal to or more than 6.0, typically, equal to or more than 6.2. For example, the cryopreserved platelets herein upon storing at about −20° C., for example, at −20° C. +/−5° C. for a period in the range of 1 month-36 months, 1 month-30 months, 1 month-24 months, 1 month-18 months, or 1 month-12 months, typically upon thawing exhibit a pH higher than 7.0. In some embodiments, the cryopreserved platelets herein upon storing at about −20° C., for example, at −20° C. +/−5° C. for a period in the range of 1-12 months, typically upon thawing exhibit a pH higher than 6.2, 6.4, 6.6, 6.8, 7.0, or 7.2. For example, the cryopreserved platelets herein upon storing at −20° C. +/−5° C. for a period in the range of 1-12 months, typically upon thawing exhibit a pH in the range of 6.2 to 7.8, 6.4 to 7.8, 6.6 to 7.8, or 7-7.8.

For example, in some non-limiting embodiments, cryopreserved platelets herein are stable when stored at a specific temperature, for example, at about −20° C., or higher, for example, stored at −20° C. +/−5° C. the total number of platelets, typically upon thawing in a cryo-vessel is equal to or more than 1.5×10¹¹, 1.6λ10¹¹, or 1.7×10¹¹. In illustrative embodiments, cryopreserved platelets herein, typically when stored at −20° C. +/−5° C. for at least 1 month, 2, 3, 4, 6, 8, 10, 12 months, 2, 3, 4, 5, 6, 7, 8, 9, or 10 years, upon thawing have a total number of platelets in a cryo-vessel equal to or more than 1.5×10¹¹, 1.6×10¹¹, or 1.7×10¹¹. In some embodiments, cryopreserved platelets herein, typically upon thawing exhibit a particle size, for example, diameter in the range of 0.5 μm to 2.5 μm, 1 μm to 5 μm, 1 μm to 4 μm, or 0.5 μm to 5.0 μm such that at least 50% of the platelets after thawing have a diameter in the range of 0.5 μm to 2.5 μm, 1 μm to 5 μm, 1 μm to 4 μm, or 0.5 μm to 5.0 μm. In some cases, the cryopreserved platelets herein, typically upon thawing exhibit a particle size, for example, diameter of 0.6 μm and above, for example, in the range of 0.6 μm to 2.5 μm, 0.7 μm to 2.5 μm, 0.7 μm to 3.0 μm, 0.7 μm to 3.5 μm, 0.7 μm to 4.0 μm, 0.7 μm to 5.0 μm, 0.88 μm to 2.5 μm, 0.8 μm to 3.0 μm, 0.8 μm to 3.5 μm, 0.8 μm to 4.0 μm, 0.8 μm to 5.0 μm. In some embodiments, cryopreserved platelets herein, in illustrative embodiments, upon storing at a temperature in the range of −40° C. to −5° C., −30° C. to −5° C., or −20° C. to −5° C., for at least 1 month, 2, 3, 4, 6, 8, 10, 12 months, 2, 3, 4, 5, 6, 7, 8, 9, or 10 years typically upon thawing exhibit a particle size, for example, diameter in the range of 0.5 μm to 2.5 μm, 1 μm to 5 μm, 1 μm to 4 μm, or 0.5 μm to 5.0 μm and have a total number of platelets in a cryo-vessel equal to or more than 1.5×10¹¹, 1.6×10¹¹, or 1.7×10¹¹. In some embodiments, at least 50%, 60%, 70%, or 75%% of the cryopreserved platelets upon thawing have a diameter in the range of 0.5 μm to 2.5 μm, 1 μm to 5 μm, 1 μm to 4 μm, or 0.5 μm to 5.0 μm. In illustrative embodiments, cryopreserved platelets herein upon thawing have a total number of platelets in a cryo-vessel equal to or more than 1.5×10¹¹, 1.6×10¹¹, or 1.7×10¹¹, and typically, the cryopreserved platelets upon thawing retain hemostatic properties, for example, generating thrombin in an in vitro condition, ability to reduce bleeding in a subject, or ability to increase platelet numbers in a subject in need thereof. In illustrative embodiments, the ability to reduce bleeding in a subject is based on administration of, for example 0.5 to 3 units of frozen platelets, frozen platelet derivatives, cryopreserved platelets, or cryopreserved platelet derivatives. In illustrative embodiments, where 1 unit corresponds to 2.5×10¹¹+/−4.2 χ10¹¹ frozen or cryopreserved platelets and/or platelet derivatives. In some embodiments, methods herein include administering liquid compositions of thawed compositions of frozen platelets, frozen platelet derivatives, cryopreserved platelets, or cryopreserved platelet derivatives provided herein and/or prepared according to any method provided herein, to a subject to restore hemostasis, reduce bleeding, or stop bleeding in the subject.

Stability can also be determined by assessing certain parameters after thawing the cryopreserved platelets that are stored at a temperature higher than or equal to −40° C., −35° C., −30° C., −25° C., −15° C., or −10° C., but less than 0° C., in illustrative embodiments, at a temperature in the range of −40° C. to −10° C. In some embodiments, thawing of cryopreserved platelets as disclosed herein, or cryopreserved platelets obtained by a process as disclosed herein, can be done by subjecting the cryopreserved platelets to a temperature above the freezing temperature. For example, subjecting the cryopreserved platelets to a temperature above 0° C., for example, at least 5° C., 10° C., 15° C., 20° C., 25° C., 30° C., 35° C. In Illustrative embodiments, thawing comprises subjecting the cryopreserved platelets to a temperature in the range of 20° C. to 40° C., 22° C. to 40° C., 25° C. to 40° C., 30° C. to 40° C., or 32° C. to 40° C. In some embodiments, thawing comprises subjecting the cryopreserved platelets to a temperature of 37° C. +/−5° C., 37° C. +/−4° C., 37° C. +/−3° C., 37° C. +/−2° C., 37° C. +/−1° C., or 37° C. +/−0.5° C. Thawing herein typically comprises subjecting a cryo-vessel or a cryo-vial having cryopreserved platelet as disclosed herein to a water-bath set at a temperature of 37° C. +/−2° C. for a time-period until the contents in the cryo-vessel are completely thawed. A skilled artisan can contemplate that the time required for the contents to thaw completely can vary according to the volume of cryopreserved platelets, dimensions of the cryo-vessel and the temperature at which the cryo-vessels were stored before the thawing. Accordingly, thawing can be done by subjecting cryo-vessels to a water-bath set at a temperature of 37° C.+/−2° C. for at least 1 minute, 2, 3, 4, 5, 6, 7, 8, 9, or 10 minutes, for example in a range of 2-10, 2-9, 2-8, 2-7, or 2-6 minutes.

In some embodiments, the cryopreserved platelets, the cryopreserved platelet compositions, or the frozen platelets disclosed herein can be thawed and resuspended using a process to prepare the cryopreserved platelets for clinical use. In some cases, thawing cryopreserved platelets herein can include placing the cryo-vessels or the cryo-bags that contain the cryopreserved platelets in a water bath set at a temperature in the ranges of 37+/−5° C. for a time period sufficient to completely thaw the cryopreserved platelets. Alternatively, in some cases, thawing cryopreserved platelets here can include subjecting the cryopreserved platelets to dry thawing, for example, a thawing method that does not include the use of any liquid medium, such as water. In such cases, dry thawing can include placing the cryopreserved platelets in a controlled-rate thawing station, for example VIA Thaw, Cytiva, Cambridge, UK for a time period sufficient to completely thaw the cryopreserved platelets. In some cases, dry thawing can include placing the cryopreserved platelets in proximity to one or more metal plates heated to a pre-set temperature, for example, 37+/−5° C. In some cases, the cryopreserved platelets herein can be thawed in dry conditions, for example, in a commercially available dry thawer, such as Zipthaw, or SaharaIII. Accordingly, the time taken for the cryopreserved platelets to thaw can depend on the type of thawing, wet or dry. In some cases, the time taken for thawing varies in the range of 3-10, 3-9, 3-8, 4-10, or 4-9 minutes, and the time is calculated from the time of placing the cryo-vessels in the thawing machine, wet or dry to a time when the contents seem to be thawed on a visual inspection. In some cases, the post-thaw temperature of the contents in the cryo-vessel, such as the thawed platelet particles is in the range of 30-37° C., 31-37° C., or 32-37° C.

In some cases, the thawing process includes wet thawing that can comprise retrieving a cryo-vessel containing a cryopreserved platelet composition or cryopreserved platelets from a freezer, for example set at −20° C. +/−10° C., −65° C. +/−10° C., or −80° C. +/−10° C., and confirming the identification number of the cryo-vessel. The cryo-vessel can then be thawed in a plasma thawer or water bath maintained at 37° C. +/−5° C. for a duration of about 8 to 15 minutes, with the ports of the cryo-vessel, for example the cryopreserved platelet bag positioned upward. Following thawing, the cryo-vessel, for example the bag can be examined for physical integrity, including any signs of rips, tears, or holes, and the cryo-vessel is discarded if such damage is detected. In some cases, the weight and/or the volume of the cryopreserved platelet composition or frozen activated platelets in a cryo-vessel, upon thawing is within a range that is maintained across batches in a collection of cryo-vessels provided herein. For example, the weight of the cryopreserved platelet composition or the frozen activated platelets in a cryo-vessel, upon thawing is between 20 and 35 grams, 20 and 30 grams, or 25 and 29.50 grams. In terms of the volume, the cryopreserved platelet composition or the frozen activated platelets in a cryo-vessel upon thawing, has a volume between 20 and 35 ml, 20 and 30 ml, 24.7 and 29.5 ml, and 25 and 28.5 ml. In some embodiments, the cryopreserved platelets, the cryopreserved platelet compositions, or the frozen platelets can be resuspended or diluted after thawing. Typically, the resuspension or the dilution is performed by aseptically introducing approximately 20-30 mL, in illustrative embodiments approximately 25 mL of sterile saline (NaCl), for example, 0.9% NaCl, via a sterile syringe into the cryo-vessel, for example the bag through a cleaned, needleless injection port. Typically, saline used for resuspending or diluting the composition after thawing is maintained at room temperature, such that the composition after thawing at a water bath maintained at 37° C. +/−5° C. for a duration of about 8 to 15 minutes is diluted or resuspended with saline that is maintained at room temperature. In some cases, thawing the composition provides a thawed composition, and the resuspension or the dilution of the thawed composition can be performed until the final volume of the thawed composition is more than 45 ml, for example, in a range of 45.1 ml to 60 ml, for example, the final volume after the dilution or the resuspension can be greater than 45 ml and less than 60 ml. In some embodiments, the resuspension can be performed by using a resuspension solution, for example, a resuspension solution that is biocompatible with the human body fluids such that the resuspension solution can be introduced into the human body. For example, the resuspension solution can be a validated, transfusable solution. Non-limiting examples of transfusable solutions include sterile NaCl, human plasma, and a platelet storage solution. After the resuspension, the cryo-vessel can be gently massaged for approximately one to three minutes to aid resuspension, followed by a visual inspection for aggregates. If aggregates are detected, the cryo-vessel is further massaged over an additional five to ten-minute period. The cryo-vessel comprising thawed and resuspended platelets can then be labeled, optionally placed in a blinding bag for transport, and maintained at a temperature in the range of 15° C. to 30° C., 20° C. to 30° C., or 20-28° C. or at room temperature until use or testing. Infusion of the thawed platelets can be initiated within about 24, 20, 18, 16, 14, 12, 10, or 8 hours of resuspension. For the purposes of in vitro testing, such as stability testing of cryopreserved platelet compositions herein, upon thawing, and resuspending or diluting as disclosed herein above, the thawed composition, such as thawed-resuspended/diluted compositions can be stored at room temperature for different timepoints for example, 2, 4, 6, 8, 10, 12, or 24 hours after which the compositions can be tested for different attributes, for example, pH, microparticle concentration, and phosphatidylserine positivity. In some cases, storing at room temperature includes storing the composition after thawing and resuspending in a laboratory, for example at a work bench for conducting stability testing. Accordingly, in some cases, storing at room temperature includes exposing the composition after thawing and resuspending to the temperature maintained in a laboratory for conducting stability testing, such as pH, microparticle concentration, thrombin generation, and different flow cytometry related experiments. In some cases, a temperature used for stability testing can include a temperature within the range of 15° C. to 30° C., 20° C. to 30° C., or 20° C. to 25° C. In some cases, the volume of cryopreserved platelets in the cryo-vessels as disclosed herein, upon thawing is approximately the same or, is same as the volume of pooled resuspension with a cryoprotectant that was introduced into the cryo-vessels before initiating the freezing of the cryo-vessels. Accordingly, upon thawing the cryopreserved platelets or the frozen activated platelets herein, the volume of such a thawed activated platelet composition formed is in the range of 20-35 ml, 20-34 ml, 20-32 ml, or 25-29 ml. For example, the volume of a thawed activated platelet composition herein can be between 20, 22, or 25 ml on the low end of a range and 28.1, 29, 30, 32, 34, or 35 ml on the high end of the range. Accordingly, the weight of such a thawed activated platelet composition can be Further, the composition upon thawing is further diluted or resuspended with a volume of 20 ml, 22 ml, or 25 ml of a saline solution, such as a sterile solution, for example, 0.9% NaCl.

In some embodiments of aspects that include process for preparing a batch of cryopreserved platelets, a process for preparing a cryopreserved platelet composition including a transition in freezing temperatures from an initial freezing temperature to a storage freezing temperature, and/or cryopreserved platelets, a cryopreserved platelet composition, a composition comprising frozen platelets, a collection or a batch comprising cryopreserved platelets, the cryopreserved platelets, or the frozen platelets herein can be stable, or functionally stable after thawing or post-thawing for at least 4, 5, 6, 7, 8, 9, 10, 12, 18, or 24 hours, for example, upon storage at room temperature, or, at a temperature in a range of 18-28° C., 20-30° C., 20-26° C., or 20-25° C. In illustrative embodiments, the cryopreserved platelets, of the frozen platelets upon thawing can be stable for at least 6, 7, or 8 hours. Stability of the cryopreserved platelets or the frozen platelets upon thawing can be assessed by a non-limiting list of parameters including visual inspection of cracks, tears, breaks of the cryo-vessel, such as a cryo-bag, visual inspection of aggregate free swirling of the cryopreserved platelets in the cryo-vessel, such as a cryo-bag, platelet counts/concentrations per cryo-vessel, microparticle, for example CD61-positive microparticle count/concentrations in a cryo-vessel, ratio of concentration of platelets to concentration of CD61-positive microparticle in a cryo-vessel, ratio of concentration of CD61-positive microparticles to ratio of concentration of platelets, and in illustrative examples, pH of the cryopreserved platelets upon thawing, including the rate of change of pH units over a period of storage time upon thawing of the cryopreserved platelets. Functional stability, for example can include the ability to retain a platelet function such as ability to aggregate in vitro or generation of thrombin in vitro.

In some cases, the stability or the functional stability of the cryopreserved platelets or frozen activated platelets, or that of the composition comprising cryopreserved platelets herein can be assessed using the pH as a parameter, such that the pH of the cryopreserved platelets, upon thawing and storing for at least 5, 6, 7, 8, 10, 12, 16, 18, or 24 hours, for example at a temperature that is above the freezing temperature but no more than 50° C., is above 6.0, in illustrative embodiments is above 6.2. In some cases, pH of the cryopreserved platelets disclosed herein remains consistent upon thawing, for example, the cryopreserved platelets herein upon thawing and storing for at least 4, 6, 8, 12, or 24 hours, or storing for 4, 6, 8, 12, or 24 hours do not change (increase or decrease) by more than 0.7, 0.6, 0.5, 0.4, or 0.3 pH units. For example, pH of the cryopreserved platelets disclosed herein upon thawing and storing for at least 4, 6, 8, 12, or 24 hours, or storing for 4, 6, 8, 12, or 24 hours is maintained within 0.05 to 0.5, 0.05 to 0.4, 0.05 to 0.3 pH units as compared to the pH of the cryopreserved platelets immediately (for example, within 15, 12, 10, 5, or 2 minutes) upon thawing. In some cases, as demonstrated herein, the pH of the cryopreserved platelets upon thawing and storing for 4 hours, 8 hours, and 24 hours, is maintained within 0.1 to 0.2 pH unit, within 0.02 to 0.07 pH unit, and within 0.02 to 0.06 pH unit, respectively, as compared to the pH of the cryopreserved platelets, or the frozen activated platelets, immediately (for example, within 15, 12, 10, 5, or 2 minutes) upon thawing.

In some cases, the stability or the functional stability of the cryopreserved platelets or frozen activated platelets can be assessed using the concentration of platelets and/or microparticles in the composition as a parameter, such that upon thawing the concentration of platelets is more than the concentration of total microparticles, for example, CD61-positive microparticles in a cryo-vessel. For example, in a cryo-vessel comprising thawed platelets, the ratio of concentration of total platelets to the concentration of total microparticles, for example, CD61-positive microparticles is more than 1.

In some cases, the stability or the functional stability of the cryopreserved platelets herein can be assessed using the total platelet count in the composition in the cryo-vessels herein. For example, the total platelet count in the composition of each cryo-vessel herein is typically above 1.7×10¹¹. The cryopreserved platelets herein, for example, across at least 2, 3, 5, or 10 batches exhibit a mean of the total number of platelets upon thawing, and storing for at least 2, 4, 6, 8, 10, 12, or 24 hours, for example, within the range of 1.85×10¹¹ to 3.5×10¹¹. For example, the cryopreserved platelets herein have a property of exhibiting the mean of total number of platelets across at least 3 batches, or 2 to 4 batches upon thawing, and storing for 4, 8, or 24 hours in the range of 1.85 to 3.0×10¹¹, or 1.75 to 3.0×10¹¹.

In some cases, the stability or the functional stability of the cryopreserved platelets or frozen activated platelets herein can be assessed using the concentration of microparticles, for example, CD61 positive-microparticles/μl. The cryopreserved platelets herein, for example, across at least 2, 3, 5, or 10 batches, upon thawing, and storing for at least 2, 4, 6, 8, 10, 12, or 24 hours, exhibits a mean concentration of CD61 positive-microparticles/μl in the range of 5.0×10⁶/μl to 9.5×10⁶/μl. For example, the cryopreserved platelets herein have a property of exhibiting a mean concentration of CD61 positive-microparticles/μl upon thawing, and storing for 4, 8, or 24 hours in the range of 5.5×10⁶/μl to 9.0×10⁶/μl.

In some cases, the stability or the functional stability of the cryopreserved activated platelets can be assessed using the phosphatidylserine positivity, for example, when measured using lactadherin binding. The cryopreserved platelets herein, for example, across at least 2, 3, 5, or 10 batches, upon thawing, and storing for at least 2, 4, 6, 8, 10, 12, or 24 hours, exhibits a mean positivity of phosphatidylserine, when measured using lactadherin binding in the range of 85% to 95%. For example, the cryopreserved platelets herein have a property of exhibiting a mean positivity of phosphatidylserine, when measured using lactadherin binding upon thawing, and storing for 4, 8, or 24 hours in the range of 85% to 95%.

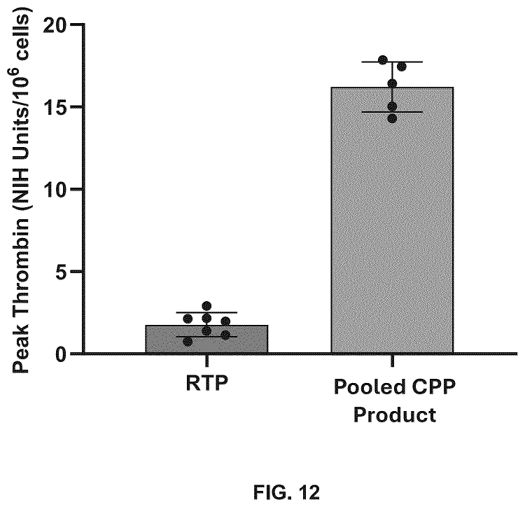

In some cases, the cryopreserved platelets or frozen activated platelets herein can have properties or attributes that are different from a single-donor cryopreserved platelet product, for example, the single-donor product prepared as per Example 1 herein. For example, the cryopreserved platelets can be activated as compared to the single-donor product. Non-limiting platelet activation markers can be selected from CD62 positivity, and phosphatidylserine positivity of cryopreserved platelets herein. In a non-limiting illustration, cryopreserved platelets herein, for example, upon thawing have a higher CD62% positivity as compared to the single donor CPP, for example, the CD62% positivity in the composition herein can be at least 55%, 56%, or 57%. In some cases, cryopreserved platelets herein, for example, upon thawing have a higher phosphatidylserine % positivity, for example, when assessed using Annexin V binding, or lactadherin binding as compared to the single donor CPP. For example, the phosphatidylserine % positivity when assessed using lactadherin binding in the composition herein can be at least 78%, or 79%. In some cases, cryopreserved platelets herein, for example, upon thawing have a higher thrombin generation ability (TGA) as compared to the single donor CPP. Non-limiting examples of assessing TGA include performing in vitro thrombin generation assays that can provide measurements in terms of thrombin peak height (TPH), or International Units (IU)/10⁶ particles. For example, when measuring the TGA using at least 20 k/μl particles, or 20 k/μl particles of the cryopreserved platelets herein, optionally, when in the presence of a reagent containing tissue factor (e.g., at 0.25 pM, 0.5 pM, 1 pM, 2 pM, 5 pM or 10 pM), and optionally phospholipids, the TPH can be at least 205, 215, or 235 nM. In other instances, when measuring the TGA using 20 k/μl particles of the cryopreserved platelets herein, optionally, when in the presence of a reagent containing tissue factor and phospholipids the IU/10⁶ particles of the cryopreserved platelets herein can be at least 1.45, or 1.5, for example when measured using Thrombinoscope instrument, or Fluoroskan Ascent instrument. In some cases, the IU can also be referred to as NIH units. In some cases, the IU/10⁶ particles of the cryopreserved platelets herein can be at least 10, 12, or 15 IU/10⁶ when measured using CLARIOstar^{plus}instrument. In some cases, compositions comprising cryopreserved platelets herein, for example, upon thawing can have a lower platelet concentration as compared to a single donor CPP, and can still exhibit a higher thrombin generation ability in terms of either TPH or IU/10⁶ particles consistent with the disclosure herein above. For example, compositions comprising cryopreserved platelets herein, upon thawing can have a platelet concentration in the range of 4×10⁶/μl to 5.5×10⁶/μl, and the TGA of 20 k/μl particles in terms of TPH can be at least 235 nM, and/or the TGA of 20 k/μl particles in terms of IU/10⁶ particles can be at least 1.5. In some cases, the thrombin generation ability of the cryopreserved platelets herein can be higher than that of liquid stored platelets, room temperature platelets, or apheresis platelets. For example, as has been demonstrated in Example 12 (FIG. 12), where it is shown that the cryopreserved platelets herein have at least 3-fold higher TGA in terms of IU/10⁶ particle or NIH Units/10⁶ particle. In some cases, the thrombin generation ability of the cryopreserved platelets herein can be higher than that of cold stored platelets (CSP), room temperature platelets (RTP), for example, at least 2-fold, or at least 3-fold higher than that of cold stored platelets. In some cases, the cryopreserved platelets herein can exhibit greater thrombin generation in less time as compared to RTP and CSP. For example, in a TGA assay using Thrombinoscope, the cryopreserved platelets herein exhibits greater thrombin generation achieving higher peak height in less time to peak as compared to CSP and RTP. In some cases, the cryopreserved platelets upon thawing promotes thrombin generation to a greater extent, mean value of TGPU of at least 10, 11, 12, 13, 14, or 15 15 as compared to CSP (approx. 7 TGPU), and RTP (less than 5 TGPU). In some cases, the cryopreserved platelets herein upon thawing can reach a thrombin peak height of at least 100, 120, 130, 140, or 150 nM in less than 10 minutes in an in vitro TGA assay performed using Thrombinoscope. In some cases, the cryopreserved platelets herein in a TGA assay using Thrombinoscope exhibits time to reach thrombin peak in 5-10, 5-9, or 5-8 minutes. For example, the cryopreserved platelets herein can exhibit time to reach thrombin peak in at least 1.2-, 1.3-, 1.4-, 1.5-, 1.7-, or 2-fold less time as compared to RTP and CSP. In some cases, the cryopreserved platelets herein can exhibit time to reach lag in at least 1.2-, 1.3-, 1.4-, 1.5-, 1.7-, or 2-fold less time as compared to RTP and CSP. In some cases, the cryopreserved platelets herein can exhibit a greater clot formation strength as compared to RTP and CSP. For example, the cryopreserved platelets herein can exhibit a greater clot formation strength, such as 1.2-, 1.3-, 1.4-, 1.5-, 1.7-, or 2-fold greater clot formation strength as compared to RTP and CSP, for example in a Thrombinoscope instrument. In some cases, the cryopreserved platelets herein can exhibit a shortened time to initiate clot formation, for example, the cryopreserved platelets herein can initiate a faster clot initiation as compared to RTP, or CSP. For example, the cryopreserved platelets herein can exhibit clot initiation in at least 1.2-, 1.3-, 1.4-, 1.5-, 1.7-, or 2-fold less time as compared to RTP or CSP. In some cases, the cryopreserved platelets herein can exhibit a faster clot formation as compared to RTP or CSP. For example, the cryopreserved platelets herein can exhibit clot formation in at least 1.2-, 1.3-, 1.4-, 1.5-, 1.7-, or 2-fold less time as compared to RTP or CSP. In some cases, the cryopreserved platelets herein can exhibit a reduced activation potential as compared to RTP or CSP, for example, when measured using light transmission aggregometry (LTA) assay using one or more agonists. In some cases, the cryopreserved platelets herein comprise a population of platelet particles that can be activated upon stimulation with an agonist, for example, when measured in vitro. In some cases, the cryopreserved platelets herein, for example, upon thawing exhibits high homogeneity as compared to CSP, and RTP. For example, the cryopreserved platelets herein, for example, in each cryo-vessel across at least 3, 4, 5, or 6 batches comprise a total platelet particle count having a coefficient of variation of less than 10%, 9%, or 8% across the batches. For example, the coefficient of variation can be in the range of 3-10%, 3-9%, or 4-8%. In some cases, the cryopreserved platelets herein, for example, in each cryo-vessel across at least 3, 4, 5, or 6 batches has a coefficient of variance of pH of less than 2%, 1.75%, 1.5%, or 1.25%, for example, the coefficient of variance of pH is in the range of 0.25-1.9%, 0.25-1.75%, 0.25-1.5%, or 0.5-1.25%. In some cases, the cryopreserved platelets herein, for example, in each cryo-vessel across at least 3, 4, 5, or 6 batches comprise platelet particles having a % phosphatidylserine positivity, for example, when measured using lactadherin binding is in the range of 70-90%, 72-90%, or 74-90%, for example, the coefficient or variance of % phosphatidylserine positivity across at least 3, 4, 5, or 6 batches is less than 35%, 20%, 25%, 20%, 18%, 15%, 12%, 10%, 7%, or 5%, for example, is in the range of 2-25%, 2-22%, 2-20%, 2-25%, 2-12%, 2-10%, 2-8%, or 3-7%. In some cases, the cryopreserved platelets herein, for example, in each cryo-vessel across at least 3, 4, 5, or 6 batches comprise platelet particles having thrombin generation activity, for example, 1.4-1.8, or 1.4-1.6 TGPU (IU/10⁶ particles), for example, the coefficient or variance of thrombin generation activity is less than 35%, 20%, 25%, 20%, 18%, 15%, 12%, 10%, 7%, or 5%, for example, is in the range of 2-25%, 2-22%, 2-20%, 2-25%, 2-12%, 2-10%, 2-8%, or 3-7%.

In some cases, the cryopreserved platelets herein can exhibit different aggregation responses in the presence of agonist, including, not limited to collagen, thrombin, arachidonic acid, and thrombin receptor activating peptide (TRAP), like TRAP-6, for example, in the absence of divalent cations as compared to fresh platelets, apheresis platelet units, or liquid stored platelets. For example, in illustrative examples the cryopreserved platelets herein do not exhibit detectable aggregation, or exhibit substantially less aggregation, for example, less than 2, 3, 4, or 5-fold, in the absence of divalent cations, and in the absence of fresh platelets, but in the presence of collagen, thrombin, arachidonic acid, and TRAP-6 as compared to the aggregation exhibited by fresh platelets, apheresis platelet units, or liquid stored platelets. In some cases, the cryopreserved platelets herein exhibit 2-6-fold, 2-5-fold, 2-4.5-fold, 2-4-fold, 2-3.5-fold, or 2-3-fold less aggregation, in the absence of divalent cations, and in the absence of fresh platelets, but in the presence of an agonist like collagen, thrombin, arachidonic acid, and TRAP-6 as compared to the aggregation exhibited by fresh platelets, apheresis platelet units, or liquid stored platelets in the absence of divalent cations and in the absence of fresh platelets. Non-limiting examples of divalent cations include magnesium (Mg²⁺), barium (Ba²⁺), copper (Cu²⁺), calcium (Ca²⁺), manganese (Mn²⁺), zinc (Zn²⁺), iron (Fe²⁺), nickel (Ni²⁺), and cobalt (Co²⁺). A skilled artisan can understand that any suitable salt of the divalent cations can be used for the aggregation assay, for example, a chloride salt of magnesium, barium, copper, calcium, manganese, zinc, iron, nickel, or cobalt.

### Preparation of Cryopreserved Platelets Using a Transition in Freezing Temperatures

Provided herein, in some aspects, is a process comprising a step of initial freezing at a temperature (i.e., initial temperature) less than or equal to −50° C., −60° C., −65° C., −70° C., −80° C., −85° C., or −90° C., or in the range of −50° C. to −85° C., or −60° C. to −85° C. to form an initial frozen platelet composition, followed by storing the initial frozen platelet composition in a frozen state at a temperature (i.e., storage temperature) equal to or greater than −30° C., but less than 0° C., to form a cryopreserved platelet composition. Surprisingly, it was found that the cryopreserved platelets formed by the process as disclosed herein have the property of being stable and retain hemostatic abilities when stored at higher temperatures as compared to that required for storing conventional cryopreserved platelets. For example, the cryopreserved platelets prepared as per a process disclosed herein can be stored at a temperature of about −30° C., −25° C., −20° C., −15° C., −10° C., or −5° C. or at a temperature in the range of −30° C. to −5° C., or −30° C. to −5° C. for at least 1 month up to 2, 3, 4, 5, 6, 7, 8, 9, 10, 11, or 12 months, or for at least 2, 3, 4, 5, 6, 7, 8, 9, 10, 11, or 12 months, or for at least, or up to 1 year, 2, 3, 4, 5, or 6 years, or for between 6 months and 1 year, 2, 3, 4, 5, or 6 years.

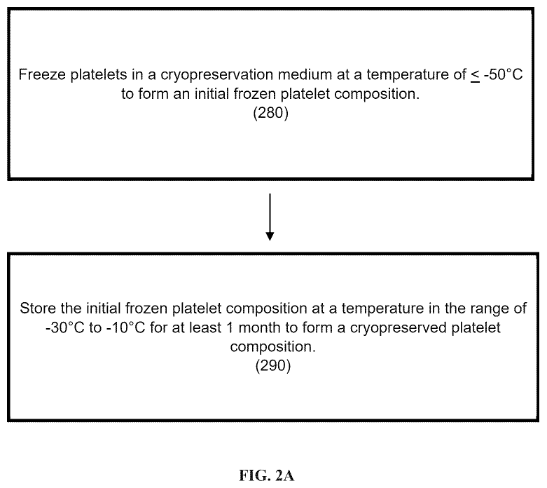

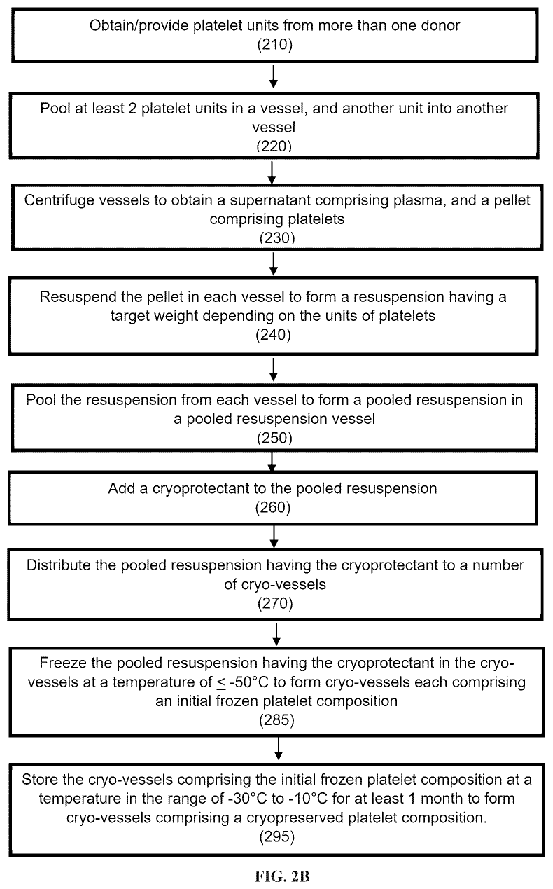

A non-limiting example of a process provided herein that includes a transition in freezing temperatures is illustrated in FIG. 2A. The steps of this non-limiting example are shown in boxes in FIG. 2A. Such non-limiting exemplary method includes the following steps:
a) freezing platelets in a cryopreservation medium at a temperature of less than or equal to −50° C. (or in some aspects, less than or equal to −55° C., or less than or equal to −60° C.) to form an initial frozen platelet composition (280); and
b) storing the initial frozen platelet composition at a temperature in the range of −30° C. to −10° C. (in some embodiments in a freezer set at, or at about −20° C.) for at least 1 month to form a cryopreserved platelet composition (290). In some embodiments, the cryopreserved platelets formed in such a method are cryopreserved platelet derivatives. Furthermore, in illustrative embodiments, the cryopreserved platelet composition or the cryopreserved platelet derivative composition have one or more recited properties provided herein for frozen platelet and/or frozen platelet derivative compositions.

Another non-limiting example of a processes for preparing a cryopreserved platelet composition is illustrated in FIG. 2B. Such non-limiting example includes the following steps:
a) obtaining or providing platelet units from more than one donor (210);
b) pooling at least 2 platelet units into one vessel and at least another platelet unit into another vessel (220), such that there will be more than one vessel at the end of the step;
c) centrifuging each vessel to obtain a supernatant comprising plasma, and a pellet comprising platelets (230);
d) resuspending the pellet in each vessel to form a resuspension wherein the resuspension has a target weight determined by the number of units pooled or provided in the vessel (240);
e) pooling the resuspension from each vessel to form a pooled resuspension in a pooled resuspension vessel (250);
f) adding a cryoprotectant to the pooled resuspension vessel having the pooled resuspension to obtain a pooled resuspension having the cryoprotectant (260); and
g) distributing the pooled resuspension having the cryoprotectant from the pooled resuspension vessel among a number of cryo-vessels (270). In some embodiments, the pooled resuspension in the cryo-vessels is frozen and stored at a temperature of less than or equal to −50° C.

Optionally, in some embodiments, a transitional temperature freezing protocol is performed on the pooled resuspension in the cryo-vessels. Accordingly, such embodiments include the following steps: h) freezing the pooled resuspension in the cryo-vessels at a temperature of less than or equal to −50° C. to form cryo-vessels each comprising an initial frozen platelet composition (285); and i) storing the cryo-vessels comprising the initial frozen platelet composition at a temperature in the range of −30° C. to −10° C. for at least 1 month to form cryo-vessels comprising a cryopreserved platelet composition (295).

Provided herein are additional processes and/or details for steps of the above processes for preparing cryopreserved platelets that include a transition in freezing temperature. Accordingly, provide herein is a process for preparing cryopreserved platelets that include an initial freezing step comprising freezing platelets in a cryopreservation medium, or freezing a pooled resuspension having a cryoprotectant as disclosed herein, at a temperature of less than or equal to −50° C., −55° C., −60° C., in illustrative embodiments, less than or equal to −65° C., −70° C., −75° C., or −80° C., to form an initial frozen platelet composition, and a second step comprising storing the initial frozen platelet composition in a frozen state at a temperature higher than or equal to −40° C., −35° C., −30° C., −25° C., in illustrative embodiments, higher than or equal to −20° C., −15° C., or −10° C., but less than 0° C. to form cryopreserved platelets, cryopreserved platelet composition, or a batch of cryopreserved platelets. In some embodiments, a composition that includes cryopreserved platelets obtained from a process using a transition in freezing temperatures from an initial temperature equal to or less than some initial target temperature set at a target initial temperature or temperature range that is no warmer than −50° C., and then stored after some period of time, at a temperature that is at a target storage temperature set at a target storage temperature or storage temperature range between about −10° C. to about −30° C., can be referred to as a transition temperature cryopreserved-product, or a transition temperature-cryopreserved composition, and such a process can be referred to as a transition temperature cryopreservation process. Also, for convenience to differentiate a transition temperature cryopreserved product from a cryopreserved product that does not involve such a transition in temperature in its preparation, in some embodiments, a cryopreserved product that is obtained by only freezing and storing at a target temperature at or below −60° C., for example about −80° C., is referred to as a single temperature cryopreserved-product. A skilled artisan will understand that such single-temperature cryopreserved product can in fact, be subjected to a variation in temperatures, but such variation does not include a transition from an initial freezing temperature at or below −50° C. to a target storage temperature at −10° C. to −30° C. Surprisingly, cryopreserved platelets obtained by a process including a transition in temperature as disclosed herein when stored at a temperature of equal to or higher than −30° C. but less than 0° C., or −5° C. upon thawing can exhibit hemostatic properties, and in illustrative embodiments are capable of reducing bleeding in a subject, increasing the platelet counts of a subject in need thereof, for example, in a thrombocytopenic subject, or generating thrombin in an in vitro thrombin generation assay, thereby addressing a long-felt need in storage conditions of cryopreserved platelets.

In some embodiments, an initial frozen platelet composition can be stored at a freezing temperature higher than −40° C., −35° C., −30° C., −25° C., −20° C. In some embodiments, storing of an initial frozen platelet composition can be done for at least 30 minutes, 1 hour, 2, 3, 6, 8, 10, 12, 18, 24 hours, 2 days, 3, 5, 7, 15, 20, 25, days, 1 month, 2, 3, 4, 5, 6, 7, 8, 9, 10, 11, or 12 months, or 1 year, 2, 3, 4, 5, or 6 years. In some embodiments, storing of an initial frozen platelet composition can be done for a time period in the range of 1 month to 10 years, 1 month to 8 years, 1 month to 6 years, 1 month to 5 years, 1 month to 3 years, 1 month to 2 years, 1 month to 1 year, 6 months to 10 years, 1 year to 10 years, 2 years to 10 years, or 3 years to 10 years. In some cases, an initial frozen platelet composition can be stored at a temperature in the range of −40° C. to −10° C. until the cryopreserved platelets are used for treating a subject in need thereof, in illustrative embodiments, for administering to a subject for reducing bleeding in the subject.

Typically, in an initial freezing step, a cryopreservation medium having platelets, or a pooled resuspension having a cryoprotectant as disclosed herein, become frozen, and achieve the temperatures as disclosed therein to form an initial frozen platelet composition. For example, freezing a cryopreservation medium having platelets, or a pooled resuspension having a cryoprotectant as disclosed herein at a temperature in the range of −50° C. to −85° C. comprises subjecting the cryopreservation medium, or the pooled resuspension to the temperature range such that an initial frozen platelet composition is formed at the end of the step, and the temperature of the initial frozen platelet composition is in the range of −50° C. to −85° C. An initial freezing step can be performed for a time period until the cryopreservation medium having the platelets or the pooled resuspension having a cryoprotectant reaches a temperature less than or equal to −50° C., −55° C., −60° C., −65° C., −70° C., −75° C., or −80° C., and the time it takes for the cryopreservation medium or the pooled resuspension to attain the temperature can depend on various factors, not limited to the volume of a cryo-vessel, dimensions of a cryo-vessel, volume of cryopreservation medium having the platelets, concentration of platelets in a cryo-vessel, and composition of a cryopreservation medium in a cryo-vessel. A cryopreservation medium that can be used in a process as disclosed herein can be a cryopreservation medium comprising a cryoprotectant. In illustrative embodiments, the cryoprotectant comprises dimethyl sulfoxide (DMSO). In other embodiments, the cryoprotectant can be any other cryoprotectant apart from DMSO. Other non-limiting examples of suitable cryoprotectants can include saccharides, such as monosaccharides and disaccharides, including sucrose, maltose, trehalose, glucose, mannose, dextrose, xylose, and a combination thereof. In some embodiments, a cryopreservation medium comprising DMSO as a cryoprotectant can have a concentration in the range of 0.001-10%, 0.5-7%, 1-8%, 2-8%, 3-8%, 4-8%, or 5-8%. In some embodiments, an initial freezing step can be done for at least for at least 30 minutes, 1 hour, 2 hours, or 3 hours. For example, an initial freezing step can be done for a time period in the range of 30 minutes to 12 hours, 30 minutes to 10 hours, 30 minutes to 8 hours, 30 minutes to 6 hours, or 30 minutes to 4 hours. In some embodiments, an initial freezing step can be done for more than 12 hours, 2 days, 3 days, 1 week, 1 month, or 6 months. In some embodiments, the temperature during an initial freezing step can be in the range of −50° C. to −90° C., −50° C. to −85° C., −50° C. to −80° C., −50° C. to −75° C., −50° C. to −70° C., −55° C. to −90° C., −60° C. to −90° C., −60° C. to −85° C., −60° C. to −80° C., −60° C. to −75° C., or −65° C. to −75° C. In illustrative embodiments, the temperature during an initial freezing step can be −65° C. +/−5° C., −65° C. +/−4° C., −65° C. +/−3° C., −65° C. +/−2° C., or −65° C. +/−1° C. In other illustrative embodiments, the temperature during an initial freezing step can be −80° C. +/−5° C., −80° C. +/−4° C., −80° C. +/−3° C., −80° C. +/−2° C., or −80° C. +/−1° C. In some embodiments, the time-period during an initial freezing step can depend on the temperature that needs to be achieved. For example, an initial freezing step can comprise a temperature in the range of −60° C. to −80° C., for a time period in the range of 1 hour to 7 hours, 1 hour to 6 hours, 1 hour to 5 hours, 1 hour to 4 hours, or 1 hour to 2 hours. In some embodiments, an initial freezing step comprises placing a cryopreservation medium having platelets, or a pooled resuspension having a cryoprotectant in a freezer set at a temperature in the range of −50° C. to −90° C., to form an initial frozen platelet composition.

In some embodiments, storing an initial frozen platelet composition comprises subjecting an initial frozen platelet composition to a temperature equal to or higher than −30° C., −25° C., −20° C., −15° C., or −10° C. but less than −5° C. In illustrative embodiments, an initial frozen platelet composition is subjected to a temperature of −20° C. +/−5° C., −20° C. +/−4° C., −20° C. +/−3° C., −20° C. +/−2° C., −20° C. +/−1° C., or −20° C. +/−0.5° C. In some embodiments, storing an initial frozen platelet composition comprises storing in a freezer that is set at a temperature in a range of −10° C. to −40° C., −10° C. to −30° C., or −15° C. to −25° C. Typically, an initial frozen platelet composition when stored at a temperature as disclosed herein or when subjected to a temperature as disclosed herein reaches the intended temperature at the end of the step to form cryopreserved platelets, and after the step, the cryopreserved platelets is stored at the temperature disclosed herein for at least 7, 10, 15, 20, 25 days, 1 month, 2, 3, 4, 6, 8, 10, 12 months, 1 year, 2, 3, 4, 5, 6, 7, 8, 9, or 10 years, or until the cryopreserved platelets are used for administering to or treating a subject in need thereof. In some embodiments, storing an initial frozen platelet composition can comprise storing at a freezing temperature of equal to or higher than −30° C. for a time of at least 30 minutes, 45 minutes, 50 minutes, 60 minutes, 70 minutes, 80 minutes, 90 minutes, 2 hours, or 3 hours to form cryopreserved platelets. In some embodiments, a process disclosed herein can comprise subjecting an initial frozen platelet composition to a temperature equal to or higher than −30° C., in illustrative embodiments, in a range of −10° C. to −30° C. for a time until the temperature of the initial frozen platelet composition reaches the temperature of equal to or higher than −30° C., or in illustrative embodiments, in a range of −10° C. to −30° C. to form cryopreserved platelets. Typically, once the temperature of the initial frozen platelet composition reaches the temperature in a range of −10° C. to −30° C. to form a cryopreserved composition, the cryopreserved composition is be stored at a temperature in a range of −10° C. to −30° C. until the cryopreserved platelets are used for treating a subject in need thereof, or are used for administrating to a subject in need thereof.

### Preparation of Cryopreserved Platelets Using a Single Freezing Temperature

In some aspects and embodiments of a process for preparing a batch of cryopreserved platelets herein, freezing the pooled resuspension herein, in illustrative embodiments the pooled resuspension having DMSO in the cryo-vessels, includes freezing the pooled resuspension at a temperature higher than −80° C., −75° C., −70° C., −65° C., −60° C., −55° C., −50° C., −45° C., −40° C., −35° C., or −30° C. to form the batch of cryopreserved platelets. In some embodiments, the process further includes storing the batch of cryopreserved platelets thus formed at the same temperature that is used to freeze the pooled resuspension herein. In some embodiments, the process herein does not include a transition in freezing temperatures. In some embodiments, the process herein that includes freezing the pooled resuspension at a temperature higher than −80° C., −75° C., −70° C., −65° C., −60° C., −55° C., −50° C., −45° C., −40° C., −35° C., or −30° C. to form the batch of cryopreserved platelets, and the process does not include a transition in freezing temperatures as disclosed herein. In some embodiments, the freezing the pooled resuspension includes freezing at a temperature in the range of −10° C. to −50° C., −10° C. to −45° C., −10° C. to −40° C., or −10° C. to −30° C. In some embodiments, the freezing the pooled resuspension includes freezing between a temperature of −10° C., −12° C., −15° C., −18° C. at a higher end of the temperature and −15° C., −20° C., −22° C., −24° C., −25° C., −27° C., −30° C., −32° C., −35° C., −37° C., −40° C., −45° C., −50° C., −55° C., or −60° C. at a lower end of the temperature. In some embodiments, the freezing includes storing the pooled resuspension in a freezer set at a temperature of −20° C. +/−1° C., −20° C. +/−2° C., −20° C. +/−3° C., −20° C. +/−4° C., −20° C. +/−5° C., −20° C. +/−6° C., −20° C. +/−7° C., −20° C. +/−8° C., −20° C. +/−9° C., or −20° C.+/−10° C. In some embodiments, the freezing and/or the storing is done at a temperature higher than −80° C., in illustrative embodiments, temperatures as disclosed above herein for at least 1 month, 2 months, 3 months, 4 months, 5 months, 6 months, 8 months, 10 months, 12 months, 16 months, 18 months, 24 months, 3 years, 4 years, 5 years, or 6 years, or for between 1 month and 1, 2, 3, 4, 5, or 6 years, or for between 3 months and 1, 2, 3, 4, 5, or 6 years, or for between 6 months and 1, 2, 3, 4, 5, or 6 years. In some embodiments, the freezing does not include exposing the pooled resuspension at a temperature of less than −50° C., −60° C., −70° C., or −80° C.

### Compositions Comprising Frozen Platelets

Compositions provided herein in some aspects and embodiments have one or more recited properties (which can also be referred to as recited attributes or recited characteristics). It will be understood that compositions that fall under such aspects or embodiments comprising one or more recited properties exhibit such one or more recited properties, but to fall under such aspects or embodiments that comprise such recited one or more properties does not require that a step is actually performed to demonstrate the one or more recited properties. However, a skilled artisan will understand that such one or more recited properties of a composition can be identified using a method that is set out by a recited property, or by performing a known method, to determine whether a test composition possesses such one or more recited properties. Frozen compositions herein that comprise platelets and/or platelet derivatives, upon thawing exhibit one or more of the following non-limiting recited properties: a) are capable of exhibiting a platelet count of at least 1.0×10¹¹ in 35 ml; b) have about 50% to about 99% of platelets and/or platelet-derived particles in the range of about 1 μm to about 2.5 μm or 5 μm; c) are in a liquid state without requiring the addition of a liquid to achieve such liquid state; d) yield a single peak that corresponds to a compromised membrane peak in a membrane integrity assay; e) exhibit a CD61-positive-microparticle content of less than 50% of the CD61 positive particles in the composition; f) exhibit an ability to generate thrombin in an in vitro thrombin generation assay; g) are capable of inducing aggregation under in vitro aggregation conditions comprising an agonist; h) exhibit swirling upon visual observation of the composition; i) exhibit lack of aggregation upon visual observation of the composition; and/or j) exhibit lactadherin positivity in the range of 80-99.5%. Typically, the lactadherin positivity is the positivity of lactadherin binding that reflects the positivity of phosphatidylserine in a platelet population. Phosphatidylserine on platelets can be detected by detecting the binding of either Annexin V or lactadherin. In some cases, the frozen composition or the composition comprising cryopreserved platelets herein exhibit a CD 62% positivity of at least 50%, and/or phosphatidylserine positivity of at least 70% when measured using lactadherin binding. In some cases, the population of particles that show positivity to phosphatidylerine when measured using lactadherin binding can include microparticles in addition to platelet particles. In some cases, the population of particles that show positivity to CD62 can include microparticles in addition to platelet particles.

Provided herein in an aspect is a composition comprising frozen platelets, in an illustrative embodiment, frozen platelet derivatives, in a cryopreservation medium in a frozen state. In some embodiments, a composition comprising frozen platelets in a cryopreservation medium is a composition comprising cryopreserved platelets and/or cryopreserved platelet derivatives. Typically, a composition comprising frozen platelets upon thawing is in a liquid state without the addition of a liquid, such as water or a buffer. Without being bound by any theory, since the process of cryopreservation does not include the step of drying, the platelets in a cryopreservation medium become frozen because the cryopreservation medium is subjected to a freezing temperature, since there is no step of drying, the cryopreservation medium having platelets when thawed is in a liquid state. In some embodiments, a composition herein upon storing for at least 1 month, 2, 3, 4, 5, 6, 7, 8, 9, 10, 11, 12 months, 1 year, 2, 4, 5, 8, or 10 years at a temperature in the range of −10° C. to −40° C. is capable of exhibiting a platelet count of at least 1.0×10¹¹, 1.2×10¹¹, 1.4×10¹¹ , 1.6×10¹¹, or 1.7×10¹¹/20-35 ml of the composition. For example, a composition herein upon storing for at least 1 month, 2, 3, 4, 5, 6, 7, 8, 9, 10, 11, 12 months, 1 year, 2, 4, 5, 8, or 10 years at a temperature in the range of −10° C. to −40° C. is capable of exhibiting a platelet count of at least 1.0×10¹¹ in a cryo-vessel, cryo-vial, or a cryo-bag having a volume of 35, 30, 25, or 20 ml, or a volume of about 20-45 ml, 20-40 ml, or 20-35 ml. In some cases, the platelet count can be measured after dilution or resuspension of the composition after thawing, with a liquid, for example, saline such as 0.9% NaCl, such that the final volume after thawing and resuspension/dilution can be in the range of 45 to 65 ml, for example 45.1 to 60 ml. Platelet counts can be performed with an automated hematology analyzer, or manually with a hemocytometer. For example, platelet counts of a sample, such as a thawed platelet sample can be determined by using a hematology analyzer, for example, a Beckman Coulter AcT Diff 2 Hematology Particle Analyzer or a Beckman Coulter D×H Hematology Analyzer (Beckman Coulter, beckmancoulter.com). Hematology analyzers are known to be based on the Coulter Principle, which is an electronic method for counting and sizing particles. Although the Coulter Principle can be used to calculate and size many types of particles, the specific application of this principle in hematology is to count and size white blood cells (WBC), red blood cells (RBC), and platelets (PLT). As a non-limiting method, the platelet count in a composition herein can be derived from an internal continuous PLT/RBC histogram. Particles between 0 and 70 fL are counted and sized as they pass through the RBC aperture. The raw data is evaluated using a proprietary platelet algorithm, such as D×H (available on the Beckman Coulter D×H Hematology Analyzer) to identify the platelet population. The system also performs feature analysis to identify patterns of interference at the low and high ends of the PLT histogram. The algorithm uses both the PLT raw data and the fitted histograms for this process to determine PLT interference patterns, correcting or flagging results, depending on the severity of the interference. The platelet histogram's evaluation improves accuracy by excluding interferences from debris, micro bubbles, red cell fragments or exceptionally small red blood cells. As a non-limiting example of platelet count techniques, Example 5 and Table 6 demonstrate the data for platelet counts per bag by using either the Beckman Coulter AcT Diff 2 Hematology Particle Analyzer or the Beckman Coulter D×H Hematology Analyzer. In some embodiments, platelets can be counted by considering platelets having a diameter in the range of 0.5-5 μm, 0.6-5 μm, 0.7-5 μm, 1-4 μm, 1-3 μm, 1-2.5 μm, 1.5-3 μm, or in illustrative embodiments, 0.5-2.5 μm, 0.7-2.5 μm or 2.5-5.0 μm, typically when measured by flow cytometry or light scattering. In some embodiments, platelets can be counted by considering platelets having a diameter of at least 0.5 μm, 0.6 μm, 0.7 μm, or at least 1 μm, typically when measured by flow cytometry or light scattering. In some embodiments, particles in a composition that are less than 1p m, 0.6 μm, or 0.5 μm in diameter are microparticles, typically when measured by flow cytometry or light scattering. In some embodiments, particles in a composition that are less than 0.7 μm, 0.6 μm, or 0.5 μm in diameter are microparticles, typically when measured by flow cytometry or light scattering. In some cases, depending on the sensitivity of the equipment such as a flow cytometer with sensitivity of 0.3 μm, microparticles can have a size in the range of 0.3 μm to 0.6 μm. As a non-limiting example of platelet count techniques, flow cytometry can be used for sorting and counting platelets, or platelet derivatives in a composition herein. As is known in the art, different techniques are available for measuring particle sizes of platelets, platelet derivatives, and microparticles, for example platelet derived microparticles. One such technique, in a non-limiting manner, that can be used for measuring particle sizes is flow cytometry. Flow Cytometry is a technique for quantifying characteristics of cells such as cell number, size and complexity, fluorescence, phenotype, and viability. In general, the forward scatter in a flow cytometry is located in line with the laser intercept and is typically considered a measure of the relative cell size. The side scatter is typically located perpendicular to the laser beam intercept and is used to measure the relative complexity of the cell. Commercially available sizing beads can be used to obtain the forward scatter values to calibrate the instrument in order to measure the sizes of the particles. The gates used to measure the size distribution of particles in a composition as disclosed herein are drawn using forward scatter height (FSC-H) signals generated by latex beads of a known diameter. For example, commercially available sizing beads of 0.5 μm, and 2.5 μm can be used to set size gate ranges in a flow cytometry equipment for counting particles that are below 0.5 μm, such as microparticles or platelet derived microparticles, for counting platelets or platelet derivatives that fall in the range of 0.5 μm and 2.5 μm. In some embodiments, a composition comprising frozen platelets or frozen platelet derivatives, or cryopreserved platelets or cryopreserved platelet derivatives in a frozen state, in illustrative embodiments upon storing for at least 1 month, 2, 3, 4, 5, 6, 7, 8, 9, 10, 11, 12 months, 1 year, 2, 4, 5, 8, or 10 years at a temperature in the range of −10° C. to −40° C., upon thawing exhibit a platelet count recovery of at least 65%, 70%, or 75%. For example, a platelet count recovery can be in a range of 60% to 95%, 65% to 95%, 70% to 95%, or 75% to 95%, 70% to 99%, 72% to 99%, or 75% to 99%. A skilled artisan can understand that platelet recovery can be performed by comparing the platelet counts in a composition before freezing and after thawing, to assess the counts after a storage time. In a non-limiting example, percentage platelet recovery can be assessed by using Beckman Coulter AcT Diff 2 Hematology Particle Analyzer or the Beckman Coulter D×H Hematology Analyzer (See Example 7).

In some embodiments, a composition comprising frozen platelets and/or platelet derivatives herein upon storing for at least 1 month, 2, 3, 4, 5, 6, 7, 8, 9, 10, 11, or 12 months at a temperature in the range of −10° C. to −40° C., upon thawing can have a CD61-positive microparticle content of less than 80%, 75%, 70%, 65%, 60%, in illustrative embodiments, less than 50%, 40%, 30%, or 25%. In some embodiments, CD61-positive microparticle content out of all the particles including platelets, platelet derivatives, and microparticles is in the range of 1-30%, 1-25%, 1-20%, 1-15%, 5-30%, 5-25%, or 5-20%. In some embodiments, microparticles, or CD 61-positive microparticles are particles that are less than 0.5 μm in diameter, typically when measured by flow cytometry or light scattering. In some embodiments, microparticles, or CD 61-positive microparticles are particles that are less than 0.25 μm in diameter, typically when measured by flow cytometry or light scattering. In some embodiments, microparticles, or CD 61-positive microparticles are particles that are less than 1 μm in diameter, typically when measured by flow cytometry or light scattering. In some embodiments, at least 70%, 75%, 80% of the particles, typically including platelet, platelet derivatives, and microparticles in the composition are positive for lactadherin. For example, lactadherin positive particles in a composition can be in the range of 70% to 99%, 75% to 99%, or 80% to 99% of the particles in the composition. In some embodiments, lactadherin positive microparticles in a composition can be in the range of 70% to 99%, 75% to 99%, or 80% to 99% of the particles in the composition. Analysing using flow cytometry-based sorting and counting is a non-limiting technique for calculating the percentage positivity of CD-61 positive microparticles, and lactadherin positive particles. Example 9 demonstrates a technique for calculating the percentage positivity of CD-61 positive microparticles, and lactadherin positive particles in a composition as disclosed herein, and the data is tabulated in Table 8. Various known techniques can be used to determine the sizes of various populations of particles in a composition as disclosed herein. For example, in some embodiments, flow cytometry forward scattering is used for determining the size of the particles. In other embodiments, light scattering, such as Thrombolux Dynamic light scattering is used for determining the size of the particles.

In some embodiments, a composition comprising frozen platelets, and/or platelet derivatives provided herein, in illustrative embodiments upon thawing, comprises platelets and/or platelet-derived particles, such as platelet derivatives having a particle size (e.g., diameter or max dimension) of at least at least about 0.4 μm, at least about 0.5 μm, at least about 0.6 μm, at least about 0.7 μm, at least about 0.8 μm, at least about 0.9 μm, or at least about 1.0 μm., about 0.5 μm to about 5.0 μm. In some embodiments, the cryopreserved platelet composition has about 50% to about 99% (e.g., about 55% to about 95%, about 60% to about 90%, about 65% to about 85, about 70% to about 80%) of platelets and/or platelet-derived particles in the range of about 0.3 μm to about 5.0 μm in diameter, about 0.5 μm to about 5.0 μm, (e.g., from about 0.4 μm to about 4.0 μm in diameter, from about 0.5 μm to about 2.5 μm in diameter, from about 0.6 μm to about 2.0 μm in diameter, about 1 μm to about 5.0 μm in diameter, about 1 μm to about 4.0 μm in diameter, about 1.5 μm to about 4.5 μm in diameter, or about 1 μm to about 3.0 μm in diameter).

In some embodiments, a composition comprising frozen platelets and/or platelet derivatives, or cryopreserved platelets and/or platelet derivatives herein upon storing for at least 1 month, 2, 3, 4, 5, 6, 7, 8, 9, 10, 11, or 12 months at a temperature in the range of −10° C. to −40° C., upon thawing can exhibit an ability to generate thrombin in an in vitro thrombin generation assay. A skilled artisan can use any known test(s) to assess thrombin generation. For example, thrombin generation can be assessed by a thrombin generation assay, and the assay can be performed by semi-automated methods for example using a calibrated automated thrombogram, or using fully automated systems. Thrombin generation assay is a type of coagulation test and is based on the potential of plasma to generate thrombin over time, following addition of activators like phospholipids, tissue factor, and calcium. The results of the assay can typically be calculated as a thrombogram, or thrombin generation curve using computer software after calculation of thrombogram parameters. A non-limiting example of assay conditions of a thrombin generation assay include incubating platelets in the presence of tissue factor, and phospholipids. In some embodiments, an in vitro assay comprises incubating platelets and/or platelet derivatives in the presence of tissue factor and phospholipids but in the absence of fresh platelets. Thus, in some embodiments, frozen platelets and/or platelet derivatives as disclosed herein can be capable of generating thrombin, for example, when in the presence of a reagent containing tissue factor and phospholipids in vitro. For example, in some cases, frozen platelets and/or platelet derivatives, or cryopreserved platelets and/or platelet derivatives (e.g., at a concentration of at least about 10×10³ particles/μL, 20×10³ particles/μL, 30×10³ particles/μL, or 44×10³ particles/μL) as described herein can generate a thrombin peak height (TPH) of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM), 50 nM, 75 nM, 100 nM, 150 nM, 175 nM, 200 nM, 250 nM, 275 nM, 300 nM, in illustrative embodiments, when in the presence of a reagent containing tissue factor (e.g., at 0.25 pM, 0.5 pM, 1 pM, 2 pM, 5 pM or 10 pM) and optionally phospholipids. For example, in some cases, frozen platelets and/or platelet derivatives, or cryopreserved platelets and/or platelet derivatives (e.g., at a concentration of at least about 10×10³ particles/μL, 20×10³ particles/μL, 30×10³ particles/μL, or 44×10³ particles/μL) as described herein can generate a TPH of about 100 nM to about 350 nM (e.g., about 125 nM to about 350 nM, or about 150 to about 350 nM), in illustrative embodiments when in the presence of a reagent containing tissue factor and (e.g., at 0.25 pM, 0.5 pM, 1 pM, 2 pM, 5 pM or 10 pM) and optionally phospholipids. In some cases, frozen platelets and/or platelet derivatives, or cryopreserved platelets and/or platelet derivatives (e.g., at a concentration of about 4.8×10³ particles/μL) as described herein can generate a TPH of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of PRP Reagent (cat #TS30.00 from Thrombinoscope), for example, using conditions comprising 20 μL of PRP Reagent and 80 μL of a composition comprising about 4.8×10³ particles/μL of platelets or platelet derivatives, or cryopreserved platelets and/or platelet derivatives. In some cases, frozen platelets and/or platelet derivatives (e.g., at a concentration of about 4.8×10³ particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of PRP Reagent (cat #TS30.00 from Thrombinoscope), for example, using conditions comprising 20 μL of PRP Reagent and 80 μL of a composition comprising about 4.8×10³ particles/μL of frozen platelets and/or platelet derivatives. In some embodiments, a composition herein can have an IU of at least 0.4, 0.5, 0.7/10⁶ particles. As a non-limiting demonstration of the thrombin generation ability, Example 9 and Table 8 demonstrate the thrombin generation ability of the platelets, or platelet derivatives upon storing. A skilled artisan can use other known techniques to assess the thrombin generation potential of the platelets, or platelet derivatives as disclosed herein. Accordingly, in some embodiments, a composition herein comprises platelets, or platelet derivatives that retain hemostatic abilities even upon storing at a temperature in the range of −10° C. to −30° C. for at least 12 months.

In some embodiments, a composition comprising frozen platelets and/or platelet derivatives, or cryopreserved platelets and/or platelet derivatives herein when stored at a temperature in the range of −10° C. to −40° C., in illustrative embodiments, upon storing for at least 1 month, 2, 3, 4, 5, 6, 7, 8, 9, 10, 11, or 12 months, upon thawing can be capable of occluding a collagen-coated microchannel, a tissue factor-coated microchannel, or a collagen- and tissue factor-coated microchannel in vitro. For example, such occluding can be determined, for example, by using a total thrombus-formation analysis system (T-TAS®). In some embodiments, a microchannel is collagen-coated microchannel. In some embodiments, a microchannel is tissue factor-coated microchannel, for example, thromboplastin-coated microchannel. In some embodiments, a microchannel is collagen- and tissue factor-coated microchannel. In some cases, frozen or cryopreserved platelets or platelet derivatives as described herein upon thawing, when at a concentration of at least 50×10³ particles/μL, 60×10³ particles/μL, or 70×10³ particles/μL (e.g., at least 73×10³, 100×10³, 150×10³, 173×10³, 200×10³, 250×10³, or 255×10³ particles/μL) can result in a T-TAS occlusion time (e.g., time to reach kPa of 60) of less than 30, 25, 20, 15, or 14 minutes, or between 5 on the low end of the range, and 15, 20, or 25 on the high end, or between 10 on the low end of the range, and 15, 20, or 25 on the high end, or between 15 on the low end of the range and 20 or 25 on the high end, for example, in platelet-reduced citrated whole blood. In some cases, frozen or cryopreserved platelets or platelet derivatives as described herein upon thawing, when at a concentration of at least 50×10³ particles/μL, 60×10³ particles/μL, or 70×10³ particles/μL (e.g., at least 73×10³, 100×10³, 150×10³, 173×10³, 200×10³, 250×10³, or 255×10³ particles/μL) can result in an area under the curve (AUC) of at least 1300 (e.g., at least 1380, 1400, 1500, 1600, or 1700), for example, in platelet-reduced citrated whole blood. The occlusion time depicts the time it takes the sample to form a thrombus. The lower the time the faster the thrombus formation occurred. The analysis can capture occlusion time (OT) and area under the curve (AUC). OT represents the lag time it takes for the flow pressure to reach a target pressure, such as 60 kPa, 70 kPa, or 80 kPa from the baseline pressure. The AUC is the area under the flow pressure versus time curve which is related to overall thrombus formation. Microchannels or capillaries having different dimensions can be used in a T-TAS system for determining the occlusion times of cryopreserved platelets or cryopreserved platelet derivatives, or frozen platelets or frozen platelet derivatives under different experimental conditions as provided by numerous commercial suppliers (See e.g., Zacros, Tokyo, JP). For example, a T-TAS PL chip, AR chip, or HD chip can be used for an occlusion (e.g., T-TAS) assay, as are commercially available. Typically, an AR chip for the purposes of T-TAS assay is coated with either collagen, or a tissue-factor, such as thromboplastin, or both. Typically an HD chip for the purposes of T-TAS assay is coated with either collagen, or a tissue-factor, such as thromboplastin, or both. For example, the PL chip can have capillary dimensions of 40 μm×40 μm; or an AR chip can have capillary dimensions of 0.3 mm×80 μm; or an HD chip can have capillary dimensions of 0.3 mm×50 μm. Therefore, it is envisioned that a T-TAS assay can be performed to test the ability to occlude a collagen-coated microchannel, utilizing a microchannel or capillary with dimensions in the range of 0.02-0.5, 0.1-0.5, 0.2-0.4, 0.1-0.3, or 0.2-0.3 mm X 25-200, 25-100, 50-100, 40-90, 40-80, or 50-80 μm.

In some embodiments, a composition comprising frozen platelets and/or platelet derivatives herein, when stored at a temperature in the range of −10° C. to −40° C., in illustrative embodiments, upon storing for at least 1 month, 2, 3, 4, 5, 6, 7, 8, 9, 10, 11, or 12 months, upon thawing can exhibit a single peak in a membrane integrity assay typically based upon retention of fluorophore in platelets and/or platelet derivatives. A skilled artisan can contemplate different techniques to study the retention of a fluorophore in particles, such as platelets, and/or platelet derivatives. One such technique is Calcein acetoxymethyl (AM) membrane integrity assay. Calcein AM is a substance that is able to cross the cell membrane and reach the cytosol where Calcein AM gets hydrolyzed by the enzyme esterase to produce fluorescence. Platelets and/or platelet derivatives that are intact are able to retain this fluorescence while non-intact platelets do not. Therefore, based on the fluorescence that is emitted particles can be assessed for their membrane integrity. Accordingly, based on Calcein AM assay, in some embodiments, platelets and/or platelet derivatives in a composition provided herein do not have intact cell membranes, i.e. have compromised membranes. Example 8 is a non-limiting example demonstrating the compromised cell membranes of platelets and/or platelet derivatives in a composition (See FIG. 9). Not to be limited by theory, the observation from FIG. 9 possibly suggests that there are two kinds of population in the single temperature cryopreserved-product (stored at −80° C.), a first population that is able to retain less fluorescence of Calcein AM (See first peak from left of “#” in FIG. 9) possibly because of compromised membrane as compared to a second population (See second peak from left of “#” in FIG. 9) showing higher retention of the fluorescence possibly because they have intact membranes. It was further observed that the single peak of the transition temperature cryopreserved-product (stored at −20° C.) of all the three batches (See “*” in FIG. 9) corresponded to the first population of the single temperature cryopreserved-product that shows less retention of Calcein AM. Therefore, it can be inferred that the single population of the transition temperature cryopreserved-product (stored at −20° C.) observed in the Calcein AM assay comprise platelets and/or platelet derivatives with compromised membranes. However, surprisingly, in spite of the composition having frozen platelets and/or platelet derivatives, the composition, in illustrative embodiments upon storing at a temperature in a range of −10° C. to −40° C., or −20° C.+/−2° C. for at least 2, 4, 6, or 12 months is able to satisfy the criteria of parameters including platelet count (at least 1×10¹¹ platelets per 20-35 ml), typically when counted using hematology analyzer as disclosed herein, pH (more than 6.5), visual observation, such as lack of aggregation and presence of swirling.

Another non-limiting technique for assessing membrane integrity is by detecting lactate dehydrogenase enzyme (LDH) that is released by the cells having a compromised membrane. LDH is a stable cytoplasmic enzyme that is found in all cells. LDH is rapidly released into the cell culture supernatant when the cell membrane is damaged. According to one of the protocols, LDH activity can be easily quantified by using the nicotinamide adenine dinucleotide (NAD)+hydrogen (NADH) produced during the conversion of lactate to pyruvate to reduce a second compound in a coupled reaction into a product with properties that are easily quantitated. This protocol measures the reduction of a yellow tetrazolium salt, Iodonitrotetrazolium (INT), by NADH into a red, water-soluble formazan-class dye by absorbance at 492 nm. The amount of formazan is directly proportional to the amount of LDH in the supernatant, which is, in turn, directly proportional to the number of cells that have compromised membrane. Accordingly, in some embodiments, frozen platelets or platelet derivatives, or cryopreserved platelets or platelet derivatives in a composition herein have compromised membrane as per LDH assay for assessing membrane integrity.

In some embodiments, a composition comprising frozen platelets and/or platelet derivatives, or cryopreserved platelets and/or platelet derivatives herein when stored at a temperature in the range of −10° C. to −40° C., in illustrative embodiments, upon storing for at least 1 month, 2, 3, 4, 5, 6, 7, 8, 9, 10, 11, or 12 months, upon thawing is capable of showing aggregation under aggregation conditions comprising an agonist, not limited to arachidonic acid, collagen, and TRAP-6. In some embodiments, aggregation conditions comprise an agonist but no fresh or apheresis platelets. In some embodiments, aggregation conditions comprise an agonist but no fresh or apheresis platelets, and no divalent cation. Non-limiting examples of aggregation agonists include, collagen, epinephrine, ristocetin, arachidonic acid, adenosine di-phosphate, and thrombin receptor associated protein (TRAP). In some embodiments, frozen platelets and/or platelet derivatives, or cryopreserved platelets and/or platelet derivatives herein upon thawing exhibit aggregation in the presence of arachidonic acid, in the range of 20-60%, 20-50%, or 30-50%, or aggregation of at least 20%, 30%, 40%, or 50%. In some embodiments, frozen platelets and/or platelet derivatives, or cryopreserved platelets and/or platelet derivatives herein upon thawing exhibit aggregation in the presence of collagen, in the range of 2-50%, 2-40%, or 2-30%. In some embodiments, frozen platelets and/or platelet derivatives, or cryopreserved platelets and/or platelet derivatives herein upon thawing exhibit aggregation in the presence of TRAP-6, in the range of 2-50%, 2-40%, or 2-30%. A non-limiting method to determine aggregation is by using PAP8 Aggregometer (Bio Data Corporation, biodatacorp.com). Example 7 and FIG. 8 demonstrate the aggregation of frozen platelets and/or platelet derivatives, or cryopreserved platelets and/or platelet derivatives herein upon thawing in the presence of collagen, TRAP-6, and arachidonic acid.

In some embodiments, a composition comprising frozen platelets and/or platelet derivatives, or cryopreserved platelets and/or platelet derivatives herein when stored at a temperature in the range of −10° C. to −40° C., in illustrative embodiments, upon storing for at least 1 month, 2, 3, 4, 5, 6, 7, 8, 9, 10, 11, or 12 months, upon thawing to form thawed platelets, and in some embodiments, upon thawing and storing at a temperature in the range of 4° C. to 45° C., 10° C. to 45° C., 15° C. to 40° C., 20° C. to 35° C., or 20° C. to 30° C., or at room temperature for at least 5, 6, 7, 8, 10, 12, 14, 18, 20, or 24 hours, the thawed platelets are capable of displaying cell-surface markers that are used to identify platelets, and are known to be displayed on fresh platelets, stored platelets, such as liquid stored platelets, and typically capable of exhibiting stability or functional stability as per the parameters disclosed elsewhere herein. Non-limiting examples of cell-surface markers include CD41 (also called glycoprotein IIb or GPIIb, which can be assayed using e.g., an anti-CD41 antibody), CD42 (which can be assayed using, e.g., an anti-CD42 antibody), CD62 (also called CD62P or P-selectin, which can be assayed using, e.g., an anti-CD62 antibody), phosphatidylserine (which can be assayed using, e.g., annexin V (AV)), and CD47 (which is used in self-recognition; absence of this marker, in some cases, can lead to phagocytosis). For example, the cryopreserved platelets herein upon thawing are capable of displaying one or more of markers selected from CD41 (GPIIb), CD42a (GPIX), CD42b (GPiba), and CD61 (GPIIIa). In some embodiments, the cryopreserved platelets herein upon thawing are capable of displaying CD62P (P-selectin). In some embodiments, the cryopreserved platelets herein upon thawing are capable of displaying phosphatidyl serine (PS), such that the cryopreserved platelets herein upon thawing are positive for detecting PS, and typically, the detecting is done using Annexin V, accordingly, in some embodiments, the cryopreserved platelets herein upon thawing are capable of binding Annexin V. In some embodiments, the cryopreserved platelets herein upon thawing are capable of displaying positivity for lactadherin.

Accordingly, cryopreserved platelets herein upon thawing, and storing as described herein can have cell surface markers. The presence of cell surface markers can be determined using any appropriate method. In some embodiments, the presence of cell surface markers can be determined using binding proteins (e.g., antibodies) specific for one or more cell surface markers and flow cytometry (e.g., as a percent positivity, e.g., using approximately 2.7×10⁵ platelets or platelet derivatives/μL; and about 4.8 μL of an anti-CD41 antibody, about 3.3 μL of an anti-CD42 antibody, about 1.3 μL of annexin V, or about 2.4 μL of an anti-CD62 antibody). The percent positivity of any cell-surface marker can be shown in terms of any appropriate percent positivity. For example, cryopreserved platelets herein, upon thawing and storing as disclosed herein can have an average CD41 percent positivity of at least 30%, 35%, 40%, 45%, 50%, or 55% (e.g., at least 60%, at least 65%, at least 67%, at least 70%, at least 75%, at least 80%, at least 85%, at least 90%, or at least 95%). In some embodiments, cryopreserved platelets herein, upon thawing and storing as disclosed herein can have an average CD41 percent positivity in the range of 70%-99%, 70%-95%, 70%-90%, 70%-86%, or 75%-86%. In some embodiments, at least 30%, 35%, 40%, 45%, 50%, or 55% (e.g., at least 60%, at least 65%, at least 67%, at least 70%, at least 75%, at least 80%, at least 85%, at least 90%, or at least 95%) of cryopreserved platelets, upon thawing and storing that are positive for CD 41 have a size (for example, diameter) in the range of 0.7-2.5 μm, 0.5-2.5 μm, 0.4-2.8 μm, or 0.3-3 μm.

As another example, cryopreserved platelets herein, upon thawing and storing as disclosed herein, can have an average CD42 percent positivity of at least 30%, 35%, 40%, 45%, 50%, 55%, 60%, or 65% (e.g., at least 67%, at least 70%, at least 75%, at least 80%, at least 85%, at least 90%, or at least 95%). In some embodiments, cryopreserved platelets herein, upon thawing and storing as disclosed herein that are positive for CD42 have a size (for example, diameter) in the range of 0.7-2.5 μm, 0.6-2.5 μm, 0.5-2.5 μm, 0.4-2.8 μm, or 0.3-3 μm. In some cases, the presence of CD42 can be detected using a mean fluorescence intensity (MFI), in such cases, the MFI of CD42 in the composition comprising cryopreserved platelets herein can be at least 10,000, 15,000, or 20,000. In some cases, the composition comprising cryopreserved platelets herein can have MFI of CD42 in the range of 10,000 to 30,000, 15,000 to 30,000, or 12,000 to 28,000.

As another example, cryopreserved platelets herein, upon thawing and storing as disclosed herein, can have an average CD62 percent positivity of at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 83%, at least 84%, at least 85%, at least 90%, or at least 95%). In some embodiments, cryopreserved platelets herein, upon thawing and storing as disclosed herein that are positive for CD62 have a size (for example, diameter) in the range of 0.7-2.5 μm, 0.5-2.5 μm, 0.4-2.8 μm, or 0.3-3 μm. In some cases, the presence of CD62 or CD62P can be detected using a mean fluorescence intensity (MFI), in such cases, the MFI of CD62 in the composition comprising cryopreserved platelets herein can be at least 2,000, 4,000, or 5,000. In some cases, the composition comprising cryopreserved platelets herein can have MFI of CD62 in the range of 2,000 to 10,000, 4,000 to 12,000, or 4,000 to 10,000. In some cases, cryopreserved platelets herein, upon thawing and storing as disclosed herein exhibit at least 1.5, 2, 3, or 5 fold higher presence of CD62 as compared to the platelets, such as, fresh platelets, liquid stored platelets, or apheresis platelets.

As yet another example, cryopreserved platelets herein, upon thawing and storing as disclosed herein, can have an average positivity for phosphatidyl serine (PS), for example tested using Annexin V, of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, cryopreserved platelets herein, upon thawing and storing as disclosed herein that are positive for PS, for example, when tested using Annexin V for the binding of Annexin V to the platelets, have a size in the range of 0.5-2.5 μm, 0.4-2.8 μm, or 0.3-3 μm. In some embodiments, the presence of phosphatidyl serine in/on the cryopreserved platelets herein, upon thawing and storing as disclosed herein is higher than the presence of phosphatidyl serine in/on the platelets, such as, fresh platelets, liquid stored platelets, or apheresis platelets. For example, cryopreserved platelets herein, upon thawing and storing as disclosed herein exhibit at least 5 fold, 10 fold, 20 fold, 25 fold, 30 fold, 40 fold, or 50 fold higher presence of phosphatidyl serine as compared to the platelets, such as, fresh platelets, liquid stored platelets, or apheresis platelets. In some embodiments, cryopreserved platelets herein, upon thawing and storing as disclosed herein have an average positivity for phosphatidyl serine (PS) in the range of 50-95%, 50-90%, 50-85%, 55-95%, 55-90%, 55-85%, 60-95%, 60-90%, 60-88%, or 60-85%. In some cases, the presence of PS can be detected using a mean fluorescence intensity (MFI), in such cases, the MFI of PS, when measured using Lactadherin in the composition comprising cryopreserved platelets herein can be at least 2,000, 4,000, 5,000, 7,000, or 8,000. In some cases, the composition comprising cryopreserved platelets herein can have MFI of PS in the range of 2,000 to 10,000, 4,000 to 12,000, or 4,000 to 10,000.

As yet another example, cryopreserved platelets herein, upon thawing and storing as disclosed herein, can have an average positivity for P-selectin, of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, cryopreserved platelets herein, upon thawing and storing as disclosed herein that are positive for P-selectin, have a size in the range of 0.7-2.5 μm, 0.5-2.5 μm, 0.4-2.8 μm, or 0.3-3 μm. In some embodiments, cryopreserved platelets herein, upon thawing and storing as disclosed herein have an average positivity for P-selectin in the range of 30-85%, 30-80%, 30-75%, 30-70%, 35-85%, 35-80%, 35-75%, or 35-70%.

As yet another example, cryopreserved platelets herein, upon thawing and storing as disclosed herein, can have an average positivity for PS, when measured using lactadherin binding, of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, cryopreserved platelets herein, upon thawing and storing as disclosed herein that are positive for PS, when measured using lactadherin binding, have a size in the range of 0.7-2.5 μm, 0.5-2.5 μm, 0.4-2.8 μm, or 0.3-3 μm. In some embodiments, cryopreserved platelets herein, upon thawing and storing as disclosed herein have an average positivity for PS, when measured using lactadherin in the range of 50-95%, 50-90%, 50-85%, 55-95%, 55-90%, 55-85%, 60-95%, 60-90%, 60-88%, or 60-85%.

As yet another example, cryopreserved platelets herein, for example, upon thawing and storing as disclosed herein, can have an average positivity for fibrinogen, of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, cryopreserved platelets herein, for example, upon thawing and storing as disclosed herein that are positive for fibrinogen, have a size in the range of 0.7-2.5 μm, 0.5-2.5 μm, 0.4-2.8 μm, or 0.3-3 μm. In some embodiments, cryopreserved platelets herein, for example, upon thawing and storing as disclosed herein have an average positivity for fibrinogen, in the range of 50-95%, 50-90%, 50-85%, 55-95%, 55-90%, 55-85%, 60-95%, 60-90%, 60-88%, or 60-85%. In some cases, the presence of fibrinogen can be detected using a mean fluorescence intensity (MFI), in such cases, the MFI of fibrinogen in the composition comprising cryopreserved platelets herein can be at least 2,000, 4,000, 5,000, 7,000, or 8,000. In some cases, the composition comprising cryopreserved platelets herein can have MFI of fibrinogen in the range of 2,000 to 10,000, 4,000 to 12,000, or 4,000 to 10,000. In some cases, cryopreserved platelets herein, for example, upon thawing and storing as disclosed herein exhibit at least 1.5, 2, 3, 5, 6, 7, 8, or 10 fold higher presence of fibrinogen as compared to the platelets, such as, fresh platelets, liquid stored platelets, or apheresis platelets.

As yet another example, cryopreserved platelets herein, for example, upon thawing and storing as disclosed herein, can have an average positivity for von Willebrand factor (vWF), of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, cryopreserved platelets herein, for example, upon thawing and storing as disclosed herein that are positive for vWF, have a size in the range of 0.7-2.5 μm, 0.5-2.5 μm, 0.4-2.8 μm, or 0.3-3 μm. In some embodiments, cryopreserved platelets herein, for example, upon thawing and storing as disclosed herein have an average positivity for vWF, in the range of 50-95%, 50-90%, 50-85%, 55-95%, 55-90%, 55-85%, 60-95%, 60-90%, 60-88%, or 60-85%. In some cases, the presence of vWF can be detected using a mean fluorescence intensity (MFI), in such cases, the MFI of vWF in the composition comprising cryopreserved platelets herein can be at least 2,000, 4,000, 5,000, 7,000, or 8,000. In some cases, the composition comprising cryopreserved platelets herein can have MFI of vWF in the range of 2,000 to 10,000, 4,000 to 12,000, or 4,000 to 15,000. In some cases, cryopreserved platelets herein, for example, upon thawing and storing as disclosed herein exhibit at least 1.5, 2, 3, 5, 6, 7, 8, or 10 fold higher presence of vWF as compared to the platelets, such as, fresh platelets, liquid stored platelets, or apheresis platelets.

As yet another example, cryopreserved platelets herein, for example, upon thawing and storing as disclosed herein, can have an average positivity for thrombospondin (TSP), of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, cryopreserved platelets herein, for example, upon thawing and storing as disclosed herein that are positive for TSP, have a size in the range of 0.7-2.5 μm, 0.5-2.5 μm, 0.4-2.8 μm, or 0.3-3 μm. In some cases, the presence of TSP can be detected using a mean fluorescence intensity (MFI), in such cases, the MFI of TSP in the composition comprising cryopreserved platelets herein can be at least 2,000, 4,000, 5,000, 7,000, or 8,000.

As yet another example, cryopreserved platelets herein, for example, upon thawing and storing as disclosed herein, can have an average positivity for CD49 (GPIaIIa), of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, cryopreserved platelets herein, for example, upon thawing and storing as disclosed herein that are positive for CD49, have a size in the range of 0.7-2.5 μm, 0.5-2.5 μm, 0.4-2.8 μm, or 0.3-3 μm. In some cases, the presence of CD49 can be detected using a mean fluorescence intensity (MFI), in such cases, the MFI of CD49 in the composition comprising cryopreserved platelets herein can be at least 2,000, 4,000, 5,000, 7,000, or 8,000.

As yet another example, cryopreserved platelets herein, for example, upon thawing and storing as disclosed herein, can have an average positivity for GPVI, of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, cryopreserved platelets herein, for example, upon thawing and storing as disclosed herein that are positive for GPVI, have a size in the range of 0.7-2.5 μm, 0.5-2.5 μm, 0.4-2.8 μm, or 0.3-3 μm. In some cases, the presence of GPVI can be detected using a mean fluorescence intensity (MFI), in such cases, the MFI of GPVI in the composition comprising cryopreserved platelets herein can be at least 2,000, 4,000, 5,000, 7,000, or 8,000.

As yet another example, cryopreserved platelets herein, for example, upon thawing and storing as disclosed herein, can have an average positivity for CD63, of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, cryopreserved platelets herein, for example, upon thawing and storing as disclosed herein that are positive for CD63, have a size in the range of 0.7-2.5 μm, 0.5-2.5 μm, 0.4-2.8 μm, or 0.3-3 μm. In some cases, the presence of CD63 can be detected using a mean fluorescence intensity (MFI), in such cases, the MFI of CD63 in the composition comprising cryopreserved platelets herein can be at least 2,000, 4,000, 5,000, 7,000, or 8,000.

In some embodiments, cryopreserved platelets, cryopreserved platelet compositions, and/or frozen activated platelets as disclosed herein, including, but not limited to those made using a process disclosed herein, can comprise a population of platelet particles that, upon thawing, can be capable of further activation when stimulated. In some embodiments, the stimulation can be performed in vitro. The population can include, in some embodiments, platelet particles that exhibit activation upon thawing, without stimulation. In such embodiments, the population can include platelet particles that can be further activated upon stimulation. The stimulation can be performed, in some embodiments, with one or more platelet agonists. In non-limiting examples, the platelet agonists can be one or more selected from TRAP-6, ADP, epinephrine (Epi), and/or any platelet agonist(s) disclosed herein. In some embodiments, the stimulation can cause a conformational change in one or more platelet cell surface markers, into an activated conformation, such as for example, CD41 (GPIIb).

In some embodiments, the further activation can be detected by detecting a platelet cell surface marker and/or an activated conformation of a platelet cell surface marker. In some embodiments, further activation can be detected by any method disclosed herein. In illustrative embodiments, the activated conformation of a platelet cell surface marker can be an activated conformation of GPIIb/IIIa. In non-limiting examples, the platelet cell surface marker, can be one or more selected from CD41 (GPIIb), CD42a (GPIX), CD42b (GPibα), CD61 (GPIIIa), and/or any platelet cell surface markers disclosed herein. In some embodiments, the detection of a surface marker and/or an activated conformation of a platelet cell surface marker can be detected by the binding of an antibody. The antibody can be virtually any antibody known to bind a platelet cell surface marker, or in illustrative embodiments, preferably or exclusively the activated form of a platelet cell surface marker. In illustrative embodiments, the antibody is the PAC-1 monoclonal antibody, which is known to preferentially bind to the activated form of GPIIb/IIIa.

In some embodiments, cryopreserved platelets, cryopreserved platelet compositions, and/or frozen activated platelets as disclosed herein, including, but not limited to those made using a process disclosed herein, can comprise at least two sub-populations of platelet particles based at least on the activated status of the platelet particles. For example, the cryopreserved platelets, cryopreserved platelet compositions, and/or frozen activated platelets comprising a population of platelet particles shows two sub-populations of platelet particles, a more activated sub-population, and a less activated sub-population upon gating a population of platelet-sized particles using a fluorescently-labeled antibody or fluorescently-tagged protein that recognizes a platelet-specific marker, such as CD41, and analyzing the population of platelet-sized particles obtained in the gating using a fluorescently-labeled antibody or fluorescently-tagged protein specific for at least one of: phosphatidylserine (PS), CD62P, activated GPIIb/IIIa, and P-selectin, and using a fluorescently-labeled antibody or fluorescently-tagged protein specific for a platelet-specific marker, for example, CD42b. In some cases, the analyzing is done by using fluorescently-tagged protein specific for PS, for example, fluorescently-tagged lactadherin specific for PS. It will be understood that any platelet cell surface marker, such as platelet-specific marker, or activated form thereof, or platelet-activation marker that is detected, and/or whose levels are measured using an antibody in any of the aspects and embodiments herein, can be detected and measured using other binding agents as well, that specifically bind the cell surface marker or its activated form, such as another type of protein or a peptide. In some cases, the gating can also include forward scatter height (FSC-H) analysis.

In some cases, the cryopreserved platelets comprising a population of platelet particles, upon thawing comprise at least two sub-populations based on the level of activation when measured for the presence of and/or quantified for at least one marker selected from the group consisting of phosphatidylserine, P-Selectin, CD62, and activated GPIIb/IIIa complex. In some cases, the level of activation is measured based on the presence of at least one marker selected from the group consisting of phosphatidylserine, P-Selectin, CD62, and activated GPIIb/IIIa complex. In some cases, the level of activation is measured based on the presence of phosphatidylserine in a flow cytometry assay, such that the population of platelet particles, upon thawing comprise two sub-populations, and the presence of phosphatidylserine can be measured by detecting binding of lactadherin to the platelet particles. In some cases, the two sub-populations comprise a first sub-population comprising platelet particles positive for phosphatidylserine, and a second sub-population comprising platelet particles negative for phosphatidylserine, for example, the first sub-population comprising platelet particles positive for phosphatidylserine is a more activated sub-population, and the second sub-population comprising platelet particles negative for phosphatidylserine is a less activated sub-population. In some cases, the level of activation is measured based on the quantification of at least one marker selected from the group consisting of phosphatidylserine, P-Selectin, CD62, and activated GPIIb/IIIa complex. For example, the level of activation is measured based on the quantification of phosphatidylserine in a flow cytometry assay, such that the population of platelet particles, upon thawing comprise two sub-populations, and wherein the phosphatidylserine is quantified by measuring the binding of lactadherin to the platelet particles. In some cases, the two sub-populations comprise a first sub-population comprising platelet particles exhibiting a mean fluorescence intensity (MFI) of lactadherin of at least 50,000, 60,000, or 80,000 when measured using a fluorescently-labeled antibody or fluorescently-tagged protein capable of binding to lactadherin, and a second sub-population comprising platelet particles exhibiting a mean fluorescence intensity (MFI) of phosphatidylserine of less than 30,000, 20,000, 10,000, or 5,000 when measured using a fluorescently-labeled antibody or fluorescently-tagged protein against capable of binding to lactadherin. In some cases, the first sub-population comprising platelet particles exhibiting a mean fluorescence intensity (MFI) of lactadherin of at least 50,000 is a more activated sub-population, and the second sub-population comprising platelet particles exhibiting a mean fluorescence intensity (MFI) of lactadherin of less than 30,000 is a less activated sub-population.

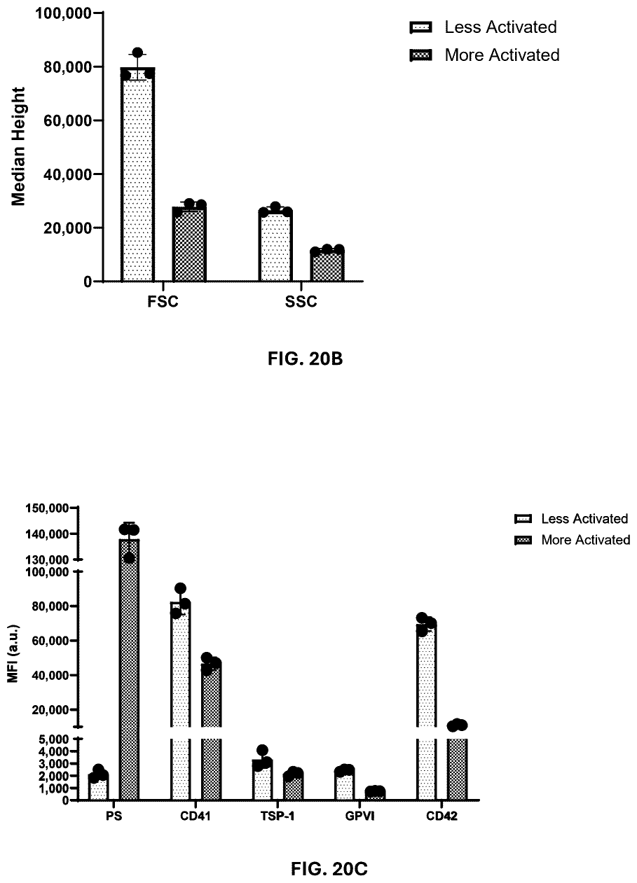

In some cases, the cryopreserved platelets or pooled CPP product herein upon thawing form thawed platelet particles such that there can be two sub-populations: a more activated sub-population; and a less activated sub-population in the thawed platelet particles, and the two sub-populations can vary in their percentages in the thawed platelet particles obtained upon thawing the cryopreserved platelets or the pooled CPP product herein. For example, the more activated sub-population can form at least 45%, 47%, 50%, 52%, 54%, or 56% of the thawed platelet particles, in some embodiments, the more activated sub-population can be in the range of 45-70%, 45-65%, 45-60%, 47-70%, 47-65%, 47-60%, 50-70%, 50-60%, 51-70%, 51-65%, 52-70%, 52-65%, 52-62%, 54-70%, or 55-70% of the thawed platelet particles. In some embodiments, the less activated sub-population can form at least 20%, 25%, 30%, or 35% of the thawed platelet particles, in some embodiments, the less activated sub-population can be in the range of 20-50%, 20-49%, 20-47%, 20-45%, 20-42%, 25-47%, 30-47%, 35-47%, or 35-45% of the thawed platelet particles. In some cases, the percentage of the less activated sub-population is lower as compared to that of the more activated sub-population in the thawed platelet particles. In some cases, the percentages of sub-populations can be measured by flow cytometry, for example, by gating the particles obtained upon thawing the cryopreserved platelets or pooled CPP product herein using fluorescently-tagged protein specific for platelet-specific marker, such as CD41 to obtain thawed platelet particles, and analyzing the thawed platelet particles thus obtained using a fluorescently-tagged protein specific for a platelet activation marker such as phosphatidylserine (PS), and a fluorescently-tagged protein specific for a platelet-specific marker such as CD42b. For example, FIG. 20A demonstrates the flow cytometry analysis of cryopreserved platelets or pooled CPP product herein and was observed that about 56.48% of the thawed platelet particles was the more activated sub-population, and about 40.18% was the less activated sub-population. The fluorescently-tagged protein for the gating step was a fluorescently-tagged antibody specific for CD41, and the analyzing step included a fluorescently-tagged antibody specific for CD42b (PE-Cy5H), and a fluorescently-tagged protein: lactadherin specific for PS (FITC-H).

In some embodiments, a composition comprising frozen platelets and/or platelet derivatives, or cryopreserved platelets, or pooled CPP product herein upon thawing, in illustrative embodiments, upon thawing and diluting have the property of exhibiting adhesion to collagen, for example, in an in vitro assay. In illustrative embodiments, the adhesion exhibited is a specific adhesion, such that the cryopreserved platelets upon thawing is not capable of adhering to albumin under the similar or same conditions as that of the adhesion to collagen. In some cases, the specific adhesion can also be exhibited by the inability of the cryopreserved platelets upon thawing to adhere to uncoated channels. In illustrative embodiments, the cryopreserved platelets upon thawing have the property of exhibiting adhesion to collagen in the absence of external platelets, for example, platelets from any external source apart from thawed platelets present in a thawed platelet composition obtained upon thawing the cryopreserved platelets herein. In some cases, the external platelets can be endogenous platelets, liquid stored platelets, room temperature platelets, apheresis platelets, or cold stored platelets. In some cases, the pooled CPP product herein can be stored at a temperature equal to or less than −65° C., −70° C., or −75° C. for at least 6 months, 1, 2, 3, or 4 years. In some embodiments, the cryopreserved platelets herein when assessed after being stored at a temperature equal to or less than −65° C., −70° C., or −75° C. for at least 6 months, 1, 2, 3, or 4 years, for example, 1 month to 6 years, 1 month to 5 years exhibits the property of adhering to collagen. In some embodiments, upon thawing and diluting the cryopreserved platelets to form thawed platelet particles, the thawed platelet particles are capable of adhering to collagen, for example, in an in vitro assay. In some cases, the adhering to collagen can be determined in the presence of plasma. In some cases, the cryopreserved platelets upon thawing, for example, thawing and diluting can be mixed with plasma for determining the adhesion to collagen. In some cases, the cryopreserved platelets upon thawing, for example, thawing and diluting can be mixed with Octaplas. Typically, Octaplas comprises a solvent/detergent treated, pooled human plasma. In some cases, the adhesion to collagen can be assessed in the presence of solvent/detergent treated, pooled plasma, for example, human plasma. In some cases, a thawed platelet composition is diluted with plasma for performing the in vitro assay, for example, a solvent/detergent pooled human plasma. In some cases, a thawed platelet composition is diluted with Octaplas. In some cases, the adhesion can be assessed in the presence of plasma and one or more of: a buffer, salt and an amino acid. In some cases, the adhesion can be assessed in the presence of plasma that comprise 0.01-0.1 g/ml, 0.01-0.08 g/ml, or 0.04-0.08 g/ml plasma proteins, for example, human plasma proteins. In some cases, the buffer and the salt can comprise one or more of sodium citrate dihydrate, and sodium dihydrogen-phosphate dihydrate. In some cases, the amino acid can be glycine. In some cases, the in vitro assay can comprise diluting a thawed platelet composition with plasma comprising 0.01-0.08 g/ml plasma proteins, and one or more of sodium citrate dihydrate, sodium dihydrogen-phosphate dihydrate, and glycine. In some cases, the in vitro assay can comprise labeling a thawed platelet composition to form a labeled platelet composition, and contacting the labeled platelet composition to a collagen-coated channel, followed by acquiring an image of the collagen-coated channel to determine the adhesion. In some cases, the adhesion can be determined by measuring an area coverage of the thawed platelets, and in some cases, the adhesion can be determined by measuring the rate of adhesion, for example, rate of adhesion/sec, or rate of adhesion/minute. In some cases, the area coverage exhibited by thawed platelets herein can be equal to or greater than 20%, 50%, or 75% of the area coverage exhibited by platelets in a platelet-rich plasma. In some cases, the area coverage exhibited by thawed platelets herein can be equal to or greater than the area coverage exhibited by platelets in a platelet-rich plasma. In some cases, the rate of adhesion to collagen exhibited by thawed platelets herein can be equal to or greater than 20%, 50%, or 75% of the rate of adhesion to collagen exhibited by platelets in a platelet-rich plasma. In some cases, the rate of adhesion to collagen exhibited by thawed platelets herein can be equal to or greater than the rate of adhesion to collagen exhibited by platelets in a platelet-rich plasma. In some cases, the in vitro assay is a BioFlux assay and can be used for determining the adhesion of the thawed platelet particles to collagen. In some cases, the thawing and diluting can be done as disclosed elsewhere in this specification. In illustrative embodiments, the thawing can be done by placing a cryo-vessel comprising the cryopreserved platelets in a water bath set at a temperature of 37° C.+/−2° C. until the frozen activated platelets are thawed to form a thawed composition or a thawed activated platelet composition. In illustrative embodiments, the diluting is done by diluting a thawed platelet composition with 15-35 ml of saline, such as 0.9% saline, in some cases, about 22-27 ml of saline. In some cases, the in vitro assay for determining the adhesion can comprise contacting a thawed activated platelet composition after the diluting, to a collagen-coated channel under a shear flow, and acquiring an image of the collagen-coated channel to determine the adhering. Typically, the cryopreserved platelets herein have the property of adhering specifically to collagen, but not to albumin, for example, when tested using an in vitro assay that can comprise contacting a thawed activated platelet composition after the diluting, to an albumin-coated channel under a shear flow, and acquiring an image of the albumin-coated channel to determine the adhering. Accordingly, in some cases, the cryopreserved platelets herein have a property to exhibit a specific binding to collagen, for example, the cryopreserved platelets herein have the property of binding to collagen but not binding to albumin under the same in vitro conditions. In some cases, the cryopreserved platelets as disclosed herein have the property such that upon thawing, thawed platelets or thawed platelet particles do not bind or adhere to uncoated surfaces, for example, not coated with collagen, of a channel in an in vitro assay as disclosed herein, but is capable of adhering to collagen-coated sections of a channel. In some cases, the contacting is done under a shear flow such that contacting the thawed activated platelet composition after the diluting, to the collagen-coated channel at a pressure in the range of 10-40 dyn/cm². In some cases, the contacting is done at a pressure in the range of 5-50, 10-40, or 15-35 dyn/cm². In some cases, the contacting is done at a pressure of about 20, 22, 25, 27, 28, 29, or 30 dyn/cm². In some cases, the adhesion of the cryopreserved platelets, for example, pooled CPP product upon thawing and diluting to collagen can be assessed by: contacting a thawed platelet composition, to a collagen-coated channel under a shear flow, and acquiring an image of the collagen-coated channel. In some cases, the adhesion can be determined by assessing area coverage of the thawed platelets from the image acquired during the assay. In some cases, a thawed platelet product can exhibit an area coverage of at least 500 μm² after contacting for 2, 3, 4, 5, 6, or 7 minutes. In some cases, a thawed platelet product can exhibit an area coverage of at least 1000 μm² after contacting for 7 minutes. In some cases, a thawed platelet product can exhibit an area coverage in the range of at least 700-25,000 μm², 700-23,000 μm², 700-21,000 μm², or 1,000-21,000 μm² after contacting for 7 minutes. In some cases, the area coverage disclosed herein can be obtained when the thawed platelet product comprises platelets or platelet particles in the range of 2×10⁵/μl to 6×10⁵/μl, or 2.5×10⁵/μl to 5.5×10⁵/l. For example, in some cases, cryopreserved platelet product, for example, a pooled CPP product herein upon thawing and diluting have the property of adhering to collagen in an in vitro assay in the presence of plasma, for example, when the concentration of platelets or platelet products in a thawed platelet product in the range of 2×10⁵/μl to 6×10⁵/μl, or 2.5×10⁵/μl to 5.5×10⁵/μl, and the thawed platelet product exhibits an area coverage in the range of 700-25,000 μm², 700-23,000 μm², 700-21,000 μm², or 1,000-21,000 μm² after contacting for 5, 6, or 7 minutes. In some cases, the channels used in the in vitro assays can be microfluidic flow channels built into the plates that are compatible with an instrument that can read the fluorescent signals to determine an adhesion. In some cases, the channels can be made up of a rigid polymer like polystyrene, or can also be made up of glass. In some cases, the channels can have a height in the range of 40-90 μm, width of 200-500 μm, and a length of about 1-4 cm.

### Pooling and Concentrating of Platelet Units

Processes for preparing a batch of cryopreserved platelets provided herein typically include platelet units as a starting material or source of platelets. Typically, the platelet units can be apheresis platelet units (APU). However, other sources of platelets can be used. The alternative sources of platelets can include whole blood-derived platelets. Platelets can be obtained from whole blood either using the known platelet-rich-plasma (PRP) method or the buffy-coat method. The platelets obtained from the buffy-coat method are known as buffy coat-derived platelet concentrates (BC-PC). A skilled artisan can use platelets from any of the sources available based on the ease of availability and from a commercial standpoint. In some cases, platelets, or platelet compositions that are used as a starting material can be present in a platelet additive solution (PAS). For example, PAS can replace plasma from a platelet composition such that the plasma protein content can be reduced by at least 50%, 60%, 70%, 75%, 80%, 90%, or 95% in the platelet composition that can be used as a starting material in a process for preparing cryopreserved platelets as disclosed herein. In some cases, the PAS completely replaces the plasma content in a platelet composition such that there are no detectable levels of plasma protein in the platelet composition that can be used as a starting material.

The platelet units, such as APUs can be accessed from any recognized blood banks or centers that process blood units. The process can be performed at a tertiary care facility that has access to the blood banks. The process can be performed at any facility or a processing center that has access to the blood banks, or the platelet units are supplied to the facility or the processing center. Typically, platelets are available in two forms: pools of whole blood-derived platelet concentrates, and platelets collected via apheresis. Platelet concentrates are prepared from donated whole blood, separated within eight hours of collection, and contain a minimum of 5.5×101 platelets in 1 unit, and individual platelet concentrate units contain about 40 to 50 ml of plasma. Apheresis platelets are collected from a single donor and contain a minimum of 3×10¹¹ platelets in 1 unit suspended in 200 to 300 mL of plasma.

Typically, the platelet units provided herein are obtained from more than 1 donor (See step 110 of FIG. 1B). For example, the platelet units can be provided from 2, 3, 4, 5, 6, 7, 8, 9, 10, 11, 12, or more donors. In some cases, the platelet units can be provided or pooled from more than 12 donors, such as 13, 14, 15, 16, 17, 18, 19, 20, or more. In some cases, the platelet units can be provided from 20 to 100, 30 to 100, 40 to 100, or 50 to 100 donors. In some cases, 1, 2, 3, or 4 platelet units, for example, apheresis platelet units (APU) can be provided from one donor. The number of APUs that can be provided from one donor can depend upon the weight and total blood volume of a donor.

In some cases, pooling of platelets, such as APUs or buffy-coat platelets can be based on the blood group of the donors, for example, pooling of platelets can be performed from the donors who have Group O blood group. In some cases, pooling of platelets, such as APUs or buffy-coat platelets can be based on the blood group of the donors, for example, pooling of platelets can be performed from the donors who have Group A blood group. In some cases, pooling of platelets, such as APUs or buffy-coat platelets can be based on the blood group of the donors, for example, pooling of platelets can be performed from the donors who have Group B blood group. In some cases, pooling of platelets, such as APUs or buffy-coat platelets can be based on the blood group of the donors, for example, pooling of platelets can be performed from the donors who have Group AB blood group. In some cases, pooling of platelets, such as APUs or buffy-coat platelets can be based on the blood group of the donors, for example, pooling of platelets can be performed from the donors having any of the blood groups, A, B, AB, or O. In some cases, the platelets like in APUs or buffy coats are irradiated.

In some aspects, a process for preparing a batch of cryopreserved platelets herein can include pooling of the platelet units based on the HLA characterization of donor platelets, typically, the characterization can be based on HLA Class I antigens, including but not limited to HLA-A, HLA-B, and HLA-C. These antigens are expressed on the surface of platelets and can elicit immune responses in allo-immunized recipients. In some cases, the HLA characterization can be done based on HLA Class II antigens, in addition to HLA Class I antigens. Accordingly, characterizing platelet products based on HLA Class I antigens, and/or HLA Class II antigens and enabling formation of a pool of platelets based on HLA-compatibility, or HLA-matching can be advantageous in mitigating transfusion-related immune reactions, such as in subjects those have undergone multiple blood transfusions and can be refractory to further platelet transfusions. HLA Class I molecules are polymorphic and their immunogenic regions, or epitopes, are further defined using eplets, which represent three-dimensional clusters of polymorphic residues accessible to alloantibodies. These eplets serve as the immunologic targets in alloimmunization. Public and private epitopes associated with HLA Class I antigens can be grouped into Cross-Reactive Groups (CREGs) based on shared antigenic determinants, which allow for broader compatibility assessments when exact antigen matches are unavailable. In contrast, HLA Class II antigens, although not typically expressed on platelets, may be present as contaminants from residual leukocytes and are considered in some embodiments for assessing HLA-compatibility in pooling of platelet units from multiple donors.

Accordingly, in one aspect, provided herein is a process for preparing a batch of cryopreserved platelets, a collection of cryo-vessels comprising cryopreserved platelets, or a composition comprising frozen platelets, wherein the cryopreserved platelets in the batch, the cryopreserved platelets in the collection of cryo-vessels, or the frozen platelets are HLA Class 1-characterized, cryopreserved platelets or frozen platelets. In some cases, the HLA Class 1 type of a plurality of platelet donors can be determined for a plurality of platelet samples (e.g., donor apheresis platelets, or buffy-coat derived platelet samples). Such determination can be performed for example at a blood collection center, a facility storing collected blood, or at a site at which the cryopreserved platelets disclosed herein will be prepared from at least some of the donor platelets. Methods are known in the art, some of which are provided herein, for determining the HLA Class 1 characteristics, typically the HLA Class 1 type and/or antigens of platelets, using platelets or blood or a blood fraction from which the platelets were isolated, or from another tissue of a donor. Thus, in certain non-limiting embodiments the platelets from individual donors considered for pooling are HLA-typed (e.g. HLA Class 1 antigen types are determined) to identify the HLAs present on the surface. In some embodiments, the entity performing the process for preparing the cryopreserved platelets herein does not actual perform the HLA Class 1 determination, but rather receives this information, for example via a computer network, such as for example the Internet. Next, the HLA Class 1 characteristics (e.g., type and/or antigen information) regarding the plurality of platelet samples (e.g., apheresis platelets, or buffy-coat derived platelet samples) from a plurality of platelet donors is used to select platelets from a subset of the plurality of platelet donors to include in a pool of platelets. HLA Class 1 characteristics (e.g., types and/or antigens) of the plurality of donors can be used in various ways to select platelet donors whose platelets are combined to form the pool as discussed herein. The process is carried out such that platelets from subsets of donors that are selected based on their HLA Class 1 characteristics are pooled to form a plurality of pools of HLA-characterized platelets. The HLA Class 1 characteristics (e.g., HLA Class 1 types or antigens) for some, most, or in illustrative embodiments all of the pools of the plurality of pools are different from each other.

In some illustrative embodiments of any of the methods herein that include a pooling step, or that include pooled platelets, platelets are pooled from platelet donors having HLA-compatible, in illustrative embodiments, HLA matched, in further illustrative embodiments HLA Class 1-matched platelets using any HLA matching strategies and/or criteria known in the art, as non-limiting examples, any of the matching strategies and/or criteria provided herein to form HLA Class 1-matched FDPDs or HLA Class 1-matched platelet derivatives. For example, platelets can be pooled from donors having cross-reactive antigens falling within the same cross-reactive group (CREG). Alternatively, platelets can be pooled from platelet donors that have HLA Class 1 antigen-matched platelets to a grade A, B1U, B1X, B2U, B2UX, B2X, C, or D match to form HLA Class 1 antigen-matched FDPDs or HLA Class 1 antigen-matched platelet derivatives as discussed in more detail herein. The HLA Class 1 matching can be done based on HLA-A, HLA-B, and HLA-C types and/or antigens. Alternatively, the matching can be done based on the HLA-A and HLA-B types and/or antigens. In other embodiments, the matching can be done based on epitope-based matching of HLA Class 1 antigens between different donors, to form HLA Class 1 eplet-matched cryopreserved platelets, or HLA Class 1 epitope-based matched cryopreserved platelets. Further disclosure regarding any of these matching strategies, criteria, and grades are discussed further herein and can be used to identify donors whose platelets can be pooled.

In some cases, a donor pool comprising at least 2, 3, 4, 5, 10, 15, 20, 30, 40, 50, 75, or 100 donors can be selected based on the HLA compatibility of the donors, such that the pool of platelets formed from the donor pool comprises HLA-compatible platelets. For example, a batch of cryopreserved platelets herein can comprise pooling platelet units from a donor platelet pool comprising HLA-characterized platelets, such that the HLA-characterized platelets in the donor platelet pool are HLA-compatible platelets. In illustrative embodiments, such HLA-compatible platelets of the donor platelet pool can be HLA-matched platelets, or can include different matching grades as disclosed herein. The formation of donor platelet pool as disclosed herein above can effectively create a batch of cryopreserved platelets prepared as per the process disclosed herein that can comprise multiple cryo-vessels, such that the cryopreserved platelets in each of the cryo-vessels comprise HLA-characterized platelets, typically, HLA-compatible platelets.

The number of donors can depend on the number of platelet units required for the preparation of cryopreserved platelets. For preparing a batch of cryopreserved platelets comprising more than 10 cryo-vessels, platelet units can be obtained from more than 8, 9, or 10 donors. For example, for preparing a batch of cryopreserved platelets comprising 12 cryo-vessels, 12 platelet units can be provided. In such cases, each unit can be from a different donor, such that the platelet units are from 12 donors. In other cases, 2 units can be from 1 donor, such that the platelet units are from 6 donors, or 3 units can be from 1 donor such that the platelet units are from 4 donors. A skilled artisan would understand that the number of donors can depend upon the number of platelet units required, and the availability of such units in a blood bank. In some cases, at least 3, 4, 5, 6, 7, 8, 9, 10, 11, 12, 15, 20 or more units can be processed in a single batch. Processes herein can include performing the process as disclosed herein more than once to form more than one batch of the cryopreserved platelets. For example, the process as disclosed herein can be performed multiple numbers of times to form multiple number of batches of cryopreserved platelets. For example, 5 batches of cryopreserved platelets are formed by performing the process 5 times. Similarly, the process can be performed any number of times as per the requirement of the number of batches of the cryopreserved platelets. For example, 2-500, 2-450, 2-400, 2-300, 2-250, 2-200, 2-150, or 2-100 batches can be formed by performing the process as disclosed herein as many numbers of times. In such embodiments, each batch can have 3-50, 3-40, 3-30, 3-25, 3-20, 3-25, 3-12, 4-12, or 5-12 number of cryo-vessels of cryopreserved platelets.

Processes herein for preparing a batch of cryopreserved platelets, in some cases, includes pooling of platelet units in a vessel, such that a minimum number of units are processed in one vessel until the step of combining the contents of all such vessels for the addition of a cryoprotectant. For example, at least 2 units or 3 units are pooled in a vessel to create a plurality of vessels having pooled platelet units. As a further example, 2, 3, or 4 units are pooled in a vessel to create a plurality of vessels. For preparation of a batch, based on the number of platelet units and the dimensions of vessel, a fixed number of platelet units that can be pooled into a vessel can be decided, for example, if there are 30 platelet units, 2 platelet units can be pooled into one vessel, such that there are a total of 15 vessels having 2 platelet units pooled in each vessel. Alternatively, if there are odd number of platelet units and 2 platelet units are pooled in a single vessel, then 1 platelet unit can be processed as it is in a separate vessel. The pooling of platelet units can be based on the number of platelet units provided, and/or the number of platelet units that can be pooled in one vessel. For example, in case 5 platelet units are provided, 2 units can be pooled in a vessel, and 1 remaining unit can be processed in a separate vessel such that two vessels can have a pooled set of 2 units each and one vessel can have the remining 1 unit. Alternatively, 5 platelet units can be pooled in a manner where 3 units can be pooled in one vessel, and 2 units can be pooled in another vessel. In case there are 6 platelet units that are provided, then 2 units can be pooled in a vessel such that three vessels can have a pooled set of 2 units each. Alternatively, 3 units can be pooled in one vessel, and the remaining 3 units can be pooled in another vessel, such that there are 2 vessels each containing 3 pooled units. Pooling of platelet units can be performed in a manner where 3 units or more can be pooled in one vessel. For example, in case 5 platelet units are provided, 3 units can be pooled in a first vessel and 2 units can be pooled in a second vessel such. Alternatively, 3 units can be pooled in a first vessel, and 1 unit each can be processed in separate vessels.

The number of units that can be pooled in a vessel can also depend on the type of vessel and the volume that can be processed in the vessel. Typically, a vessel can be apheresis platelet unit (APU) bags. For example, if an APU bag can hold a volume of about 800 to 900 ml for processing, then 2 to 3 units can be pooled into one vessel, such as an APU bag. For example, APU can hold a volume in the range of 800 to 1600 mL, 800 to 1000 mL, or 1000 mL to 1500 mL. For example, considering APU bags as vessels as per the process disclosed herein, the pooling can be done by using an SCD to weld a plasma transfer set onto an APU bag and then a second APU bag is welded onto the other end of the plasma transfer set. The plasma transfer set is added to extend the working length of the tubing. The two APUs can then be pooled together into a single APU bag. This can be done a multiple number of times to create a plurality of APU bags having pools (2 platelet units) of APU from the initial platelet units. The sterile connecting device used can be a Terumo, TSCD II Sterile Tubing Welder, model number 3me-SC203a (or equivalent). The plasma transfer sets used can be Charter Medical, 24″ Tubing, Roller Clamp and Two Piercing Pins, product number 03-220-00 (or equivalent). If there is an odd number of initial platelet units, such as APUs then a plasma transfer set can be welded onto the odd APU and a 600 mL transfer bag (Terumo, TeruFlex Transfer Bag, catalog number: 1BB*T060CB71, or equivalent) can be welded onto the other end of the plasma transfer set. The APC of the odd APU remains in the APU bag.

After pooling the platelet units or separating 1 platelet unit in a separate vessel, the weight of apheresis platelet concentrate (APC) can be determined for each vessel, such as each APU bag. For example, a non-limiting equation for calculating the APC weight of each vessel is:

$$Pooled or single APC Weight = Pooled or single APU Weight - Empty Vessel / Bag Weight$$

The pooled APU weights can be determined with a scale (Ohaus Adventurer Precision Balance, product number AX8201/E, or equivalent). The empty bag weight is a known value that corresponds to the type of bag that was used for the apheresis platelet collection. Accordingly, processes herein for preparing a batch of cryopreserved platelets, in some cases, include determining the weight of pooled APU, for example, weight of the platelet units pooled into a vessel. In some cases, there can be 2, or more than 2, for example, 3, 4, or more units that can be pooled into a vessel. The weight of such pooled units in a vessel can be determined by subtracting the weight of the empty vessel, for example, a bag from the weight of the vessel containing the pooled platelet units. In some cases, such a weight that is determined can be referred to as a pooled APC weight. After determining the pooled APC weight of all the vessels, the process herein further includes concentrating the pooled platelet units of each of the vessels. The concentrating can be done by a centrifugation-based process, or by a tangential flow filtration (TFF).

Processes herein for preparing a batch of cryopreserved platelets, in some cases, includes a step of centrifugation of vessels comprising pooled platelet units, or vessel comprising 1 platelet unit. The centrifugation step is to separate platelets from plasma, and as such can be achieved by centrifuging the vessel at 1000 g to 2000 g, 1000 g to 1500 g, or 1100 g to 1400 g for a time period in the range of 5-30 minutes, 5-25 minutes, or 5-20 minutes. Typically, the vessels can be APU bags and the APU bags can be kept in centrifuge cups that can be centrifuged at 1250 g for 10 minutes with maximum acceleration, and with 10 minutes of deceleration.

In some cases, processes herein for preparing a batch of cryopreserved platelets can utilize tangential flow filtration (TFF) for concentrating the platelets in pooled platelet units, or platelets provided in another vessel. In some cases, TFF can be employed instead of centrifugation of the vessels comprising pooled platelet units, or vessel comprising 1 platelet unit. In some cases, TFF can be employed along with the centrifugation steps. The TFF process can be employed to remove soluble protein components (e.g., immunogenic antibodies), perform buffer exchange, and/or concentrate the platelet-containing material. The starting material can comprise donor blood products, including but not limited to donor apheresis material, buffy-coat derived platelet products, and pooled donor plasma containing platelets. The TFF process can include one or more of the following steps: concentrating step, diafiltration, and buffer exchange. For example, the TFF process can include the concentrating step to concentrate the pooled platelet units, or a composition comprising pool of platelets, for example, platelet units provided or pooled in a vessel to obtain a pooled platelet resuspension with a target weight based on the number of platelet units (105 of FIG. 1A). In some cases, the TFF process can include concentrating the platelet units provided or pooled in a vessel to obtain a pooled resuspension as disclosed herein having a target weight depending on the units of platelets provided or pooled (150 of FIG. 1B). The TFF can be carried out using a membrane with a pore size ranging from about 0.2 μm to about 1 μm, or in some embodiments, from about 0.2 μm to about 0.45 μm.

In some cases, TFF can be used whenever platelets need to be concentrated or otherwise separated from a composition which comprises such platelets during the process for preparing a batch of cryopreserved platelets as disclosed herein. In a non-limiting illustration TFF process to concentrate platelet units provided or pooled, the required number of platelet units (X number of units), for example, irradiated APUs, can be pooled into 5 L pooling bags (˜15 or less APUs per pooling bag) using the pooling manifold, such as a pooling tree disclosed herein. In a non-limiting illustration, platelet units can be welded (in sterile conditions) onto the PVC tube lines of the pooling manifold and then the pooling manifold can be connected to the 5 L pooling bag via aseptic connector. The pooled units can then be introduced into the TFF system and concentrated to a target a post-concentration weight of, for example, 23.3 g/unit. In some cases, the target weight of 23.3 g/unit is kept the same as post-centrifugation used in the plasma expression step as disclosed below herein. Therefore, for a 30-unit batch, the post-concentration target weight is 30 units×23.3 g/unit=699 g. Once the weight target is reached, the concentrated material, for example, the pooled platelet resuspension, or the pooled resuspension herein can then be harvested from the TFF system. The cryoprotectant, such as DMSO can then be added to the pooled platelet resuspension, or the pooled resuspension to obtain ˜6% DMSO in the resuspension using 27% DMSO in saline. The addition of DMSO can comprise using the same constant of 0.2946 that is disclosed herein with the centrifugation steps to calculate the necessary amount of 27% DMSO required to be added. Therefore, for a 30 unit-batch, the amount of 27% DMSO that needs to be added is 0.2946×699 g=205.9 g of 27% DMSO solution. Typically, the TFF process herein includes the concentration step, and not a diafiltration step. The remaining process can comprise the steps similar to the steps disclosed herein for the centrifugation process for concentrating the platelet units. For example, the weight of the post-DMSO product can be divided by the number of APUs used in the process and the cryo-vessels, such as cryo-bags can be appropriately filled. For example, for a 30 unit-batch, the fill range can be calculated as: 904.9 g/30 units=30.2 g max fill weight; min. fill weight=max-2 g=30.2 g-2 g=28.2 g; fill range=28.2 g to 30.2 g for the 30 CPP units. Accordingly, the fill volume range would be 27.4 mL to 29.3 mL. In some cases, cryo-vessels herein comprising cryopreserved platelets or frozen activated platelets after thawing can have a volume in the range of 20 to 45 ml, 20 to 40 ml, 20 to 37 ml, or 25 to 35 ml. In some cases, a pre-spin equilibrium step was added to ensure all cups in the centrifugation machine were properly balanced. For example, after the re-distribution of platelets, the weight of pooled units can be taken, and average weight be calculated. Using the heavier pooled unit, each unit can be adjusted to be within +/−10 g, 8 g, 6 g, or 5 g of the average weight of the pooled units or bags. The pre-spin equilibrium step was added to ensure all cups in the centrifugation machine were properly balanced. This step can be repeated until all bags are within the range.

### Plasma Expression and Resuspension

Processes for preparing a batch of cryopreserved platelets, herein can include a step of resuspending a pellet that is obtained after a centrifugation step, or a TFF step as disclosed herein to achieve a target weight of the resuspension within a specific range. For example the target weight can be within a range of 12 g to 32 g, 13 g to 30 g, 14 g to 29 g, or 15.9 g to 27.9 g times the number of platelet units pooled or provided in the vessel that was processed or centrifuged. As a non-limiting specific example, when 2 units are pooled in a vessel, then the target weight of the resuspension can be two times any specific target between 12 g to 32 g, 13 g to 30 g, 14 g to 29 g, or 15.9 g to 27.9 g, such that the target weight of the resuspension becomes a target weight between 24 g to 64 g, 26 g to 60 g, 28 g to 58 g, or 31.8 g to 55.7 g. The target weight of the resuspension can be achieved by removing a part of the supernatant comprising plasma. This step is also known as plasma expression. Plasma expression or removing supernatant that includes plasma, can be performed to achieve a target weight of the remainder supernatant and pellet. Alternatively, removing a part of the supernatant can be performed until a target weight of the supernatant that is removed is achieved. The target weight of the supernatant that is to be removed can be determined based on the weight of the remainder supernatant and pellet that is required. For example, when 2 units are pooled in a vessel, then the target weight of the resuspension can be two times of 12 g to 32 g, 13 g to 30 g, 14 g to 29 g, or 15.9 g to 27.9 g, such that the target weight of the resuspension becomes 24 g to 64 g, 26 g to 60 g, 28 g to 58 g, or 31.8 g to 55.7 g. Typically, in case when 2 units are provided in a vessel, the target weight of the plasma that needs to be removed can be determined by the weight of the resuspension that is platelet pellet and the remainder plasma in the range of 31.8 g to 55.7 g, and in one non-limiting example the target weight of the plasma that needs to be removed is determined by the weight of the resuspension that is about 45.6 g. In illustrative embodiments, the target weight of the plasma that needs to be removed is based on the amount needed to get the final weight of the resuspension to about 46.5 g. The resuspension obtained in each vessel after the removal of plasma or the plasma expression step comprises a concentration of platelets that is higher than the concentration of platelets in the starting material, such as apheresis platelet units (APU). In some cases, the resuspension in each vessel can be referred to as a concentrated pool of platelets since it typically has a higher concentration of platelets as compared to the APUs that were used to create the pool. For example, the concentrations of platelets in the resuspension (for example, 140 of FIG. 1B) in each vessel can be at least 1.2, 1.5, 1.75, 2, 2, 5, or 3-fold higher than that of the APUs. For example, the concentrations of platelets in the resuspension can be 1.2-5.0, 1.2-4.75, 1.2-4.50, 1.2-4.25, 1.2-4.0, or 1.2-3.50-fold higher as compared to the concentration of platelets in the APUs, or any other starting material.

Typically, the plasma removal target weight for expression can be determined using the below Equation by subtracting 46.5 g from the pooled APC weight. This is done to leave behind approximately 46.5 g of platelet pellet and plasma after the pooled APU is expressed.

### Determination of Expression Endpoint

$$Plasma Removal Target = Pooled APC Weight ( 2 platelet units ) - 46.5 g$$

Each vessel after pooling the platelet units can be taken out of the centrifuge and expressed one-by-one (removing a part of the supernatant). The vessel can be carefully removed from the centrifuge cup, as not to disturb the platelet pellet, and then placed in the plasma expressor (Fenwal Inc., manual plasma extractor, product code 4R4414, or equivalent). The empty APU bag is placed on a scale and tared to weigh the expressed plasma. The pooled APU is then expressed. Once the plasma removal target is reached (±1.0 g) the expression is stopped. The post-expression pellet weights are determined to ensure that the weight of the platelet pellet and remainder supernatant is within range for further processing (31.8 g-55.7 g). If the post-expression weight is outside of the range, the supernatant can be added or removed accordingly, until the post-expression weight is within range. Once the post-expression weight is within range, the pellet is resuspended by gently rocking and massaging the APU bag until the pellet is no longer visible. After the pellet is no longer visible there is a 5-minute ambient temperature resting of the resuspended platelet pellet. The resuspended pellet is then visually inspected for aggregates. If aggregates are observed at this point in the process, and they do not disappear after further resting and agitation, manufacturing management is informed, processing continues, and a 30-minute ambient temperature rest with gentle agitation is added to the process after the addition of 27% DMSO. To accommodate the APU bag having 1 platelet unit, the plasma removal target can be determined by the following equation.

$$Plasma Removal Target ( for single Platelet unit ) = Odd APC Weight ( 1 platelet unit ) - 23.3 g$$

The post-expression pellet weight range (weight of the remainder supernatant and the pellet) is changed to 15.9 g-27.9 g, to account for the odd APU being a single APU and not a pooled APU.

Process for preparing a batch of cryopreserved platelets herein, in some aspects can include resuspending the pellet with a buffer composition, such that the target weight range of 15.9 g-27.9 g times the number of platelet units is that of the resuspension with the buffer composition. Typically, in such resuspension with the buffer composition, 90%-99.9% of supernatant comprising plasma can be removed and then the pellet can be resuspended with a buffer composition. A buffer composition can comprise a buffering agent, a base, one or more saccharide, optionally a salt, and optionally, an organic solvent. The buffering agent can be any buffer that is non-toxic to the platelets and provides adequate buffering capacity at the temperatures at which the resuspension will be exposed during the process provided herein. Thus, the buffer composition can comprise any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TBS). Likewise, it may comprise one or more of the following buffers: propane- 1,2,3-tricarboxylic (tricarballylic); benzenepentacarboxylic; maleic; 2,2-dimethylsuccinic; EDTA; 3,3-dimethylglutaric; bis(2-hydroxyethyl)imino- tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene- 1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES).

In some embodiments, one or more buffering agents can be present in the resuspension in any suitable amount. In some embodiments, the buffering agent can be present in an amount of 1 mM to 1 M. In some embodiments, one or more buffering agents can be present in about 0.2 to about 20 mg/ml, or about 0.2 to about 2 mg/ml, or about 2 mg/ml to about 20 mg/ml in the resuspension. In some embodiments the buffer composition can comprise one or more salts in about 0.08 to about 8 mg/ml, such as about 0.08 to about 0.8 mg/ml, or about 0.8 mg/ml to about 8 mg/ml in the resuspension.

The process of preparing the cryopreserved platelets provided herein can also comprise adding to the resuspension one or more salts, such as phosphate salts, sodium salts (e.g., NaCl), potassium salts (e.g., KCl), calcium salts, magnesium salts, and any other salt that can be found in blood or blood products, or that is known to be useful in cryopreserving platelets, or any combination of two or more of these.

In some embodiments, the salts are present in the resuspension at a concentration of about 1 mM to about 1000 mM, such as about 0.01 M to about 0.2 M. In some embodiments, one or more salts are present in about 0.4 to about 40 mg/ml, or about 0.4 to about 4 mg/ml, or about 4 mg/ml to about 40 mg/ml in the resuspension. In some embodiments, one or more salts are present in about 0.03 to about 3 mg/ml, or about 0.03 to about 0.3 mg/ml, or about 0.3 mg/ml to about 3 mg/ml in the resuspension.

In some embodiments, these salts are present in the resuspension in an amount that is about the same as is found in whole blood.

In some embodiments, the process for preparing a cryopreserved platelet composition includes adding to the suspension medium an organic solvent, such as an alcohol, such as ethanol, to the suspension medium. The organic solvent can include one or more alcohols, e.g., short-chain alcohols, such as ethanol. Short-chain alcohols are alcohols having 1 to 6 carbon atoms, in particular 2, 3 or 4 carbon atoms, such as methanol, ethanol, and propanol including 1-propanol and 2-propanol, preferably ethanol. The organic solvent may also be a mixture of different organic solvents. In such a resuspension medium, the solvent can range from 0.1% to 5.0% (v/v). In some embodiments, one or more organic solvents are present in about 0.08% (v/v) to about 8% (v/v), or about 0.08% (v/v) to about 0.8% (v/v), or about 0.8% (v/v) to about 8% (v/v) in the resuspension.

In some embodiments, processes herein include resuspending the pellet after the removal of plasma or plasma expression, in a buffer composition. Such a buffer composition can include one or more saccharides. The saccharides can include monosaccharides, disaccharides, or polysaccharides including sucrose, maltose, trehalose, glucose, mannose, dextrose, xylose, and combinations thereof. In some embodiments, the saccharide for use in the process of preparing cryopreserved platelets provided herein is trehalose. In some embodiments, the polysaccharide is polysucrose, or a combination of any of the above saccharides, in illustrative embodiments trehalose, and polysucrose. Thus, in one embodiment, the first mixture comprises platelets, a cryoprotectant, such as a cryoprotectant comprising trehalose, a polysucrose, or a combination thereof, and a solvent, such as ethanol.

In some embodiments, a suitable saccharide can include one or more sugar alcohols. Non-limiting examples of sugar alcohols can include mannitol, sorbitol, xylitol, maltitol, maltitol syrup, lactitol, erythritol, and combinations thereof.

In various embodiments, one or more saccharides can be present in the resuspension in any suitable amount. Such saccharides can be the cryoprotectant or can be one of the cryoprotectants, for examples when one or more saccharides are present in the resuspension along with DMSO. In some embodiments, the saccharide can be present at about 1 mM to about 1 M. In embodiments, the saccharide is present at about 10 mM to about 500 mM. In some embodiments, the saccharide is present at about 20 mM to about 200 mM. In embodiments, the saccharide is present at about 40 mM to about 100 mM. In some embodiments, one or more saccharides are present in about 0.04 mg/ml to about 4 mg/ml, about 0.04 mg/ml to about 0.4 mg/ml, or about 0.4 mg/ml to about 4 mg/ml in the resuspension. In some embodiments, one or more saccharides are present in about 3 mg/ml to about 300 mg/ml, about 3 mg/ml to about 30 mg/ml, or about 30 mg/ml to about 300 mg/ml in the resuspension.

In various embodiments, the saccharide is present in different specific concentrations within the ranges recited above, and one of skill in the art can immediately understand the various concentrations without the need to specifically recite each herein. Where more than one saccharide is present in the resuspension, each saccharide can be present in an amount according to the ranges and particular concentrations disclosed herein.

### Pooling of the Resuspension

Processes for preparing a batch of cryopreserved platelets herein, after the step of resuspending the pellet to form a resuspension, can include pooling of the resuspension from each vessel, such as an APU bag. Pooling can be done by pooling the resuspension from each vessel, such as APU bags one by one into one pooled resuspension vessel to form a pooled resuspension in a pooled resuspension vessel. In terms of understanding the pooling concept of forming a concentrated pooled platelet resuspension, 150 of FIG. 1B), or a concentrated pooled platelet resuspension can be understood as a pool of pools of platelet units. In some cases, formation of the pool of pools of platelet units can also be referred to or understood as a second pooling of the platelets. The first pooling can be the step where platelet units are pooled to form a plurality of vessels, followed by concentrating and plasma expression to form a resuspension. In some cases, the pool of pools of platelets thus formed can have a higher concentration of platelets as compared to the concentration of platelets in APUs, or any other starting material used. For example, the concentration of platelets in the pooled platelet resuspension, or the pooled resuspension herein can be at least 1.5, 2.0, 2.25, 2.50, or 3.0-fold higher than that of the APUs. In some cases, the concentration of platelets in the pooled platelet resuspension, or the pooled resuspension herein can be in the range of 1.25-5.0 fold higher than that of the APUs. Typically, pooling of resuspension from each vessels can be achieved using a pooling tree system. Such a pooled resuspension vessel can be an APU bag that can hold the volumes of the resuspension from all of the vessels. Typically, a pooled resuspension vessel is a single vessel that can contain the pooled resuspension to which a cryoprotectant is added in a consecutive step. However, in order to accommodate a high number such as 15, 20, 30, 40, 50 or more number of platelet units in a single batch the pooled resuspension from all of the vessels can be pooled and split into two, three, or more number of pooled resuspension vessel.

A pooling tree system can be designed based on the requirements and platelet units/vessels that are to be processed. An illustrative non-limiting example of a pooling tree system is shown in FIG. 1D. Referring to FIG. 1D, one non-limiting example of a pooling tree system or a tubing tree system comprises: a first four port cross style manifold (5) connecting a first 14 inch tubing (1), a second 14 inch tubing (1), a third 14 inch tubing (1), and fourth 4 inch tubing (2); a second four port cross style manifold (5) connecting the fourth 4 inch tubing (2), with a fifth 20 inch tubing (3), a sixth 14 inch tubing (1), and a seventh 4 inch tubing (2); a third four port cross style manifold (5) connecting the seventh 4 inch tubing (2) with a eighth 14 inch tubing (1), a ninth 14 inch tubing (1), and a tenth 14 inch tubing (1), and one on/off rachet clamp (4) on each of the tubing. A non-limiting example of the tubing is TYGON ND 100-65 tubing 3/32″ ID X 5/32″ OD of different sizes as per the requirement. A further non-limiting description is provided in the table below.

TABLE
Tubing Tree

| Item# | Description | Quantity |
|---|---|---|
| 1* | 3/32″ ID × 5/32″ OD Tygon ND 100-65 tubing | 14″ length (×7) |
| 2 | 3/32″ ID × 5/32″ OD Tygon ND 100-65 tubing | 4″ length (×2) |
| 3* | 3/32″ ID × 5/32″ OD Tygon ND 100-65 tubing | 20″ length (×1) |
| 4 | Pinch Clamp | ×10 |
| 5** | 3/32″ ID Hose Barb Cross Connector | ×3 |

*RF Seal on Tygon Tubing Ends.
**Zip ties on each barb and tubing connection (×12).

For example, in a process in which 12 platelet units are provided, 6 vessels, for example, 6 APU bags can be processed, and after the step of removing of plasma (plasma expression), resuspension from each bag can be pooled in a single bag. Once the 6 pellets are resuspended, the 6 APU bags can be welded onto a sterile tubing tree (Optimum Processing, Inc., part number 02817, or equivalent) to create a “pooling tree system” and the resuspension from each bag can then be pooled into a single APU bag. It is understood that a skilled artisan can use or custom design any such pooling tree system for the pooling of the resuspension as provided herein. In some cases, after the pooling of resuspension a step of plasma rinse can be included. For example, an amount of plasma rinse was calculated according to the following equation.

Plasma rinse target(g)[+/−1g]=pre-DMSO target(g)−pellet pool(g)

A pre-DMSO target is evaluated based on the pellet pool estimate (on a per APU basis), for example, from Example 2 herein plus any small aliquot(s) that were removed for in-process testing.

### Pathogen Reduction

In some embodiments of aspects that include a process for preparing cryopreserved platelets, or a batch of cryopreserved platelets, a pathogen reduction (PR) step can be applied to a platelet-containing composition, such as pooled platelet units, a resuspension having a target weight depending on the units of platelets, a pooled resuspension, or a concentrated pooled platelet resuspension (CPR) to reduce or eliminate viable pathogens, including but not limited to bacteria, viruses, and protozoa. The PR step is implemented in a manner that maintains the structural and functional properties of the platelet components, which may subsequently be processed into cryopreserved platelets (CPP).

The PR step can be conducted using ultraviolet (UV) light-based platforms, optionally in combination with intercalating or photosensitizing agents. The specific platform, parameters, and treatment conditions may be selected based on the intended use of the platelet-derived product, processing constraints, or pathogen reduction targets.

### Amotosalen/UVA-Based Photochemical Treatment (INTERCEPT® System)

In some embodiments, the pathogen reduction can include treatment with an intercalating agent, for example, amotosalen (S-59) or an alternate psoralen derivative, such as 8-methoxypsoralen, in combination with ultraviolet A (UVA) light. The platelet-containing composition can be combined with the intercalating agent at a concentration ranging from about 75 μM to about 200 μM. The composition can then be illuminated using UVA light at wavelengths between about 320 nm and about 400 nm. A cumulative energy dose ranging from approximately 2.5 J/cm² to 3.5 J/cm² can be delivered during treatment.

The illumination step can be performed under controlled temperature conditions, typically from about 20° C. to about 26° C., with continuous mixing or gentle agitation to ensure uniform exposure. Following illumination, the treated composition can be processed through a compound adsorption device (CAD) that removes residual psoralen and its photoproducts. The resulting product can be subjected to further processing in accordance with the steps of a process for preparing cryopreserved platelets, or a batch of cryopreserved platelets as disclosed herein.

The treatment achieves broad-spectrum inactivation of pathogens, including Gram-positive and Gram-negative bacteria such as Staphylococcus aureus and Escherichia coli, enveloped viruses such as HIV, HCV, and Zika virus, and protozoan parasites such as Plasmodium falciparum; and Trypanosoma cruzi. Leukocyte inactivation of the platelet-containing composition also occurs, thereby reducing the risk of transfusion-associated graft-versus-host disease (TA-GvHD) and potentially obviating the need for gamma irradiation of the downstream product, for example, cryopreserved platelets.

### Riboflavin/UV-Based Photochemical Treatment (MIRASOL® System)

In some embodiments, the pathogen reduction can include treatment with riboflavin (vitamin B2) in combination with ultraviolet light in the UVA and UVB spectrum of the platelet-containing composition. In some embodiments, riboflavin is added to the platelet-containing composition at a concentration ranging from about 35 μM to about 75 μM. The composition can then be irradiated using UV light within a wavelength range of about 280 nm to about 360 nm, with a total energy dose ranging from approximately 5.0 J/cm² to 7.0 J/cm².

Treatment can be conducted under temperature-controlled conditions, generally from about 20° C. to about 25° C., with continuous agitation. Typically, a chemical removal step is not required following illumination possibly due to the non-toxic nature of riboflavin and its photoproducts. In some embodiments, a chemical removal step can be applied to remove riboflavin, and/or other photoproducts formed as a result of the pathogen reduction step. The resulting platelet product can subjected to further processing in accordance with the steps of a process for preparing cryopreserved platelets, or a batch of cryopreserved platelets as disclosed herein.

This method enables effective inactivation of a broad array of pathogens including enveloped viruses (e.g., HIV, West Nile virus), bacteria, and protozoa such as Plasmodium spp.. The technology is particularly advantageous in contexts requiring rapid deployment, such as epidemic or outbreak settings.

### UVC-Based Pathogen Reduction Without Additives (THERAFLEX® System)

In some embodiments, the pathogen reduction can include exposure of the platelet-containing composition to ultraviolet C (UVC) light without the addition of photosensitizing or intercalating agents. The platelets, for example, pooled platelet units, a resuspension having a target weight depending on the units of platelets, a pooled resuspension, or a concentrated pooled platelet resuspension (CPR) can be irradiated using UVC light with wavelengths ranging from about 200 nm to about 280 nm. A total energy dose in the range of about 0.15 J/cm² to about 0.4 J/cm² is typically applied.

Illumination is conducted with continuous mixing to ensure uniform exposure, and temperature is maintained between about 20° C. and about 25° C. As no chemical additives are introduced, no removal step is required post-illumination. The resulting composition can be subjected to further processing in accordance with the steps of a process for preparing cryopreserved platelets, or a batch of cryopreserved platelets as disclosed herein.

This approach has demonstrated efficacy in bacterial inactivation (e.g., Klebsiella pneumoniae, and Pseudomonas aeruginosa), limited activity against certain non-enveloped viruses (e.g., HEV, adenovirus), and inactivation of leukocytes in the platelet-containing composition to mitigate TA-GvHD risk. Its additive-free workflow can be advantageous in manufacturing environments with simplified processing requirements.

Any one or a combination of pathogen reduction steps can be included as a step in a process for preparing cryopreserved platelets, cryopreserved platelet composition, or a batch of cryopreserved platelets. In illustrative embodiments, a pathogen reduction step can be applied to platelets, and pooled platelets in a vessel, for example, at step 120 of FIG. 1B, or at step 220 of FIG. 2B. In some embodiments, a pathogen reduction step can be applied to a resuspension having a target weight depending on the units of platelets, for example, at step 140 of FIG. 1B, or at step 240 of FIG. 2B. In some embodiments, a pathogen reduction step can be applied to a pooled resuspension, for example, at step 150 of FIG. 1B, or at step 250 of FIG. 2B. In some embodiments, a pathogen reduction step can be applied to a pooled resuspension having a cryoprotectant, for example, at step 160 of FIG. 1B, or at step 260 of FIG. 2B. In some embodiments, a pathogen reduction step can be applied to a concentrated pooled platelet resuspension (CPR) with a weight based on the number of platelet units, for example, at step 105 of FIG. 1A. In some embodiments, a pathogen reduction step can be applied to a CPR with a weight based on the number of platelet units having a cryoprotectant, for example, at step 160 of FIG. 1A.

### Addition of Cryoprotectant

Processes for preparing a batch of cryopreserved platelets herein can include adding a cryoprotectant to a pooled resuspension as disclosed herein. Without being bound by any theory or mechanism, the cryoprotectant stabilizes proteins and other biological substances in the interior of the platelets. The identity of the cryoprotectant is not limited as long as it can enter the platelets and provide a cryoprotectant property. In some embodiments, the cryoprotectant comprises dimethyl sulfoxide (DMSO). In other embodiments, the cryoprotectant is any other cryoprotectant apart from DMSO. Other non-limiting examples of suitable cryoprotectants can include saccharides, such as monosaccharides and disaccharides, including sucrose, maltose, trehalose, glucose, mannose, dextrose, xylose, and combinations thereof. In some embodiments, the saccharide for use in the method of preparing a cryopreserved platelet composition provided herein is trehalose.

In some embodiments, a cryoprotectant can include one or more sugar alcohols. Non-limiting examples of sugar alcohols can include mannitol, sorbitol, xylitol, maltitol, maltitol syrup, lactitol, erythritol, and combinations thereof.

In various embodiments, one or more saccharides can be present in the pooled resuspension or in the cryopreserved platelets in any suitable amount. For example, the saccharide can be present at about 1 mM to about 1 M, about 10 mM to about 500 mM, about 20 mM to about 200 mM, or about 40 mM to about 100 mM. As further non-limiting examples, one or more saccharides can be present in about 0.04 mg/ml to about 4 mg/ml, about 0.04 mg/ml to about 0.4 mg/ml, or about 0.4 mg/ml to about 4 mg/ml in the pooled resuspension or the cryopreserved platelets. In some embodiments, one or more saccharides are present in about 3 mg/ml to about 300 mg/ml, about 3 mg/ml to about 30 mg/ml, or about 30 mg/ml to about 300 mg/ml in the pooled resuspension or the cryopreserved platelets.

In some embodiments, the cryoprotectant can include one or more polyols. For example, suitable cryoprotectants can include glycerol (glycerin), ethylene glycol, polyethylene glycol, sorbitol, propylene glycol, pentaerythritol, a saccharide, hydroxypropyl-p-cyclodextrin, a glycerol oligomer, or combinations thereof. In some embodiments, the cryoprotectant is no more than about 10% (v/v) (e.g., no more than about 9% (v/v), 8% (v/v), 7% (v/v), 6% (v/v), 5% (v/v), 4% (v/v), 3% (v/v), 2% (v/v), 1% (v/v), 0.5% (v/v), or 0.1% (v/v). In some embodiments, the cryoprotectant is in an amount of at least about 1% (w/v) (e.g., at least about 2% (v/v), 3% (v/v), 4% (v/v), 5% (v/v), 6% (v/v), 7% (v/v), 8% (v/v), 9% (v/v), or 10% (v/v)). For example, the cryoprotectant is in an amount of about 0.1% (v/v) to about 10% (v/v), about 0.5% (v/v) to about 7% (v/v), about 1% (v/v) to about 5% (v/v), or about 0.1% (v/v) to about 1% (v/v). Glycerol can also be used as a cryoprotectant.

Typically, process for preparing a batch of cryopreserved platelets herein includes using DMSO as a cryoprotectant. For example, adding DMSO can be performed until a target weight of DMSO is added to a pooled resuspension as disclosed herein. DMSO can be added to the pooled resuspension until the concentration of DMSO in a pooled resuspension is in the range of 0.001-10% in the pooled resuspension. For example, DMSO concentration can be in the range of 0.005-10%, 0.1-10%, 1-10%, 2-10%, 3-10%, 4-10%, 5-10%, 5.5-10%, 6-10%, 6-9%, 6-8%, 0.001-9%, 0.001-8%, or 0.001-7%. In some embodiments of the process herein, DMSO concentration in the pooled resuspension can be at least 0.001%, 0.005%. 0.05%, 0.1%, 0.2%, 0.5%, 1%, 2%, 3%, 4%, 5%, 6%, 7%, 8%, 9%, or 10%. During performing the process herein, when the suspension from each vessels are pooled to form a pooled resuspension in a pooled resuspension vessel, the amount of a cryoprotectant or DMSO to be added can be calculated based on the weight of the pooled resuspension obtained after pooling the resuspension from each vessel, for example, APU bags. Typically, weight of the pooled resuspension can be determined by adding up weights of all the resuspension from each vessel or APU bag. Pooled APC weight after removal of plasma from all the vessels that initially had 2 platelet units can be added along, if applicable, with the APC weight from the vessel that initially had 1 platelet unit after removal of plasma to arrive at a summation of APC weight, or a total APC weight. The total APC weight at this step can be the post-expression weight and can be arrived at by summing up the resuspension weight of all the vessels/APU bags after removal of plasma.

$$\begin{matrix}Total Post Expression ( after removal of plasma ) Weight = \\ Sumation of resuspension Weights = Total APC Weight\end{matrix}$$

An amount of DMSO that needs to be added at this step can vary based on the total APC weight, the stock concentration of DMSO, and the target DMSO concentration that needs to be achieved in the cryopreserved platelets. An illustrative, non-limiting equation for calculating the weight of DMSO that needs to be added to the pooled resuspension is shown below. A stock concentration of 27% DMSO has been considered, and a target DMSO concentration of 6.085% in the cryopreserved platelets in a cryo-vessel has been considered:

$$Target weight of 27 \% DMSO = Total APC Weight * 0.2946$$

After the determination of the target weight of DMSO that needs to be added, DMSO can be added to the pooled resuspension by any means that facilitate the addition of DMSO with minimal wastage and leading to uniform mixing of DMSO. The addition of DMSO can be performed directly or indirectly to the pooled resuspension vessel. Typically, a bag or a vessel containing sterile, injectable grade 27% DMSO and 0.66% sodium chloride in water (Bio Life Solutions, BloodStor® 27 NaCl Biopreservation Media, part number 327207, or equivalent) can be welded onto the pooling tree system that was initially used to pool the resuspension from each vessel/APU bags. The 27% DMSO bag can be placed on a scale to weigh how much 27% DMSO is leaving the 27% DMSO bag and entering the pooling tree system. The 27% DMSO bag can be placed higher than the pooling tree system to allow the 27% DMSO to flow gravimetrically. The addition of 27% DMSO Target Weight to the pooling tree system can be performed until ±3.0 g, ±2.0 g, or ±1.0 g is added to the pooling tree system. The tubing to the 27% DMSO bag can be clamped to prevent additional 27% DMSO from entering the system. The step comprising addition of a cryoprotectant such as DMSO can be performed using a pooling tree system that was initially used to pool the resuspension from each vessel/bag. In such steps, DMSO can be used to rinse the pooling tree system, and the vessels/APU bags that had the resuspension to effectively flush out any platelets that can be stuck to the pooling tree system. Typically, the 27% DMSO that entered the pooling tree system is used to rinse the system to recoup any residual platelet material that remains in the APU bags and in the tubing of the pooling tree system. The 27% DMSO can then be added to the pooled resuspension vessel, for example, an APU bag that contains the pooled resuspension. Tube strippers can be used to strip any 27% DMSO solution that may remain in the pooling tree system, to ensure that all 27% DMSO is added to the platelets. It can be understood that a skilled artisan can manipulate the DMSO constant of 0.2946 disclosed in the above equation based on the stock solution of DMSO, and target concentration of DMSO that needs to be achieved in the cryopreserved platelets. For example, the DMSO constant will change if a stock of 10% DMSO is considered rather than 27% DMSO.

### Distribution and Freezing

Processes for preparing a batch of cryopreserved platelets, herein can include distributing a pooled resuspension having a cryoprotectant, such as DMSO to a number of cryo-vessels for a further freezing step. A cryo-vessel can be any appropriate sealable vessel (e.g., a container closure system) in which platelets can be frozen. A cryo-vessel can be an appropriate vial (cryo-vial), ampule, or a bag (e.g., cryo-bag) For example, a cryo-vessel can be a cryo-bag such as a fluorinated ethylene propylene (FEP) bag or a polyvinyl chloride (PVC) bag. A cryo-vessel can be a borosilicate serum vial. A non-limiting example of a cryo-vessel or a cryobag is 250 mL ethyl vinyl acetate (EVA) thermoplastic container (CryoStore CS250 series, Origen, Austin, TX). The CS250 has a recommended freeze volume of 30-70 mL. The EVA container is also suitable for delivery of the intravenous dosage form being like any other blood container being the appropriate flexibility and transparency. The EVA container is placed in a polyethylene overwrap bag (Helmer, Noblesville, IN) as a secondary container, then placed into a corrugated cardboard box (Mission City Container, San Antonio, TX), internal dimensions 7″×5 1/4″×1 5/8″, 200 pound test bursting strength, to protect the product from both light and damage.

In some embodiments of a process for preparing a batch of cryopreserved platelets herein, distributing a pooled resuspension having a cryoprotectant, such as DMSO can be performed by determining a fill-weight or a fill-volume that needs to be distributed to a number of cryo-vessels. For example, a fill-weight can be determined by dividing the weight of pooled resuspension by the number of platelet units provided or processed, such that the pooled resuspension can be distributed to a number of cryo-vessels that is equal to the number of platelet units provided or processed. In some embodiments, a fill-weight can be determined by dividing the weight of pooled resuspension by 1/3, 1/2, 2/3, or 2 times the number of platelet units provided or processed. As a non-limiting example, when 12 platelet units are processed, then the weight of pooled resuspension can be divided by 12 to form a batch comprising 12 cryo-vessels comprising cryopreserved platelets. Accordingly, in some cases, a cryo-vessel containing cryopreserved platelets can contain an equivalent of 1 unit of cryopreserved platelets or frozen activated platelets, such that 1 unit equivalent is an amount equal to the starting number of pooled platelet units divided by number of pooled units. For example, in case 12 platelet units are pooled to prepare cryopreserved platelets or frozen activated platelets, the collection of cryopreserved platelets thus obtained can have 12 cryo-vessels, and each of the cryo-vessels comprising an equivalent of 1 unit of cryopreserved platelets or frozen activated platelets. Alternatively, when 12 platelet units are processed, by dividing the total weight of pooled resuspension having cryoprotectant with appropriate numbers, the pooled resuspension having cryoprotectant can be distributed into 3, 6, 8, 15, 18, or 24 cryo-vessels. In some cases, the distributing can be done to achieve 1 platelet unit (PU) equivalent of platelets in each cryo-vessel. Having such a tightly controlled number of platelets in each cryo-vessels provides an advantage in terms of providing a uniform amount of platelets in each cryo-vessel. The uniform amount of platelets further ensures that the dosage remains accurate when administering to the subjects in need thereof.

Distributing herein can be performed using any means that allow maintaining a sterile environment, and a clear passage of the pooled resuspension having cryoprotectant. In illustrative embodiments, distributing can be performed by means of a dosing tree system. A dosing tree system can be same or different in design to the tubing tree system. Typically, in cases where a number of cryo-vessels equal to the number of platelets units are desired, and 12 platelet units are provided for processing then a pooled resuspension vessel, such as an APU bag containing the pooled resuspension (final product) can be welded onto a new tubing tree to fill 12 cryo-bags. This creates a “filling tree system.” The weight of the pooled resuspension having the cryoprotectant (final product weight) can then be determined by weighing the pooled resuspension vessel/APU bag and subtracting the empty bag weight. The pooled resuspension (final product) weight can then be divided by 12 to determine the maximum fill weight of the cryo-bags. The minimum fill weight can be determined by subtracting 2.0 g from the maximum fill weight. This determines the fill weight range of the cryo-bags. Further, 12 cryo-bags (250 mL EVA CryoStore freezing bag, Origen Reference CS250, or equivalent) can then be welded onto a dosing tree system. The filling procedure for a cryo-bag takes place by placing the cryo-bag on the scale, priming the lines of the cryo-bag until just before the pooled resuspension having cryoprotectant enters the cryo-bag, and then taring the cryo-bag. The cryo-bag can then be filled with the pooled resuspension having cryoprotectant until it is within range. The fill weight of each cryo-bag can then be recorded and their volume can be determined by dividing the weight by 1.03 g/mL. In some cases, cryo-vessels, for example, cryo-bags herein can have a volume of pooled resuspension having a cryoprotectant, for example, DMSO in the range of 20-35 ml, 21-35 ml, 22-35 ml, 24-35 ml, 20-34 ml, 20-32 ml, 20-30 ml, 24-30 ml, 24.7-29.5 ml, 25-29 ml, or 25-28.5 ml. It can be understood that the volume of pooled resuspension that will be frozen in a cryo-vessel can be similar to the volume of cryopreserved platelet composition or frozen activated platelets, upon thawing in a cryo-vessel. For example, the volume of the composition upon thawing can be at least 95%, 96%, 98%, 99%, or 99.5%, or the same as the volume of the pooled resuspension that is frozen in the cryo-vessel.

In any of the processes herein for preparing CPP or batches thereof, the step(s) after the step of distributing the pooled resuspension having a cryoprotectant can include freezing the pooled resuspension having a cryoprotectant. Freezing can be performed by subjecting the pooled resuspension having a cryoprotectant to low temperatures to about or less than about −1° C. (e.g., about or less than about −5° C., about −10° C., about −20° C., about −30° C., about −40° C., about −50° C., about −60° C., about −70° C., about −80° C., or about of less than about −90° C.). For example, the pooled resuspension having a cryoprotectant in cryo-vessels can be subjected to a temperature of about −70° C. to about −90°, −50° C. to about −70°, −30° C. to about −50°, −10° C. to about −30°, or about −10° C. to about −20° C.). Typically, the cryo-vessels can be subjected to a temperature of about −80° C. Typically, each cryo-bag can be placed into a thawing bag and freezer carton for storage in a −80° C. freezer. The units can then be placed in the freezer and the start time of freezing is recorded. The time elapsed from the end of 27% DMSO addition to the start time of freezing, in illustrative embodiments can be equal to or less than 3 hours. The cryopreserved platelets can be thawed for further use by known methods such as exposing the cryopreserved platelets to a non-freezing temperature. For example, the thawing can include submersing the cryopreserved platelets in a 37° C. water bath for a suitable amount of time, e.g., about 8 minutes to about 10 minutes.

In any of the processes herein for preparing CPP or batches thereof, in some embodiments, the step(s) after the step of distributing the pooled resuspension having a cryoprotectant can include freezing the pooled resuspension having a cryoprotectant with a transition in freezing temperatures from an initial freezing temperature to a storage freezing temperature. In some embodiments, processes herein comprise a step of initial freezing at a temperature (i.e., initial temperature) less than or equal to −50° C., −60° C., −65° C., −70° C., −80° C., −85° C., or −90° C., or in the range of −50° C. to −85° C., or −60° C. to −85° C. to form an initial frozen platelet composition, followed by storing the initial frozen platelet composition in a frozen state at a temperature (i.e., storage temperature) equal to or greater than −30° C., but less than 0° C., to form cryopreserved platelets, or a cryopreserved platelet composition, thereby forming a batch of cryopreserved platelets.

In some embodiments of a process for preparing a batch of cryopreserved platelets, freezing platelets in a cryopreservation medium, or freezing a pooled resuspension having a cryoprotectant, or freezing a pooled resuspension having a cryoprotectant in cryo-vessels as disclosed herein, can be carried out using various freezing methods or protocols as disclosed herein. In illustrative embodiments, the platelets, or pooled resuspension, or cryo-vessels can be placed in a freezer, freezing environment, freezer rack, freezer shelf, or freezing container configured to maintain or achieve freezing. The time it takes for the pooled resuspension in the cryo-vessels to achieve freezing can depend on various non-limiting parameters, in illustrative embodiments, the volume of the cryo-vessel, the dimensions of the cryo-vessel, as well as the temperature at the start of freezing and during the process.

In some embodiments of a process for preparing a batch of cryopreserved platelets, a transition freezing method can be used. In illustrative embodiments, the transition freezing method can have one or more transition temperatures as disclosed herein. In some embodiments, a controlled-rate freezing method can be used, in illustrative embodiments, the temperature of the pooled resuspension or cryo-vessels can be changed incrementally, in non-limiting examples, in a range of 0.5° C. to 5.0° C., 0.5° C. to 4.0° C., 0.5° C. to 3.0° C., 0.5° C. to 2.0° C., 1.0° C. to 5.0° C., 2.0° C. to 5.0° C., 3.0° C. to 5.0° C., 4.0° C. to 5.0° C. per minute. In some embodiments, a single freezing temperature, or a snap-freezing method can be used. In some embodiments, uncontrolled-rate freezing can be used. In some embodiments, any one of the disclosed freezing methods that results in a composition comprising cryopreserved platelets that exhibit one or more recited properties can be used.

In some cases of the processes herein for preparing batches of cryopreserved platelets, or CPP, the process provides a reduced time exposure of platelets to a cryoprotectant, such as DMSO before the initiation of freezing. The time period is reduced by at least 1.2, 1.50, 1.75, 2.0, 2.25, or 2.50-fold as compared to a single-donor process for preparing cryopreserved platelets before the initiation of freezing. For example, the process herein includes 1.2-3.0, 1.2-2.75, 1.2-2.50 fold reduced time exposure to DMSO before the initiation of the freezing step, such as placing the cryo-vessels in the freezer pre-set at a freezing temperature. In any of the processes herein for preparing CPP or batches thereof, in some embodiments, the step(s) between adding a cryoprotectant to a platelet resuspension (e.g., a pooled platelet resuspension, or a CPR) and freezing the resuspension having the cryoprotectant, can be completed within 4, 3, 2, or in illustrative embodiments, 1 hour after addition of the cryoprotectant until the freezing step, which will be understood to be the start of the freezing step (e.g., placing the cryo-vessels in a freezer). For example, the process can be completed at a time in the range of 1-3 hours, 1.5-3 hours, 2-3 hours, or 1.5 to 2.5 hours after the addition of cryoprotectant until the freezing step.

From a more detailed perspective regarding the time elapsed between adding a cryoprotectant (e.g., DMSO) to a platelet resuspension and the initiation or start of freezing the resuspension, such initiation can be the moment the pooled resuspension having a cryoprotectant, such as the concentrated pooled platelet resuspension (CPR) having a cryoprotectant is placed in a freezer, or freezing environment configured to freeze the liquid composition comprising the pooled resuspension or the CPR having the cryoprotectant, or pre-set to a freezing temperature to freeze the liquid. Thus, the initiation is typically the moment the pooled resuspension having cryoprotectant (e.g., DMSO) is subjected to a temperature at or below freezing. Such an elapsed time can be, for example, less than or equal to 4, 3, 2, 1 hour, 45 or 30 minutes. For example, the time elapsed from the addition of a cryoprotectant to the initiation of freezing can be in the range of 15 minutes to 1 hour, 15 minutes to 1.5 hours, 15 minutes to 2 hours, 15 minutes to 2.5 hours, or 15 minutes to 3 hours. In some embodiments, the time elapsed from the addition of the cryoprotectant (e.g., DMSO) to the pooled resuspension, until the pooled resuspension having cryoprotectant becomes frozen, can be, for example, less than or equal to 4, 3, 2, or 1 hour.

A batch of cryo-vessels comprising cryopreserved platelets obtained from process herein can be assessed for a number of post-manufacturing specifications. A non-limiting set of such specifications can include the following:
Freeze Volume: 20 mL-35 mL
Time from addition of 27% DMSO to freezer: less than or equal to 3 hours
Frozen by the end of day 2 of platelet age
Visible aggregates in cryo-bag: none
% DMSO: 5.65%-6.52%
Maximum DMSO in a CPP unit: 2.53 g (the mass corresponding to the maximum freeze volume with the maximum % DMSO).
   Variance of Compositions Comprising Cryopreserved Platelets within and Across Batches

Processes for preparing a batch of cryopreserved platelets herein, including processes that comprise a transition in freezing temperatures from an initial freezing temperature to a storage freezing temperature can provide cryopreserved platelet composition, cryopreserved platelets, or a batch of cryopreserved platelets having a plurality of cryo-vessels having cryopreserved platelets that can be more homogenous than those of prior methods, and can have a reduced batch-to-batch variability. Also, collections of cryo-vessels provided herein can be homogenous, and have a reduced batch-to-batch variability. Such improved homogeneity and reduced batch-to-batch variation, in some embodiments, can be displayed by low coefficient of variance within a batch or across batches of parameters not limited to resuspension weight or volume (post-expression weight or volume) in a vessel, such as APU bag within 15%, 14%, 13%, 12%, 11%, or 10%. For example, resuspension volume in a vessel can have a coefficient of variance in the range of 0.1-15%, 01-14%, 0.1-13%, 0.1-12%, 0.1-11%, 0.1-10%, 0.1-9%, 0.1-8%, or 0.1-7% within a batch or across batches. For example, resuspension volume in a vessel can have a coefficient of variance in the range of 0.1-15%, 01-14%, 0.1-13%, 0.1-12%, 0.1-11%, 0.1-10%, 0.1-9%, 0.1-8%, or 0.1-7% across at least 2 batches. For example, resuspension volume in a vessel can have a coefficient of variance in the range of 0.1-15%, 01-14%, 0.1-13%, 0.1-12%, 0.1-11%, 0.1-10%, 0.1-9%, 0.1-8%, or 0.1-7% across at least 5 batches. For example, resuspension volume in a vessel can have a coefficient of variance in the range of 0.1-15%, 01-14%, 0.1-13%, 0.1-12%, 0.1-11%, 0.1-10%, 0.1-9%, 0.1-8%, or 0.1-7% across at least 15 batches. For example, resuspension volume in a vessel can have a coefficient of variance in the range of 0.1-15%, 01-14%, 0.1-13%, 0.1-12%, 0.1-11%, 0.1-10%, 0.1-9%, 0.1-8%, or 0.1-7% across at least 2, 3, 4, 5, or 10 lots, or between 2, 3, 4, or 5 lots on the low end to 100 lots on the high end. For example, resuspending the pellet in each vessel leads to a resuspension in each vessel such that the volume of the resuspension across at least 2, 3, 4, 5, or 10 lots, or between 2, 3, 4, or 5 lots on the low end to 100 lots on the high end has a mean intra-batch coefficient of variance (mean of intra-batch CV) of less than 15%, 14%, 13%, 12%, 11%, 10%, 9%, 8%, or 7%. In some embodiments, the mean intra-batch coefficient of variance of the resuspension in each vessel across at least 10 batches can be in the range of 1-20%, 1-15%, 1-10%, 1-8%, 2-8%, 3-8%, or 4-8%. For example, resuspending the pellet in each vessel leads to a resuspension in each vessel such that the volume of the resuspension across 2-12 batches has a mean intra-batch coefficient of variance (mean of intra-batch CV) of less than 15%, 14%, 13%, 12%, 11%, 10%, 9%, 8%, or 7%. In some embodiments, the mean intra-batch coefficient of variance of the resuspension in each vessel across 2-12 batches can be in the range of 1-20%, 1-15%, 1-10%, or 1-8%, 2-8%, 3-8%, or 4-8%. In some embodiments, resuspending the pellet in each vessel leads to a resuspension in each vessel such that the volume of the resuspension in a vessel across at least 5 batches or within a batch varies within 20%, 15%, 12%, 10%, 9%, or 8%. For example, resuspending the pellet in each vessel leads to a resuspension in each vessel such that the volume of the resuspension in a vessel across at least 5 batches or within a batch varies in the range of 3-25%, 7-20%, 7-15%, 7-14%, 7-12%, 7-10%, 3-20%, 3-15%, 3-12%, or 3-10%. In some embodiments, resuspending the pellet in each vessel leads to a resuspension in each vessel such that the volume of the resuspension in a vessel across at least 10 batches or within a batch varies within 20%, 15%, 12%, 10%, 9%, or 8%. For example, resuspending the pellet in each vessel leads to a resuspension in each vessel such that the volume of the resuspension in a vessel across at least 10 batches or within a batch vanes in the range of 3-25%, 7-20%, 7-15%, 7-14%, 7-12%, 7-10%, 3-20%, 3-15%, 3-12%, or 3-10%. In some embodiments, resuspending the pellet in each vessel leads to a resuspension in each vessel such that the volume of the resuspension in a vessel across at least 20 batches or within a batch varies within 20%, 15%, 12%, 10%, 9%, or 8%. For example, resuspending the pellet in each vessel leads to a resuspension in each vessel such that the volume of the resuspension in a vessel across at least 5 batches or within a batch vanes in the range of 3-25%, 7-20%, 7-15%, 7-14%, 7-12%, 7-10%, 3-20%, 3-15%, 3-12%, or 3-10%. In some embodiments, resuspending the pellet in each vessel leads to a resuspension in each vessel such that the volume of the resuspension in a vessel across 5-100 batches or within a batch varies within 20%, 15%, 12%, 10%, 9%, or 8%. For example, resuspending the pellet in each vessel leads to a resuspension in each vessel such that the volume of the resuspension in a vessel across 5-100 batches or within a batch varies in the range of 3-25%, 7-20%, 7-15%, 7-14%, 7-12%, 7-10%, 3-20%, 3-15%, 3-12%, or 3-10%.

Another parameter for assessment of the homogeneity can be the volume or weight of a pooled resuspension having a cryoprotectant, or a cryopreservation medium having platelets, such as DMSO in a cryo-vessel, also known as freeze volume or weight, that has low coefficient of variance within a batch or across batches within 15%, 14%, 13%, 12%, 11%, 10%, 9%, 8%, 7%, 6%, 5%, 3%, 2%, or 1%. For example, the volume or weight of a pooled resuspension having a cryoprotectant in a cryo-vessel, or a cryopreservation medium having platelets can have a coefficient of variance in the range of 0.1-5%, 0.1-4%, 0.1-3%, 0.1-2%, 0.1-1%, 0.1-0.5%, 0.1%-0.4%, 01%-0.3%, or 0.1%-0.2% within a batch or across batches. For example, the volume or weight of pooled resuspension having a cryoprotectant in a cryo-vessel, or a cryopreservation medium having platelets can have a coefficient of variance in the range of 0.1-15%, 01-14%, 0.1-13%, 0.1-12%, 0.1-11%, 0.1-10%, 0.1-9%, 0.1-8%, 0.1-7%, 0.1-6%, 0.1-5%, 0.1-4%, 0.1-3%, 0.1-2%, 0.1-1%, 0.1-0.5%, 0.1%-0.4%, 01%-0.3%, or 0.1%-0.2% across at least 2 batches. For example, the volume or weight of pooled resuspension having a cryoprotectant in a cryo-vessel, or a cryopreservation medium having platelets can have a coefficient of variance in the range of 0.1-15%, 01-14%, 0.1-13%, 0.1-12%, 0.1-11%, 0.1-10%, 0.1-9%, 0.1-8%, 0.1-7%, 0.1-6%, 0.1-5%, 0.1-4%, 0.1-3%, 0.1-2%, 0.1-1%, 0.1-0.5%, 0.1%-0.4%, 01%-0.3%, or 0.1%-0.2% across at least 5 batches. For example, the volume or weight of pooled resuspension having a cryoprotectant in a cryo-vessel, or a cryopreservation medium having platelets can have a coefficient of variance in the range of 0.1-15%, 01-14%, 0.1-13%, 0.1-12%, 0.1-11%, 0.1-10%, 0.1-9%, 0.1-8%, 0.1-7%, 0.1-6%, 0.1-5%, 0.1-4%, 0.1-3%, 0.1-2%, 0.1-1%, 0.1-0.5%, 0.1%-0.4%, 01%-0.3%, or 0.1%-0.2% across at least 15 batches. For example, the volume or weight of pooled resuspension having a cryoprotectant in a cryo-vessel, or a cryopreservation medium having platelets can have a coefficient of variance in the range of 0.1-15%, 01-14%, 0.1-13%, 0.1-12%, 0.1-11%, 0.1-10%, 0.1-9%, 0.1-8%, 0.1-7%, 0.1-6%, 0.1-5%, 0.1-4%, 0.1-3%, 0.1-2%, 0.1-1%, 0.1-0.5%, 0.1%-0.4%, 01%-0.3%, or 0.1%-0.2% across 5-100 batches. For example, the volume or weight of the pooled resuspension in a cryo-vessel, or a cryopreservation medium having platelets across at least 10 batches has a mean intra-batch coefficient of variance of less than 15%, 14%, 13%, 12%, 11%, 10%, 9%, 8%, 7%, 6%, 5%, 4%, 3%, or 2%. For example, the volume or weight of the pooled resuspension having a cryoprotectant in a cryo-vessel, or a cryopreservation medium having platelets across at least 10 batches has a mean intra-batch coefficient of variance in the range of 0.1-15%, 01-14%, 0.1-13%, 0.1-12%, 0.1-11%, 0.1-10%, 0.1-9%, 0.1-8%, 0.1-7%, 0.1-6%, 0.1-5%, 0.1-4%, 0.1-3%, 0.1-2%, 0.1-1%, 0.1-0.5%, 0.1%-0.4%, 01%-0.3%, or 0.1%-0.2%. For example, the volume or weight of the pooled resuspension having a cryoprotectant in a cryo-vessel across 2-12 batches, or a cryopreservation medium having platelets has a mean intra-batch coefficient of variance (mean of intra-batch CV) of less than 15%, 14%, 13%, 12%, 11%, 10%, 9%, 8%, 7%, 6%, 5%, 4%, 3%, or 2%. In some embodiments, the volume or weight of the pooled resuspension having a cryoprotectant in a cryo-vessel, or a cryopreservation medium having platelets across 2-12 batches can be in the range of 0.05-20%, 0.1-20%, 0.5-20%, 1-20%, 1-15%, 1-10%, or 1-8%, 1-7%, 1-6%, 1-5%, 1-4%, 1-3%, 1-2%, 0.1-2%, 0.1-1%, 0.1-0.5%, 0.1%-0.4%, 01%-0.3%, or 0.1%-0.2%. In some embodiments, the volume or weight of the pooled resuspension having a cryoprotectant in a cryo-vessel across at least 5 batches or within a batch varies within 20%, 15%, 12%, 10%, 9%, 8%, 7%, 6%, 5%, 4%, 3%, or 2%. For example, the volume or weight of the pooled resuspension having a cryoprotectant in a cryo-vessel across, or a cryopreservation medium having platelets at least 5 batches or within a batch varies in the range of 0.05-20%, 0.1-20%, 0.5-20%, 1-20%, 1-15%, 1-10%, or 1-8%, 1-7%, 1-6%, 1-5%, 1-4%, 1-3%, 1-2%, 0.1-2%, 0.1-1%, 0.1-0.5%, 0.1%-0.4%, 01%-0.3%, or 0.1%-0.2%. In some embodiments, the volume or weight of the pooled resuspension having a cryoprotectant in a cryo-vessel, or a cryopreservation medium having platelets across at least 10 batches or within a batch varies within 20%, 15%, 12%, 10%, 9%, 8%, 7%, 6%, 5%, 4%, 3%, 2%, or 1%. For example, the volume or weight of the pooled resuspension having a cryoprotectant in a cryo-vessel, or a cryopreservation medium having platelets across at least 10 batches or within a batch varies in the range of 0.05-20%, 0.1-20%, 0.5-20%, 1-20%, 1-15%, 1-10%, or 1-8%, 1-7%, 1-6%, 1-5%, 1-4%, 1-3%, 1-2%, 0.1-2%, 0.1-1%, 0.1-0.5%, 0.1%-0.4%, 01%-0.3%, or 0.1%-0.2%. In some embodiments, the volume or weight of the pooled resuspension having a cryoprotectant in a cryo-vessel, or a cryopreservation medium having platelets across at least 20 batches or within a batch varies within 20%, 15%, 12%, 10%, 9%, 8%, 7%, 6%, 5%, 4%, 3%, 2%, or 1%. For example, the volume or weight of the pooled resuspension having a cryoprotectant in a cryo-vessel, or a cryopreservation medium having platelets across at least 20 batches or within a batch varies in the range of 0.05-20%, 0.1-20%, 0.5-20%, 1-20%, 1-15%, 1-10%, or 1-8%, 1-7%, 1-6%, 1-5%, 1-4%, 1-3%, 1-2%, 0.1-2%, 0.1-1%, 0.1-0.5%, 0.1%-0.4%, 01%-0.3%, or 0.1%-0.2%. In some embodiments, the volume or weight of the pooled resuspension having a cryoprotectant in a cryo-vessel, or a cryopreservation medium having platelets across 5-100 batches or within a batch varies within 20%, 15%, 12%, 10%, 9%, 8%, 7%, 6%, 5%, 4%, 3%, 2%, or 1%. For example, the volume or weight of the pooled resuspension having a cryoprotectant in a cryo-vessel, or a cryopreservation medium having platelets across at least 5-100 batches or within a batch varies in the range of 0.05-20%, 0.1-20%, 0.5-20%, 1-20%, 1-15%, 1-10%, or 1-8%, 1-7%, 1-6%, 1-5%, 1-4%, 1-3%, 1-2%, 0.1-2%, 0.1-1%, 0.1-0.5%, 0.1%-0.4%, 01%-0.3%, or 0.1%-0.2%.

In some embodiments of aspects that include process for preparing a batch of cryopreserved platelets, a process for preparing a cryopreserved platelet composition including a transition in freezing temperatures from an initial freezing temperature to a storage freezing temperature, and a collection or a batch comprising cryopreserved platelets, process herein provide an improved homogeneity in terms of concentrations of the cryoprotectant that is present in the cryopreserved platelets in a cryo-vessel of a batch, and across batches such that the mean cryoprotectant concentration, such as DMSO concentration in the cryopreserved platelets, or in a cryopreservation medium having platelets across at least 2, 3, 4, 5, 6, 7, 8, 9, 10, 15, 20, 25, 50, 75, or 100 batches has a coefficient of variance of less than 10%. In case of collections provided herein, batches in the such collections display improved homogeneity, for example, when compared to single-donor cryopreserved platelet products. For example, the DMSO concentration in the cryopreserved platelets across at least 2, 3, 4, 5, 6, 7, 8, 9, 10, 15, 20, 25, 50, 75, or 100 batches has a coefficient of variance of less than 5%. For example, the DMSO concentration in the cryopreserved platelets across at least 2, 3, 4, 5, 6, 7, 8, 9, 10, 15, 20, 25, 50, 75, or 100 batches has a coefficient of variance of less than 3%. For example, the DMSO concentration in the cryopreserved platelets across at least 2, 3, 4, 5, 6, 7, 8, 9, 10, 15, 20, 25, 50, 75, or 100 batches has a coefficient of variance of less than 2%. For example, the DMSO concentration in the cryopreserved platelets across at least 2, 3, 4, 5, 6, 7, 8, 9, 10, 15, 20, 25, 50, 75, or 100 batches has a coefficient of variance of less than 1%. For example, the DMSO concentration in the cryopreserved platelets across at least 2, 3, 4, 5, 6, 7, 8, 9, 10, 15, 20, 25, 50, 75, or 100 batches has a coefficient of variance of less than 0.5%. For example, the DMSO concentration in the cryopreserved platelets across at least 2, 3, 4, 5, 6, 7, 8, 9, 10, 15, 20, 25, 50, 75, or 100 batches has a coefficient of variance of less than 0.4%. Accordingly, in some embodiments of aspects that include a process, or a collection of cryopreserved platelets, DMSO concentration in the cryopreserved platelets across batches or within a batch can be in the range of 0.01-2%, 0.01-1.8%, 0.01-1.6%, 0.01-1.5%, 0.01-1.3%, 0.01-1.1%, 0.01-1%, 0.01-0.8%, 0.01-0.7%, 0.01-0.5%, 0.01-0.4%, 0.02-0.4%, or 0.04-0.4%. For example, the mean cryoprotectant concentration (e.g., DMSO concentration) in the cryopreserved platelets across at least 2, 3, 4, 5, 6, 7, 8, 9, 10, 15, 20, 25, 50, 75, or 100 batches has a coefficient of variance of less than 5%. In some embodiments, the mean cryoprotectant concentration (e.g., DMSO concentration) in the cryopreserved platelets across at least 2, 3, 4, 5, 6, 7, 8, 9, 10, 15, 20, 25, 50, 75, or 100 batches has a coefficient of variance of less than 1%. For example, the mean cryoprotectant concentration (e.g., DMSO concentration) in the cryopreserved platelets across at least 2, 3, 4, 5, 6, 7, 8, 9, 10, 15, 20, 25, 50, 75, or 100 batches has a coefficient of variance of less than 0.8% 0.5%, or 0.4%. Typically, the mean DMSO concentration in the cryopreserved platelets across at least 10 batches can be less than 0.5%. In some embodiments, the cryoprotectant is DMSO, the mean concentration of DMSO in the cryopreserved platelets in a cryo-vessel across at least 10 batches has a coefficient of variance of less than 5%, 4%, 3%, 2%, 1%, or 0.5%. In some embodiments, DMSO concentration in the cryopreserved platelets across at least 10 batches or within a batch can be in the range of 0.01-2%, 0.01-1.8%, 0.01-1.6%, 0.01-1.5%, 0.01-1.3%, 0.01-1.1%, 0.01-1%, 0.01-0.8%, 0.01-0.7%, 0.01-0.5%, 0.01-0.4%, 0.02-0.4%, or 0.04-0.4%. For example, the mean concentration of DMSO in the cryopreserved platelets in a cryo-vessel across at least 20 batches has a coefficient of variance of less than 5%, 4%, 3%, 2%, 1%, or 0.5%. In some embodiments, DMSO concentration in the cryopreserved platelets across at least 20 batches or within a batch can be in the range of 0.01-2%, 0.01-1.8%, 0.01-1.6%, 0.01-1.5%, 0.01-1.3%, 0.01-1.1%, 0.01-1%, 0.01-0.8%, 0.01-0.7%, 0.01-0.5%, 0.01-0.4%, 0.02-0.4%, or 0.04-0.4%. For example, the mean concentration of DMSO in the cryopreserved platelets in a cryo-vessel across at least 50 batches has a coefficient of variance of less than 5%, 4%, 3%, 2%, 1%, or 0.5%. In some embodiments, DMSO concentration in the cryopreserved platelets across 50 batches or within a batch can be in the range of 0.01-2%, 0.01-1.8%, 0.01-1.6%, 0.01-1.5%, 0.01-1.3%, 0.01-1.1%, 0.01-1%, 0.01-0.8%, 0.01-0.7%, 0.01-0.5%, 0.01-0.4%, 0.02-0.4%, or 0.04-0.4%. For example, the mean concentration of DMSO in the cryopreserved platelets in a cryo-vessel across at least 100 batches has a coefficient of variance of less than 5%, 4%, 3%, 2%, 1%, or 0.5%. In some embodiments, DMSO concentration in the cryopreserved platelets across at least 100 batches or within a batch can be in the range of 0.01-2%, 0.01-1.8%, 0.01-1.6%, 0.01-1.5%, 0.01-1.3%, 0.01-1.1%, 0.01-1%, 0.01-0.8%, 0.01-0.7%, 0.01-0.5%, 0.01-0.4%, 0.02-0.4%, or 0.04-0.4%. For example, the mean concentration of DMSO in the cryopreserved platelets in a cryo-vessel across 5-100 batches has a coefficient of variance of less than 1%, or 0.5%. In some embodiments, DMSO concentration in the cryopreserved platelets across 5-100 batches or within a batch can be in the range of 0.01-2%, 0.01-1.8%, 0.01-1.6%, 0.01-1.5%, 0.01-1.3%, 0.01-1.1%, 0.01-1%, 0.01-0.8%, 0.01-0.7%, 0.01-0.5%, 0.01-0.4%, 0.02-0.4%, or 0.04-0.4%. For example, the mean concentration of DMSO in the cryopreserved platelets in a cryo-vessel across 5-500 batches has a coefficient of variance of less than 1%, or 0.5%. In some embodiments, DMSO concentration in the cryopreserved platelets across 5-500 batches can be in the range of 0.01-2%, 0.01-1.8%, 0.01-1.6%, 0.01-1.5%, 0.01-1.3%, 0.01-1.1%, 0.01-1%, 0.01-0.8%, 0.01-0.7%, 0.01-0.5%, 0.01-0.4%, 0.02-0.4%, or 0.04-0.4%. In some embodiments, the concentration of DMSO in the cryopreserved platelets in a cryo-vessel within a batch has a coefficient of variance of less than 10%, 9%, 8%, 7%, 5%, 3%, 1%, 0.8%, 0.6%, or 0.5%. In some embodiments, the concentration of DMSO in the cryopreserved platelets in a cryo-vessel within a batch or across at least 5 batches varies within 5%, 4%, 3%, 2%, 1%, 0.5%, or 0.4%. In some embodiments, the concentration of DMSO in the cryopreserved platelets in a cryo-vessel within a batch or across at least 10 batches varies within 5%, 4%, 3%, 2%, 1%, 0.5%, or 0.4%. In some embodiments, the concentration of DMSO in the cryopreserved platelets in a cryo-vessel within a batch or across 5-100 batches varies within 5%, 4%, 3%, 2%, 1%, 0.5%, or 0.4%.

In some embodiments of aspects that include process for preparing a batch of cryopreserved platelets, a process for preparing a cryopreserved platelet composition including a transition in freezing temperatures from an initial freezing temperature to a storage freezing temperature, and a collection or a batch comprising cryopreserved platelets, the concentration of platelets in the cryopreserved platelets in a cryo-vessel within a batch or across batches varies within 20%, 15%, 12%, 10%, or 8%. For example, the concentration of platelets in the cryopreserved platelets in a cryo-vessel within a batch or across batches varies in the range of 2-20%, 2-15%, 2-12%, 2-10%, 5-20%, 7-20%, or 10-20%. For example, the concentration of platelets in the cryopreserved platelets in a cryo-vessel within a batch or across 5-100 batches varies within 20%, 15%, 12%, 10%, or 8%. For example, the concentration of platelets in the cryopreserved platelets in a cryo-vessel within a batch or across 5-100 batches varies in the range of 2-20%, 2-15%, 2-12%, 2-10%, 5-20%, 7-20%, or 10-20%. For example, the concentration of platelets in the cryopreserved platelets in a cryo-vessel across at least 5 batches has a mean intra-batch coefficient of variance of less than 20%, 18%, 1%, 12%, 10%, 8%, 5%, or 4%. In some embodiments, the concentration of platelets in the cryopreserved platelets in a cryo-vessel across at least 5 batches has a mean intra-batch coefficient of variance in the range of 2-20%, 2-15%, 2-12%, 2-10%, 2-8%, 2-6%, or 2-5%. For example, the concentration of platelets in the cryopreserved platelets in a cryo-vessel within a batch has a coefficient of variance of less than 20%, 18%, 15%, 12%, 10%, 8%, 5%, or 4%. In some embodiments, the concentration of platelets in the cryopreserved platelets in a cryo-vessel within a batch has a coefficient of variance in the range of 2-20%, 2-15%, 2-12%, 2-10%, 2-8%, 2-6%, 3-6%, 3-20%, 3-15%, 3-12%, 3-10%, or 3-6%.

In some embodiments of aspects that include process for preparing a batch of cryopreserved platelets, a process for preparing a cryopreserved platelet composition including a transition in freezing temperatures from an initial freezing temperature to a storage freezing temperature, and a collection or a batch comprising cryopreserved platelets, the total number of platelets in the cryopreserved platelets in a cryo-vessel within a batch or across batches varies within 20%, 15%, 12%, 10%, or 8%. For example, the total number of platelets in the cryopreserved platelets in a cryo-vessel within a batch or across batches varies in the range of 2-20%, 2-15%, 2-12%, 2-10%, 5-20%, 7-20%, or 10-20%. For example, the total number of platelets in the cryopreserved platelets in a cryo-vessel within a batch or across 5-100 batches varies within 20%, 15%, 12%, 10%, or 8%. For example, the total number of platelets in the cryopreserved platelets in a cryo-vessel within a batch or across 5-100 batches varies in the range of 2-20%, 2-15%, 2-12%, 2-10%, 5-20%, 7-20%, or 10-20%. For example, the total number of platelets in the cryopreserved platelets in a cryo-vessel across at least 5 batches has a mean intra-batch coefficient of variance of less than 20%, 18%, 15%, 12%, 10%, 8%, 5%, or 4%. In some embodiments, the total number of platelets in the cryopreserved platelets in a cryo-vessel across at least 5 batches has a mean intra-batch coefficient of variance in the range of 2-20%, 2-15%, 2-12%, 2-10%, 2-8%, 2-6%, or 2-5%. For example, the total number of platelets in the cryopreserved platelets in a cryo-vessel within a batch has a coefficient of variance of less than 20%, 18%, 15%, 12%, 10%, 8%, or 7%. In some embodiments, the total number of platelets in the cryopreserved platelets in a cryo-vessel within a batch has a coefficient of variance in the range of 2-20%, 2-15%, 2-12%, 2-10%, 2-8%, 2-7%, 3-7%, 3-20%, 3-15%, 3-12%, or 3-10%.

Methods for Selecting Cryopreserved Platelets for a Recipient with Platelet Alloantibodies

A collection of HLA-characterized (e.g., matched) cryopreserved platelets, for example as prepared by processes herein, which in illustrative embodiments is a collection of cryopreserved platelets, can be used herein in methods of selecting, administering, and treating disclosed herein. For example, a collection of HLA-characterized cryopreserved platelets can be used in methods for selecting HLA Class 1 compatible cryopreserved platelets for a recipient in need thereof. It is noteworthy that a “recipient”, “subject” or “patient” can be used interchangeably herein. A recipient is typically a mammalian subject, and in illustrative embodiments is a human subject. Such recipient in illustrative embodiments is in need thereof because they have anti-HLA alloantibodies and/or because they are refractory to platelet transfusion (platelet transfusion refractory). In some embodiments the recipient is refractory to platelet transfusion (platelet refractory) but they do not have detectable anti-HLA alloantibodies. In some embodiments, the recipient is not refractory to platelet transfusion. Typically, alloantibodies are antibodies that recognize foreign antigens in a subject, and anti-HLA alloantibodies are antibodies that recognize HLA of platelets of a foreign subject. In some embodiments, HLA Class 1 compatible cryopreserved platelets herein are HLA characterized cryopreserved platelets that can be compatible to a recipient or a subject. For example, HLA Class 1 compatible cryopreserved platelets can be HLA Class 1 antigen-matched cryopreserved platelets to the platelets of the recipient of the subject. In some cases, HLA Class 1 compatible cryopreserved platelets can be HLA Class 1 antigen epitope-based matched to the platelets of the recipient or the subject. In some embodiments, such epitope-based matched cryopreserved platelets can be HLA Class 1 eplet-matched cryopreserved platelets, or HLA Class 1 eplet-based mismatch acceptable cryopreserved platelets. In some cases, HLA Class 1 compatible cryopreserved platelets can be matched based on the HLA Class 1 antigens of cryopreserved platelets falling within the same cross-reactive groups (CREGs) as the HLA Class 1 antigens of the platelets of the recipient or the subject. In some cases, HLA Class 1 compatible cryopreserved platelets can be HLA-characterized cryopreserved platelets against which there is no cross reactivity or least cross reactivity in a cross-match assay between an antibody-containing sample of a recipient and the HLA-characterized cryopreserved platelets. In some embodiments, methods herein include after selecting a desired batch, administering an effective dose of the HLA compatible cryopreserved platelets as a single dose, or a multiple dose, for example, at least 2, 3, 4, 5, 6, 7, 8, 9, 10 dose over a span of 12 hours, 24 hours, 36 hours, 48 hours, 60 hours, 72 hours, 84 hours, 96 hours, 5 days, 6, days, 7 days, 8 days, 9 days, 10 days, or more. For example, 2-200, 2-150, 2-100, 1-75, 2-50, 2-25, 10-200, 25-200, 50-200, 100-200, or 150-200 doses. Once a desired batch of cryopreserved platelets is selected a single or multiple doses as per requirement can be given from different vials or vessels of the same batch.

Such methods for selecting can utilize HLA characterization information about batches of cryopreserved platelets to select a batch of cryopreserved platelets for a recipient using HLA Class 1 virtual cross-matching. In other aspects, such methods for selecting HLA Class 1 compatible cryopreserved platelets for a recipient in need thereof can use HLA matching, which in illustrative embodiments, HLA match grades are used for such matching. In other embodiments, epitope matching is used to select a batch of cryopreserved platelets to administer to a recipient in need thereof. Furthermore, some aspects herein for selecting a batch of cryopreserved platelets to administer to a subject do not utilize previously known HLA characteristic information about a donor cryopreserved platelets batch, but rather utilize an immunological physical cross-matching reaction, as discussed further herein.

Methods for selecting cryopreserved platelets herein, can include virtual cross-matching, also known as antigen-restricted method for selecting the cryopreserved platelets. For performing a virtual cross-matching of the recipient and the batches of HLA Class 1-characterized cryopreserved platelets, the information regarding the type of antibodies (for example, alloantibodies) produced in a recipient can be obtained by known techniques including, but not limited to Luminex-based phenotypic beads such as, beads coated with specific Class 1 or Class 2 HLA antigens. In some embodiments, the Luminex-based beads having only single type of antigen, such as, single-antigen bead assay (SAB) can also be used. In SAB, Luminex beads are coated with a single HLA on their surface and incubated with the serum of the recipient. If the recipient has antibodies against the specific HLA that is coated on the beads, they will bind to the bead that is coated with the respective antigen. The beads are then washed and incubated with phycoerythrin (PE)-labeled anti-human IgG antibodies. This antibody will bind to the Fc region of antibodies bound to beads. The mixture is then washed and analyzed using a Luminex instrument. Each bead is identified through a unique combination of two fluorescent dyes (red and infrared) impregnated into the bead. The anti-human IgG complex will emit fluorescence upon exposure to laser light. This fluorescence is detected on a Luminex platform and antibody reactivity is recorded as mean fluorescence intensity.

Another non-limiting example of a method for analyzing a recipient's alloantibodies includes generating a calculated panel-reactive antibody (cPRA or PRA) percentage. A cPRA can be generated by a commonly known test that employs Luminex-based phenotypic beads to screen for HLA antibodies, typically, HLA Class-1 antibodies. A cPRA provides a percentage of antibody in a test serum, typically from the recipient that are reactive against a panel of known HLA antigens. Therefore, higher cPRA percentage denotes higher number of antibodies in the serum of the recipient that are reactive against a known panel of HLA Class-1 antigens. Accordingly, in some embodiments, cPRA can be used to guide the selection and/or administration of cryopreserved platelets herein to a subject either independently or in combination with the HLA-matching between the cryopreserved platelets and the platelets of the recipient, or with the virtual cross-matching assay as disclosed herein. Accordingly, subjects, patients, or recipients in methods herein can have a PRA score of greater than 10, 15, 20, 25, 30, 40, 50, 60, 70, 75 80, 85, or 90%, or between 15% on the low end and 50, 60, 70, 75 80, 85, or 90% on the high end. In some embodiments, the subject, patient or recipient is refractory to platelet transfusions. In one illustrative embodiment, the subject, patient, or recipient has a PRA score of greater than 15% and less than 70%. In embodiment, the subject, patient, or recipient has a PRA score of greater than 70% and platelets of the subject, patient, or recipient are matched to cryopreserved platelets, that are administered to the subject. In embodiment, the subject, patient, or recipient has a PRA score of greater than 70% and platelets of the subject, patient, or recipient are not matched to cryopreserved platelets, that are administered to the subject. Once the list of HLA antigens against which the recipient produces alloantibodies is determined, the recipient can be provided the cryopreserved platelets which have at most 0, 1, 2, 3, 4, or 5 of the antigens against which the recipient produces alloantibodies. For example, among HLA Class 1 antigens, the type of HLA-A, and HLA-B against which the recipient produces the alloantibodies is determined, then the batches of HLA Class 1-characterized cryopreserved platelets can be screened for selecting a desired batch of cryopreserved platelets based on the absence of 1, 2, 3, 4 or 5 antigens recognized by alloantibodies of the recipient. In illustrative embodiments, the desired batch of cryopreserved platelets has none of the one or more allogeneic HLA Class 1 antigens recognized by the alloantibodies of the recipient.

Methods for selecting cryopreserved platelets herein, can include cross-matching the alloantibodies produced in a recipient directly with the cryopreserved platelets, to select cross-match compatible cryopreserved platelets. Typically, performing a cross-matching assay does not require or include identifying the specific HLA against which the recipient produces alloantibodies. In a cross-matching assay, HLA Class 1-characterized cryopreserved platelets from each of at least 2, 3, 4, 5, or more batches of a collection herein, are contacted with an antibody-containing sample of the recipient, wherein the recipient is in need of a platelet transfusion; and a desired batch of HLA Class 1-characterized cryopreserved platelets from the collection is selected based on a batch from the collection having HLA Class-1 characterized cryopreserved platelets against which there is the lowest, in illustrative embodiments, no observed cross-reaction in the cross-match assay. In some embodiments, cryopreserved platelets from each batch, wherein the number of batches range from 2-20, 2-15, or 2-10 are contacted in the cross-match assay. Cross-matching can be performed by known techniques, including, but not limited to solid-phase red cell adherence assay (SPRCA), modified antigen capture ELISA, and flow cytometry. In the SPRCA method, thawed and resuspended cryopreserved platelets are bound to the bottom of a microtiter plate. Recipient's serum is added to each well, incubated, and washed before the addition of the AHG-coated RBCs for detecting the antigen and antibody cross-reaction.

Methods herein for selecting cryopreserved platelets herein, can include a method for matching HLA of the recipient with the batches of cryopreserved platelets. Such methods can include a step of selecting a composition of HLA-matched cryopreserved platelets, such as in a batch of HLA-matched cryopreserved platelets, that matches the HLA type of a potential recipient subject in need of a hemostatic agent, such as for example, platelets. Thus, in order to identify and select a batch of cryopreserved platelets from the collection that are an HLA match for the potential recipient (e.g., the subject in need of a hemostatic agent), HLA characteristics (e.g., HLA Class 1 antigen type) typically are, or have been determined for the potential recipient subject. For example, the HLA Class 1 type of the potential recipient can be determined.

In these embodiments, this information regarding the HLA class of the potential recipient, is transmitted or otherwise communicated to and received by a cryopreserved platelets provider, manufacturer, and/or distribution center that has, stores and/or has access to information regarding the physical collection of HLA-matched cryopreserved platelets and information regarding the identity and location of cryo-vessel(s) that contain a batch of cryopreserved platelets in the collection. For example, such cryo-vessels can be stored frozen, or in illustrative embodiments, at ambient temperature, for example in a warehouse or other storage facility that meets government regulatory (e.g. FDA) requirements for such storage. Information regarding the HLA type of the potential recipient is then used to search similar information regarding batches in the collection. One or more batches with a matching HLA characteristic (e.g. HLA Class 1 type) is identified, if present in the collection, and selected, for example for delivery to the site of administering to the potential recipient. Thus, the selecting can be part of a method that includes shipping cryo-vessel(s) containing cryopreserved platelets of the selected batch, as part of a commercialization process to fulfill an order for such cryopreserved platelets. In some embodiments, matching HLA characteristic can be matching HLA Class 1 type comprising HLA-A, HLA-B, and HLA-C antigens. In illustrative embodiments, matching HLA characteristic can be matching Class 1 type comprising HLA-A, and HLA-B antigens. A batch of cryopreserved platelets herein can have 2, 3, 4, 5, 6, 7, 8, 9, or 10, or between 2 and 10, 9, 8, 7, 6, 5, 4, 3, or 2 HLA Class 1 antigens. In illustrative embodiments, cryopreserved platelets have between 2 and 4 HLA Class 1 antigens.

It is envisioned that there can be different grades of matching between the platelets of a plurality of donors forming a pooled platelet composition, or between the cryopreserved platelets herein and the platelets of a recipient. As per a standard definition of HLA-matched platelets (Mittal K K et al. Matching of histocompatibility (HL-A) antigens for platelet transfusion. Blood. 1976; 47:31-41, incorporated herein by reference in its entirety; Schmidt, Amy E et al. “HLA-Mediated Platelet Refractoriness.” American journal of clinical pathology vol. 151, 4 (2019): 353-363, incorporated herein by reference in its entirety), HLA matching can be determined for HLA-A and HLA-B antigens, and different match grades can be assigned. The HLA genes are co-dominantly expressed in an individual, therefore, an individual heterozygous for HLA genes can inherit a maximum of two alleles for each locus. Therefore, considering HLA matching of platelets for HLA-A and HLA-B genes, an individual can typically express a maximum of 4 different antigens (2 each for HLA-A and HLA-B). For example, grade A is assigned where there is a “4-antigen match” between the donor (or FDPDs) and recipient/subject (or other donor in case of plurality of donors for forming a pooled platelet composition). Grade B is assigned when there are no mismatched antigens. Grade B has different specific grades that can be assigned based on further classification. For example, grade B1U is assigned when only 3 antigens are detected in a donor (homozygous at 1 HLA allele), and all 3 antigens are identical to the recipient, grade B1X is assigned when out of the 3 antigens, 2 antigens are identical to the recipient and 1 antigen is cross-reactive. Grade B2U is assigned when only 2 antigens are detected in a donor (homozygous at 2 HLA alleles), and both the detected antigens are identical to the recipient. Grade B2UX is assigned when only 3 antigens are detected in the donor (homozygous at 1 HLA allele), 2 antigens are identical with the recipient, and 1 antigen is cross-reactive. Grade B2X is assigned when 2 antigens are identical with the recipient and 2 antigens are cross-reactive. Grade C is assigned when 1 antigen of donor is not present in the recipient and are not cross-reactive with the subject. Grade D is assigned when 2 antigens of donor are not present in the recipient and are not cross-reactive with the recipient. Grade R is assigned when there is a random donor. In general, platelets with a higher degree of match have shown improved survival after platelet transfusion. For example, grades A, B1U, and B2U matches have been shown to provide the relative best increases in platelet counts post-transfusion (Schmidt, Amy E et al. “HLA-Mediated Platelet Refractoriness.” American journal of clinical pathology vol. 151, 4 (2019): 353-363). Platelets from donors having HLA antigens from the same cross-reactive group as the recipient's platelets have also been shown to produce better results post-transfusion (Duquesnay, R J et al. “Transfusion therapy of refractory thrombocytopenic patients with platelets from donors selectively mismatched for cross-reactive HLA antigens.” Transplantation proceedings vol. 9, 1 Suppl 1 (1977): 221-4) which can be partially due to the inability of the recipient's immune system to recognize the cross-reactive groups as foreign antigens (for example, grades B2UX, B2×). Cross-reactive groups (CREGs) have been identified in the art, for example, in the publications Rodey, G E et al. “Epitope specificity of HLA class I alloantibodies. I. Frequency analysis of antibodies to private versus public specificities in potential transplant recipients.” Human immunology vol. 39, 4 (1994): 272-80; and Wade J A, et al. “HLA mismatching within or outside of cross-reactive groups (CREGs) is associated with similar outcomes after unrelated hematopoietic stem cell transplantation”. Blood. 2007 May 1; 109(9):4064-70. Further, HLA-matched platelets with grades B2X, C, and D have been shown to provide platelet responses similar to transfusion of platelets from random donors.

CREGs are generally defined by sharing of a public epitope, therefore, HLA Class 1 matching can be based on cross-reactive HLA Class 1 antigens rather than matching exactly the same HLA Class 1 antigens. Accordingly, in some embodiments, platelets, or cryopreserved platelets herein can be matched by identifying the CREGs between the platelets in a platelet composition obtained from a plurality of donors, or between the cryopreserved platelets herein and the platelets of the recipient. A skilled artisan would understand that identifying CREGs can be a step that can be considered alternative to matching HLA antigens on the surface of different platelets, or cryopreserved platelets, in case there is no suitable match found between the antigens using exact matching or more stringent matching grades not based on CREG matching. Alternatively, identifying CREGs can be an independent step to assess the HLA characteristic for pooling platelets from a plurality of donors, or for matching the cryopreserved platelets to a recipient in need thereof. Accordingly, HLA-matched platelets herein can be HLA-matched because they include cross-reactive HLA antigens, typically, Class-1 antigens on the surface of cryopreserved platelets that fall within the same group as that of the platelets of the recipient or subject. In some embodiments, HLA-matched platelets herein can be HLA-matched for purposes of matching a recipient to cryopreserved platelets to be administered to the recipient, or for identifying donors whose platelets to include in a pool for cryopreserved platelets production, because they include cross-reactive HLA Class-1 antigens, that fall within the same cross-reactive group.

In some embodiments, instead of matching entire HLA antigens between platelets of the recipient and cryopreserved platelets to be administered to the recipient, or between platelets to pool before using them to make cryopreserved platelets, HLA matching is performed, or cryopreserved platelets are HLA matched cryopreserved platelets, based on epitope matching (see e.g., Marsh et al. “An epitope-based approach of HLA-matched platelets for transfusion: a noninferiority crossover randomized trial.” Blood 2021; 137 (3): 310-32). HLA epitopes can be described or identified in the form of eplets. An eplet is a polymorphic amino acid tridimensional configuration within the epitope that is recognized by alloantibodies. In case of an HLA mismatch, a recipient can develop alloantibodies against the HLA. However, in order to produce the alloantibodies, the HLA has to be accessible at the protein level in its quaternary structure. Eplet information can provide details regarding the quaternary structure of the epitope that is recognized by alloantibodies of the recipient. Accordingly, in some embodiments, HLA typing of cryopreserved platelets from a collection herein can be compared with the HLA typing of the recipient to assess the epitope and/or eplet matching and the information can then be used to select the desired batch of cryopreserved platelets having higher number of epitope and/or eplet matching with the HLA type of the recipient. In some embodiments, the identity of HLA Class 1 antigens against which the recipient is capable of generating alloantibodies can also be used to decide the acceptability of epitope and/or eplet mismatch between cryopreserved platelets and the HLA type of the recipient.

Accordingly, in certain embodiments methods for selecting cryopreserved platelets herein include receiving an HLA Class 1 type of the recipient, wherein the recipient is in need of a platelet transfusion, and comparing the HLA Class 1 type of the recipient to the HLA Class 1 type of the batches in a collection of cryopreserved platelets, and a comparing step is performed to select a desired batch of HLA Class 1-characterized cryopreserved platelets from the collection by comparing epitopes and/or eplets of the batches of the collection with the one or more HLA Class-1 epitopes and/or eplets in the recipient. A desired batch can then be selected based on the cryopreserved platelets having higher number of epitope and/or eplet matching between cryopreserved platelets and the HLA Class-1 antigens in the recipient as compared to the cryopreserved platelets in other batches. Methods herein can further include receiving an identity of one or more HLA Class-1 epitopes recognized by alloantibodies in the recipient, and a desired batch of cryopreserved platelets can then be selected based on eplet-based mismatch acceptability of one or more allogeneic HLA Class 1 antigens. In some embodiments, the desired batch of HLA Class 1-characterized cryopreserved platelets has at least 1 mismatch of the HLA Class 1 type of the recipient. Typically, the desired batch of HLA Class1-characterized cryopreserved platelets is premade, before a recipient in need of a hemostatic agent is identified as such.

Epitopes are conformational arrangements of one or more continuous chains of amino acids that are targeted by antibodies. The epitopes can be private, i.e., on a single antigen, or public, shared by two or more HLA antigens. Cross-reactive Groups (CREGs) can be defined by a shared public epitope and can be grouped by serologic cross-reactivity patterns. To perform epitope-based matching, a Luminex HLA Class-1 single antigen bead (SAB) assay can be coupled with an epitope generating computer instruction code (e.g., software program), for example, HLA Matchmaker (www.hlamatchmaker.net). This software program takes into account the patient's HLA antibodies and generates epitopes that are possibly recognized by these antibodies. The software identifies immunogenic epitopes represented by amino acids located within approximately 3 to 3.5A& radius of a polymorphic residue in antibody accessible regions of HLA antigens. This software also uses the patient's HLA typing at higher resolution levels than the serological level. Each HLA antigen has multiple epitopes that can be recognized by specific antibodies. These epitopes are characterized using three-dimensional models and despite the high polymorphism content of HLA antigens, it has become possible to determine HLA compatibility at the structural level. Each recipient has his own repertoire of self-epitopes. HLA molecules share the same sequence comprising respective amino acid motifs which bring them a certain level of similarity. If the recipient shares the same motifs creating structural similarities with the donor's antigens or the antigens of HLA Class 1-characterized cryopreserved platelets from the collection, then the patient will not develop HLA antibodies, or may develop a very limited number of, typically non-harmful HLA antibodies against the donor. This approach of epitope matching, or structural compatibility, aims to determine the number of differences between the recipient and donor or cryopreserved platelets to assess the risk of HLA antibody development. The method focuses on epitopes recognized by HLA antibodies and the determination of epitope-based mismatch acceptability for sensitized recipients considered for compatible platelet transfusions. The method converts each HLA antigen into a string of potentially immunogenic triplets and then determines which triplets on mismatched donor HLA antigens or Class 1-characterized cryopreserved platelets from the collection are shared or not shared with the recipient's HLA antigens. In other words, a cross-reactivity pattern is to identify the acceptable HLA mismatches because the information received by the cross-reactivity pattern offers a window of opportunity of finding more suitable donors for a recipient, for example, a recipient refractory to platelet transfusion. The HLA Matchmaker-based analysis of serum reactivity patterns can determine which alleles have eplets that do not react with the recipient's antibodies and these can be considered acceptable mismatches. In some embodiments, epitope-based matching can be performed by using a software not limited to HLA Matchmaker, for screening the HLA Class 1 types of HLA-characterized cryopreserved platelets in a batch or a collection herein to determine HLA-characterized cryopreserved platelets having no or least number of epitope mismatches. A person of skill in the art can understand that depending upon the policies or restrictions in a geographical location any software that is available can be used to identify epitopes (including various structural properties).

Typically, regardless of the method of selecting that is employed, and for any method of administering herein, the desired batch is premade, and thus made before the identity is received. After selecting the desired batch, the desired batch is packaged for shipment, and labeled for delivery to a location where the HLA Class 1 compatible cryopreserved platelets in the desired batch will be delivered to the recipient. For administering to the recipient, the cryopreserved platelets from the required number of cryo-vessels of the desired batch can be thawed, or thawed and resuspended as disclosed herein to form HLA Class 1 compatible platelets for the recipient, and the platelets can be administered to the recipient.

In some illustrative embodiments, the platelets in a pooled platelet composition, which can be used to make a collection of cryopreserved platelets as disclosed herein, can be HLA-matched for HLA Class-1 antigens. The HLA Class-1 antigens that are known in the art to be present on the surface of platelets are HLA-A, HLA-B, and HLA-C. In illustrative embodiments, the platelets in the platelet composition can be HLA-matched for HLA-A and HLA-B antigens between a plurality of donors. Typically, cryopreserved platelets as disclosed herein, obtained using HLA-matched pooled platelets are also HLA-matched, for example having the same HLA-A and HLA-B antigens if the platelet donors each expressed the same HLA-A and HLA-B antigens. Methods of treating a subject herein, in some embodiments can include cryopreserved platelets that are prepared from a platelet composition obtained from a single donor as a starting material. In illustrative embodiments, such methods of treating a subject are performed using HLA-matched, cryopreserved platelets prepared using HLA-matched, pooled platelets. Methods of treating a subject herein, in some embodiments can further include HLA-matching between the cryopreserved platelets administered to a subject, and the platelets of the subject. In illustrative embodiments, the HLA-matching between the cryopreserved platelets and the platelets of the subject can be done for HLA Class-1 antigens. In illustrative embodiments, HLA-matching between the cryopreserved platelets and the platelets of the subject are done for HLA-A and HLA-B antigens.

As discussed in more detail herein, in some embodiments, methods herein include steps to determine whether the subject produces anti-HLA antibodies to a defined HLA antigen, in illustrative embodiments, HLA Class-1 antigens and/or anti-platelet antibodies, in illustrative embodiments, anti-human platelet antibodies. Compositions herein, as well as methods that include or produce such compositions, including methods for administering such compositions to subjects with anti-HLA antibodies, can include cryopreserved platelets that have no mismatch of HLA Class-1 antigens within the population of cryopreserved platelets. In some embodiments, the compositions herein can include a population of cryopreserved platelets such that the cryopreserved platelets have no more than two mismatches, in illustrative embodiments, no more than one mismatch of HLA Class-1 antigens, in illustrative embodiments, HLA-A and HLA-B antigens within the population of cryopreserved platelets.

Compositions herein, as well as processes that include or produce such compositions, can include a population of cryopreserved platelets that include cross-reactive HLA antigens that are in the same cross-reactive groups (CREGs) as other cryopreserved platelets within the population of cryopreserved platelets.

Compositions herein, as well as processes that include or produce such compositions, can include a population of cryopreserved platelets that are HLA-matched within the population of cryopreserved platelets, and the HLA-matched cryopreserved platelets have HLA-matched eplets. A skilled artisan will understand that the degree of HLA-matched eplets is subjected to variation depending upon a specific donor-recipient pair.

Compositions herein in illustrative embodiments, include cryopreserved platelets that are prepared using platelets from a plurality of donors (e.g. pooled platelets). Thus, compositions herein, and processes using and for preparing the same, can include a population of cryopreserved platelets that have a biomolecule profile indicative of more than 1 platelet donor. A skilled artisan will understand that there are various molecular tests that can be used to confirm that a cryopreserved platelet composition includes cryopreserved platelets from a plurality of donors. In some embodiments, the biomolecule profile indicative of more than 1 platelet donor is the presence of two or more alleles/versions/amino acid sequences of at least a first protein from at least a first gene that are significantly different than 50% in frequency within the composition. A 50% frequency would be expected, for example, if such composition was from a single donor that was heterozygous for alleles at the first gene. In certain embodiments, the first protein is not an HLA Class-1 antigen, for example, not HLA-A and/or HLA-B antigens, since in illustrative embodiments platelet derivatives in a composition herein, are from a plurality of donors who are identically HLA-matched. In illustrative embodiments, the first protein is an HLA Class1 antigen, for example, an HLA-A and/or HLA-B antigen, and the plurality of donors do not have identical HLA Class 1 antigens.

The process of HLA-typing to characterize the HLA-A and HLA-B antigens on the surface of platelets, and/or freeze-dried platelet derivatives can be done by known techniques. For example, such typing methods include serological typing to determine HLA phenotypes, and HLA typing by nucleic acid-based molecular techniques. Serological HLA typing can be based on the detection of expressed HLA antigens on the surface of platelets using defined panels of antisera. Serological HLA typing can be used to determine whether a particular allele is actually expressed on the surface of platelets. Nucleic-acid based molecular techniques for HLA typing can include RNA or DNA-based HLA typing, for example mRNA typing or mitochondrial DNA typing, since such DNA has been identified in platelets. Nucleic acid-based HLA typing can employ either nucleic acid-based (e.g. DNA-based) probes or primers, to perform, for example, PCR (e.g. qPCR), or nucleic acid sequencing, for example next-generation sequencing, or test for the presence or absence of sequence motifs. Commercial kits that utilize this technology can define the HLA alleles present in an individual to a variable level of resolution dependent on a number of factors. These include the number of probes or primers employed, the number of alleles defined for a given locus and the HLA alleles present in the individual. Nucleic acid-based HLA typing can resolve allele-level differences in HLA genes that cannot be detected by serology. Several approaches to nucleic acid (e.g. DNA) based HLA typing are used that offer a range of reported typing resolution levels from low (antigen-level) to high (allele-level). A non-limiting list of techniques for DNA-based HLA typing can include PCR with sequence specific primers (PCR-SSP), Sequence specific oligonucleotide probing (PCR-SSOP), Sanger sequencing-based typing (SBT), and Next-generation sequencing. Nucleic acid-based molecular techniques for HLA typing can include techniques that exploit the presence of RNA molecules (e.g. mRNA) on a platelet product.

In some embodiments of any aspects or embodiments herein that include a method for selecting cryopreserved platelets for a recipient or subject, include methods for selecting cryopreserved platelets for a recipient from different batches of HLA-characterized cryopreserved platelets, each prepared from a single platelet donor, or from plurality of platelet donors. In illustrative embodiments, the HLA-characterized cryopreserved platelets in vial(s) or cryo-vessel(s) of such batches prepared from a single platelet donor have at least one rare allele. In other embodiments, the HLA-characterized FDPDs in such sets or batches prepared from a single platelet donor do not have at least one rare allele.

In some embodiments, methods herein include subjects that are refractory to platelet administration or transfusion, the refractoriness can be because of immune-related refractoriness, non-immune-related refractoriness, or idiopathic refractoriness. Accordingly, in some embodiments, subjects, or recipients who are set to receive cryopreserved platelets as disclosed herein, in illustrative embodiments, HLA-characterized cryopreserved platelets as disclosed herein are refractory to platelet transfusion. Non-immune-related refractoriness (non-limiting list) can include refractoriness due to sepsis, infection, fever, splenomegaly, disseminated intravascular coagulation (DIC), bleeding, any medications, and hepatic sinusoidal obstruction syndrome. Immune-related refractoriness can include alloimmunization that may or may not be because of prior transplantation procedures. The alloimmunization can include the presence of anti-HLA and/or anti-HPA antibodies in the blood of the subject. The course of treatment of a subject can be based on the observation of an expected amount in a target platelet increase timeframe after administering platelets, typically, platelet transfusion. The expected amount obtained after a platelet transfusion can further be correlated with the refractoriness of the subject, for example, immune-related refractoriness, non-immune-related refractoriness, or idiopathic refractoriness, and can further decide the treatment course of the subject. For example, as per one of the treatment methods, after administering platelets, typically, 2 platelet transfusions to a subject, a platelet count increment of less than 10,000/μl after one or both platelet transfusions can be indicative of immune-related refractoriness. In case of immune-related refractoriness, the subject can be checked for panel reactive antibodies (PRA), and the subject can be typed for the HLA type. In case the PRA of the subject is elevated, immune-related refractoriness can be confirmed, and the subject can be transfused with cross-matched platelets, or antigen-restricted platelets (also known as antigen-avoidance platelets), in illustrative embodiments with HLA-matched platelets. In case the PRA of the subject is not elevated, then the possibility of HPA-mediated refractoriness or non-immune-related refractoriness can be investigated. In a different scenario, for example, after administering platelets, typically, 2 platelet transfusions to a subject, in case a platelet count increment of more than 10,000/μl after both platelet transfusions are observed, then the platelet count after 24 hours post-transfusion can be checked. If the platelet count increment after 24 hours is more than 10,000/μl, then the observation can be investigated as being inconsistent with platelet refractoriness. Alternatively, if the platelet count increment after 24 hours is less than 10,000/μl, then the observation can be consistent with non-immune-related refractoriness, and the subject can be treated accordingly. In case of using CCI as the expected amount of increase in platelet counts after platelet transfusion, one of the treatment schemes can be as follows: after administering platelets, typically, 2 platelet transfusions to a subject, if CCI is less than 5,000, then HLA/HPA antibody screening can be done of the subject, and in case the presence of antibodies against HLA and/or HPA is observed in the subject's blood, then cross-matched platelets can be administered to the subject and CCI can be then monitored. If CCI remains below 5,000 then the subject can be typed for HLA, and then HLA-matched platelets, or antigen-restricted platelets (antibody avoidance platelets) can be administered to the subject. In case the CCI still remains below 5,000 then the subject can be administered random platelets. Alternatively, during the treatment an improvement in CCI post administering cross-matched platelets, HLA-matched platelets, or antigen-restricted platelets (antibody avoidance platelets) is an indication of improved platelet count and the subject can be supported by transfusing the respective platelet types. In some embodiments, methods herein include determining whether a subject or a recipient is refractory to platelet transfusions before receiving the cryopreserved platelets herein.

Accordingly, methods herein include subjects or recipients who have undergone at least one platelet transfusion before receiving cryopreserved platelets as disclosed herein, in illustrative embodiments, HLA-characterized cryopreserved platelets. Platelet transfusion in rare congenital platelet disorders such as Bernard-Soulier syndrome, Hermansky-Pudlak syndrome, Glanzmann's thrombasthenia, thrombocytopenia with absent radii (TAR), Wiskott-Aldrich syndrome, Fanconi anaemia, amegakaryocytic thrombocytopenia can provoke the development of multi-specific HLA or platelet specific antibodies. Accordingly, in some embodiments, subjects herein would have already undergone at least 1 round of platelet transfusion before receiving the cryopreserved platelets according to the methods disclosed herein. In some embodiments, a recipient or a subject has undergone at least 1 round, or more than 1 round of platelet transfusion. For example, a recipient, or a subject has undergone 1-200, 1-175, 1-150, 1-125, 1-100, 1-75, or 1-50 rounds of platelet transfusion before receiving the cryopreserved platelets herein. In some embodiments, a recipient, or a subject has undergone at least 2, 5, 10, 25, 50, 75, 100, 125, 150, 175, 200, 225, 250, 275, or 300 rounds of platelet transfusion before receiving the cryopreserved platelets herein. In some embodiments, methods herein include determining the status of platelet transfusions of a subject or a recipient before receiving the FDPDs cryopreserved platelets.

### Collections And HLA Class

In some embodiments of any of the aspects and embodiments herein that include a batch of cryopreserved platelets, a collection of cryo-vessels, or a composition comprising frozen platelets, the collection can include, a first cryo-vessel, in illustrative embodiments from a first batch, comprising a first population of HLA Class 1-characterized cryopreserved platelets having a biomolecule profile indicative of more than 1 platelet donor; and a second cryo-vessel, in illustrative embodiments from a second batch, comprising a second population of HLA Class 1-characterized cryopreserved platelets having a biomolecule profile indicative of more than 1 platelet donor, wherein the HLA Class 1 characteristics of the first population is different than the HLA Class 1 characteristics of the second population. In some embodiments, a first cryo-vessel comprising a first population of HLA Class 1-characterized cryopreserved platelets obtained from more than 1 donor; and a second cryo-vessel comprising a second population of HLA Class 1-characterized cryopreserved platelets obtained from more than 1 donor. In some embodiments, a collection of cryo-vessels comprises a third cryo-vessel, in illustrative embodiments, from a third batch, comprising a third population of HLA Class 1-characterized cryopreserved platelets having a biomolecule profile indicative of more than 1 platelet donor, or obtained from more than 1 donor, wherein the HLA Class 1 characteristics are different for each of the first population, the second population, and the third population. A collection of cryo-vessels as disclosed herein can comprise a plurality of 3, 4, 5, 6, 7, 8, 10, 20, 50, 100, 500, 1000 or more batches of cryo-vessels, each vessel of a batch comprising a population of HLA Class 1-characterized cryopreserved platelets, and the HLA Class 1 characteristics for each batch of cryo-vessels is different from the HLA Class 1 characteristics for any other batch of cryo-vessels in the collection. In some embodiments, a collection of cryo-vessels can comprise 2-5000, 2-2000, 2-1000, 2-750, 2-500, 2-400, 2-300, 2-250, 100-5000, 500-5000, 1000-5000, or 2000-5000 batches of cryo-vessels. In illustrative embodiments, a collection of cryo-vessels can comprise 2-50, 2-45, 2-40, 2-35, 2-30, 2-25, 2-20, 2-15, or 2-10 batches of cryo-vessels. In some embodiments, a collection of cryo-vessels herein can comprise 2, 3, 5, 10, 20, 20, 100, or more cryo-vessels in each batch. There can be 2-1000, 2-750, 2-500, 2-400, 2-300, 2-200, 2-100, 100-1000, 200-1000, 500-1000, or 700-1000 cryo-vessels in each batch. Thus, a large number of readily available HLA Class 1-characterized cryopreserved platelets can be produced and stored. Whenever a requirement of HLA-matched platelets arises, a desired batch of HLA Class 1-characterized cryopreserved platelets from a collection herein can be selected based, for example, on HLA Class 1 typing of the recipient and can be provided much more readily than is currently available. Accordingly, in a collection herein, a method herein that includes a donor and a recipient, or a process for preparing a batch or a collection herein having a plurality of cryo-vessels having HLA-characterized cryopreserved platelets, HLA Class-1 matched cryopreserved platelets can comprise cryopreserved platelets that are HLA Class 1 antigen-matched cryopreserved platelets, for example, the cryopreserved platelets in the cryo-vessel have HLA Class 1 antigens matched to a grade A, B1U, B1X, B2U, B2UX, B2X, C, or D match. Such matching can relate to HLA type of platelet donors used to make a pool and/or the HLA type of a donor(s) compared to the HLA type of a recipient. In some embodiments, HLA Class-1 matched cryopreserved platelets in a collection can comprise cryopreserved platelets that are matched based on epitope-based matching of HLA Class 1 antigens. In some embodiments, HLA Class-1 matched cryopreserved platelets in a collection can comprise cryopreserved platelets that have either no mismatch of HLA Class-1 antigens or no more than one mismatch of HLA Class-1 antigens. In some embodiments, HLA Class-1 matched cryopreserved platelets in a collection can comprise cryopreserved platelets that have HLA Class 1 cross-reactive antigens falling within the same cross-reactive group.

In illustrative embodiments, each batch of cryo-vessels comprises HLA Class 1-characterized cryopreserved platelets having a defined HLA characteristics obtained from a common pooled platelet composition comprising HLA Class 1-characterized platelets with the same or similar defined HLA characteristics. In some embodiments, the HLA characteristics of the HLA Class 1-characterized cryopreserved platelets of a batch of cryo-vessels remain identical amongst all the cryo-vessels from one batch. In some embodiments, the HLA characteristics of HLA Class 1-characterized cryopreserved platelets of one batch of cryo-vessels differs from that of another batch. It is envisioned that different batches/lots of HLA Class 1-characterized cryopreserved platelets can be prepared, and each batch has a defined HLA characteristic that is different from one another. For example, in some embodiments 5 or less, 4 or less, 3 or less, 2 or less, 1 or 0 antigens, HLA antigens, for example HLA Class 1 antigens, found in one batch are found in another batch, or 1 on the low end, to 10, 15 or 20 antigens, for example HLA Class 1 antigens, can be found in one batch that are not found in another batch. The HLA characteristics of all such batches can be categorically maintained, for example, in a database such that when a request for HLA-matched platelets is received, the database can be searched against the desired HLA characteristics matching with the recipient, and upon finding the specific batch, containers/vials, for example a requested or required number of vials of the batch can be shipped to the healthcare facility. In some embodiments, the number of batches can depend upon the most common type of HLA Class-1 antigens expressed on the platelets in the population of a geographical location. In other embodiments, the number of batches can also depend upon the HLA Class-1 antigens that are rare amongst the population of a geographical location, such that, for making a repository/bank of HLA-matched platelets expressing rare Class-1 antigens.

The cryopreserved platelets, or the collection/batch/lot of cryopreserved platelets, in illustrative embodiments, HLA Class 1-characterized cryopreserved platelets as described herein are prepared from a starting material that is obtained from a plurality of donors (pool of platelets) having HLA Class 1-characterized platelets. As discussed hereinabove, one or more HLA characteristics (e.g. HLA Class 1 type) can be determined, identified, and/or defined before donor platelets are optionally pooled. In some embodiments, the HLA characteristics, for example, HLA Class 1 characteristics of cryopreserved platelets herein do not change significantly when compared to the HLA characteristics of the platelet composition or the platelet pool from which the cryopreserved platelets were prepared. Accordingly, in illustrative embodiments, cryopreserved platelets prepared from a starting material of single-donor or pooled-donor platelets, can have one or more, two or more, some, most, or all of the same HLA characteristics (e.g. HLA Class 1 antigen type) as the HLA Class 1-characterized platelets of the starting material. Accordingly, in such illustrative embodiments, the HLA characteristic of the HLA Class 1-characterized platelets of the starting material is maintained in the cryopreserved platelets. For example, a starting material comprising pooled platelets that have a defined HLA characteristic, in a hypothetical non-limiting example, such as, A1, and A3 of HLA-A, and B8, and B27 of HLA-B, used to prepare cryopreserved platelets herein, results in a batch, or a collection of cryo-vessels comprising cryopreserved platelets having the same HLA characteristic(s) (e.g., HLA Class 1 type) of the starting material. Accordingly, in some embodiments, types and/or characteristics of HLA Class 1 antigens of HLA-characterized cryopreserved platelets of a batch is the sum of some, and in illustrative embodiments all the types and/or characteristics of HLA Class 1 antigens of platelets in a pool of platelets used to prepare the batch of HLA-characterized cryopreserved platelets.

Accordingly, the cryo-vessels that contain a batch of cryopreserved platelets are identifiable, for example by a label on the cryo-vessel. The location of such cryo-vessel can be stored for example in a computer database that can be searched and retrieved at a later time, for example when a potential recipient in need of a hemostatic agent is identified.

Cryopreserved Platelets for Reducing and/or Treating Bleeding

In some aspects and embodiments, provided herein is a method for reducing and/or treating bleeding in a subject, the method comprising administering a dose, or an effective amount of frozen platelets, cryopreserved platelets, or cryopreserved platelet derivatives including, but not limited to, those of any of the aspects or embodiments herein, or the composition prepared by any of the processes described herein, such as in aspects or embodiments herein. In some embodiments, the administering includes administering a composition comprising frozen platelets, such that the composition comprises frozen platelets in a cryopreservation medium. In some embodiments, the composition comprises cryopreserved platelets and/or cryopreserved platelet derivatives. Typically, the administering includes thawing the composition comprising cryopreserved platelets in the cryopreservation medium, to form a thawed composition. Such a thawed composition can further be resuspended using a sterile solution, for example, saline, such as 0.9% saline solution to form a thawed-resuspended composition comprising platelets. The administering can include administering the thawed-resuspended composition comprising platelets to the subject in need thereof. The thawed, or thawed-resuspended composition comprising platelets as disclosed herein can then be administered to the subject without the requirement of any waiting period, such as that associated with other single-donor CPP. Therefore, the immediate usability of the composition comprising cryopreserved platelets provided herein is one of the advantages over that of some if not all single-donor CPP. Accordingly, the thawed, or thawed-resuspended composition can be administered to the subject almost immediately after thawing, or thawing and resuspending, for example, within 1 hour, 45, 30, 20, 15, 10, or 5 minutes. In other cases, as described elsewhere in the disclosure, the composition herein after thawing, or thawing and resuspending can be stable for at least 4, 6, 8, 12, 16, 18, or 24 hours when stored at room temperature, or for a temperature in the range of 15° C. to 30° C., 20° C. to 30° C., or 20° C. to 28° C. therefore, providing the flexibility of time for administering the composition to a subject depending upon the urgency, and also other parameters. In some embodiments, the subject is bleeding at the start of administering, and the administering leads to a decrease in bleeding within 15, 30, 45 minutes, 1, 2, 3, 4, 5, 6, 8, 10, 12, 14, 16, 18, 20, 22, or 24 hours after the start of the administering. In some embodiments, the administering is performed until the bleeding stops. In some embodiments, the administering is performed for more than 2, 3, 4, 5, 6, 8, 10, 12, 14, 16, 18, 20, 22, or 24 hours from the start of a first dose of frozen platelets, cryopreserved platelets, cryopreserved platelet derivatives, or platelet derivatives. In some embodiments, the administering is performed for less than 15 minutes, 30 minutes, 45 minutes, 60 minutes, 2, 3, 4, 5, 6, 8, 10, 12, 14, 16, 18, 20, 22, or 24 hours from the start of the first dose of frozen platelets, cryopreserved platelets, cryopreserved platelet derivatives, or platelet derivatives. In some embodiments, the administering is performed until there is cessation of bleeding at a primary bleeding site. In some embodiments, the administering is performed until there is cessation of bleeding at a bleeding site other than the primary bleeding site. In some embodiments, the administering is performed until there is cessation of bleeding at all the bleeding sites. In some embodiments, the administering is performed until there is a decrease in bleeding to less than WHO grade 2, or 2A at a primary bleeding site. In some embodiments, the administering is performed until there is a decrease in bleeding to less than WHO grade 2, or 2A at a bleeding site other than the primary bleeding site. In some embodiments, the administering is performed until there is a decrease in bleeding to less than WHO grade 2, or 2A at all the bleeding sites. In some embodiments, the administering is performed until there is a decrease in bleeding to less than WHO grade 2, or 2A at least one of the bleeding sites. In some embodiments, the administering leads to a decrease in bleeding to less than WHO grade 2, or 2A, or cessation of bleeding in a primary bleeding site, or any site other than the primary bleeding site within 15, 30, 45, 60 minutes, 1, 2, 4, 6, 8, 10, 12, 14, 16, 18, 20, 22, or 24 hours after the administering of frozen platelets, cryopreserved platelets, cryopreserved platelet derivatives, or platelet derivatives herein. In some embodiments, a method of treatment or a composition for use as a medicament as described herein, a method or a medicament leads to survival of the subject without WHO Grade 2, or 2A or greater bleeding during the first 12 hours, 24 hours, 48 hours 3, 4, 5, 6, 7, 8, 9, or 10 days after administering of a composition comprising frozen platelets, cryopreserved platelets, cryopreserved platelet derivatives.

Collections, compositions, or processes provided herein that include cryopreserved platelets that have a biomolecule profile indicative of more than one platelet donor can be used in methods provided herein for reducing bleeding in a subject herein. Since the cryopreserved platelets are derived from more than one platelet donor, typically the methods herein include administering an effective amount of frozen platelets, cryopreserved platelets, or cryopreserved platelet derivatives herein that are allogenic in nature to the recipients (subjects receiving the platelets). Accordingly, the methods for reducing bleeding in a subject herein includes administering an allogenic platelet product, such as the composition comprising cryopreserved platelets as disclosed herein that has a biomolecule profile indicative of more than one platelet donor.

In some aspects and embodiments, a composition comprising frozen platelets, cryopreserved platelets, or cryopreserved platelet derivatives including, but not limited to, those of any of the aspects or embodiments herein, or the composition prepared by any of the processes described herein, such as in aspects or embodiments herein, can be administered, or delivered to a subject having an indication and thus afflicted with a disorder or disease that could benefit from delivery of such compositions comprising frozen platelets, cryopreserved platelets, or cryopreserved platelet derivatives, in illustrative embodiments for reducing and/or stopping bleeding in the subject. In some embodiments, such indication can be any one or a combination of Von Willebrand disease, immune thrombocytopenia (ITP), intracranial hemorrhage (ICH), traumatic brain injury (TBI), Hermansky Pudlak Syndrome (HPS), chemotherapy induced thrombocytopenia (CIT), Scott syndrome, Evans syndrome, hematopoietic stem cell transplantation, fetal and neonatal alloimmune thrombocytopenia, Bernard Soulier syndrome, acute myeloid leukemia, Glanzmann thrombasthenia, myelodysplastic syndrome, hemorrhagic shock, coronary thrombosis (myocardial infarction), ischemic stroke, arterial thromboembolism, Wiskott Aldrich syndrome, venous thromboembolism, MYH9 related disease, acute lymphoblastic lymphoma (ALL), acute coronary syndrome, chronic lymphocytic leukemia (CLL), acute promyelocytic leukemia, cerebral venous sinus thrombosis (CVST), liver cirrhosis, factor v deficiency (Owren Parahemophilia), thrombocytopenia absent radius syndrome, Kasabach Merritt syndrome, Gray platelet syndrome, aplastic anemia, chronic liver disease, acute radiation syndrome, Dengue hemorrhagic fever, pre-eclampsia, snakebite envenomation, HELLP syndrome, haemorrhagic cystitis, multiple myeloma, disseminated intravascular coagulation, heparin induced thrombocytopenia, pre-eclampsia, labor and delivery, hemophilia, cerebral (fatal) malaria, Alexander's disease (Factor VII Deficiency), hemophilia C (Factor XI Deficiency), familial hemophagocytic lymphohistiocytosis, acute lung injury, hemolytic uremic syndrome, menorrhagia, chronic myeloid leukemia. In illustrative embodiments, a composition comprising frozen platelets, cryopreserved platelets, or cryopreserved platelet derivatives of any of the aspects or embodiments herein, or the composition prepared by any of the process described in the aspects or embodiments herein can be administered, or delivered to a subject afflicted by Immune thrombocytopenia. In certain illustrative embodiments, a composition comprising frozen platelets, cryopreserved platelets, or cryopreserved platelet derivatives of any of the aspects or embodiments herein, or the composition prepared by any of the process described in the aspects or embodiments herein can be administered, or delivered to a subject afflicted by Von Willebrand disease. In some embodiments, a method of treating of any of the aspects or embodiments herein, can include a method of treating bleeding such that the bleeding is reduced to a level that is not considered life threatening, or such that the bleeding is stopped in the subject afflicted with any of the indications as described herein.

In some aspects and embodiments, provided herein is a method for reducing and/or treating bleeding in a subject, the method comprising administering a dose, or an effective amount of frozen platelets, cryopreserved platelets, or cryopreserved platelet derivatives herein, including, but not limited to, those of any of the aspects or embodiments herein, or the composition prepared by any of the process described in the aspects or embodiments herein, and the subject is preparing for a surgery, undergoing a surgery, or in a post-surgery stage. In illustrative embodiments, the subject has at least one risk factor for post-surgical bleeding. In some embodiments, the subject is undergoing cardiopulmonary bypass (CPB) surgery with at least one risk factor for post-surgical bleeding including: all re-operative cardiac procedures; expected bypass >120 minutes; any combined cardiac surgery procedures (e.g. multiple valve, valve/CABG); and any procedure that in the estimation of the surgical attending, has a high likelihood of receiving platelets. In some embodiments, the subject is not undergoing coronary artery bypass surgery alone. In some embodiments, the subject is not undergoing implantation of ventricular assist device. In some embodiments, the subject is not undergoing thoracoabdominal aortic aneurysm repair. In some embodiments, the subject does not have heparin-inducted thrombocytopenia. In some embodiments, the administering includes administering a dose, an effective dose, or an effective amount of frozen platelets, cryopreserved platelets, or cryopreserved platelet derivatives, in a range of 2-30, 2-25, 2-20, 2-15, 2-10, 2-8, 2-5, or 2-4 units. In some embodiments, the administering includes administering at least 2, 3, 4, 5, 6, 7, 8, 9, or 10 units of frozen platelets, cryopreserved platelets, or cryopreserved platelet derivatives to the subject. In some embodiments, the administering includes administering the frozen platelets, cryopreserved platelets, or cryopreserved platelet derivatives intraoperatively to the subject. In some embodiments, the administering includes administering the frozen platelets, cryopreserved platelets, or cryopreserved platelet derivatives post-operatively to the subject, in illustrative embodiments, the administering includes administering at a post-heparin reversal stage. In some embodiments, the post-operative stage includes administering after chest closure in the subject. In some embodiments, the post-heparin reversal stage includes the stage in which the active clotting time (ACT) is returned in the subject. In some embodiments, the active clotting time (ACT) can be within 250 seconds, 200 seconds, 150 seconds, or 140 seconds. In some embodiments, the administering includes administering before a post-heparin reversal stage, for example, before the active clotting time is returned in the subject. In some embodiments, the administering includes administering during a heparin reversal stage, for example, during the time when the ACT is returned in the subject. In some embodiments, the administering includes administering the frozen platelets, cryopreserved platelets, or cryopreserved platelet derivatives intraoperatively and/or post operatively up to 1, 2, 3, 4, or 5 units, for example, 1-5 units, 1-4 units, 1-3 units, or 2-3 units. In some embodiments, each unit comprises at least 1.0×10¹¹ platelets, 1.2×10¹¹ platelets, 1.5×10¹¹ platelets, 1.7×10¹¹ platelets, or 1.9×10¹¹ platelets, for example in the range of 1.5×10¹¹ platelets to 1.9×10¹¹ platelets, 1.5×10¹¹ platelets to 1.8×10¹¹ platelets, or 1.6×10¹¹ platelets to 1.8×10¹¹ platelets. In some embodiments, the administering includes, 1-4 units, for example, 1.5-3.5, 2.5 to 3.5, or 1.25 to 3 units. In some embodiments, the administering includes administering the frozen platelets, cryopreserved platelets, or cryopreserved platelet derivatives post-operatively, in illustrative embodiments, post-heparin reversal up to 1, 2, 3, 4, or 5 units. In some embodiments, the frozen platelets, cryopreserved platelets, or cryopreserved platelet derivatives herein is capable of controlling blood loss, reducing bleeding, or stopping bleeding in a subject undergoing a surgery, in illustrative embodiments, CPB surgery in a manner superior to liquid stored platelets, or apheresis platelets. In some embodiments, the superiority can be assessed in terms of the dose of the frozen platelets, cryopreserved platelets, or cryopreserved platelet derivatives herein as compared to the dose of liquid stored platelets, or apheresis platelets, such that a lower dose of the frozen platelets, cryopreserved platelets, or cryopreserved platelet derivatives herein, for example, 1.5, 2, 3, 4, or 5 fold lower dose is capable of providing an effect similar to that of liquid stored platelets, or apheresis platelets. In some embodiments, the superiority can be assessed in terms of a time-frame during which the frozen platelets, cryopreserved platelets, or cryopreserved platelet derivatives herein controls blood loss, reducing bleeding, or stopping bleeding in the subject, for example, the frozen platelets, cryopreserved platelets, or cryopreserved platelet derivatives herein reduces or stops the bleeding within 5 minutes, 10 minutes, 15 minutes, 30 minutes, 45 minutes, 60 minutes, 2 hours, 4 hours, 6 hours, 8 hours, 10 hours, 12 hours, 16 hours, 20 hours, 24 hours, 2 days, 4 days, or 6 days after administering a first dose, or an effective dose/amount of the frozen platelets, cryopreserved platelets, or cryopreserved platelet derivatives herein, and the apheresis platelet or the liquid stored platelets is not able to achieve the reduction in bleeding at one or more than one of the time-frames provided herein. In some embodiments, the frozen platelets, cryopreserved platelets, or cryopreserved platelet derivatives herein reduces or stops the bleeding at any site in a subject within a time-frame that is at least 1.5 times, 2 times, 2.5 times, 3 times, 3.5 times, 4 times, 4.5 times, or 5 times shorter than a time-frame taken by an equivalent amount of apheresis platelets or liquid stored platelets. In some embodiments, the frozen platelets, cryopreserved platelets, or cryopreserved platelet derivatives herein reduces or stops the bleeding at any site in a subject within a time-frame that is between 1.5 and 10 times, 1.5 and 8 times, 1.5 and 7 times, 1.5 and 5 times, or 1.5 and 3 times shorter than a time-frame taken by an equivalent amount of apheresis platelets or liquid stored platelets. In some embodiments, the time-frame herein can be assessed from a time zero until the drain tubes are removed from the subject, or 6 hours, 12 hours, or 24 hours post time zero, in some embodiments, the time zero can be a timepoint at which a first dose or an effective dose of the frozen platelets, cryopreserved platelets, or cryopreserved platelet derivatives herein is administered to the subject. In some embodiments, the assessment can include measuring a total volume of chest tube drainage assessed by measurement of the volume of blood collected from the mediastinal and pleural drains from a time zero, and the time zero can be the time of 1) chest closure or equivalent, 2) chest tubes or equivalent are attached to a graduated post drainage system, and 3) with suction (defined as time zero for analytical purposes) until the drain tubes are removed or 24 hours post time zero, whichever is earlier. In some embodiments, the assessment of bleeding can include assessing the bleeding at 6 hours interval through 24, 48, or 72 hours post time zero or when the chest tubes are removed from the subject. In some embodiments, the bleeding in a subject can be assessed within 6 hours, 12 hours, or 24 hours post heparin reversal. In some embodiments, the bleeding can be assessed from a first protamine administration to the time of first suture for incision closure on day 1 in the subject, for example, the day of a surgery. In some embodiments, the bleeding can be assessed through 6 hours, 12 hours, or 24 hours post heparin reversal, for example, efficacy follow-up period.

In some embodiments of any of the aspects and embodiments herein that include administering a dose, or an effective amount of frozen platelets, cryopreserved platelets, or cryopreserved platelet derivatives herein, including, but not limited to, those of any of the aspects or embodiments herein, or the composition prepared by any of the process described in the aspects or embodiments herein, to a subject, in some cases where the subject is preparing for a surgery, undergoing a surgery, or in a post-surgery stage, the administering does not lead to an increase in the platelet count in the subject. In some embodiments, the administering does not lead to an increase in the platelet count such that the increase is greater than the increase in case of administering RTP or LSP to the subject. Accordingly, even if there is an increase, it is lower than the increase in case of administering RTP or LSP to the subject. In some cases, the administering the thawed composition to the subject leads to an increase in a mean platelet count in the subject such that the increase in the mean platelet count is less than or equal to 25%, 22%, 20%, 19%, 18%, 17%, 15%, 12%, 10%, 9%, 8%, 5%, or 3% as compared to a mean platelet count in the subject before the administering of the thawed composition, for example, in the range of 1-25%, 1-20%, 1-18%, 1-17%, 1-15%, 1-12%, 2-15%, 3-15%, 1-10%, or 1-5%. In some cases, the administering the thawed composition to the subject leads to a change increase in a mean platelet count in the subject such that the change in the mean platelet count is in the range of +/10%, +/−8%, +/−6%, +/−5%, +/−4%, +/−3%, or +/−2% as compared to a mean platelet count in the subject before the administering of the thawed composition. In illustrative embodiments, no LSP or CSP or any other form of platelets are administered to the subject who was administered the thawed composition herein.

In some aspects and embodiments, the subject experiences no adverse events attributable to the administering the frozen platelets, cryopreserved platelets, cryopreserved platelet derivatives, or the platelet derivatives herein when the decreased bleeding is detected. In some embodiments, the subject experiences no grade 3 or grade 4 adverse events attributable to the administering the frozen platelets, cryopreserved platelets, cryopreserved platelet derivatives, or the platelet derivatives herein, when the decreased bleeding is detected. In some embodiments, the subject experiences no adverse events or no grade 2 or higher adverse events, and in illustrative embodiments no grade 3 or grade 4 adverse events, attributable to the administering the frozen platelets, cryopreserved platelets, cryopreserved platelet derivatives, or the platelet derivatives herein, within 2, 4, 5, 7, 8, 10, 12, 14, 15, 20, 25, or 30 days of the first dose. In some embodiments, the subject experiences no adverse events, or no grade 2 or higher adverse events, and in illustrative embodiments no grade 3 or grade 4 adverse events, attributable to the administering the frozen platelets, cryopreserved platelets, cryopreserved platelet derivatives, or the platelet derivatives herein, within 15 minutes, 30 minutes, 45 minutes, 60 minutes, 1 hour, 2, 4, 6, 8, 10, 12, 14, 16, 18, 20, 22, or 24 hours of the first dose. In some embodiments, the subject experiences no adverse events, or no grade 2 or higher adverse events, and in illustrative embodiments no grade 3 or grade 4 adverse events, attributable to the administering the frozen platelets, cryopreserved platelets, cryopreserved platelet derivatives, or the platelet derivatives herein when the decreased bleeding is detected. In some embodiments, the subject experiences no adverse events, or no grade 2 or higher adverse events, and in illustrative embodiments no grade 3 or grade 4 adverse events, attributable to the administering the frozen platelets, cryopreserved platelets, cryopreserved platelet derivatives, or the platelet derivatives herein, within 15 minutes, 30 minutes, 45 minutes, 60 minutes, 1 hour, 2, 4, 6, 8, 10, 12, 14, 16, 18, 20, 22, 24, 28, 30, 32, 34, 36, 48, or 72 hours of the first dose.

In some aspects, and embodiments, the subject, at the time of the administering, has a WHO bleeding score of Grade 2, Grade 2A, Grade 3, or a higher bleeding score. In some embodiments, within 15 minutes, 30 minutes, 45 minutes, 60 minutes, 2, 4, 6, 8, 10, 12, 14, 16, 18, 20, 22, or 24 hours, 2 days, or 3 days after the administering to the subject, the bleeding of the subject is decreased to a WHO bleeding score of less than Grade 2 or 2A, less than Grade 1, or decreased to Grade 0, at a primary site, at a bleeding site other than the primary site, or at all the bleeding sites. In illustrative embodiments, after the administering, the bleeding of the subject is decreased to a WHO bleeding score of Grade 1, less than Grade 1, or decreased to Grade 0, at all the bleeding sites within 15 minutes, 30 minutes, 45 minutes, 1, 2, 4, 6, 8, 10, 12, 14, 16, 18, 20, 22, or 24 hours after the administering of a dose, a first dose, or an effective dose/amount of the frozen platelets, cryopreserved platelets, cryopreserved platelet derivatives, or the platelet derivatives herein.

A person of skill in the art can contemplate treating a subject, reducing or decreasing bleeding in a subject or using frozen platelets, cryopreserved platelets, cryopreserved platelet derivatives, or the platelet derivatives herein as a medicament in a single dose or several doses in a span of time for treating or reducing bleeding in the subject. In some embodiments, the administering as disclosed herein, is performed for at least, or at a maximum of 2, 3, 4, 5, 6, 7, 8, 9, or 10 doses in a 72-hour period of treatment. The dose can be any of the doses as disclosed herein. For example, a dose can be a first dose, an effective amount, a therapeutically effective dose, or an effective amount between 0.5 units and 10, 9, 8, 7, 6, 5, 4, 3, 2 or 1 unit. In some embodiments, a first dose can be in any one of the amounts as disclosed herein, and subsequent doses can be in the same or different amounts as disclosed herein. In some embodiments, a dose, a first dose, or an effective dose can include between 0.25 unit and 10 units, 0.25 unit and 9 units, 0.25 unit and 8 units, 0.25 unit and 7 units, 0.25 unit and 6 units, 0.25 unit and 5 units, 0.25 unit and 4 units, 0.5 unit and 5 units, 0.5 unit and 4 units, 1 unit and 5 units, 1 unit and 4 units, or 2 units and 4 units. In some embodiments, 1 unit of a composition herein can comprise at least 5.0×10¹⁰, 5.5×10¹⁰, 6.0×10¹⁰, 6.5×10¹⁰, or 7.0×10¹⁰ frozen platelets, cryopreserved platelets, or cryopreserved platelet derivatives. In some embodiments, 1 unit of the composition herein can comprise between 4.0×10¹⁰ frozen platelets, cryopreserved platelets, or cryopreserved platelet derivatives at a lower end of a range, and 4.5×10¹⁰5.0×10¹⁰, 5.5×10¹⁰, 6.0×10¹⁰, 6.5×10¹⁰, 7.0×10¹⁰, 7.5×10¹⁰, or 8.0×10¹⁰ frozen platelets, cryopreserved platelets, or cryopreserved platelet derivatives at a higher end of a range. A particular dose can be any dose as disclosed herein, and the dose can vary during the time interval for which the cryopreserved platelets are administered to a subject or a recipient in need thereof. In some embodiments, the administering can be performed at regular intervals. For example, a single, double, triple or more doses of the cryopreserved platelets herein and as per the requirement of a subject, for example to reduce or stop bleeding of the subject, can be administered to the recipient subject every 10 minutes, 20 minutes, 30 minutes, 1 hour, 2 hours, 3 hours, 4 hours, 5 hours, 6 hours, 7 hours, 8 hours, 9 hours, 10 hours, 11 hours, or 12 hours depending on whether bleeding is reduced to a satisfactory level, for example such that it is no longer considered life-threatening, or no longer considered severe or serious, or continued until the bleeding is mild, or stops. In some embodiments, the doses can be administered at a regular interval for 36 hours, 48 hours, or 72 hours from the start of the first dose In some embodiments, the administering can be performed in a maximum of 2, 3, 4, 5, 6, 7, 8, 9, or 10 doses in a 24-hour period. In some embodiments, the administering can be performed in a maximum of 2, 3, 4, 5, 6, 7, 8, 9, or 10 doses in a 72-hour period of treatment. In some embodiments, the administering can be performed at a frequency of at least one dose every 15 minutes or more frequently. For example, the administering can be performed at a frequency of at least one dose every 15 minutes or more frequently starting from the first dose until the bleeding of the subject is reduced or stopped as compared to the bleeding before the administering. In some embodiments, the administering can be performed until the bleeding stops. In some embodiments, the administering can be performed for at least 1, 10, 15, 30, 45, or 60 minutes. In some embodiments, the administering can be performed at a frequency of at least one dose in every 20 minutes, 30 minutes, 45 minutes, 60 minutes, 2 hours, 3 hours, 4 hours, 5 hours, 6 hours, 7 hours, 8 hours, 9 hours, 10 hours, 12 hours, 15 hours, 18 hours, 24 hours, 30 hours, or 36 hours or more frequently.

In some aspects and embodiments, the administering herein includes administering a composition that comprises frozen platelets, cryopreserved platelets, or cryopreserved platelet derivatives herein that is prepared by a process herein that includes storing a batch of frozen platelets, cryopreserved platelets, or cryopreserved platelet derivatives at a temperature higher than −80° C., −70° C., −60° C., −50° C., −40° C., or −30° C., for example at a temperature in the range of −10° C. to −50° C., −10° C. to −45° C., −10° C. to −40° C., or −10° C. to −30° C. for at least 1 month, 2, 3, 4, 5, 6, 7, 8, 9, 10, 11, or 12 months. In some embodiments, the process does not comprise a transition in freezing temperatures. In some embodiments, the process does not comprise exposing the platelets to a freezing temperature of less than −60° C., −70° C., or −80° C.

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

In any of the aspects and embodiments herein that include a property of cryopreserved platelets, a collection of cryo-vessels, or a cryopreserved platelet composition obtained from a process for preparing a batch of a cryopreserved platelet composition as disclosed herein, the property exhibited by cryopreserved platelets can be studied after thawing, and optionally diluting. Accordingly, it will be understood that if the property is a property of the cryopreserved platelets in a frozen form, actually performing a test related to the property is not required to fall under such an aspect or embodiment if the test is only recited as a property of the cryopreserved platelet composition or thawed cryopreserved platelet compositions. Thus, steps of thawing and optionally diluting in some aspects and embodiments are some of the steps for assessing the property, and do not necessarily need to be performed to fall under such an aspect or embodiment.

Provided herein, in one aspect is a cryopreserved platelet composition, comprising frozen activated platelets comprising a population of platelet particles in a cryopreservation medium in a frozen state in a cryo-vessel,
wherein the frozen activated platelets exhibit the following property upon thawing:
   are capable of specific adhesion to a collagen-coated channel in the presence of plasma, in illustrative embodiments, in the absence of external platelets, when tested in an in vitro assay. In illustrative embodiments, the thawing comprises placing the cryo-vessel in a water bath set at a temperature of 37° C.+/−2° C. until the frozen activated platelets are thawed to form a thawed activated platelet composition. In illustrative embodiments, the in vitro assay comprises labeling thawed frozen activated platelets in the thawed activated platelet composition to form labeled, thawed frozen activated platelets. In illustrative embodiments, the in vitro assay comprises contacting the labeled, thawed activated platelets in the presence of plasma but in the absence of external platelets, to the collagen-coated channel under a shear flow, and acquiring an image of the collagen-coated channel to detect, and or quantify the labeled, thawed activated platelets that bound to the collagen-coated channel. In illustrative embodiments, the thawed activated platelets can be diluted with saline. It will be understood herein that thawed frozen activated platelets can be referred to as thawed activated platelets.

Provided herein, in one aspect is a collection of cryo-vessels, comprising a plurality of cryo-vessels, wherein the frozen activated platelets in each cryo-vessel in the collection, exhibit the following property, upon thawing: are capable of specific adhesion to a collagen-coated channel in the presence of plasma, in illustrative embodiments, in the absence of external platelets, when tested in an in vitro assay. In illustrative embodiments, the in vitro assay comprises testing one cryo-vessel each from at least three, four, five or six different batches in the collection of cryo-vessels, and wherein the specific adhesion exhibited by the frozen activated platelets in at least one cryo-vessel exhibits a rate of adhesion to collagen that is equal to or more than 10%, 15%, 20%, 25%, 30%, 35%, 40%, 45%, or 50% of a rate of adhesion of platelets in a platelet-rich plasma under the same conditions. In illustrative embodiments, each cryo-vessel in the collection comprising 1 unit equivalent of frozen activated platelets comprising a population of platelet particles in a cryopreservation medium in a frozen state. In illustrative embodiments, the population of platelet particles in the frozen activated platelets in each cryo-vessel have a set of biomolecule profiles indicative of more than 1 platelet donor. In illustrative embodiments, the thawing comprises placing the cryo-vessel in a water bath set at a temperature of 37° C.+/−2° C. until the frozen activated platelets are thawed to form a thawed activated platelet composition. In illustrative embodiments, the in vitro assay comprises labeling thawed frozen activated platelets in the thawed activated platelet composition to form labeled, thawed frozen activated platelets. In illustrative embodiments, the in vitro assay comprises contacting the labeled, thawed activated platelets in the presence of plasma but in the absence of external platelets, to the collagen-coated channel under a shear flow, and acquiring an image of the collagen-coated channel to detect, and/or quantify the labeled, thawed activated platelets that bound to the collagen-coated channel, for example by measuring the fluorescing area in the channel. In illustrative embodiments, the thawed activated platelets can be diluted with saline. In illustrative embodiments, the frozen activated platelets in each cryo-vessel in the collection have a capacity to generate thrombin in an in vitro thrombin generation assay. In illustrative embodiments, the frozen activated platelets in each cryo-vessel in the collection have less than 15×10⁶, or 10×10⁶ CD61-positive microparticles/μl of a thawed activated platelet composition.

Provided herein, in one aspect is a process for preparing a batch of cryopreserved platelets, comprising:
a) forming a concentrated pooled platelet resuspension (CPR) with a weight or volume based on the number of platelet units (PU) to form the CPR;
b) adding a cryoprotectant to the CPR to obtain a CPR having the cryoprotectant;
c) distributing the CPR having DMSO among more than 1 vessel of cryo-vessels; and
d) freezing the collection of cryo-vessels to prepare the batch of cryopreserved platelets. In illustrative embodiments, forming the CPR is done with a weight based on the number of PUs to form the CPR. In illustrative embodiments, the cryoprotectant comprises dimethyl sulfoxide (DMSO), such that adding DMSO to obtain a CPR having DMSO. In some embodiments, adding DMSO is done to obtain a CPR having DMSO in the range of 1% to 10%, in illustrative embodiments, in the range of 4% to 8%. In illustrative embodiments the distributing is done to obtain 1 PU equivalent weight of the CPR having DMSO in each cryo-vessel. In some embodiments, the freezing is initiated within 4, 3, in illustrative embodiments, within 2, 1.5 hours, or within 1 hour after adding the cryoprotectant, such as DMSO. In illustrative embodiments, the initiation of the freezing comprises placing the cryo-vessels in a freezer pre-set at a freezing temperature. In illustrative embodiments, the freezing comprises storing the cryo-vessels in the freezer for at least a time period required to freeze the contents of the cryo-vessels. In illustrative embodiments, the forming the CPR comprises pooling 2 or 3 platelet units in one vessel, and optionally providing 1 platelet unit in another vessel to form a plurality of vessels. In illustrative embodiments, the forming the CPR uses at least 5 platelet units, and the platelet units are from two or more donors. In illustrative embodiments, the forming the CPR comprises removing some plasma from pooled platelet units to achieve a weight or a volume based on the number of PUs to form the CPR. In illustrative embodiments, the plasma is removed to achieve a weight that is in the range of 23-25, 23-24, 23.10-23.75, or about 23.25 grams/platelet unit that is pooled in a vessel for forming the CPR.

Provided herein, in one aspect is a process for preparing a cryopreserved platelet composition comprising cryopreserved platelets, or a cryopreserved platelet derivative composition comprising cryopreserved platelet derivatives, said process comprising:
i) freezing a population of platelets in a cryopreservation medium at a temperature of equal to or less than −50° C., in illustrative embodiments for a time until the cryopreservation medium is frozen, to form an initial frozen platelet composition;
ii) transferring the initial frozen platelet composition to a freezer set at a temperature of equal to or higher than −30° C., but less than 0° C.; and
iii) storing the initial frozen platelet composition in the freezer for at least 90 minutes, to form the cryopreserved platelet composition comprising the cryopreserved platelets or the cryopreserved platelet derivative composition comprising the cryopreserved platelet derivatives.

Provided herein, in one aspect is a process for preparing a cryopreserved platelet composition comprising cryopreserved platelets, said process comprising:
i) freezing a population of platelets in a cryopreservation medium at a temperature of equal to or less than −50° C. to form an initial frozen platelet composition; and
ii) storing the initial frozen platelet composition at a temperature in the range of −10° C. to −30° C. for at least 1 month to form the cryopreserved platelet composition.

Provided herein, in one aspect is a process for preparing a cryopreserved platelet composition comprising cryopreserved platelets, said process comprising:
i) freezing a population of platelets in a cryopreservation medium at a temperature of less than −50° C. to form an initial frozen platelet composition; and
ii) storing the initial frozen platelet composition in a freezer set at a temperature of −20° C. +/−2° C. for at least one month to form the cryopreserved platelet composition.

Provided herein, in one aspect, is a process for preparing a batch of a cryopreserved platelets, comprising:
a) pooling at least 2 platelet units into one vessel and at least another platelet unit into another vessel, in illustrative embodiments, there are at least 3, 4, or 5 platelet units, and the platelet units are from more than one donor, for example, 2, 3, 4, or more donors;
b) centrifuging each vessel to obtain a supernatant comprising plasma, and a pellet comprising platelets;
c) resuspending the pellet in each vessel to form a resuspension;
d) pooling the resuspension from each vessel to form a pooled resuspension in a pooled resuspension vessel;
e) adding a cryoprotectant to the pooled resuspension vessel having the pooled resuspension to obtain a pooled resuspension having the cryoprotectant;
f) distributing the pooled resuspension having the cryoprotectant from the pooled resuspension vessel among a number of cryo-vessels; and
g) freezing the pooled resuspension having the cryoprotectant in the cryo-vessels, to form the batch of cryopreserved platelets. In illustrative embodiments, the cryoprotectant is dimethyl sulfoxide (DMSO), and the pooled resuspension is with DMSO, to form a pooled resuspension having DMSO. In some embodiments, the resuspension has a target weight that is X times the number of units pooled or provided in the vessel. In some embodiments, the X has a range of 10 g to 40 g times the number of units pooled or provided in the vessel. Accordingly, the resuspension has a target weight in the range of 10 g to 40 g times the number of units pooled or provided in the vessel. In illustrative embodiments, the resuspension has a target weight in the range of 15.9 g to 27.9 g times the number of units pooled or provided in the vessel. In illustrative embodiments, the freezing the pooled resuspension includes freezing at a temperature of higher than −80° C., −70° C., or −60° C. In illustrative embodiments, the freezing the pooled resuspension includes freezing at a temperature in the range of −10° C. to −50° C., −10° C. to −45° C., −10° C. to −40° C., or −10° C. to −30° C. In illustrative embodiments, the freezing includes storing the pooled resuspension in a freezer set at a temperature of −20° C. +/−5° C. In illustrative embodiments, the freezing includes the freezing the pooled resuspension to form the batch of cryopreserved platelets and storing the batch of the cryopreserved platelets at a temperature of higher than −80° C., −70° C., or −60° C., for example, in a range of −10° C. to −50° C., −10° C. to −45° C., −10° C. to −40° C., or −10° C. to −30° C., or freezing the pooled resuspension to form the batch of cryopreserved platelets and storing the batch of the cryopreserved platelets in a freezer set at a temperature of −20° C. +/−5° C. In illustrative embodiments, the freezing the pooled resuspension is initiated within 4, 3, or 2 hours of adding the cryoprotectant, for example, DMSO.

Provided herein, in one aspect, is a process for preparing a batch of cryopreserved platelets, comprising:
a) pooling at least 2 platelet units into one vessel and at least another platelet unit into another vessel, in illustrative embodiments, there are at least 3, 4, or 5 platelet units, and the platelet units are from more than one donor, for example, 2, 3, 4, or more donors;
b) centrifuging each vessel to obtain a supernatant comprising plasma, and a pellet comprising platelets;
c) resuspending the pellet in each vessel to form a resuspension;
d) pooling the resuspension from each vessel to form a pooled resuspension in a pooled resuspension vessel;
e) adding a cryoprotectant to the pooled resuspension vessel having the pooled resuspension to obtain a pooled resuspension having the cryoprotectant;
f) distributing the pooled resuspension having the cryoprotectant from the pooled resuspension vessel among a number of cryo-vessels;
g) freezing the pooled resuspension having the cryoprotectant in the cryo-vessels at a temperature of less than or equal to −50° C., to form an initial frozen platelet composition in the cryo-vessels; and
h) storing the initial frozen platelet composition in the cryo-vessels at a temperature higher than or equal to −30° C., but less than 0° C., for example by storing in a −20° C. freezer, to form the batch of cryopreserved platelets. In illustrative embodiments, storing is done for at least 7, 10, 15, 20, 25 days, 1 month, 2, 4, 6, 8, 10, 12 months, 2, 4, 6, 8, or 10 years.

Provided herein, in one aspect, is a collection of cryo-vessels, wherein each cryo-vessel in the collection comprises cryopreserved platelets,
wherein the cryopreserved platelets in each cryo-vessel have a set of biomolecule profiles indicative of more than 1 platelet donor,
wherein the cryopreserved platelets in each cryo-vessel of a batch of cryo-vessels of the collection have an indistinguishable set of biomolecular profiles comprising a phosphatidylserine positivity, when measured using lactadherin or annexin V binding of at least 30%, 40%, or 50%,
wherein each batch of the collection has a different set of biomolecular profiles than any other batch in the collection,
wherein the collection comprises a plurality of at least 2 batches of cryo-vessels,
wherein the cryopreserved platelets in the collection, upon thawing have a capacity to generate thrombin in an in vitro thrombin generation assay, and
wherein the cryopreserved platelets in the collection have the property of exhibiting a pH of greater than 6.0, upon thawing and storing for at least 4 hours, or 6 hours. In illustrative embodiments, the CD62 positivity of the cryopreserved platelets is in the range of 30% to 90%, or 50% to 75%. In illustrative embodiments, the phosphatidylserine positivity, when measured using lactadherin binding is in the range of 30% to 90%, or 50% to 90%. In illustrative embodiments, the cryopreserved platelets in the collection have the property of exhibiting a pH of greater than 6.0, upon thawing and storing at a temperature in the range of 18° C. to 30° C., 20° C. to 30° C., or 20° C. to 26° C., or at room temperature. In illustrative embodiments, the cryopreserved platelets in the collection have the property of exhibiting a pH of greater than 6.0, upon thawing and storing at the temperature for at least 6, 8, 12, 24, or 36 hours.

Provided herein, in one aspect, is a collection of cryo-vessels, wherein each cryo-vessel in the collection comprises cryopreserved platelets,
wherein the cryopreserved platelets in each cryo-vessel of a batch of cryo-vessels of the collection have an indistinguishable set of biomolecular profiles comprising a phosphatidylserine positivity, when measured using lactadherin binding of at least 40%, 50%, or 60%,
wherein each batch of the collection has a different set of biomolecular profiles than any other batch in the collection,
wherein the collection comprises a plurality of at least 2 batches of cryo-vessels,
wherein the cryopreserved platelets in the collection, upon thawing have a capacity to generate thrombin in an in vitro thrombin generation assay, and
wherein the cryopreserved platelets in the collection have the property of exhibiting a pH of greater than 6.0, in illustrative embodiments, greater than 6.2, upon thawing and storing for at least 4 hours, or at least 6 hours. In illustrative embodiments, the cryopreserved platelets in each cryo-vessel have a set of biomolecule profiles indicative of more than 1 platelet donor. In some embodiments, the storing is done for 6 to 24 hours, and the pH is greater than 6.2. In some embodiments, the cryopreserved platelets can be stored at a temperature in the range of 15° C. to 45° C., or at room temperature. In illustrative embodiments, the phosphatidylserine positivity of the cryopreserved platelets, when measured using lactadherin binding is in the range of 30% to 90%, 50% to 90%, or 50% to 75%. In illustrative embodiments, the cryopreserved platelets in the collection have the property of exhibiting a pH of greater than 6.0, upon thawing and storing at a temperature in the range of 18° C. to 30° C., 20° C. to 40° C., 22° C. to 38° C., or 20° C. to 26° C., or at room temperature. In illustrative embodiments, the cryopreserved platelets in the collection have the property of exhibiting a pH of greater than 6.0, upon thawing and storing at the temperature for at least 6, 8, 12, 24, or 36 hours. In some embodiments, the cryopreserved platelets have the property, upon thawing, of having less than 10×10⁶, 9.9×10⁶, 9.8×10⁶, 9.7×10⁶, 9.5×10⁶, 9.2×10⁶, 9.0×10⁶, 8.8×10⁶, 8.5×10⁶, 8.0×10⁶ or 7.5×10⁶ CD61-positive microparticles/μl. In some embodiments, the cryopreserved platelets, upon thawing have a concentration of CD61-positive microparticles in the range of 2.0×10⁶ to 9.8×10⁶, 2.0×10⁶ to 9.5×10⁶, 2.0×10⁶ to 9.3×10⁶, 2.0×10⁶ to 9.0×10⁶, 2.0×10⁶ to 8.7×10⁶, 2.0×10⁶ to 8.5×10⁶, or 2.0×10⁶ to 8.0×10⁶ CD61-positive microparticles/μl.

Provided herein, in one aspect, is a frozen platelet composition comprising frozen activated platelets in a cryopreservation medium in a frozen state,
wherein the frozen platelet composition comprises frozen activated platelets displaying a CD62 positivity of at least 50%, or a phosphatidylserine positivity of at least 50% when measured using lactadherin binding,
wherein the frozen platelet composition comprising frozen activated platelets, upon thawing has a capacity to generate thrombin in an in vitro thrombin generation assay, and
wherein the frozen platelet composition comprising frozen activated platelets has a property of exhibiting a pH of greater than 6.0, upon thawing. In illustrative embodiments, the frozen activated platelets in each cryo-vessel have a set of biomolecule profiles indicative of more than 1 platelet donor. In illustrative embodiments, the CD62 positivity of the frozen activated platelets is in the range of 40% to 90%, or 50% to 75%. In illustrative embodiments, the frozen activated platelets has a property of exhibiting a pH of greater than 6.0, typically, greater than 6.2 upon thawing and storing for at least 4 hours, typically for at least 6 hours, 8 hours, or 24 hours. In illustrative embodiments, the frozen activated platelets has a property of exhibiting a pH of greater than 6.0, typically, greater than 6.2 upon thawing and storing for 4 hours, 6 hours, 8 hours, or 24 hours, at a temperature in the range of 20° C. to 30° C., or 20° C. to 26° C., or at room temperature. In illustrative embodiments, the frozen activated platelets have a property of exhibiting a pH of greater than 6.0, typically, greater than 6.2 upon thawing and storing for a time-period as disclosed herein at a temperature in the range of 18° C. to 30° C., or 20° C. to 26° C. In illustrative embodiments, the frozen activated platelets upon thawing have less than 15×10⁶, 12×10⁶, or 10×10⁶ CD61-positive microparticles/μl.

Provided herein, in one aspect, is a frozen platelet composition comprising frozen activated platelets in a cryopreservation medium in a frozen state,
wherein the frozen platelet composition comprises frozen activated platelets displaying a phosphatidylserine positivity, when measured using lactadherin binding of at least 50%, or 60%,
wherein the frozen activated platelets have a set of biomolecule profiles indicative of more than 1 platelet donor,
wherein the frozen platelet composition comprising frozen activated platelets, upon thawing has a capacity to generate thrombin in an in vitro thrombin generation assay, and
wherein the frozen platelet composition comprising frozen activated platelets has a property of exhibiting a pH of greater than 6.0, upon thawing and storing for at least 4 hours, or 6 hours. In illustrative embodiments, the phosphatidylserine positivity of the composition comprising frozen platelets, when measured using lactadherin binding is in the range of 50% to 90%, or 50% to 75%. In illustrative embodiments, the frozen activated platelets have a property of exhibiting a pH of greater than 6.0, upon thawing and storing at a temperature in the range of 18° C. to 30° C., or 20° C. to 26° C., or at room temperature. In illustrative embodiments, the frozen activated platelets have a property of exhibiting a pH of greater than 6.0, upon thawing and storing at the temperature for at least 6, 8, 10, 12, 24, 36 hours. In some embodiments, the storing is done for 6 to 24 hours, and the pH is greater than 6.2. In some embodiments, the frozen platelet composition can be stored at a temperature in the range of 15° C. to 45° C. In some embodiments, the frozen platelet composition comprising frozen activated platelets, upon thawing have less than 10×10⁶, 9.9×10⁶, 9.8×10⁶, 9.7×10⁶, 9.5×10⁶, 9.2×10⁶, 9.0×10⁶, 8.8×10⁶, 8.5×10⁶, 8.0×10⁶ or 7.5×10⁶ CD61-positive microparticles/μl. In some embodiments, the frozen platelet composition comprising frozen activated platelets, upon thawing have a concentration of CD61-positive microparticles in the range of 2.0×10⁶ to 9.8×10⁶, 2.0×10⁶ to 9.5×10⁶, 2.0×10⁶ to 9.3×10⁶, 2.0×10⁶ to 9.0×10⁶, 2.0×10⁶ to 8.7×10⁶, 2.0×10⁶ to 8.5×10⁶, or 2.0×10⁶ to 8.0×10⁶ CD61-positive microparticles/μl.

Provided herein, in one aspect, is a collection of cryo-vessels, wherein each cryo-vessel in the collection comprises cryopreserved platelets,
wherein the cryopreserved platelets in each cryo-vessel have a set of biomolecule profiles indicative of more than 1 platelet donor,
wherein the cryopreserved platelets in each cryo-vessel of a batch of cryo-vessels of the collection have an indistinguishable set of biomolecular profiles comprising a CD62 positivity of at least 30%, 40%, or 50%, and/or a phosphatidylserine positivity of at least 50%, when measured using lactadherin binding,
wherein each batch of the collection has a different set of biomolecular profiles than any other batch in the collection,
wherein the collection comprises a plurality of at least 2 batches of cryo-vessels,
wherein each cryo-vessel in the collection comprising the cryopreserved platelets, upon thawing have a capacity to generate thrombin in an in vitro thrombin generation assay, and
wherein the cryopreserved platelets in the collection, upon thawing have less than 15×10⁶, 12×10⁶, or 10×10⁶ CD61-positive microparticles/μl. In illustrative embodiments, the cryopreserved platelets in the collection, upon thawing and storing for at least 4 hours, 6 hours, 8 hours, or 24 hours have less than 10×10⁶ CD61-positive microparticles/μl. In illustrative embodiments, the CD62 positivity of the cryopreserved platelets is in the range of 30% to 90%, or 50% to 75%. In illustrative embodiments, the cryopreserved platelets in the collection, upon thawing and storing for at least 4 hours, typically for at least 6 hours, 8 hours, or 24 hours have less than 10×10⁶ CD61-positive microparticles/μl. In illustrative embodiments, the cryopreserved platelets have a property of exhibiting a pH of greater than 6.0, typically, greater than 6.2 upon thawing and storing for 4 hours, 6 hours, 8 hours, or 24 hours, at a temperature in the range of 20° C. to 30° C., or 20° C. to 26° C., or at room temperature, have less than 10×10⁶ CD61-positive microparticles/μl. In illustrative embodiments, the cryopreserved platelets have a property of exhibiting a pH of greater than 6.0, typically, greater than 6.2 upon thawing and storing for a time-period as disclosed herein at a temperature in the range of 18° C. to 30° C., or 20° C. to 26° C., or at room temperature have less than 10×10⁶ CD61-positive microparticles/μl.

Provided herein in one aspect is a collection of cryo-vessels, comprising
a plurality of cryo-vessels, each cryo-vessel in the collection comprising a population of platelet particles in a cryopreservation medium in a frozen state,
wherein the frozen activated platelets in each cryo-vessel in the collection, exhibit the following properties per unit equivalent in each cryo-vessel upon thawing:
form a thawed activated platelet composition weighing between 24.50 and 30.50 grams when diluted with 25 ml of saline;
have a capacity to generate thrombin in an in vitro thrombin generation assay, and
have, comprise, and/or contain less than 15×10⁶, 12×10⁶, 10×10⁶, 9.5×10⁶, or 9.0×10⁶ CD61-positive microparticles/μl of the thawed activated platelet composition,
   wherein the thawing comprises placing the cryo-vessel in a water bath set at a temperature of 37° C.+/−2° C. until the frozen activated platelets are thawed to form the thawed activated platelet composition. In illustrative embodiments, the population of platelet particles in the frozen activated platelets in each cryo-vessel have a set of biomolecule profiles indicative of more than 1 platelet donor. In illustrative embodiments, each cryo-vessel in the collection comprises 1 unit equivalent of frozen activated platelets. In illustrative embodiments, the phosphatidylserine positivity, when measured using lactadherin binding, is in the range of 50% to 90%, 55% to 90%, or 50% to 75%. In some embodiments, the cryopreserved platelet composition comprising frozen activated platelets, upon thawing have a concentration of CD61-positive microparticles in the range of 2.0×10⁶ to 9.8×10⁶, 2.0×10⁶ to 9.5×10⁶, 2.0×10⁶ to 9.3×10⁶, 2.0×10⁶ to 9.0×10⁶, 2.0×10⁶ to 8.7×10⁶, 2.0×10⁶ to 8.5×10⁶, or 2.0×10⁶ to 8.0×10⁶ CD61-positive microparticles/μl. In some embodiments, the cryopreserved platelet composition comprising frozen activated platelets, upon thawing and storing at a temperature in the range of 15° C. to 45° C., 20° C. to 40° C., 25° C. to 28° C., or 20° C. to 28° C., or at room temperature for at least 4, 6, 8, 10, 12, or 24 hours, or 6 hours to 24 hours have the property of exhibiting a pH of more than 6.0, in illustrative embodiments, more than 6.2, 6.3, 6.4, or 6.5. In some embodiments, the collection can comprise at least 2, 3, 4, 5, 6, 7, 8, 9, 10, 15, 20, 30, 40, 50, 60, 70, 80, 990, or 100 batches.

Provided herein, in one aspect, is a collection of cryo-vessels, wherein each cryo-vessel in the collection comprises a cryopreserved platelet composition comprising a population of frozen activated platelets,
wherein the population of the frozen activated platelets in each cryo-vessel of a batch of cryo-vessels of the collection have an indistinguishable set of biomolecular profiles comprising a phosphatidylserine positivity, when measured using lactadherin binding of at least 50%, or 60%,
wherein each batch of the collection has a different set of biomolecular profiles than any other batch in the collection,
wherein the collection comprises a plurality of at least 2 batches of cryo-vessels,
wherein each cryopreserved platelet composition comprising the frozen activated platelets in the collection, upon thawing have a capacity to generate thrombin in an in vitro thrombin generation assay, and
wherein each cryopreserved platelet composition comprising the frozen activated platelets in the collection, upon thawing have, comprise, and/or contain less than 15×10⁶, 12×10⁶, 10×10⁶, 9.5×10⁶, or 9.0×10⁶ CD61-positive microparticles/μl. In illustrative embodiments, the population of platelet particles in the frozen activated platelets in each cryo-vessel have a set of biomolecule profiles indicative of more than 1 platelet donor. In illustrative embodiments, the phosphatidylserine positivity, when measured using lactadherin binding, is in the range of 50% to 90%, 55% to 90%, or 50% to 75%. In some embodiments, the cryopreserved platelet composition comprising frozen activated platelets, upon thawing have a concentration of CD61-positive microparticles in the range of 2.0×10⁶ to 9.8×10⁶, 2.0×10⁶ to 9.5×10⁶, 2.0×10⁶ to 9.3×10⁶, 2.0×10⁶ to 9.0×10⁶, 2.0×10⁶ to 8.7×10⁶, 2.0×10⁶ to 8.5×10⁶, or 2.0×10⁶ to 8.0×10⁶ CD61-positive microparticles/μl. In some embodiments, the cryopreserved platelet composition comprising frozen activated platelets, upon thawing and storing at a temperature in the range of 15° C. to 45° C., 20° C. to 40° C., 25° C. to 28° C., or 20° C. to 28° C., or at room temperature for at least 4, 6, 8, 10, 12, or 24 hours, or 6 hours to 24 hours have the property of exhibiting a pH of more than 6.0, in illustrative embodiments, more than 6.2, 6.3, 6.4, or 6.5. In some embodiments, the collection can comprise at least 2, 3, 4, 5, 6, 7, 8, 9, 10, 15, 20, 30, 40, 50, 60, 70, 80, 990, or 100 batches.

Provided herein, in one aspect, is a cryopreserved platelet composition,
comprising frozen activated platelets comprising a population of platelet particles in a cryopreservation medium in a frozen state in a cryo-vessel,
wherein the composition comprises frozen platelets displaying a CD62 positivity of at least 50%, or 60%, and/or a phosphatidylserine positivity of at least 50%, when measured using lactadherin binding,
wherein the composition comprising frozen platelets, upon thawing has a capacity to generate thrombin in an in vitro thrombin generation assay, and
wherein the cryopreserved platelet composition comprising frozen platelets has a property of exhibiting a pH of greater than 6.0, upon thawing and storing at a room temperature, or at a temperature in the range of 15° C. to 30° C., 20° C. to 30° C., or 20° C. to 28° C. for at least 24 hours. In illustrative embodiments, the CD62 positivity of the composition comprising cryopreserved platelets is in the range of 50% to 90%, or 55% to 75%.

Provided herein, in one aspect, is a collection of cryo-vessels comprising cryopreserved platelets, wherein the cryopreserved platelets in each cryo-vessel have a set of biomolecule profiles indicative of more than 1 platelet donor, in illustrative embodiments, a batch of cryo-vessels has an identical set of biomolecular profiles, and wherein each batch of the collection has a different set of biomolecular profiles than any other batch in the collection,
wherein the collection comprises a plurality of at least 2, 3, 4, or more batches of cryo-vessels,
wherein each batch of cryo-vessels comprises at least 2, 3, 4, or 5 cryo-vessels, and wherein the coefficient of variance of a mean DMSO concentration in the cryopreserved platelets across the batches is less than 10%, 9%, 8%, 7%, 6%, 5%, 4%, 3%, 2%, or 1%. In illustrative embodiments, the coefficient of variance of a mean DMSO concentration in the cryopreserved platelets across the batches is in the range of 0.001-10%, 0.001-8%, 0.001-6%, 0.001-5%, 0.001-3%, 0.001-2%, 0.001-1%, 0.001-0.5%, or 0.001-0.1%.

Provided herein, in one aspect, is a collection of cryo-vessels comprising cryopreserved platelets, wherein the cryopreserved platelets in each cryo-vessel have a biomolecule profile indicative of more than 1 platelet donor, and
wherein the concentration of DMSO in the cryopreserved platelets of a first cryo-vessel is within 25%, 20%, 15%, 10%, 5%, or 1% of the concentration of DMSO in the cryopreserved platelets of a second cryo-vessel.

Provided herein, in one aspect, is a composition comprising frozen platelets in a cryopreservation medium in a frozen state, wherein the composition is capable of yielding one or more of the following properties after storage for at least 1 month, 2, 3, 4, 5, 6, 8, 10, or 12 months, in an illustrative embodiments at a temperature in a range of −10° C. to −30° C., or −20° C. +/−5° C., upon thawing:
a) is in a liquid state without the addition of a liquid;
b) exhibits a platelet count of at least 1.0×10¹¹/35 ml of the composition;
c) yields a single peak that corresponds to a compromised membrane peak in a membrane integrity assay;
d) exhibits a CD61-positive-microparticle content of less than 50% of the CD61 positive particles in the composition, and
e) generates thrombin in an in vitro thrombin generation assay. In some embodiments, a composition is capable of yielding two or more, three or more, four, or all of the properties. In illustrative embodiments, a composition is capable of yielding all of the properties. In some embodiments, a composition is capable of yielding properties a), b), and d). In some embodiments, a composition is capable of yielding properties a), b), d), and e).

Provided herein, in one aspect, is a composition comprising frozen platelets in a cryopreservation medium in a frozen state, wherein the composition is capable of yielding one or more of the following properties after storage for at least 1 month, 2, 3, 4, 5, 6, 8, 10, or 12 months, in an illustrative embodiments at a temperature in a range of −10° C. to −30° C., or −20° C. +/−5° C., upon thawing:
a) exhibits a platelet count of at least 1.0×10¹¹/35 ml of the composition;
b) yields a single peak that corresponds to a compromised membrane peak in a membrane integrity assay;
c) exhibits a CD61-positive-microparticle content of less than 50% of the CD61 positive particles in the composition, and
d) generates thrombin in an in vitro thrombin generation assay, and wherein the composition does not comprise freeze-dried platelet derivatives, or lyophilized platelet derivatives. In some embodiments, a composition is capable of yielding one or more, two or more, three, or all of the properties. In some embodiments, a composition is capable of yielding a), c) and d) of the properties.

Provided herein, in one aspect, is a cryopreserved platelet composition comprising cryopreserved platelets, wherein the cryopreserved platelets are stored at about −20° C. for a time period of at least 12, 18, or 24 months.

Provided herein, in one aspect, is a cryopreserved platelet composition comprising cryopreserved platelets, wherein the cryopreserved platelets are stored at about −20° C. for a time period in the range of 1 month to 12, 18, 24, or 36 months.

Provided herein, in one aspect, is a method of administering cryopreserved platelet composition to a subject, wherein the method includes thawing a cryo-vial of cryopreserved platelets (CPPs) from a collection from any one of the aspects or embodiments herein, or thawing a cryo-vial comprising the cryopreserved platelet composition of any one of the aspects or embodiments herein, to prepare a liquid comprising CPPs, and administering the liquid comprising CPPs to the subject. In illustrative embodiments, after the administering bleeding of the subject is reduced.

Provided herein, in one aspect, is a collection of cryo-vessels, comprising
a plurality of cryo-vessels, each cryo-vessel in the collection comprising frozen activated platelets comprising a population of platelet particles in a cryopreservation medium in a frozen state,
wherein the population of platelet particles in the frozen activated platelets in each cryo-vessel have a set of biomolecule profiles indicative of more than 1 platelet donor,
wherein the frozen activated platelets in each cryo-vessel in the collection, upon thawing have a capacity to generate thrombin in an in vitro thrombin generation assay. In some embodiments, the collection comprises at least 2, 3, 5, 10, 20, or 30 cryo-vessels. In illustrative embodiments, the frozen activated platelets in each cryo-vessel in the collection, have the property of exhibiting a pH of greater than 6.0 upon thawing, and storing at room temperature for 2 hours to 48 hours, 4 hours to 36 hours, or 6 hours to 24 hours. In illustrative embodiments, the thawing comprises placing the cryo-vessel in a water bath set at a temperature of 37° C.+/−2° C. until the frozen activated platelets are thawed to form a thawed activated platelet composition. In illustrative embodiments, the storing at room temperature comprises diluting the thawed activated platelet composition with saline, for example with a volume of 10 to 30 ml or 20 to 25 ml saline, typically 25 ml saline, and incubating the cryo-vessel at room temperature. In illustrative embodiments, the population of platelet particles in the frozen activated platelets in each cryo-vessel of a batch of cryo-vessels of the collection have an indistinguishable set of biomolecular profiles, wherein the population of platelet particles in each batch of the collection has a different set of biomolecular profiles than any other batch in the collection, and wherein the collection comprises a plurality of at least 2, 3, 4, 5, 10, 15, 20, 30, 50, 60, 70, 80, 90, or 100 batches of cryo-vessels,

Provided herein, in one aspect, is a cryopreserved platelet composition, comprising
frozen activated platelets comprising a population of platelet particles in a cryopreservation medium in a frozen state in a cryo-vessel,
wherein the platelet particles in the population display a phosphatidylserine positivity of at least 50%, or 60%, when measured using lactadherin binding,
wherein the platelet particles in the population have a set of biomolecule profiles indicative of more than 1 platelet donor,
wherein the frozen activated platelets, upon thawing have a capacity to generate thrombin in an in vitro thrombin generation assay. In illustrative embodiments, the frozen activated platelets have the property of exhibiting a pH of greater than 6.0 upon thawing and storing at room temperature for 2 hours to 48 hours, 4 hours to 36 hours, or 6 hours to 24 hours. In illustrative embodiments, the thawing comprises placing the cryo-vessel in a water bath set at a temperature of 37° C.+/−2° C. until the frozen activated platelets are thawed to form a thawed activated platelet composition. In illustrative embodiments, the storing at room temperature comprises diluting the thawed activated platelet composition with saline, for example with a volume of 15 to 30 ml or 20 to 25 ml saline, typically 25 ml saline, and incubating the cryo-vessel at room temperature.

Provided herein, in one aspect is a cryopreserved platelet composition, frozen activated platelets, or a pooled CPP product, comprising a population of platelet particles, wherein the population of platelet particles has the property, upon thawing and when analyzed using flow cytometry, of forming two detectable sub-populations of platelet particles, a more activated sub-population, and a less activated sub-population upon
gating a population of platelet-sized particles using a fluorescently-tagged antibody that recognizes CD41 and forward scatter height (FSC-H); and
analyzing the population of platelet-sized particles obtained in the gating using a fluorescently-tagged protein specific for phosphatidylserine (PS) and a fluorescently-tagged antibody specific for CD42b. In illustrative embodiments, the platelet-sized particles in the more activated sub-population are PS positive

In some embodiments of any of the aspects and embodiments, the volume of the thawed activated platelet composition after the thawing but before diluting is between 22 and 32 ml, 23 and 31 ml, 24 and 30 ml, 24.5 and 29.75 ml, 24.7 and 29.5 ml, 25 and 29 ml, or 25 and 28.5 ml. In some embodiments, the weight of the thawed activated platelet composition after the thawing but before diluting is between 22 and 32 grams, 23 and 31 grams, 24 and 30 grams, 24.50 and 30.50 grams, 24.50 and 29.75 grams, 25.40 and 29 grams, or 25.75 and 28.94 grams.

Provided herein, in one aspect, is a collection of cryo-vessels, comprising
a plurality of cryo-vessels, in illustrative embodiments, each cryo-vessel in the collection comprising 1 unit equivalent of frozen activated platelets comprising a population of platelet particles in a cryopreservation medium in a frozen state platelet particles,
wherein the population of platelet particles in the frozen activated platelets in each cryo-vessel have a set of biomolecule profiles indicative of more than 1 platelet donor,
wherein the frozen activated platelets in each cryo-vessel in the collection, upon thawing have a capacity to generate thrombin in an in vitro thrombin generation assay. In illustrative embodiments, the frozen activated platelets in each cryo-vessel in the collection, have the property of having less than 15×10⁶, 12×10⁶, or 10×10⁶ CD61-positive microparticles/μl of frozen activated platelets, upon thawing to form a thawed activated platelet composition and when diluted with saline, for example 15 to 30 ml, 20 to 25 ml, typically 25 ml saline. In illustrative embodiments, the thawing comprises placing the cryo-vessel in a water bath set at a temperature of 37° C.+/−2° C. until the frozen activated platelets are thawed to form the thawed activated platelet composition. In illustrative embodiments, the population of platelet particles in the frozen activated platelets in each cryo-vessel of a batch of cryo-vessels of the collection have an indistinguishable set of biomolecular profiles comprising a phosphatidylserine positivity of at least 50%, or 60%, when measured using lactadherin binding, wherein the population of platelet particles in each batch of the collection has a different set of biomolecular profiles than any other batch in the collection, and wherein the collection comprises a plurality of at least 2, 3, 5, 10, 15, 20, 40, 50, 70, 80, 90, or 100 batches of cryo-vessels.

In some embodiments of any of the aspects or embodiments herein, that include a collection of cryo-vessels, a cryopreserved platelet composition, or a process for preparing a batch of a cryopreserved platelet composition, the cryopreserved platelets or frozen activated platelets, upon thawing exhibit a property of specific adhesion to collagen. In some embodiments, the specific binding is to a collagen-coated channel. Such a property can be measured for example by measuring the area of a collagen-coated channel that fluoresces, referred to herein as area coverage, after applying a labeled sample to the channel. In some embodiments, the specific adhesion exhibited by the frozen activated platelets in at least one cryo-vessel exhibits a rate of adhesion to collagen (e.g., a collagen-coated channel) that is equal to or more than 10%, 15%, 20%, 25%, 30%, 35%, 40%, 45%, or 50% of a rate of adhesion of platelets in a platelet-rich plasma under the same conditions. In some embodiments, the rate of adhesion to collagen (e.g., a collagen-coated channel) is greater than the rate of adhesion exhibited by platelets in a platelet-rich plasma. In some embodiments, the in vitro assay comprises testing one cryo-vessel each from at least 3, 4, 5, 6, 7, 8, 9, or 10 different batches in the collection of cryo-vessels, and wherein at least 1, 2, 3, 4, or 5 cryo-vessels, or at least 1/5, 1/4, 1/3 or 1/2 of the cryo-vessels, or between 1/5 and 1/2 of the cryo-vessels exhibit a rate of adhesion to collagen (e.g., a collagen-coated channel) that is equal to or more than 10%, 15%, 20%, 25%, 30%, 35%, 40%, 45%, 50%, 60%, 70%, 75%, 80%, or 90% of a rate of adhesion of platelets in a platelet-rich plasma under the same conditions, for example, 10% to 90%, 20% to 90%, 30% to 95%, 35% to 95%, or 40% to 99% of a rate of adhesion of platelets in a platelet-rich plasma under the same conditions. Such rate can be measured at a 1 minute, 2 minute, 3 minute, 4 minute, 5 minute, 6 minute 7 minute, 8 minute, or 9 minute, 10 minute time point as non-limiting examples. In some embodiments, the specific adhesion of the cryopreserved platelets herein exhibits an area coverage of adhesion to the collagen-coated channel that is equal to or more than 10%, 15%, 20%, 25%, 30%, 35%, 40%, 45%, 50%, 60%, 70%, 75%, 80%, or 90% of an area coverage to a collagen-coated channel exhibited by platelets in a platelet-rich plasma under the same conditions. In some embodiments, the specific adhesion of the cryopreserved platelets herein exhibits an area coverage of adhesion to the collagen-coated channel that is 10% to 90%, 20% to 90%, 30% to 95%, 35% to 95%, or 40% to 99% of an area coverage to a collagen-coated channel exhibited by platelets in a platelet-rich plasma under the same conditions. In some embodiments, the specific adhesion of the cryopreserved platelets herein exhibits an area coverage of adhesion to the collagen-coated channel that is at least 1.2, 1.5, 2, 2.5, 3, 3.5, or 4 fold, or in the range of 1.2-10, 1.2-9, 1.2-8, 1.2-6, 2-10, 3-10, or 1.2-5 fold of the area coverage to a collagen-coated channel exhibited by platelets in a platelet-rich plasma under the same conditions, for example, after 1, 2, 3, 4, 5, 6, or 7 minutes of contacting with a collagen-coated channel, for example under similar concentration of platelets like in a range of 2.0×10⁵/μl to 7.0×10⁵/μl, or 2.0×10⁵/μl to 5.5×10⁵/μl, or about 2.5×10⁵/μl, or 5.0×10⁵/μl. In some embodiments, the in vitro assay comprises testing one cryo-vessel each from at least 3, 4, 5, 6, 7, 8, 9, or 10 different batches in the collection of cryo-vessels, and wherein less than 1/5, 1/4, 1/3, 1/2, 34 or all, but at least 1, 2, 3, 4, or 5 cryo-vessels exhibits an area coverage of collagen adhesion that is equal to or more than 10%, 15%, 20%, 25%, 30%, 35%, 40%, 45%, 50%, 60%, 70%, 75%, 80%, or 90% of an area coverage of collagen adhesion of platelets in a platelet-rich plasma under the same conditions, for example, 10% to 90%, 20% to 90%, 30% to 95%, 35% to 95%, or 40% to 99% of an area coverage of collagen adhesion of platelets in a platelet-rich plasma under the same conditions. For example, at least 1, 2, 3, 4, or 5 cryo-vessels, but less than all, less than 3/4, less than 1/2 and in some embodiments less than 1/3 of the cryo-vessels exhibits an area coverage that is at least 1.2, 1.5, 2, 2.5, 3, 3.5, or 4 fold or in the range of 1.2-10, 1.2-9, 1.2-8, 1.2-6, 2-10, 3-10, or 1.2-5 fold of the area coverage to a collagen-coated channel exhibited by platelets in a platelet-rich plasma under the same conditions, for example, after 1, 2, 3, 4, 5, 6, or 7 minutes of contacting with a collagen-coated channel, for example under similar concentration of platelets like in a range of 2.0×10⁵/μl to 7.0×10⁵/μl, or 2.0×10⁵/μl to 5.5×10⁵/μl, or about 2.5×10⁵/μl, or 5.0×10⁵/μl.

In some embodiments of any of the aspects or embodiments herein, that include a collection of cryo-vessels, a cryopreserved platelet composition, or a process for preparing a batch of a cryopreserved platelet composition, the cryopreserved platelets or frozen activated platelets, have/exhibit a property that upon thawing they specifically adhere to collagen. In some embodiments, the specific adherence/adhesion of the frozen activated platelets upon thawing to collagen indicates that the frozen activated platelets upon thawing have the property of adhering to a collagen-coated channel but not adhering to an albumin-coated channel in an in vitro assay under the same conditions. In some embodiments, the specific adherence/adhesion of frozen activated platelets upon thawing to collagen indicates that the frozen activated platelets upon thawing have the property of adhering to the collagen-coated channel but not adhering to an uncoated channel in the in vitro assay under the same conditions. In some embodiments, the specific adhesion exhibits an area coverage of adhesion to the collagen-coated channel that is equal to or more than 50%, 75%, or 90% of an area coverage to a collagen-coated channel exhibited by platelets in a platelet-rich plasma under the same conditions, for example at 2 minutes, 5 minutes, or 7 minutes incubation after being added to the collagen-coated channel. In some embodiments, the specific adhesion exhibits an area coverage of adhesion to the collagen-coated channel that is equal to or more than 1.1, 2, 3, 4, or 5-fold, or 1.1-10, 1.1-8, 2-10, 1.1-7 fold of an area coverage to a collagen-coated channel exhibited by platelets in a platelet-rich plasma under the same conditions. In some embodiments, the specific adhesion exhibits a rate of adhesion to the collagen-coated channel that is equal to or more than 50%, 75%, or 90% of a rate of adhesion to a collagen-coated channel exhibited by platelets in a platelet-rich plasma under the same conditions. In some embodiments, the specific adhesion exhibits a rate of adhesion to the collagen-coated channel that is equal to or more than 1.1, 2, 3, 4, or 5-fold, or 1.1-10, 1.1-8, 2-10, 1.1-7 fold of a rate of adhesion to a collagen-coated channel exhibited by platelets in a platelet-rich plasma under the same conditions. In some embodiments, the specific adhesion is exhibited in the absence of tissue factor (TF) and one or more external calcium sources. In some embodiments, the specific adhesion is exhibited further in the presence of one or more of: sodium citrate dihydrate, sodium dihydrogen-phosphate dihydrate, and glycine. In some embodiments, the plasma comprises plasma protein in a concentration range of 0.040-0.09 g/ml, 0.003-0.10 g/ml, or 0.045-0.07 g/ml. In some embodiments, the frozen activated platelets upon thawing, and optionally upon diluting have a concentration of at least 1×10⁵ thawed platelet particles/μl, or in the range of 1×10⁵ to 7.5×10⁵ thawed platelet particles/μl, or 2.5 ×10⁵ to 5.0×10⁵ thawed platelet particles/μl. In some embodiments, the shear flow indicates contacting the thawed activated platelet composition, to the collagen-coated channel at a pressure and shear stress in the range of 10-40 dyn/cm², in illustrative embodiments, at a pressure and shear stress of 30 dyn/cm². In some embodiments, the contacting is done for a time in the range of 5-15 minutes, 6-10 minutes, or for 7 minutes. In some embodiments, the acquiring the image is done every 10, 15, 20, 35, 30, 35, or 40 seconds. In some embodiments, the thawed activated platelets can be labeled by a stain to enable detecting/imaging/quantifying platelets that are adhered to a collagen-coated channel, for example to measure fluorescence area coverage. In illustrative embodiments, the stain is Calcein AM. In some embodiments, the frozen activated platelets upon thawing have the property of adhering to a collagen-coated channel in the presence of plasma such that the frozen activated platelets upon thawing show an area coverage of at least 500 μm² after 1, 2, 3, 4, 5, 6, or 7 minutes of the contacting. In some embodiments, the frozen activated platelets upon thawing have the property of not adhering to albumin in the presence of plasma such that the frozen activated platelets upon thawing do not show a detectable area coverage after 1, 2, 3, 4, 5, 6, or 7 minutes of the contacting, for example, less than 100, 90, 80, 50, 40, 30, or 20 μm² after 7 minutes of the contacting. In some embodiments, the area coverage is a mean area coverage of the specific adhesion of thawed platelets to collagen. In some embodiments, the rate of adhesion of thawed platelets to collagen is a mean rate of adhesion. Such measurements can be taken after loading thawed activated platelets at a concentration of at least 1×10⁵ platelet particles/μl, or in the range of 1×10⁵ to 7.5×10⁵ platelet particles/μl, or 2.5×10⁵ to 5.0×10⁵ platelet particles/μl.

In some embodiments of any of the aspects or embodiments herein, that include a collection of cryo-vessels, a cryopreserved platelet composition, or a process for preparing a batch of a cryopreserved platelet composition, the frozen activated platelets, upon thawing and storing at room temperature for a range between 2, 4, or 6 hours on the low end to 24, 36, or 48 hours on the high end have the property of exhibiting a pH of greater than 6.0, 6.2, or 6.4. In some embodiments, the frozen activated platelets in each cryo-vessel in the collection have the property of exhibiting a pH of greater than 6.0, 6.2, or 6.4 upon thawing, and in illustrative embodiments, when diluted with 15 to 30 ml, 20 to 25 ml, or 25 ml saline, and storing at room temperature for 6 hours to 24 hours. In some embodiments, the frozen activated platelets, or the cryopreserved platelet composition, have the property of maintaining the pH of the frozen activated platelets within the range of 6.0 to 7.5, 6.1 to 7.5, 6.4 to 7.5, or 6.6 to 6.9 upon thawing and storing at room temperature for 6 to 24 hours, wherein the thawing comprises placing the cryo-vessel in a water bath set at a temperature of 37° C.+/−2° C. until the frozen activated platelets are thawed to form the thawed activated platelet composition, and diluting the thawed activated platelet composition with 25 ml of saline. In some embodiments, the pH of the frozen activated platelets, or the cryopreserved platelet composition, within 30, 15, 10, or 5 minutes after thawing, or immediately upon thawing, exhibit a pH in the range of 6.0 to 7.5, or 6.0 to 7.0. In some embodiments, the pH of the frozen activated platelets, or the cryopreserved platelet composition, upon thawing and storing at the room temperature is maintained within +/−0.025 to 1.0, or 0.05 to 0.5 pH units as compared to the pH of the frozen activated platelets, or the cryopreserved platelet composition, within 30, 15, 10, or 5 minutes after thawing, or immediately upon thawing. In some embodiments, the pH of the frozen activated platelets, or the cryopreserved platelet composition, upon thawing and storing at the room temperature for 6 hours, 8 hours, 12 hours, 24 hours, 36 hours, or 48 hours does not increase or decrease by greater than 0.5, 0.4, 0.3, or 0.2 pH unit. In some embodiments, the frozen activated platelets, or the cryopreserved platelet composition, have the property of exhibiting a mean pH across at least 3, 4, 5, 10, 20 batches in the range of 6.0 to 7.5, 6.2 to 7.2, or 6.40 to 6.95, upon thawing and when diluted with 10, 15, 20, or 25 ml saline, and storing at room temperature for 4 hours, 8 hours, or 24 hours, wherein the thawing comprises placing the cryo-vessel in a water bath set at a temperature of 37° C.+/−2° C. until the frozen activated platelets or the cryopreserved platelet composition are thawed to form a thawed activated platelet composition. In some embodiments, the frozen activated platelets, or the cryopreserved platelet composition, in each of the cryo-vessels across at least 2, 3, 4, 5, 10, 12 batches have the property of exhibiting a pH greater than 6.0, or 6.2 upon thawing and storing at room temperature for 6, 8, or 24 hours, wherein the thawing comprises placing the cryo-vessel in a water bath set at a temperature of 37° C.+/−2° C. until the frozen activated platelets are thawed to form the thawed activated platelet composition, and the storing at room temperature comprises diluting the thawed activated platelet composition with saline and incubating the cryo-vessel at room temperature.

In some embodiments of any of the aspects or embodiments herein, that include a collection of cryo-vessels, a cryopreserved platelet composition, or a process for preparing a batch of a cryopreserved platelet composition, in illustrative embodiments, wherein each cryo-vessel has the equivalent of 1 unit of frozen activated platelets, and in illustrative embodiments, the collection comprises at least 5, 10, 15, 20, or 30 cryo-vessels, and each cryo-vessel has the property of having less than 15.0×10⁶, 12.0×10⁶, 10.0×10⁶, 9.5×10⁶, or 9.0×10⁶ CD61-positive microparticles/μl of frozen activated platelets, upon thawing the frozen activated platelets, and when diluted with 15, 20, 25, or 30 ml of saline, in illustrative embodiments when the weight of the cryopreserved platelet composition in the cryo-vessel is adjusted to 20.0 to 30.0 g, 21.0 to 29.0 g, 25.0 to 30.0 g, 25.0 to 29.0 g, or 25.50 to 29.0 g, 22.0 to 27.0 g, 22.5 to 26 g, or 23.0 g to 23.5 g upon thawing and before diluting with the saline. In some embodiments, the collection comprises at least 2, 5, 10, 15, 20, 25, 30, 40, or 50 cryo-vessels each having 1 unit equivalent of frozen activated platelets, and has the property of having a concentration of CD61-positive microparticles in the range of 2.0×10⁶ to 10×10⁶, 2.0×10⁶ to 9.5×10⁶, or 2.0×10⁶ to 9.0×10⁶ CD61-positive microparticles/μl of frozen activated platelets upon thawing the frozen activated platelets, and when diluted with 15, 20, 25, or 30 ml of saline. In some embodiments, the frozen activated platelets in each cryo-vessel in the collection, have the property of having less than 9.0×10⁶ CD61-positive microparticles/μl, or in the range of 2.0×10⁶ to 9.0×10⁶ CD61-positive microparticles/μl of frozen activated platelets, upon thawing and when diluted with 15, 20, 25, or 30 ml of saline. In some embodiments, each cryo-vessel has the equivalent of 1 unit of the frozen activated platelets or the cryopreserved platelet composition, and has the property of having less than 15×10⁶, 12×10⁶, 10×10⁶, 9.75×10⁶, 9.50×10⁶, 9.25×10⁶, 9.0×10⁶, 8.8×10⁶ CD61-positive microparticles/μl, or in the range of 0.5×10⁶ to 10×10⁶, 1.5×10⁶ to 9.5×10⁶, or 2.0×10⁶ to 9×10⁶ CD61-positive microparticles/μl, of the frozen activated platelets or the cryopreserved platelet composition upon thawing, and storing at room temperature for 6 to 24 hours, wherein the thawing comprises placing the cryo-vessel in a water bath set at a temperature of 37° C.+/−2° C. until the frozen activated platelets or the cryopreserved platelet composition are thawed to form a thawed activated platelet composition, and diluting the thawed activated platelet composition with 15, 20, 25, or 30 ml of saline. In some embodiments, each cryo-vessel has the equivalent of 1 unit of the frozen activated platelets, or the cryopreserved platelet composition, and the frozen activated platelets or the cryopreserved composition of the collection has the property of having a mean concentration of CD61-positive microparticles across at least 3, 4, 5, 10, 15, 20 batches in the range of 5.5×10⁶ to 9.5×10⁶ CD61-positive microparticles/μl of the frozen activated platelets or the cryopreserved platelet composition upon thawing, and storing at room temperature for 8, 12, 24, 36, 48 hours, wherein the thawing comprises placing the cryo-vessel in a water bath set at a temperature of 37° C.+/−2° C. until the frozen activated platelets or the cryopreserved platelet composition are thawed to form a thawed activated platelet composition, and diluting the thawed activated platelet composition with 15, 20, 25, or 30 ml of saline. In some embodiments, each cryo-vessel has the equivalent of 1 unit of frozen activated platelets, and has the property of exhibiting a coefficient of variance of the ratio of concentration of CD61-positive microparticles to concentration of thawed platelet particles in the frozen activated platelets, or the cryopreserved platelet composition in the cryo-vessels, across at least 2, 5, 10, 15, 20 batches, of less than 35%, 30%, 25%, or 20% within the batch of the cryo-vessels, upon thawing and when diluted with 15, 20, 25, or 30 ml saline, wherein the thawing comprises placing the cryo-vessel in a water bath set at a temperature of 37° C.+/−2° C. until the frozen activated platelets or the cryopreserved platelet composition are thawed to form a thawed activated platelet composition. In some embodiments, each cryo-vessel has the equivalent of 1 unit of frozen activated platelets, and has the property of exhibiting a mean concentration of CD61 positive-microparticles across at least 5, 10, 15, 20 batches in the range of 4.5×10⁶/μl to 6.5×10⁶/μl, 4.5×10⁶/μl to 5.60×10⁶/μl, or 4.7×10⁶/μl to 5.5×10⁶/μl of the frozen activated platelets or the cryopreserved platelet composition upon thawing, and when diluted with 15, 20, 25, or 30 ml saline, wherein the thawing comprises placing the cryo-vessel in a water bath set at a temperature of 37° C.+/−2° C. until the frozen activated platelets or the cryopreserved platelet composition are thawed to form a thawed activated platelet composition. In some embodiments, each cryo-vessel has the equivalent of 1 unit of frozen activated platelets, and has the property of exhibiting a mean CD61-positive microparticle concentration/μl across at least 3, 5, 10, 15, 20 batches in the range of 5.5×10⁶ to 8.5×10⁶/μl, upon thawing and when diluted with 15, 20, 25, or 30 ml saline, and storing at room temperature for 8, 12, 24, 36, 48 hours, wherein the thawing comprises placing the cryo-vessel in a water bath set at a temperature of 37° C.+/−2° C. until the frozen activated platelets or the cryopreserved platelet composition are thawed to form a thawed activated platelet composition. In some embodiments, each cryo-vessel across at least 2, 3, 4, 5, 10, 15, 20 batches has the equivalent of 1 unit of frozen activated platelets, and has the property of having less than 15×10⁶, 12×10⁶, or 10×10⁶/μl CD61 positive microparticle concentration of the frozen activated platelets or the cryopreserved platelet composition, upon thawing the frozen activated platelets or the cryopreserved platelet composition, and when diluted with 15, 20, 25, or 30 ml saline. In some embodiments, each cryo-vessel across at least 2, 3, 4, 5, 10, 20 batches has the equivalent of 1 unit of frozen activated platelets, and has the property of having less than 20.0%, 25.0%, 30.0%, 35.0%, 40.0%, 42.5%, 45.0%, 50.0%, 55.0%, or 60.0% of the cryo-vessels in the collection with a CD61 positive microparticles to thawed platelet particles ratio of greater than 0.75, 1.0, or 1.25. In some embodiments, the collection has at least 5, 10, 15, 20, 30, 40, 50, 60, 70, 80, 90, or 100 cryo-vessels, or in the range of 2-90, 5-90, or 10-90 cryo-vessels, or 88 cryo-vessels, and the collection has the property of having less than 35.0%, 40.0%, 42.5%, 45.0% or 50.0% of the cryo-vessels in the collection with a CD61 positive microparticles to thawed platelet particles ratio of greater than 1.0. In some embodiments, the collection has the property of having the % of cryo-vessels having a CD61 positive microparticles to thawed platelet particles ratio of greater than 1.0 in the range of 5-54%, 10-54%, 15-54%, 20-54%, 25-54%, 30-54%, 30-50%, 32-50%, or 32-54%. In some embodiments, the collection has at least 5, 10, 15, 20, 30, 40, 50, 60, 70, 80, 90, or 100 cryo-vessels, or in the range of 2-90, 5-90, or 10-90 cryo-vessels, or 88 cryo-vessels, and the collection has the property of having less than 22%, 25%, 30%, 32%, 35%, or 38% coefficient of variance of the ratio of CD61 positive microparticles to thawed platelet particles. In some embodiments, the collection has the property of exhibiting a coefficient of variance of the ratio of CD61 positive microparticles to thawed platelet particles in the range of 5-38%, 5-35%, 10-35%, 15-35%, 20-35%, or 25-35%.

In some embodiments of any of the aspects or embodiments herein, that include a collection of cryo-vessels, a cryopreserved platelet composition, or a process for preparing a batch of cryopreserved platelet composition, the collection comprises at least 2, 3, 4, 5, 10, 20, 50, 100 cryo-vessels from at least 3, 4, 5, 10, 15 batches of cryo-vessels comprising frozen activated platelets. In some embodiments, each batch comprises at least 2, 3, 5, 10, 15, 20 cryo-vessels. In some embodiments, each batch comprises 3 to 300, 2 to 200, 5 to 200, 5 to 150, 5 to 100, or 5 to 50 cryo-vessels. In some embodiments, the cryopreservation medium comprises dimethyl sulfoxide (DMSO) having a concentration in the range of 4% to 8%. In some embodiments the DMSO is in the range of 6%+/−1%, 6%+/−0.75%, 6%+/−0.5%, 6%+/−0.25%, 6%+/−0.1%, or 6%+/−0.5%. In some embodiments, the mean DMSO concentration in the frozen activated platelets, or the cryopreserved platelet composition in the cryo-vessel across at least 3, 5, 10, 15, 20, 30, 40, 50, 75, or 100 batches has a coefficient of variance in the range of 0.05-1%. In some embodiments, the concentration of DMSO in the frozen activated platelets, or the cryopreserved platelet composition in a cryo-vessel within a batch or across at least 5 batches varies by no more than 0.5%, 0.4%, 0.3%, 0.2%, 0.1%, or 0.05%.

In some embodiments of any of the aspects or embodiments herein, that include a collection of cryo-vessels, a cryopreserved platelet composition, or a process for preparing a batch of cryopreserved platelet composition, each cryo-vessel has the equivalent of 1 unit of frozen activated platelets, and has the property of exhibiting a total count of thawed platelet particles in the range of 1.8×10¹¹ to 3.5×10¹¹, upon thawing and when diluted with 15, 20, 25, or 30 ml saline, and storing at room temperature for 6 to 24 hours, wherein the thawing comprises placing the cryo-vessel in a water bath set at a temperature of 37° C.+/−2° C. until the frozen activated platelets or the cryopreserved platelet composition are thawed to form a thawed activated platelet composition. In some embodiments, each cryo-vessel has the equivalent of 1 unit of frozen activated platelets, and has the property of exhibiting a thrombin generation in the range of 1.5 to 2.0, or 1.6 to 1.9 IU/10⁶ particles, upon thawing and when diluted with 15, 20, 25, or 30 ml saline, and storing at room temperature for 6 to 24 hours. In some embodiments, each cryo-vessel has the equivalent of 1 unit of frozen activated platelets, and has the property of exhibiting a mean thrombin generation ability across at least 5, 10, 15, 20 batches, in the range of 1.5 to 1.9, 1.5 to 1.8, 1.5 to 1.7, 1.425 to 1.75, or 1.475 to 1.65 IU/10⁶ platelet particles, upon thawing and when diluted with 25 ml saline. In some embodiments, each cryo-vessel has the equivalent of 1 unit of frozen activated platelets, and has the property of exhibiting a mean thrombin generation ability across at least 5, 10, 15, 20 batches, in the range of 230 to 260, 235 to 255, 240 to 250, 220 to 275, or 241.5 to 247.5 nM thrombin peak height, upon thawing and when diluted with 25 ml saline. In some embodiments, each cryo-vessel has the equivalent of 1 unit of frozen activated platelets, and has the property of exhibiting a mean thrombin generation across at least 3, 4, 5, 10, 15, 20 batches in the range of 1.50 to 1.80 IU/10⁶ particles frozen activated platelets, or the cryopreserved platelet composition, upon thawing and when diluted with 25 ml saline, and storing at room temperature for 8, 12, 24, 36, 48 hours. In some embodiments, the platelet particles in the population, or platelet particles in the cryopreserved platelet composition exhibit phosphatidylserine positivity of between 50% to 99%, 60% to 95%, or 78.5% to 95%, when measured using lactadherin binding. In some embodiments, the platelet particles in the population, or platelet particles in the cryopreserved platelet composition have the property of exhibiting a phosphatidylserine positivity, when measured using lactadherin binding in the range of 80% to 95, or 75% to 95%, upon thawing and storing at room temperature for 6 hours, 8 hours, 12 hours, 24 hours or in the range of 6 to 24 hours. In some embodiments, the platelet particles in the population, or platelet particles in the cryopreserved platelet composition have a mean phosphatidylserine % positivity, when measured using lactadherin binding across at least 5, 10, 15, 20 batches in the range of 70% to 99%, 78% to 95%, 78% to 90%, 78.5% to 85%, or 79% to 80.5%. In some embodiments, the platelet particles in the population, or platelet particles in the cryopreserved platelet composition have the property of exhibiting a mean phosphatidylserine positivity, when measured using lactadherin binding, across at least 3, 5, 10, 15, 20 batches in the range of 70% to 99%, or 80% to 95%, upon thawing and when diluted with 25 ml saline, and storing at room temperature for 8 hours.

In some embodiments of any of the aspects or embodiments herein, that include a collection of cryo-vessels, a cryopreserved platelet composition, or a process for preparing a batch of cryopreserved platelet composition, the collection or the process can include wherein the platelet particles in the population, or platelet particles in the cryopreserved platelet composition have a mean CD62% positivity across at least 5, 10, 15, 20 batches in the range of 54% to 75%, 54.5% to 75%, 55% to 75%, 54% to 75%, or 57% to 60%. In some embodiments, each cryo-vessel has the equivalent of 1 unit of frozen activated platelets, and has the property of exhibiting a mean concentration of thawed platelet particles across at least 5, 10, 15, 20 batches in the range of 4.0×10⁶ to 6.0×10⁶, 4×10⁶ to 5.8×10⁶, 4×10⁶ to 5.5×10⁶, 4.2×10⁶ to 5.0×10⁶, or 4.40×10⁶ to 4.70×10⁶ platelets/μl upon thawing and when diluted with 25 ml saline, wherein the thawing comprises placing the cryo-vessel in a water bath set at a temperature of 37° C.+/−2° C. until the frozen activated platelets or the cryopreserved platelet composition are thawed to form a thawed activated platelet composition. In some embodiments, each cryo-vessel has the equivalent of 1 unit of frozen activated platelets, and has the property of exhibiting a mean total count of thawed platelet particles across at least 3, 5, 10, 15, 20 batches in the range of 1.75×10¹¹ to 3.0×10¹¹, upon thawing and when diluted with 25 ml saline, and stored at room temperature for 4 hours, 8 hours, or 24 hours.

In some embodiments of any of the aspects or embodiments herein, that include a collection of cryo-vessels, a cryopreserved platelet composition, or a process for preparing a batch of cryopreserved platelet composition, the frozen activated platelets, or the cryopreserved platelet composition in the cryo-vessels, upon thawing have the property of exhibiting less than 60%, 55%, 50%, 45%, 40% aggregation in the presence of an agonist, in illustrative embodiments, the agonist can be selected from the group consisting of collagen, thrombin, arachidonic acid, and thrombin receptor activator peptide (TRAP-6), but in the absence of fresh platelets, liquid stored platelets, or apheresis platelets, and in the absence of divalent cations, wherein the thawing comprises placing the cryo-vessel in a water bath set at a temperature of 37° C.+/−2° C. until the frozen activated platelets or the cryopreserved platelet composition are thawed to form a thawed activated platelet composition. In some embodiments, upon thawing, the thawed activated platelet composition can be resuspended or diluted with 10-20 ml, 20-30 ml, typically 25 ml saline. In some embodiments, the frozen activated platelets, or the cryopreserved platelet composition in the cryo-vessels, upon thawing have the property of exhibiting 2-20%, 2-18%, 2-15%, 3-15%, or 5-15% aggregation in the presence of TRAP-6, but in the absence of fresh platelets, and in the absence of divalent cations. In some embodiments, the frozen activated platelets, or the cryopreserved platelet composition in the cryo-vessels, upon thawing have the property of exhibiting 2-20%, 2-18%, 2-15%, 3-15%, or 5-15% aggregation in the presence of arachidonic acid, but in the absence of fresh platelets, and in the absence of divalent cations. In some embodiments, the frozen activated platelets, or the cryopreserved platelet composition in the cryo-vessels, upon thawing have the property of exhibiting 10-50%, 15-50%, 15-45%, 15-40%, 20-45%, or 25-35% aggregation in the presence of thrombin, but in the absence of fresh platelets, and in the absence of divalent cations. In some embodiments, the frozen activated platelets, or the cryopreserved platelet composition in the cryo-vessels, upon thawing have the property of exhibiting 2-20%, 5-20%, 5-20%, 5-18%, or 5-15% aggregation in the presence of collagen, but in the absence of fresh platelets, and in the absence of divalent cations. In illustrative embodiments, the frozen activated platelets, or the cryopreserved platelet composition in the cryo-vessels, upon thawing have the property of exhibiting above 20% aggregation, such as 21-40%, 21-37%, or 21-35% in the presence of thrombin, but in the absence of fresh platelets, and in the absence of divalent cations, and exhibiting less than 20% aggregation, such as 3-19%, 3-17%, or 3-15% in the presence of other agonists like collagen, arachidonic acid, and TRAP-6. In illustrative embodiments, the frozen activated platelets, or the cryopreserved platelet composition in the cryo-vessels, upon thawing have the property of exhibiting at least 1.5-fold, 2-fold, 2.5-fold, or 3-fold higher aggregation in the presence of thrombin but in the absence of fresh platelets, and in the absence of divalent cations as compared to the aggregation in the presence of collagen, arachidonic acid, or TRAP-6. In illustrative embodiments, the frozen activated platelets, or the cryopreserved platelet composition in the cryo-vessels, upon thawing have the property of exhibiting 1.25-3-fold, 1.25-2.75-fold, 1.50-3-fold, 1.25-2.50-fold, or 1.25-2.25-fold higher aggregation in the presence of thrombin, but in the absence of fresh platelets, and in the absence of divalent cations as compared to the aggregation in the presence of collagen, arachidonic acid, or TRAP-6.

Provided herein, in one aspect, is a method for reducing bleeding in a subject, comprising:
thawing the frozen activated platelets, or the cryopreserved platelet composition obtained from any of the processes herein, to obtain a thawed composition comprising thawed platelet particles, and
administering the thawed composition comprising a dose, a first dose, or an effective amount of the thawed platelet particles to the subject,
wherein the administering leads to the subject having reduced bleeding such that after the administering the bleeding in the subject is reduced as compared to a bleeding in the subject before the administering. In some embodiments, the method comprises resuspending the thawed composition with saline to obtain a thawed-resuspended composition comprising thawed platelet particles. In some embodiments, the subject is undergoing, or has undergone surgery. In some embodiments, the surgery is cardiopulmonary bypass surgery, and the administering is done intraoperatively, or post-surgery. In some embodiments, the administering is done intraoperatively and post-operatively. In some embodiments, the post-surgery administration comprises administering the composition to the subject wherein the subject has an active clotting time (ACT) of less than 250 seconds. In some embodiments, the administering is done within 1 hour, 45, 30, or 15 minutes of thawing the frozen activated platelets or the cryopreserved platelet composition.

In some embodiments of any of the aspects or embodiments herein that include a processes for preparing cryopreserved platelets or batches thereof, can include a step of adding a cryoprotectant (e.g., DMSO) to a platelet resuspension, in illustrative embodiments, a concentrated pooled platelet resuspension (CPR), distributing the platelet resuspension into a collection of cryo-vessels and freezing the collection of cry-vessels. In some embodiments, the freezing is initiated within 3, 2, or 1 hour after adding the cryoprotectant, thus in an elapsed time of 3, 2, or 1 hour between the adding the cryoprotectant and the freezing. In some embodiments, such an elapsed time can be in the range of 0.5 to 4.0, 1.0 to 3.5, 1.0 to 3.0, 1.25 to 3.0, 1.5 to 3.0, 1.5 to 2.5, 2.0 to 2.5, 0.5 to 3.5, 0.5 to 3.0, 0.5 to 2.5, 0.5 to 2.0, 0.5 to 1.5, or 0.5 to 1.0 hours. In some embodiments, the time elapsed from the addition of the cryoprotectant to the pooled resuspension, until the pooled resuspension having cryoprotectant becomes frozen, can be less than or equal to 4, 3, 2, or 1 hour. In illustrative embodiments, the time elapsed from the addition of the cryoprotectant to the pooled resuspension, until the pooled resuspension having cryoprotectant becomes frozen can be in the range of 0.5 to 4.0, 1.0 to 3.5, 1.5 to 3.0, 2.0 to 2.5, 0.5 to 3.5, 0.5 to 3.0, 0.5 to 2.5, 0.5 to 2.0, 0.5 to 1.5, or 0.5 to 1.0 hours. In some embodiments, the room temperature is a temperature between 20° C. and 25° C.

In some embodiments of any of the aspects and embodiments herein the cryopreserved platelets herein exhibits the properties upon thawing, and storing at a temperature in the range of 15° C. to 30° C., 20° C. to 30° C., or 20° C. to 28° C., in a manner similar to that of the properties exhibited upon storing at a room temperature.

In some embodiments of any of the aspects or embodiments herein that include a process for preparing a batch of cryopreserved platelets, or for preparing a cryopreserved platelet composition, the process comprises centrifuging the vessels to obtain a supernatant comprising plasma, and a pellet comprising platelets. In some embodiments, the process comprises resuspending the pellet in each vessel to form a resuspension. In some embodiments, the resuspension has a target weight based on the number of platelet units pooled or provided in the vessel. In some embodiments, the process comprises pooling the resuspension from each vessel to form the CPR. In some embodiments, the process comprises removing a part of the supernatant comprising plasma until a target weight of the pellet and a remainder plasma is achieved. In some embodiments, the target weight is in the range of 15.9 g to 27.9 g times the number of units pooled or provided in the vessel. In some embodiments, removing the part of the supernatant is performed until a weight of +/−1 g of the target weight of the pellet and remainder supernatant is achieved. In some embodiments, the process comprises introducing the platelet units from the plurality of vessels to a tangential flow filtration (TFF) system. In some embodiments, the process comprises concentrating the platelet units in the TFF system to form the CPR having a target weight based on the number of platelet units pooled or provided in the plurality of vessels. In some embodiments, the process leads to the preparation of a plurality of 2, 3, 4, 5, 10, 15, 20, 30, 40, 50, 60, 70, 80, 90, 100 or more batches of the composition comprising cryopreserved platelets.

In some embodiments of any of the aspects or embodiments herein that include a process for preparing a batch of cryopreserved platelets, or for preparing a cryopreserved platelet composition, distributing is performed, in illustrative embodiments distributing in step f) is performed to a number of cryo-vessels that are equivalent to the total number of units provided in a first step, in illustrative embodiments step a). In illustrative embodiments, the distributing can be done to achieve 1 platelet unit equivalent of platelets in each cryo-vessels. In some embodiments, distributing is performed, in illustrative embodiments distributing in step f) is performed until a target fill weight is achieved in each cryo-vessel, and wherein the target fill weight is determined by the weight of the pooled resuspension having DMSO, in illustrative embodiments the target fill weight is determined by dividing the weight of the pooled resuspension having DMSO by the number of platelet units provided in a first step, in illustrative embodiments, step a). In some embodiments, before resuspending, in illustrative embodiments step c), removing a part of the supernatant comprising plasma is performed until a target weight of the pellet and a remainder plasma is achieved, wherein the target weight is in the range of 15.9 g to 27.9 g times the number of units pooled or provided in the vessel. In some embodiments, removing the part of the supernatant is performed until a weight of +/−10 g, +/−9 g, +/−8 g, +/−7 g, +/−6 g, +/−5 g, +/−4 g, +/−3 g, +/−2 g, or +/−1 g of the target weight of the pellet and remainder supernatant is achieved. In some embodiments, in step d) pooling the resuspension from each vessel is performed using a tubing tree system, in illustrative embodiments in step e) adding DMSO to the pooled resuspension vessel is performed using the tubing tree system. In some embodiments, in step f), distributing the pooled resuspension having DMSO from the pooled resuspension vessel among a number of cryo-vessels is performed using a dosing tree system. In some embodiments, the process is performed more than once to form more than one batch of the cryopreserved platelets. In some embodiments, the process is performed multiple times to form multiple batches of the cryopreserved platelets.

In some embodiments of any of the aspects and embodiments herein, that include a collection of cryo-vessels, or a process for preparing a batch of a cryopreserved platelet composition, the collection can include between 5, 10, 15, or 20 cryo-vessels on the low end and 25 cryo-vessels on the high end of the range. In some embodiments, the collection can include between 5, 10, 15, 20, 25, or 30 cryo-vessels on the low end and 50 cryo-vessels on the high end of the range. In some embodiments, the collection can include between 5, 10, 15, 20, 25, 30, 40, or 50 cryo-vessels on the low end and 100 cryo-vessels on the high end of the range. In some embodiments, the collection can include between 25, 30, 40, 50, or 100 cryo-vessels on the low end of the range and 1,000 cryo-vessels on the high end of the range. In some embodiments, the CD61 positive microparticle concentration in the frozen activated platelets, or the cryopreserved platelet composition, in each of the cryo-vessels across at least 2, 3, 4, or 5 batches, upon thawing is less than 12×10⁶/μl, 11×10⁶/μl, 10×10⁶/μl, 9.7×10⁶/μl, 9.5×10⁶/μl, 9.3×10⁶/μl, 9.0×10⁶/μl, or 8.8×10⁶/μl of the frozen activated platelets or the cryopreserved platelet composition, in illustrative embodiments, the collection is between 5, 10, 15, 20, 25, or 30 cryo-vessels on the low end and 50 cryo-vessels on the high end of the range, the collection can include between 5, 10, 15, 20, 25, or 30 cryo-vessels on the low end and 50 cryo-vessels on the high end of the range, or the collection can include between 5, 10, 15, 20, 25, 30, 40, or 50 cryo-vessels on the low end and 100 cryo-vessels on the high end of the range. In some embodiments, the pH of the frozen activated platelets, or the cryopreserved platelet composition, in each of the cryo-vessels across at least 2, 3, 4, or 5 batches, upon thawing and storing for 6, 8, or 24 hours is more than 6.0, in illustrative embodiments more than 6.2, in illustrative embodiments, the collection is between 5, 10, 15, 20, 25, or 30 cryo-vessels on the low end and 50 cryo-vessels on the high end of the range, the collection can include between 5, 10, 15, 20, 25, or 30 cryo-vessels on the low end and 50 cryo-vessels on the high end of the range, or the collection can include between 5, 10, 15, 20, 25, 30, 40, or 50 cryo-vessels on the low end and 100 cryo-vessels on the high end of the range.

Provided herein, in an aspect, is a method for reducing/decreasing, or treating bleeding in a subject, comprising:
thawing a cryo-vessel of cryopreserved platelets from the batch of cryopreserved platelets obtained from any of the aspects or embodiments herein, a cryopreserved platelet composition obtained from any of the aspects or embodiments herein, a cryo-vessel from a collection of cryo-vessels comprising cryopreserved platelets of any of the aspects or embodiments herein, or a composition of any of the aspects or embodiments herein, to form a thawed composition comprising thawed platelet particles, and
administering the thawed composition comprising a dose, a first dose, or an effective amount of the cryopreserved platelets, in illustrative embodiments, the thawed platelet particles to the subject. In illustrative embodiments, the administering leads to the subject having reduced bleeding such that after the administering the bleeding in the subject is reduced as compared to a bleeding in the subject before the administering. In some embodiments, the method further comprises resuspending the thawed composition with a sterile solution that can be infused to a subject, in illustrative embodiments, saline to obtain a thawed-resuspended composition comprising thawed platelet particles. In some embodiments, the subject is undergoing, or has undergone surgery. In some embodiments, the surgery is cardiopulmonary bypass surgery, and the administering is done intraoperatively, or post-surgery. In some embodiments, the administering is done within 4, 3, 2, 1 hour, 45, 30, or 15 minutes of thawing the cryopreserved platelet composition. In some embodiments, the post-surgery administration comprises administering the composition to the subject wherein the subject has an active clotting time (ACT) of less than 350, 300, or 250 seconds.

In any of the aspects and embodiments, the administering leads to the subject having decreased bleeding without a bleeding score of WHO Grade 2 or greater at 15 minutes, 30 minutes, 45 minutes, 1 hour, 2 hours, 4 hours, 6 hours, 8 hours, 10 hours, 12 hours, 24 hours, 48 hours 3, 4, 5, 6, 7, 8, 9, or 10 days after the administering the thawed composition to the subject, in illustrative embodiments, after the administering of a first dose of the thawed composition, in other embodiments, after the administering of a dose, or an effective amount of the cryopreserved platelets. In some embodiments, the administering leads to the subject having decreased bleeding to a bleeding score of WHO Grade 1 or lower at a primary site of bleeding within 48, 36, 24, 12, 10, 8, 6, 4, 3, 2 hours, 1 hour, 45 minutes, 30 minutes, or 15 minutes after the administering the thawed composition to the subject. In some embodiments, the administering leads to the subject having decreased bleeding to a bleeding score of WHO Grade 1 or lower at all primary and secondary sites of bleeding within 48, 36, 24, 12, 10, 8, 6, 4, 3, 2 hours, 1 hour, 45 minutes, 30 minutes, or 15 minutes after the administering the thawed composition to the subject. In some embodiments, the subject is undergoing surgery, or has undergone surgery. In some embodiments, the surgery is cardiopulmonary bypass surgery. In some embodiments, the subject is undergoing the surgery, and the administering is done intraoperatively. In some embodiments, the subject has undergone surgery, and the administering is done post-surgery. In some embodiments, the post-surgery administration comprises administering the composition to the subject wherein the subject has an active clotting time (ACT) of less than 500, 400, 300, 250, 200, 175, 150, 125, 100 seconds.

In any of the aspects and embodiments herein that include a process for preparing a batch of cryopreserved platelets, or a cryopreserved platelet composition, the freezing comprises freezing the pooled resuspension having a cryoprotectant, in illustrative embodiments, DMSO in the cryo-vessels at a temperature in the range of −10° C. to −50° C., −10° C. to −45° C., −10° C. to −40° C., −10° C. to −30° C., or −15° C. to −25° C. In some embodiments, the freezing comprises freezing the pooled resuspension at a temperature between −10° C., −12° C., −15° C., −18° C. on the high end of the temperature and −22° C., −24° C., −25° C., −27° C., −30° C., −32° C., −35° C., −37° C., −40° C., −42° C., −45° C., −47° C., −50° C., −52° C., −55° C., −57° C., or −60° C., on the low end of the temperature. In some embodiments, the freezing comprises freezing the pooled resuspension having DMSO in the cryo-vessels at a temperature of −20° C. +/−5° C., −20° C. +/−4° C., −20° C. +/−3° C., −20° C. +/−2° C., −20° C. +/−1° C., or −20° C. +/−0.5° C. In some embodiments, the batch of cryopreserved platelets are stored for between 1 month to 6 years. In some embodiments, the batch of cryopreserved platelets are stored for between 6 months to 2 years. In some embodiments, the batch of cryopreserved platelets are stored for at least 1 month, 2 months, 3 months, 4 months, 5 months, 6 months, 8 months, 10 months, 12 months, 16 months, 18 months, 24 months, 3 years, 4 years, 5 years, or 6 years, or for between 1 month and 1, 2, 3, 4, 5, or 6 years, or for between 3 months and 1, 2, 3, 4, 5, or 6 years, or for between 6 months and 1, 2, 3, 4, 5, or 6 years.

In any of the aspects and embodiments herein that include cryopreserved platelets, a cryopreserved platelet composition, a composition comprising frozen platelets in a cryopreservation medium in a frozen state, a batch of cryopreserved platelets, a collection of cryo-vessels comprising cryopreserved platelets, a process for preparing a batch of cryopreserved platelets or a cryopreserved platelet composition, the cryopreserved platelets or the frozen platelets described herein can demonstrate stability, or functional stability across a range of durations post-thawing, for example, when stored at a temperature that is more than the freezing temperature but within 50° C.

In some embodiments, the cryopreserved platelets, the cryopreserved platelet compositions, or the frozen platelets can retain stability or functional stability post-thawing for a duration in the range of 1 hour to 72 hours, 2 to 71 hours, 3 to 70 hours, 4 to 69 hours, 5 to 68 hours, 6 to 67 hours, 7 to 66 hours, or 8 to 65 hours.

In some embodiments, the cryopreserved platelets, the cryopreserved platelet compositions, or the frozen platelets can retain stability or functional stability post-thawing for a duration that can be between 5 to 8 hours, 5 to 9 hours, 5 to 10 hours, 5 to 11 hours, 5 to 12 hours, 5 to 13 hours, 5 to 14 hours, 5 to 15 hours, 5 to 16 hours, 5 to 17 hours, 5 to 18 hours, 5 to 19 hours, 5 to 20 hours, 5 to 21 hours, 5 to 22 hours, 5 to 23 hours, 5 to 24 hours, 5 to 25 hours, 5 to 26 hours, 5 to 27 hours, 5 to 28 hours, 5 to 29 hours, 5 to 30 hours, 5 to 31 hours, 5 to 32 hours, 5 to 33 hours, 5 to 34 hours, 5 to 35 hours, 5 to 36 hours, 5 to 37 hours, 5 to 38 hours, 5 to 39 hours, 5 to 40 hours, 5 to 41 hours, 5 to 42 hours, 5 to 43 hours, 5 to 44 hours, 5 to 45 hours, 5 to 46 hours, 5 to 47 hours, 5 to 48 hours, 48 hours to 2 days, 48 hours to 3 days, 48 hours to 4 days, 48 hours to 5 days, 48 hours to 6 days, 48 hours to 7 days, 48 hours to 8 days, 48 hours to 9 days, 48 hours to 10 days, 48 hours to 11 days, 48 hours to 12 days, 48 hours to 13 days, 48 hours to 14 days, or 48 hours to 15 days. In some embodiments, the cryopreserved platelets, the cryopreserved platelet compositions, or the frozen platelets can retain stability or functional stability post-thawing for a duration that can be between 5 hours on the low end of the range and 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours, 2, 3, 4, 5, 6, 7, 8, 9, 10, 11, 12, 13, 14, or 15 days on the high end of the range.

In some embodiments, the cryopreserved platelets, the cryopreserved platelet compositions, or the frozen platelets can retain stability or functional stability post-thawing for a duration that can be between 6 to 8 hours, 6 to 9 hours, 6 to 10 hours, 6 to 11 hours, 6 to 12 hours, 6 to 13 hours, 6 to 14 hours, 6 to 15 hours, 6 to 16 hours, 6 to 17 hours, 6 to 18 hours, 6 to 19 hours, 6 to 20 hours, 6 to 21 hours, 6 to 22 hours, 6 to 23 hours, 6 to 24 hours, 6 to 25 hours, 6 to 26 hours, 6 to 27 hours, 6 to 28 hours, 6 to 29 hours, 6 to 30 hours, 6 to 31 hours, 6 to 32 hours, 6 to 33 hours, 6 to 34 hours, 6 to 35 hours, 6 to 36 hours, 6 to 37 hours, 6 to 38 hours, 6 to 39 hours, 6 to 40 hours, 6 to 41 hours, 6 to 42 hours, 6 to 43 hours, 6 to 44 hours, 6 to 45 hours, 6 to 46 hours, 6 to 47 hours, 6 to 48 hours, 48 hours to 2 days, 48 hours to 3 days, 48 hours to 4 days, 48 hours to 5 days, 48 hours to 6 days, 48 hours to 7 days, 48 hours to 8 days, 48 hours to 9 days, 48 hours to 10 days, 48 hours to 11 days, 48 hours to 12 days, 48 hours to 13 days, 48 hours to 14 days, or 48 hours to 15 days. In some embodiments, the cryopreserved platelets, the cryopreserved platelet compositions, or the frozen platelets can retain stability or functional stability post-thawing for a duration that can be between 6 hours on the low end of the range and 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours, 2, 3, 4, 5, 6, 7, 8, 9, 10, 11, 12, 13, 14, or 15 days on the high end of the range.

In some embodiments, the cryopreserved platelets, the cryopreserved platelet compositions, or the frozen platelets can retain stability or functional stability post-thawing for a duration that can be between 7 to 8 hours, 7 to 9 hours, 7 to 10 hours, 7 to 11 hours, 7 to 12 hours, 7 to 13 hours, 7 to 14 hours, 7 to 15 hours, 7 to 16 hours, 7 to 17 hours, 7 to 18 hours, 7 to 19 hours, 7 to 20 hours, 7 to 21 hours, 7 to 22 hours, 7 to 23 hours, 7 to 24 hours, 7 to 25 hours, 7 to 26 hours, 7 to 27 hours, 7 to 28 hours, 7 to 29 hours, 7 to 30 hours, 7 to 31 hours, 7 to 32 hours, 7 to 33 hours, 7 to 34 hours, 7 to 35 hours, 7 to 36 hours, 7 to 37 hours, 7 to 38 hours, 7 to 39 hours, 7 to 40 hours, 7 to 41 hours, 7 to 42 hours, 7 to 43 hours, 7 to 44 hours, 7 to 45 hours, 7 to 46 hours, 7 to 47 hours, 7 to 48 hours, 48 hours to 2 days, 48 hours to 3 days, 48 hours to 4 days, 48 hours to 5 days, 48 hours to 6 days, 48 hours to 7 days, 48 hours to 8 days, 48 hours to 9 days, 48 hours to 10 days, 48 hours to 11 days, 48 hours to 12 days, 48 hours to 13 days, 48 hours to 14 days, or 48 hours to 15 days. In some embodiments, the cryopreserved platelets, the cryopreserved platelet compositions, or the frozen platelets can retain stability or functional stability post-thawing for a duration that can be between 7 hours on the low end of the range and 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours, 2, 3, 4, 5, 6, 7, 8, 9, 10, 11, 12, 13, 14, or 15 days on the high end of the range.

In some embodiments, the cryopreserved platelets, the cryopreserved platelet compositions, or the frozen platelets can retain stability or functional stability post-thawing for a duration that can be between 8 to 9 hours, 8 to 10 hours, 8 to 11 hours, 8 to 12 hours, 8 to 13 hours, 8 to 14 hours, 8 to 15 hours, 8 to 16 hours, 8 to 17 hours, 8 to 18 hours, 8 to 19 hours, 8 to 20 hours, 8 to 21 hours, 8 to 22 hours, 8 to 23 hours, 8 to 24 hours, 8 to 25 hours, 8 to 26 hours, 8 to 27 hours, 8 to 28 hours, 8 to 29 hours, 8 to 30 hours, 8 to 31 hours, 8 to 32 hours, 8 to 33 hours, 8 to 34 hours, 8 to 35 hours, 8 to 36 hours, 8 to 37 hours, 8 to 38 hours, 8 to 39 hours, 8 to 40 hours, 8 to 41 hours, 8 to 42 hours, 8 to 43 hours, 8 to 44 hours, 8 to 45 hours, 8 to 46 hours, 8 to 47 hours, 8 to 48 hours, 48 hours to 2 days, 48 hours to 3 days, 48 hours to 4 days, 48 hours to 5 days, 48 hours to 6 days, 48 hours to 7 days, 48 hours to 8 days, 48 hours to 9 days, 48 hours to 10 days, 48 hours to 11 days, 48 hours to 12 days, 48 hours to 13 days, 48 hours to 14 days, or 48 hours to 15 days. In some embodiments, the cryopreserved platelets, the cryopreserved platelet compositions, or the frozen platelets can retain stability or functional stability post-thawing for a duration that can be between 8 hours on the low end of the range and 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours, 2, 3, 4, 5, 6, 7, 8, 9, 10, 11, 12, 13, 14, or 15 days on the high end of the range.

In some embodiments, the cryopreserved platelets, the cryopreserved platelet compositions, or the frozen platelets can retain stability or functional stability post-thawing for a duration that can be between 1, 2, 3, 4, 5, 6, 7, or 8 hours on the low end of the range and 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days on the high end of the range.

In some embodiments, the cryopreserved platelets, the cryopreserved platelet compositions, or the frozen platelets can retain stability or functional stability post-thawing for a duration that can be between 0.5, 1, 2, 3, 4, 5, 6, 7, or 8 hours on the low end of the range and 25 days on the high end of the range.

In some embodiments, the cryopreserved platelets, the cryopreserved platelet compositions, or the frozen platelets can retain stability or functional stability post-thawing for a duration that can be between 0.5, 1, 2, 3, 4, 5, 6, 7, or 8 hours on the low end of the range and 20 days on the high end of the range.

In some embodiments, the cryopreserved platelets, the cryopreserved platelet compositions, or the frozen platelets can retain stability or functional stability post-thawing for a duration that can be between 0.5, 1, 2, 3, 4, 5, 6, 7, or 8 hours on the low end of the range and 15 days on the high end of the range.

In some embodiments, the cryopreserved platelets, the cryopreserved platelet compositions, or the frozen platelets can retain stability or functional stability post-thawing for a duration that can be between 0.5, 1, 2, 3, 4, 5, 6, 7, or 8 hours on the low end of the range and 10 days on the high end of the range.

In some embodiments, the cryopreserved platelets, the cryopreserved platelet compositions, or the frozen platelets can retain stability or functional stability post-thawing for a duration that can be between 0.5, 1, 2, 3, 4, 5, 6, 7, or 8 hours on the low end of the range and 5 days on the high end of the range.

In some embodiments, the cryopreserved platelets, the cryopreserved platelet compositions, or the frozen platelets can retain stability or functional stability post-thawing for a duration in the range of 30 minutes to 48 hours, 1 to 48, 2 to 48, 3 to 48, 4 to 48, 5 to 48, 6 to 48, 7 to 48, or 8 to 48 hours.

In some embodiments, the cryopreserved platelets, the cryopreserved platelet compositions, or the frozen platelets can retain stability or functional stability post-thawing for a duration of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

In any of the aspects and embodiments herein that include cryopreserved platelets, a cryopreserved platelet composition, a batch of cryopreserved platelets, a collection of cryo-vessels comprising cryopreserved platelets, a process for preparing a batch of cryopreserved platelets described herein can demonstrate stability, or functional stability across ranges of durations post-thawing, or upon thawing as per the ranges disclosed in the above embodiments, wherein the temperature of storing post-thawing is across a range of temperatures, the lowest being about above freezing temperature, for example, about 1° C., and the highest being about 50° C.

In some embodiments, the cryopreserved platelets, the cryopreserved platelet compositions, or the frozen platelets can retain stability or functional stability post-thawing at a temperature that can be between 4° C. on the low end of the range and 38° C., 39° C., 40° C., 41° C., 42° C., 43° C., 44° C., or 45° C. on the high end of the range.

In some embodiments, the cryopreserved platelets, the cryopreserved platelet compositions, or the frozen platelets can retain stability or functional stability post-thawing at a temperature that can be between 15° C. on the low end of the range and 38° C., 39° C., 40° C., 41° C., 42° C., 43° C., 44° C., or 45° C. on the high end of the range.

In some embodiments, the cryopreserved platelets, the cryopreserved platelet compositions, or the frozen platelets can retain stability or functional stability post-thawing at a temperature that can be between 20° C. on the low end of the range and 38° C., 39° C., 40° C., 41° C., 42° C., 43° C., 44° C., or 45° C. on the high end of the range.

In some embodiments, the cryopreserved platelets, the cryopreserved platelet compositions, or the frozen platelets can retain stability or functional stability post-thawing at a temperature that can be between 25° C. on the low end of the range and 38° C., 39° C., 40° C., 41° C., 42° C., 43° C., 44° C., or 45° C. on the high end of the range.

In some embodiments, the cryopreserved platelets, the cryopreserved platelet compositions, or the frozen platelets can retain stability or functional stability post-thawing at a temperature that can be between 1° C., 3° C., 5° C., 7° C., 9° C., 11° C., 13° C., 15° C., 17° C., 19° C., 21° C., 23° C., 25° C., 27° C., or 29° C. on the low end of the range and 48° C. on the high end of the range.

In some embodiments, the cryopreserved platelets, the cryopreserved platelet compositions, or the frozen platelets can retain stability or functional stability post-thawing at a temperature that can be between 1° C., 3° C., 5° C., 7° C., 9° C., 11° C., 13° C., 15° C., 17° C., 19° C., 21° C., 23° C., 25° C., 27° C., or 29° C. on the low end of the range and 45° C. on the high end of the range.

In some embodiments, the cryopreserved platelets, the cryopreserved platelet compositions, or the frozen platelets can retain stability or functional stability post-thawing at a temperature that can be between 1° C., 3° C., 5° C., 7° C., 9° C., 11° C., 13° C., 15° C., 17° C., 19° C., 21° C., 23° C., 25° C., 27° C., or 29° C. on the low end of the range and 42° C. on the high end of the range.

In some embodiments, the cryopreserved platelets, the cryopreserved platelet compositions, or the frozen platelets can retain stability or functional stability post-thawing at a temperature that can be between 1° C., 3° C., 5° C., 7° C., 9° C., 11° C., 13° C., 15° C., 17° C., 19° C., 21° C., 23° C., 25° C., 27° C., or 29° C. on the low end of the range and 40° C. on the high end of the range.

In some embodiments, the cryopreserved platelets, the cryopreserved platelet compositions, or the frozen platelets can retain stability or functional stability post-thawing at a temperature that can be between 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., or 27° C. on the low end of the range and 37° C., 38° C., 39° C., 40° C., 41° C., 42° C., 43° C., 44° C., 45° C., 46° C., 47° C., or 48° C. on the high end of the range.

In some embodiments, the cryopreserved platelets, the cryopreserved platelet compositions, or the frozen platelets can retain stability or functional stability post-thawing at a temperature in the range of 1° C. to 37° C., 3° C. to 39° C., 5° C. to 41° C., 7° C. to 43° C., 9° C. to 45° C., 11° C. to 47° C., 13° C. to 48° C., 15° C. to 48° C., 17° C. to 48° C., 19° C. to 48° C., 21° C. to 48° C., 23° C. to 48° C., 25° C. to 48° C., or 27° C. to 48° C.

In some embodiments, the cryopreserved platelets, the cryopreserved platelet compositions, or the frozen platelets can retain stability or functional stability post-thawing at a temperature in the range of 1° C. to 48° C., 2° C. to 48° C., 3° C. to 48° C., 4° C. to 48° C., 5° C. to 48° C., 6° C. to 48° C., 7° C. to 48° C., 8° C. to 48° C., 9° C. to 48° C., 10° C. to 48° C., 11° C. to 48° C., 12° C. to 48° C., 13° C. to 48° C., 14° C. to 48° C., 15° C. to 48° C., 16° C. to 48° C., 17° C. to 48° C., 18° C. to 48° C., 19° C. to 48° C., 20° C. to 48° C., 21° C. to 48° C., 22° C. to 48° C., 23° C. to 48° C., 24° C. to 48° C., 25° C. to 48° C., 26° C. to 48° C., 27° C. to 48° C., 28° C. to 48° C., 29° C. to 48° C., 30° C. to 48° C., 31° C. to 48° C., 32° C. to 48° C., 33° C. to 48° C., or 34° C. to 48° C.

In some embodiments, the cryopreserved platelets, the cryopreserved platelet compositions, or the frozen platelets can retain stability or functional stability post-thawing at a temperature in the range of 10° C. to 48° C., 11° C. to 48° C., 12° C. to 48° C., 13° C. to 48° C., 14° C. to 48° C., 15° C. to 48° C., 16° C. to 48° C., 17° C. to 48° C., 18° C. to 48° C., 19° C. to 48° C., 20° C. to 48° C., 21° C. to 48° C., 22° C. to 48° C., 23° C. to 48° C., 24° C. to 48° C., 25° C. to 48° C., 26° C. to 48° C., 27° C. to 48° C., 28° C. to 48° C., 29° C. to 48° C., 30° C. to 48° C., 31° C. to 48° C., 32° C. to 48° C., 33° C. to 48° C., or 34° C. to 48° C.

In some embodiments, the cryopreserved platelets, the cryopreserved platelet compositions, or the frozen platelets can retain stability or functional stability post-thawing at a temperature in the range of 15° C. to 48° C., 16° C. to 48° C., 17° C. to 48° C., 18° C. to 48° C., 19° C. to 48° C., 20° C. to 48° C., 21° C. to 48° C., 22° C. to 48° C., 23° C. to 48° C., 24° C. to 48° C., 25° C. to 48° C., 26° C. to 48° C., 27° C. to 48° C., 28° C. to 48° C., 29° C. to 48° C., 30° C. to 48° C., 31° C. to 48° C., 32° C. to 48° C., 33° C. to 48° C., or 34° C. to 48° C.

In some embodiments, the cryopreserved platelets, the cryopreserved platelet compositions, or the frozen platelets can retain stability or functional stability post-thawing at a temperature in the range of 22° C. to 48° C., 23° C. to 48° C., 24° C. to 48° C., 25° C. to 48° C., 26° C. to 48° C., 27° C. to 48° C., 28° C. to 48° C., 29° C. to 48° C., 30° C. to 48° C., 31° C. to 48° C., 32° C. to 48° C., 33° C. to 48° C., or 34° C. to 48° C.

In some embodiments, the cryopreserved platelets, the cryopreserved platelet compositions, or the frozen platelets can retain stability or functional stability post-thawing at a temperature of at least 1° C., at least 3° C., at least 5° C., at least 7° C., at least 9° C., at least 11° C., at least 13° C., at least 15° C., at least 17° C., at least 19° C., at least 21° C., at least 23° C., at least 25° C., at least 27° C., at least 29° C., at least 31° C., at least 33° C., at least 35° C., or at least 37° C.

In some embodiments, the cryopreserved platelets, the cryopreserved platelet compositions, or the frozen platelets can retain functional stability post-thawing for a duration that can be between 5 hours on the low end of the range and 8, 12, 24, 36, 48 hours, 2, 3, 5, 7, 10, 15, 20, 25, 30, 40, 50, or 60 days on the high end of the range, when stored at a temperature that can be between 4° C. on the low end of the range and 25° C., 30° C., 35° C., 37° C., 39° C., 41° C., 43° C., 45° C., or 48° C. on the high end of the range.

In some embodiments, the cryopreserved platelets, the cryopreserved platelet compositions, or the frozen platelets can retain functional stability post-thawing for a duration that can be between 6 hours on the low end of the range and 8, 12, 24, 36, 48 hours, 2, 3, 5, 7, 10, 15, 20, 25, 30, 40, 50, or 60 days on the high end of the range, when stored at a temperature that can be between 4° C. on the low end of the range and 25° C., 30° C., 35° C., 37° C., 39° C., 41° C., 43° C., 45° C., or 48° C. on the high end of the range.

In some embodiments, the cryopreserved platelets, the cryopreserved platelet compositions, or the frozen platelets can retain functional stability post-thawing for a duration that can be between 8 hours on the low end of the range and 12, 24, 36, 48 hours, 2, 3, 5, 7, 10, 15, 20, 25, 30, 40, 50, or 60 days on the high end of the range, when stored at a temperature that can be between 4° C. on the low end of the range and 25° C., 30° C., 35° C., 37° C., 39° C., 41° C., 43° C., 45° C., or 48° C. on the high end of the range.

In some embodiments, the cryopreserved platelets, the cryopreserved platelet compositions, or the frozen platelets upon thawing and storing at temperatures for durations as disclosed in the above embodiments can retain stability or functional stability as per one or more than one parameter as disclosed herein. In some embodiments, the parameter can be the pH of the thawed platelets, composition comprising thawed platelets, cryopreserved platelets upon thawing and storing at temperatures for durations as disclosed in the above embodiments. In some embodiments, the cryopreserved platelets, the cryopreserved platelet compositions, or the frozen platelets upon thawing can retain stability or functional stability, such that the pH post-thawing that can be between 6.0 on the low end of the range and 6.1, 6.2, 6.3, 6.4, 6.5, 6.6, 6.7, 6.8, 6.9, 7.0, 7.1, 7.2, 7.3, 7.4, or 7.5 on the high end of the range. In some embodiments, the cryopreserved platelets, the cryopreserved platelet compositions, or the frozen platelets upon thawing can retain stability or functional stability, such that the pH post-thawing that can be between 6.2 on the low end of the range and 6.3, 6.4, 6.5, 6.6, 6.7, 6.8, 6.9, 7.0, 7.1, 7.2, 7.3, 7.4, or 7.5 on the high end of the range. In some embodiments, the cryopreserved platelets, the cryopreserved platelet compositions, or the frozen platelets upon thawing can retain stability or functional stability, such that the pH post-thawing that can be between 6.4 on the low end of the range and 6.5, 6.6, 6.7, 6.8, 6.9, 7.0, 7.1, 7.2, 7.3, 7.4, or 7.5 on the high end of the range. In some embodiments, the cryopreserved platelets, the cryopreserved platelet compositions, or the frozen platelets upon thawing can retain stability or functional stability, such that the pH post-thawing that can be between 6.6 on the low end of the range and 6.7, 6.8, 6.9, 7.0, 7.1, 7.2, 7.3, 7.4, or 7.5 on the high end of the range.

In some embodiments, the cryopreserved platelets, the cryopreserved platelet compositions, or the frozen platelets upon thawing can retain stability or functional stability, such that the pH post-thawing can be between 6.0, 6.1, 6.2, 6.3, 6.4, 6.5, 6.6, or 6.7 on the low end of the range and 6.7, 6.8, 6.9, 7.0, 7.1, 7.2, 7.3, 7.4, 7.5, 7.6, 7.7, 7.8, 7.9, or 8.0 on the high end of the range.

In some embodiments, the cryopreserved platelets, the cryopreserved platelet compositions, or the frozen platelets upon thawing can retain stability or functional stability, such that the pH post-thawing can be in the range of 6.0 to 6.9, 6.0 to 7.0, 6.0 to 7.1, 6.0 to 7.2, 6.0 to 7.3, 6.0 to 7.4, 6.0 to 7.5, 6.0 to 7.6, 6.0 to 7.7, 6.0 to 7.8, 6.0 to 7.9, 6.0 to 8.0, 6.1 to 6.9, 6.1 to 7.0, 6.1 to 7.1, 6.1 to 7.2, 6.1 to 7.3, 6.1 to 7.4, 6.1 to 7.5, 6.1 to 7.6, 6.1 to 7.7, 6.1 to 7.8, 6.1 to 7.9, 6.1 to 8.0, 6.2 to 6.9, 6.2 to 7.0, 6.2 to 7.1, 6.2 to 7.2, 6.2 to 7.3, 6.2 to 7.4, 6.2 to 7.5, 6.2 to 7.6, 6.2 to 7.7, 6.2 to 7.8, 6.2 to 7.9, 6.2 to 8.0, 6.3 to 6.9, 6.3 to 7.0, 6.3 to 7.1, 6.3 to 7.2, 6.3 to 7.3, 6.3 to 7.4, 6.3 to 7.5, 6.3 to 7.6, 6.3 to 7.7, 6.3 to 7.8, 6.3 to 7.9, 6.3 to 8.0, 6.4 to 6.9, 6.4 to 7.0, 6.4 to 7.1, 6.4 to 7.2, 6.4 to 7.3, 6.4 to 7.4, 6.4 to 7.5, 6.4 to 7.6, 6.4 to 7.7, 6.4 to 7.8, 6.4 to 7.9, 6.4 to 8.0, 6.5 to 6.9, 6.5 to 7.0, 6.5 to 7.1, 6.5 to 7.2, 6.5 to 7.3, 6.5 to 7.4, 6.5 to 7.5, 6.5 to 7.6, 6.5 to 7.7, 6.5 to 7.8, 6.5 to 7.9, 6.5 to 8.0, 6.6 to 6.8, 6.6 to 6.9, 6.6 to 7.0, 6.6 to 7.1, 6.6 to 7.2, 6.6 to 7.3, 6.6 to 7.4, 6.6 to 7.5, 6.6 to 7.6, 6.6 to 7.7, 6.6 to 7.8, 6.6 to 7.9, 6.6 to 8.0, 6.7 to 6.8, 6.7 to 6.9, 6.7 to 7.0, 6.7 to 7.1, 6.7 to 7.2, 6.7 to 7.3, 6.7 to 7.4, 6.7 to 7.5, 6.7 to 7.6, 6.7 to 7.7, 6.7 to 7.8, 6.7 to 7.9, or 6.7 to 8.0.

In some embodiments, the cryopreserved platelets, the cryopreserved platelet compositions, or the frozen platelets upon thawing can retain stability or functional stability, such that the pH post-thawing can be in the range of 6.0 to 6.8, 6.0 to 6.9, 6.0 to 7.0, 6.0 to 7.1, 6.0 to 7.2, 6.0 to 7.3, 6.0 to 7.4, 6.0 to 7.5, 6.0 to 7.6, 6.0 to 7.7, 6.0 to 7.8, 6.0 to 7.9, or 6.0 to 8.0. In some embodiments, the pH values post-thawing can be in the range of 6.2 to 6.8, 6.2 to 6.9, 6.2 to 7.0, 6.2 to 7.1, 6.2 to 7.2, 6.2 to 7.3, 6.2 to 7.4, 6.2 to 7.5, 6.2 to 7.6, 6.2 to 7.7, 6.2 to 7.8, 6.2 to 7.9, or 6.2 to 8.0. In some embodiments, the pH values post-thawing can be in the range of 6.4 to 6.8, 6.4 to 6.9, 6.4 to 7.0, 6.4 to 7.1, 6.4 to 7.2, 6.4 to 7.3, 6.4 to 7.4, 6.4 to 7.5, 6.4 to 7.6, 6.4 to 7.7, 6.4 to 7.8, 6.4 to 7.9, or 6.4 to 8.0.

In some embodiments, the cryopreserved platelets, the cryopreserved platelet compositions, or the frozen platelets upon thawing, and storing, for example for at least 6, 7, 8, 12, 16, or 24 hours, or for any duration as disclosed in the above embodiments, at a temperature in the range of 4° C. to 45° C., 4° C. to 40° C., 10° C. to 45° C., or 15 to 45° C., or for any temperature range as disclosed in the above embodiments, can retain stability or functional stability, such that the pH post-thawing can be of at least 6.0, at least 6.1, at least 6.2, at least 6.3, at least 6.4, at least 6.5, or at least 6.6.

In some embodiments of any of the aspects and embodiments that include cryopreserved platelets, or a cryopreserved platelet composition, or a process or a method that include cryopreserved platelets, the cryopreserved platelets, the cryopreserved platelet compositions, or the frozen platelets upon thawing, and storing, for example for at least 6, 7, 8, 12, 16, or 24 hours, or for any duration as disclosed in the above embodiments, at a temperature in the range of 4° C. to 45° C., 4° C. to 40° C., 10° C. to 45° C., or 15 to 45° C., or for any temperature range as disclosed in the above embodiments, can maintain the pH within a range of 6.0 to 8.5, 6.0 to 8.2, 6.0 to 8.0, 6.0 to 7.8, 6.0 to 7.5, or 6.0 to 7.0. In some embodiments, the cryopreserved platelets, or the frozen platelets, immediately, for example, within 30, 25, 20, 15, 10, or 5 minutes upon thawing exhibit a pH in the range of 6.0 to 7.5, and the pH is maintained within +/−0.05 to 0.5, +/−0.05 to 0.4, +/−0.05 to 0.3, +/−0.01 to 0.7, +/−0.01 to 0.6, +/−0.01 to 0.5, +/−0.01 to 0.4, +/−0.01 to 0.3, or +/−0.01 to 0.2 pH units upon storing for at least 4, 6, 8, 12, 16, 18, or 24 hours, wherein the pH after the storing period is above 6.0, in illustrative embodiments, within the range of 6.0 to 7.5, 6.1 to 7.5, 6.4 to 7.5, or 6.60 to 6.9. In some embodiments, the pH of the composition comprising cryopreserved platelets herein upon thawing and storing for at least 4, 6, 8, 12, 16, 18, or 24 hours, does not increase or decrease by more than 0.7, 0.6, 0.5, 0.4, 0.3, 0.2, or 0.1 pH unit. In some embodiments, the pH of the composition comprising cryopreserved platelets herein upon thawing and storing for 4, 6, 8, 12, 16, 18, or 24 hours at a temperature disclosed herein, for example, 18° C.-30° C. does not increase or decrease by more than 0.7, 0.6, 0.5, 0.4, 0.3, 0.2, or 0.1 pH unit. In some embodiments, the composition in the cryo-vessels, upon thawing and storing at a temperature in the range of 18° C. to 28° C. for 4 hours, 8 hours, or 24 hours have the property of exhibiting a mean pH across at least 3 batches, or 2-10, 2-8, or 2-5 batches in the range of 6.45 to 7.0, 6.45 to 6.95, 6.50 to 7.2, 6.50 to 7.0, 6.60 to 7.0, 6.60 to 6.90, or 6.40 to 6.95.

In some embodiments of any of the aspects and embodiments that include cryopreserved platelets, or a cryopreserved platelet composition, or a process or a method that include cryopreserved platelets, the cryopreserved platelets, the cryopreserved platelet compositions, or the frozen platelets upon thawing, in some embodiments, upon thawing and storing at temperatures for durations as disclosed in the above embodiments have less than 15×10⁶, 12×10⁶, 10×10⁶, 9.5×10⁶, or 9.0×10⁶, 8.8×10⁶, 8.5×10⁶, 8.0×10⁶, 7.5×10⁶, or 7.2×10⁶ CD61-positive microparticles/μl. In some embodiments, the cryopreserved platelets, the cryopreserved platelet compositions, or the frozen platelets upon thawing and storing at a temperature in the range of 20° C. to 40° C., or 20° C. to 28° C. for 6 to 24 hours do not comprise more than 10×10⁶, 9.75×10⁶, 9.50×10⁶, 9.25×10⁶, or 9.0×10⁶ CD61-positive microparticles/μl. In some embodiments, the cryopreserved platelets, the cryopreserved platelet compositions, or the frozen platelets upon thawing have CD61-positive microparticles in the range of 0.5×10⁶ to 12×10⁶, 0.5×10⁶ to 10×10⁶, in illustrative embodiments, in the range of 0.5×10⁶ to 9.9×10⁶, 0.5×10⁶ to 9.0×10⁶, 2×10⁶ to 9.0×10⁶, or 2.5×10⁶ to 8.8×10⁶, 8.0×10¹ to 8.8×10⁶, 8.5×10¹ to 8.0×10⁶, or 8.8×10⁶ to 7.5×10⁶ CD61-positive microparticles/μl. In some embodiments, the concentration of CD61-positive microparticles in the cryopreserved platelets, the cryopreserved platelet compositions, or the frozen platelets upon thawing can be between 0.5×10⁶, 1.0×10⁶, 1.5×10⁶, 2.0×10⁶, 2.5×10⁶, 3.0×10⁶, 3.5×10⁶, or 4.0×10⁶ on the low end of the range and 9.9×10⁶ CD61-positive microparticles/μl on the high end. In some embodiments, the concentration of CD61-positive microparticles in the cryopreserved platelets, the cryopreserved platelet compositions, or the frozen platelets upon thawing can be between 0.5×10⁶, 0.8×10⁶, 0.85×10⁶, 0.9×10⁶, 1.0×10⁶, 1.5×10⁶, 2.0×10⁶, 2.5×10⁶, 3.0×10⁶, 3.5×10⁶, or 4.0×10⁶ on the low end of the range and 8.8×10⁶ CD61-positive microparticles/μl on the high end. In some embodiments, the concentration of CD61-positive microparticles in the cryopreserved platelets, the cryopreserved platelet compositions, or the frozen platelets upon thawing can be between 2.5×10⁶, 3.0×10⁶, 3.5×10⁶, 4.0×10⁶, 4.5×10⁶, 5.0×10⁶, 5.5×10⁶ or 6.0×10⁶ on the low end of the range and 8.5×10⁶, 8.8×10⁶, or 9.0×10⁶ CD61-positive microparticles/μl on the high end. In some embodiments, the concentration of CD61-positive microparticles in the cryopreserved platelets, the cryopreserved platelet compositions, or the frozen platelets upon thawing can be between 0.5×10⁶, 1.0×10⁶, 1.5×10⁶, 2.0×10⁶, 2.5×10⁶, 3.0×10⁶, 3.5×10⁶, or 4.0×10⁶ on the low end of the range and 8.5×10⁶ CD61-positive microparticles/μl on the high end. In some embodiments, the concentration of CD61-positive microparticles in the cryopreserved platelets, the cryopreserved platelet compositions, or the frozen platelets upon thawing can be between 0.5×10⁶, 1.0×10⁶, 1.5×10⁶, 2.0×10⁶, 2.5×10⁶, 3.0×10⁶, 3.5×10⁶, or 4.0×10⁶ on the low end of the range and 8.0×10⁶ CD61-positive microparticles/μl on the high end. In some embodiments, the composition comprising cryopreserved platelets or the frozen platelets, upon thawing and the storing comprise CD61-positive microparticles in the range of 0.5×10⁶ to 10×10⁶, 1.5×10⁶ to 9.5×10⁶, 2.0×10⁶ to 9×10⁶ CD61-positive microparticles/μl. In some embodiments, the composition comprising cryopreserved platelets or the frozen platelets, upon thawing and storing for 8 hours comprise CD61-positive microparticles in the range of 6.0×10⁶ to 9.0×10⁶ CD61-positive microparticles/μl. In some embodiments, the concentration comprising CD61-positive microparticles in the cryopreserved platelets do not comprise more than 10×10⁶, 9.75×10⁶, 9.50×10⁶, 9.25×10⁶, 9.0×10⁶, or 8.8×10⁶ CD61-positive microparticles/μl. In some embodiments, the ratio of concentration of CD61-positive microparticles to the concentration of platelets in the composition comprising cryopreserved platelets in the cryo-vessels, across at least 2, 3, 4, 5, 10, 12, 15, 20 batches, upon thawing exhibit a coefficient of variance of less than 50%, 45%, 40%, 35%, 30%, 25%, or 20% within the batch of the cryopreserved platelets, or the cryo-vessels. In some embodiments, the composition comprising cryopreserved platelets comprises a mean concentration of CD61 positive-microparticles across at least 2, 3, 4, 5, 10, 12, 15, 20 batches in the range of 4.5×10⁶/μl to 6.5×10⁶/μl, 4.5×10⁶/μl to 5.60×10⁶/μl, or 4.7×10⁶/μl to 5.5×10⁶/μl. In some embodiments, composition in the cryo-vessels, upon thawing and storing at a temperature in the range of 18° C. to 28° C., 18° C. to 40° C., or 20° C. to 30° C. for 8 hours have the property of exhibiting a mean CD61-positive microparticle concentration/μl across at least 3 batches in the range of 5.5×10⁶ to 8.5×10⁶/μl, 5.5×10⁶ to 8.2×10⁶/μl, or 5.5×10⁶ to 8.0 ×10⁶/μl.

In some embodiments, the cryopreserved platelets, the cryopreserved platelet compositions, or the frozen platelets upon thawing and storing at temperatures for durations as disclosed in the above embodiments can retain stability or functional stability as per one or more than one parameter as disclosed herein, and in some embodiments, the parameter can be the ratio of total platelet concentration to that of total microparticle concentration, for example, CD61-positive microparticles. In illustrative embodiments, the ratio of total platelet concentration to that of total microparticle concentration, for example, CD61-positive microparticles of the cryopreserved platelets, the cryopreserved platelet compositions, or the frozen platelets upon thawing and storing at temperatures for durations as disclosed in the above embodiments is more than 1.0. In some embodiments, the cryopreserved platelets, the cryopreserved platelet compositions, or the frozen platelets upon thawing, and storing, for example for at least 6, 7, 8, 12, 16, or 24 hours, or for any duration as disclosed in the above embodiments, at a temperature in the range of 4° C. to 45° C., 4° C. to 40° C., 10° C. to 45° C., or 15 to 45° C., or for any temperature range as disclosed in the above embodiments, can retain stability or functional stability, such that the ratio of total platelet concentration to that of total microparticle concentration, for example, CD61-positive microparticles of the cryopreserved platelets, the cryopreserved platelet compositions, or the frozen platelets upon thawing and storing at temperatures for durations as disclosed in the above embodiments is more than 1.0. In some embodiments, the ratio of CD61 positive microparticles and thawed platelet particles in cryo-vessels in a collection or a batch herein has a coefficient of variance, for example, across at least 2, 5, or 10 batches, of less than 38%, 35%, 34%, 32%, 30%, 27%, 25%, or 23%. In some embodiments, the ratio of CD61 positive microparticles and thawed platelet particles in cryo-vessels in a collection or a batch herein has a coefficient of variance, for example, across at least 2, 5, or 10 batches, and each batch comprising at least 3, or 5 cryo-vessels, in the range of 18% to 38%, 18% to 35%, 18% to 32%, 18% to 30%, 18% to 27%, 18% to 25%, 18% to 24%, 18% to 23%, or 18% to 22.5%. In some embodiments, the lower end of the CV can be 5%, 10%, 15%, 18%, or 20%, and the higher end of the CV can be 22.2%, 23%, 25%, 26%, 28%, 30%, 32%, 35%, 37%, or 38%.

In some embodiments of any of the aspects and embodiments, that include a cryopreserved platelet composition, cryopreserved platelets, or frozen platelets, including a collection, a batch, or a process, the composition comprising cryopreserved platelets or the frozen platelets, upon thawing and storing at a temperature in the range of 20° C. to 40° C., or 20° C. to 28° C. for at least 6, 8, 12, or 24 hours, or 6 to 24 hours exhibit thrombin generation of at least 1.2, 1.4, 1.5 IU/10⁶ particles, or in the range of 1.4 to 3.0, 1.5 to 3.0, 1.5 to 2.5, or 1.5 to 2.0 IU/10⁶ particles. In some embodiments, the composition comprising cryopreserved platelets or the frozen platelets, upon thawing and storing at a temperature in the range of 20° C. to 40° C., or 20° C. to 28° C. for 8 hours exhibit thrombin generation in the range of 1.6 to 1.9 IU/10⁶ particles.

In some aspects and embodiments, the subject, or the subject in need of a reduction or cessation in bleeding, has one or more indication selected from the group consisting of Von Willebrand disease, immune thrombocytopenia (ITP), intracranial hemorrhage (ICH), traumatic brain injury (TBI), Hermansky Pudlak Syndrome (HPS), chemotherapy induced thrombocytopenia (CIT), Scott syndrome, Evans syndrome, hematopoietic stem cell transplantation, fetal and neonatal alloimmune thrombocytopenia, Bernard Soulier syndrome, acute myeloid leukemia, Glanzmann thrombasthenia, myelodysplastic syndrome, hemorrhagic shock, coronary thrombosis (myocardial infarction), ischemic stroke, arterial thromboembolism, Wiskott Aldrich syndrome, venous thromboembolism, MYH9 related disease, acute lymphoblastic lymphoma (ALL), acute coronary syndrome, chronic lymphocytic leukemia (CLL), acute promyelocytic leukemia, cerebral venous sinus thrombosis (CVST), liver cirrhosis, factor v deficiency (Owren Parahemophilia), thrombocytopenia absent radius syndrome, Kasabach Merritt syndrome, Gray platelet syndrome, aplastic anemia, chronic liver disease, acute radiation syndrome, Dengue hemorrhagic fever, pre-eclampsia, snakebite envenomation, HELLP syndrome, haemorrhagic cystitis, multiple myeloma, disseminated intravascular coagulation, heparin induced thrombocytopenia, pre-eclampsia, labor and delivery, hemophilia, cerebral (fatal) malaria, Alexander's disease (Factor VII Deficiency), hemophilia C (Factor XI Deficiency), familial hemophagocytic lymphohistiocytosis, acute lung injury, hemolytic uremic syndrome, menorrhagia, chronic myeloid leukemia.

In some embodiments of any of the aspects or embodiments herein that include a process for preparing a batch of cryopreserved platelets, or for preparing a cryopreserved platelet composition, in step a) an odd number of platelet units are provided, and wherein one platelet unit is processed in a separate vessel. In some embodiments, in step a) 2 platelet units are pooled in one vessel to form a plurality of vessels. In some embodiments, in step c), the target weight is in the range of 31.8 g to 55.7 g for the vessels that have 2 platelet units, and in the range of 15.9 g to 27.9 g for the vessel that has 1 platelet unit. In some embodiments, in step c), the target weight is in the range of 43.5 g to 49.5 g for the vessels that have 2 units, and in the range of 20.3 g to 26.3 g for the vessel that has 1 unit. In some embodiments, in step c), the target weight is in the range of 45.5 g to 47.5 g for the vessels that have 2 units, and in the range of 22.3 g to 24.3 g for the vessel that has 1 unit. In some embodiments, in step c), the target weight is about 46.5 g for the vessel that had 2 units of the platelets, and is about 23.3 g for the vessel that had 1 unit of the platelets. In some embodiments, in step a) 5, 7, 9, or 11 units are provided, and wherein each unit is from a different donor. In some embodiments, in step a) 5 units from 5 donors are provided. In some embodiments, in step a) an even number of units are provided. In some embodiments, in step a) an even number of units are provided, and 2 platelet units are pooled in one vessel, to form a plurality of vessels each having 2 platelet units. In some embodiments, in step a) an even number of units are provided, and the target weight is in the range of 31.8 g to 55.7 g for each of the vessels. In some embodiments, in step a) an even number of units are provided, and the target weight is in the range of 43.5 g to 49.5 g for each of the vessels. In some embodiments, in step a) an even number of units are provided, and the target weight is in the range of 45.5 g to 47.5 g for each of the vessels. In some embodiments, in step a) an even number of units are provided, and in step a) 6, 8, 10, 12, 14, 16, 18, 20, or more units are provided, and wherein each unit is from a different donor. In some embodiments, wherein an even number of units are provided, the pooling is performed for 12 units that are from 12 donors. In some embodiments, at least 2, 3, 4, 5, 6, 7, or 8 units are pooled in a vessel.

In some embodiments of any of the aspects or embodiments herein that include a process for preparing a batch of cryopreserved platelets, or for preparing a cryopreserved platelet composition, in step e) adding the DMSO is performed to achieve a concentration of DMSO in the range of 0.001-10%, 0.001-9%, 0.001-8%, 0.001-7%, 0.001-6%, 0.001-5%, 0.001-4%, 0.001-3%, 0.001-2%, 0.001-1%, 0.001-0.9%, 0.001-0.8%, 0.001-0.7%, 0.001-0.6%, 0.001-0.5%, 0.001-0.4%, 0.001-0.3%, 0.001-0.2%, 0.001-0.1%, 0.001-0.05%, 0.001-0.01%, 0.01-10%, 0.01-9%, 0.01-5.5%, 0.05-10%, 0.1-10%, 0.5-10%, 1-10%, 1-8%, 1-7%, 1-6%, 1-4%, 2-10%, 2-8%, 2-7%, 3-10%, 3-8%, 3-7%, 4-10%, 4-8%, 4-7%, 5-10%, 5-9%, 5-8%, 5-7%, or 6-10% in the pooled resuspension. In some embodiments, adding the DMSO is performed using a stock solution of 27% DMSO. In some embodiments, the time after the addition of DMSO until the cryopreservation is no more than 1 hour, 2, 3, 4, 5, or 6 hours. In some embodiments, wherein across at least 2, 3, 4, 5, 6, 7, 8, 9, 10, 15, 20, 25, 50, 75, or 100 batches, a) a coefficient of variance of a mean resuspension volume across the batches is less than 10%; and/or b) a coefficient of variance of a mean pooled resuspension volume having DMSO in a cryo-vessel across the batches is less than 5%. In some embodiments, resuspending the pellet in each vessel leads to a resuspension in each vessel such that the volume of the resuspension across at least 10 batches has a mean intra-batch coefficient of variance (mean of intra-batch CV) of less than 30%, 25%, 20%, 15%, 12%, 10%, 8%, 6%, 5%, 4%, 3%, 2%, or 1%, or can be in the range of 0.1-30%, 0.1-20%, 0.1-15%, 0.1-12%, 0.1-10%, 0.1-8%, 0.1-6%, 0.1-4%, 0.1-2%, 1-10%, 2-10%, 3-10%, 4-10%, 5-10%, or 6-10%. In some embodiments, resuspending the pellet in each vessel leads to a resuspension in each vessel such that the volume of the resuspension across at least 20 batches has a mean intra-batch coefficient of variance (mean of intra-batch CV) of less than 30%, 25%, 20%, 15%, 12%, 10%, 8%, 6%, 5%, 4%, 3%, 2%, or 1%, or can be in the range of 0.1-30%, 0.1-20%, 0.1-15%, 0.1-12%, 0.1-10%, 0.1-8%, 0.1-6%, 0.1-4%, 0.1-2%, 1-10%, 2-10%, 3-10%, 4-10%, 5-10%, or 6-10%. In some embodiments, resuspending the pellet in each vessel leads to a resuspension in each vessel such that the volume of the resuspension across vessels within a batch has a coefficient of variance of less than 30%, 25%, 20%, 15%, 12%, 10%, 8%, 6%, 5%, 4%, 3%, 2%, or 1%, or can be in the range of 0.1-30%, 0.1-20%, 0.1-15%, 0.1-12%, 0.1-10%, 0.1-8%, 0.1-6%, 0.1-4%, 0.1-2%, 1-10%, 2-10%, 3-10%, 4-10%, 5-10%, or 6-10%. In some embodiments, resuspending the pellet in each vessel leads to a resuspension in each vessel such that the volume of the resuspension in a vessel across at least 10 batches or within a batch varies no more than 30%, 25%, 20%, 1%, %12%, 10% 8%, 6%, 5%, 4%, 3%, 2%, or 1%, or can be in the range of 0.1-30%, 0.1-20%, 0.1-15%, 0.1-12%, 0.1-10%, 0.1-8%, 0.1-6%, 0.1-4%, 0.1-2%, 1-10%, 2-10%, 3-10%, 4-10%, 5-10%, or 6-10%. In some embodiments, the volume of the pooled resuspension having DMSO in a cryo-vessel across at least 10 batches has a mean intra-batch coefficient of variance of less than 30%, 25%, 20%, 15%, 12%, 10%, 8%, 6%, 5%, 4%, 3%, 2%, or 1%, or can be in the range of 0.1-30%, 0.1-20%, 0.1-15%, 0.1-12%, 0.1-10%, 0.1-8%, 0.1-6%, 0.1-4%, 0.1-2%, 1-10%, 2-10%, 3-10%, 4-10%, 5-10%, or 6-10%. In some embodiments, the volume of the pooled resuspension having DMSO in a cryo-vessel across at least 20 batches has a mean intra-batch coefficient of variance of less than 30%, 25%, 20%, 15%, 12%, 10%, 8%, 6%, 5%, 4%, 3%, 2%, or 1%, or can be in the range of 0.1-30%, 0.1-20%, 0.1-15%, 0.1-12%, 0.1-10%, 0.1-8%, 0.1-6%, 0.1-4%, 0.1-2%, 1-10%, 2-10%, 3-10%, 4- 10%, 5-10%, or 6-10%. In some embodiments, the volume of the pooled resuspension in a cryo-vessel having DMSO within a batch has a coefficient of variance of less than 30%, 25%, 20%, 15%, 12%, 10%, 8%, 6%, 5%, 4%, 3%, 2%, or 1%, or can be in the range of 0.1-30%, 0.1-20%, 0.1-15%, 0.1-12%, 0.1-10%, 0.1-8%, 0.1-6%, 0.1-4%, 0.1-2%, 1-10%, 2-10%, 3-10%, 4-10%, 5-10%, or 6-10%. In some embodiments, the volume of the pooled resuspension having DMSO in a cryo-vessel across at least 10 batches or within a batch varies no more than 30%, 25%, 20%, 15%, 12%, 10%, 8%, 6%, 5%, 4%, 3%, 2%, or 1%, or can be in the range of 0.1-30%, 0.1-20%, 0.1-15%, 0.1-12%, 0.1-10%, 0.1-8%, 0.1-6%, 0.1-4%, 0.1-2%, 1-10%, 2-10%, 3-10%, 4-10%, 5-10%, or 6-10%. In some embodiments, in step c) the resuspending is performed using a buffer composition comprising a buffering agent, a base, and one or more of a saccharide. In some embodiments, the saccharide comprises one or more of a monosaccharide, disaccharide, polysaccharide, or a combination thereof. In some embodiments, the saccharide comprises trehalose, and polysucrose. In some embodiments, the buffer further comprises a salt, and an organic solvent. In some embodiments, the vessel in step a) is an apheresis platelet unit (APU) bag. In some embodiments, the APU bag has a volume of at least 600, 700, or 800 mL. In some embodiments, the APU bag has a maximum volume of at least 1000 or at least 1500 mL. In some embodiments, the APU bag has a maximum volume of 1600 mL. In some embodiments, the pooled resuspension vessel is one or more than one APU bag.

In some embodiments of any of the aspects or embodiments herein that include a collection comprising cryopreserved or frozen platelets, or a process for preparing batches thereof, the cryopreserved or frozen platelets can have a mean CD62% positivity across at least 5, 10, 15, or 20 batches, or 5-30, 10-30, 15-30, 20-30, or 25-30 batches, or for example 27 batches in the range of 54% to 75%, 54.5% to 75%, 55% to 75%, 57% to 65%, or 57% to 60%. In some embodiments, the cryopreserved or frozen platelets can have a mean CD62% positivity across at least 3, 4, 5, 10, 15, 20, 50, 75, or 100 batches, in the range of 54% to 95%, 54% to 90%, 54% to 85%, 54% to 80%, 54% to 75%, 54% to 70%, 54% to 65%, 54% to 60%, 54% to 94%, 54.5% to 90%, 55% to 86%, 55.5% to 82%, 56% to 78%, 56.5% to 74%, 57% to 70%, 57% to 69%, 57% to 68%, 57% to 67%, 57% to 66%, 57% to 65%, 57% to 64%, 57% to 63%, 57% to 62%, 57% to 61%, 57% to 60%, or 57% to 59%. In some embodiments, at least 90%, or 95% of cryo-vessels, or each cryo-vessel in the collection herein have cryopreserved platelets or frozen activated platelets having a CD62% positivity in the range of 56% to 60%, 56.5% to 59.8%, or 57.1% to 59.3%. In some embodiments, each cryo-vessel in the collection herein have cryopreserved platelets or frozen activated platelets having a CD62% positivity of at least 45%, 48%, or 50%.

In some embodiments, the phosphatidylserine positivity, when measured using lactadherin binding is between 40% to 95%, 50% to 95%, 60% to 95%, 70% to 95%, 78.5% to 95%, or 80% to 95%. In some embodiments, the cryopreserved or frozen platelets can have a mean phosphatidylserine % positivity, when tested using lactadherin binding across at least 5, 10, 15, or 20 batches, or 5-30, 10-30, 15-30, 20-30, or 25-30 batches, or for example 27 batches in the range of 78% to 95%, 78% to 90%, or 78% to 85%, 78.5% to 85%, or 79% to 80.5%. In some embodiments, the cryopreserved or frozen platelets have a mean phosphatidylserine % positivity, when tested using lactadherin binding across at least 3, 4, 5, 10, 15, 20, 50, 75, or 100 batches, in the range of 78% to 95%, 78% to 94%, 78% to 93%, 78% to 92%, 78% to 91%, 78% to 90%, 78% to 89%, 78% to 88%, 78% to 87%, 78% to 86%, 78% to 85%, 78% to 84%, 78% to 83%, or 78% to 82%. In some embodiments, at least 90%, or 95% of cryo-vessels, or each cryo-vessel in the collection herein have cryopreserved platelets or frozen activated platelets having a phosphatidylserine positivity, when measured using lactadherin binding in the range of 77% to 81.5%, 77.3% to 81.05%, 77.9% to 80.45%, or 78% to 80.5%. In some embodiments, each cryo-vessel in the collection herein have cryopreserved platelets or frozen activated platelets having a phosphatidylserine positivity, when measured using lactadherin binding of at least 55%, 58%, 60%, or 62%.

In some embodiments, the composition comprising cryopreserved platelets or the frozen platelets, upon thawing and storing at a temperature, for example, in the range of 20° C. to 40° C., or 20° C. to 28° C. for at least 6, 8, 12, or 24 hours, or 6 to 24 hours exhibit phosphatidylserine positivity, when measured using lactadherin binding in the range of 60% to 99%, 65% to 99%, 70% to 99%, or 75% to 95%. In some embodiments, the composition comprising cryopreserved platelets or the frozen platelets, upon thawing and storing at a temperature in the range of 20° C. to 28° C. for 8 hours exhibit phosphatidylserine positivity, when measured using lactadherin binding in the range of 80% to 99%, 80% to 98%, or 80% to 95%. In some embodiments, the composition in the cryo-vessels, upon thawing and storing at a temperature in the range of 18° C. to 28° C., 18° C. to 40° C., 20° C. to 40° C., or 20° C. to 30° C. for 8 hours have the property of exhibiting a mean phosphatidylserine positivity, when measured using lactadherin binding, across at least 3 batches in the range of 70% to 99%, 75% to 97%, 80% to 99%, 80% to 95%, or 82% to 95%.

In some embodiments, the composition comprising cryopreserved platelets across at least 5, 10, 15, or 20 batches, or 5-30, 10-30, 15-30, 20-30, or 25-30 batches, or for example 27 batches, upon thawing comprises a mean concentration of platelets in the range of 4.0×10⁶ to 6.0×10⁶, 4×10⁶ to 5.8×10⁶, 4×10⁶ to 5.5×10⁶, 4.2×10⁶ to 5.0×10⁶, 4.2×10⁶ to 4.8×10⁶ platelets/μl, 4.2×10⁶ to 5.0×10⁶, or 4.40×10⁶ to 4.70×10⁶. In some embodiments, the composition comprising cryopreserved platelets across least 3, 4, 5, 10, 15, 20, 50, 75, or 100 batches, upon thawing comprises a mean concentration of platelets in the range of 4.0×10⁶ to 5.5×10⁶, 4.1×10⁶ to 5.4×10⁶, 4.2×10⁶ to 5.3×10⁶, 4.3×10⁶ to 5.2×10⁶, 4.4×10⁶ to 5.1×10⁶, 4.4×10⁶ to 5.0×10⁶, 4.4×10⁶ to 4.9×10⁶, or 4.4×10⁶ to 4.8×10⁶ platelets/μl.

In some embodiments, the composition comprising cryopreserved platelets, upon thawing have a mean thrombin generation ability, in terms of peak thrombin across at least 3, 4, 5, 10, 15, 20, 50, 75, or 100 batches, or 5-30, 10-30, 15-30, 20-30, or 25-30 batches, or for example 27 batches, in the range of 1.5 to 2.5, 1.5 to 2.3, 1.5 to 2.1, 1.5 to 2.0, 1.5 to 1.9, 1.6 to 1.9, 1.5 to 1.8, or 1.5 to 1.7, 1.425 to 1.75, or 1.475 to 1.65 IU/10⁶ platelet particles, for example, when measured using Thrombinoscope instrument, or Fluoroskan Ascent instrument. In some embodiments, when measured using CLARIOstar^{plus} instrument, the thrombin generation ability can be in the range of 10-20, 12-20, or 14-20 IU/10⁶ platelet particles or NIH units/10⁶ platelet particles. In some embodiments, the composition in the cryo-vessels, upon thawing and storing at a temperature in the range of 18° C. to 28° C., 18° C. to 40° C., or 20° C. to 50° C. for 8 hours have the property of exhibiting a mean thrombin generation across at least 3 batches in the range of 1.50 to 1.90, 1.60 to 1.85, 1.60 to 1.80, or 1.50 to 1.80 IU/10⁶ particles. In some embodiments, the composition comprising cryopreserved platelets, upon thawing, have a mean thrombin generation ability across at least 3, 4, 5, 6, 10, 15, 20, 25, 50, 75, 100 batches, or 5-30, 10-30, 15-30, 20-30, or 25-30 batches, or for example 27 batches, in the range of 218 to 276, 220 to 274, 222 to 272, 224 to 270, 226 to 268, 228 to 266, 230 to 264, 232 to 262, 234 to 260, 236 to 258, 238 to 256, 240 to 254, 240 to 252, 240 to 250, 240 to 248, 230 to 260, 235 to 255, 240 to 250, 220 to 275, or 241.5 to 247.5 nM thrombin peak height. In some embodiments, the peak thrombin of the composition comprising cryopreserved platelets, upon thawing, is in the range of 10-30, 10-25, 10-20, 12-20, or 15-25 NIH units/10⁶ platelets/cells/particles. In some embodiments, the peak thrombin of the composition comprising cryopreserved platelets, upon thawing, is 1.5, 2.0, 2.5, 2.75, or 3-fold higher than that of apheresis platelet units or liquid stored platelets. In some embodiments, the composition comprising cryopreserved platelets, upon thawing, have a coefficient of variance of the mean thrombin generation ability across at least 10, 20, 30, 40, 50, 60, 70, or 80 batches less than 40%, 30%, 25%, 20%, or in the ranges of 1-40%, 10-30%, 15-25%, 17-22%, or 18-20%. In some embodiments of any of the aspects or embodiments that include a collection of cryo-vessels comprising cryopreserved platelets, a process for preparing a batch of cryopreserved platelets, a process for preparing a cryopreserved platelet composition, the composition comprising cryopreserved platelets, upon thawing have a coefficient of variance of the mean amount of total platelets in the cryo-vessel across at least 10, 20, 40, 60, or 80 batches less than 12%, 11%, 10%, or 9%, or in the ranges of 1-12%, 1-11%, 1-10%, 1-9%, 1-8%, 2-9%, 3-10%, or 3-9%. In some embodiments, the composition comprising cryopreserved platelets, upon thawing have a coefficient of variance of the mean platelet concentration in the cryo-vessel across at least 10, 20, 40, 60, or 80 batches less than 14%, 13%, 12%, 11%, 10%, or 9%, or in the ranges of 1-12%, 1-11%, 1-10%, 1-9%, 1-8%, 2-9%, 3-10%, or 3-9%. In some embodiments, the composition comprising cryopreserved platelets, upon thawing have a coefficient of variance of the mean CD61-positive concentration in the cryo-vessel across at least 10, 20, 40, 60, or 80 batches less than 35%, 30%, 28%, 26%, or 25%, or in the ranges of 1-35%, 1-30%, 1-28%, 1-25%, 5-35%, 5-30%, 5-28%, or 5-25%. In some embodiments, the composition comprising cryopreserved platelets, upon thawing have a coefficient of variance of the mean phosphatidylserine positivity when measured using lactadherin binding in the cryo-vessel across at least 10, 20, 40, 60, or 80 batches less than 7%, 6.75%, 6.5%, or 6.25%, or in the ranges of 1-6.5%, 1-6.25%, 1-6%, 1-5.5%, 2-6.5%, 2-6.25%, 2-6%, or 2-5.5%. In some embodiments, the composition comprising cryopreserved platelets, upon thawing have a mean CD61-positive concentration in the cryo-vessel across at least 10, 20, 40, 60, or 80 batches of less than 9.5×10⁶/μl, 9.0×10⁶/μl, 8.5×10⁶/μl, 8.0×10⁶/μl, 7.0×10⁶/μl, 6.0×10⁶/μl, or 5.5×10⁶/μl, and have a coefficient of variance of the mean CD61-positive concentration in the cryo-vessel across at least 10, 20, 40, 60, or 80 batches less than 35%, 30%, 28%, 26%, or 25%. In some embodiments, the composition comprising cryopreserved platelets, upon thawing have a mean CD61-positive concentration in the cryo-vessel across 20 batches of less than 9.5×10⁶/μl, 9.0×10⁶/μl, 8.5×10⁶/μl, 8.0×10⁶/μl, 7.0×10⁶/μl, 6.0×10⁶/μl, or 5.5×10⁶/μl, and have a coefficient of variance of the mean CD61-positive concentration in the cryo-vessel across 20 batches less than 30%, 28%, 26%, or 25%. In some embodiments, the composition comprising cryopreserved platelets, upon thawing across at least 20, 40, 50, 50, or 80 batches have a mean pH in the range of 6.2 to 6.8, and wherein the % coefficient of variance of the mean pH is less than 3.75%, 3.50%, 3.25%, 3%, 2.75%, or 2.5%.

In some embodiments of any of the aspects or embodiments that include a collection of cryo-vessels comprising cryopreserved platelets, a process for preparing a batch of cryopreserved platelets, a process for preparing a cryopreserved platelet composition, or a composition comprising frozen platelets, the collection comprises a plurality of at least 3, 4, 5, 10, 15, 20, 50, 75, or 100 batches of the cryo-vessels. In some embodiments, the biomolecule profile indicative of more than 1 platelet donor, is two amino acid sequences of a first protein from a first gene that are significantly different in frequency within the cryopreserved platelets than 50%, or the presence of more than two amino acid sequences of the first protein. In some embodiments, the set of biomolecule profiles of one batch is different from the set of biomolecule profiles of another batch. In some embodiments, the coefficient of variance of a mean DMSO concentration in the cryopreserved platelets across the batches is less than 15%, 10%, 8%, 6%, 5%, 4%, in illustrative embodiments, less than 3%, 2.5%, 2%, 1.5%, 1%, 0.75%, 0.5%, 0.25%, or 0.1%. For example, the coefficient of variance of a mean DMSO concentration across the batches can be in the range of 15-0.01%, 10-0.01%, 8-0.01%, 6-0.01%, 5-0.01%, 4-0.01%, 3-0.01%, 2-0.01%, or 1-0.01%. In some embodiments, the concentration of DMSO in the cryopreserved platelets of a first cryo-vessel is within 20%, 15%, 12%, 1%, 80%, 6%, in illustrative embodiments within 5%, 3%, 1% of the concentration of DMSO in the cryopreserved platelets of a second cryo-vessel. In some embodiments, the biomolecule profile indicative of more than 1 platelet donor, is the presence of two amino acid sequences of a first protein from a first gene that are significantly different in frequency within the cryopreserved platelets than 50%, or the presence of more than two amino acid sequences of the first protein. In some embodiments, the collection comprises a plurality of at least 2, 3, 4, 5 or more batches of the cryo-vessels wherein the cryo-vessels of each batch have the same biomolecular profile, and the cryo-vessels of each batch have a different biomolecular profile from the cryo-vessels of any other batch in the collection. In some embodiments, the cryopreserved platelets in a first cryo-vessel when thawed has a volume that varies by no more than 30%, 25%, 20%, 15%, in illustrative embodiments, 10%, 8%, 6%, 5%, 3%, or 1% of the volume in a second cryo-vessel. In some embodiments, the first and the second cryo-vessels are from a different batch of the collection of the cryo-vessels. In some embodiments, the mean DMSO concentration in the cryopreserved platelets across at least 2, 3, 4, 5, 6, 7, 8, 9, 10, 15, 20, 25, 50, 75, or 100 batches has a coefficient of variance of less than 10%, 8%, 5%, 3%, 2%, in illustrative embodiments, less than 1%, 0.9%, 0.8%, 0.75%, 0.7%, 0.6%, 0.5%, 0.4%, 0.3%, 0.2%, or 0.1%. In some embodiments, the mean DMSO concentration in the cryopreserved platelets across at least 2, 3, 4, 5, 6, 7, 8, 9, 10, 15, 20, 25, 50, 75, or 100 batches has a coefficient of variance in the range of 0.01-5%, 0.01-4%, 0.01-3%, 0.01-2%, 0.01-1%, 0.05-1%, 0.05-0.75%, 0.05-0.50%, or 0.05-0.4%. In some embodiments, the concentration of DMSO in the cryopreserved platelets in a cryo-vessel within a batch or across at least 5 batches varies by no more than 5%, 4%, 3%, 2%, 1%, in illustrative embodiments, no more than 0.5%, 0.4%, 0.3%, 0.2%, 0.1%, or 0.05%. In some embodiments, the cryo-vessels in the batch or the collection comprise the cryopreserved platelets with DMSO in the range of 0.001-10%, 0.01-5.5%, 1-6%, 5-7%, 0.001-8%, 1-10%, 1-8%, 1-7%, 2-10%, 2-8%, 2-7%, 3-10%, 3-8%, 3-7%, 4-10%, 4-9%, 4-8%, or 4-7%.

In some embodiments of any of the aspects or embodiments herein that include a process for preparing cryopreserved platelets or a cryopreserved platelet composition, for preparing a batch of cryopreserved platelets, a collection of cryo-vessels comprising cryopreserved platelets, or a composition comprising frozen platelets or cryopreserved platelets, the cryopreserved platelets are stable for at least 1 month, 2, 3, 4, 6, 8, 10 months, 1 year, 2, 3, 4, 5, 6, 7, 8, 9, or 10 years at a temperature of −20° C., or −20° C. +/−5° C. for example, the cryopreserved platelet can be stable for 1 month to 5 years, 1 month to 12 months, 3 months to 5 years, 6 months to 5 years, 1-5 years, or 2-5 years. In some embodiments, the concentration of platelets in the cryopreserved platelets in a cryo-vessel within a batch or across at least 5 batches varies no more than 20%, 15%, in illustrative embodiments, no more than 10%, 8%, 5%, 3%, 2%, or 1%. In some embodiments, the concentration of platelets in the cryopreserved platelets in a cryo-vessel across at least 5 batches has a mean intra-batch coefficient of variance of less than 20%, 15%, in illustrative embodiments, less than 10%, 8%, 6%, 5%, 3%, or 1%. In some embodiments, the concentration of platelets in the cryopreserved platelets in a cryo-vessel within a batch has a coefficient of variance of less than 20%, 15%, in illustrative embodiments, less than 10%, 8%, 6%, 5%, 3%, or 1%. In some embodiments, the total number of platelets in the cryopreserved platelets in a cryo-vessel within a batch or across at least 5 batches vanes no more than 20%, 15%, 10%, in illustrative embodiments, less than 8%, 6%, 5%, 3%, or 1%. In some embodiments, the total number of platelets in the cryopreserved platelets in a cryo-vessel across at least 5 batches has a mean intra-batch coefficient of variance of less than 20%, 15%, 10%, in illustrative embodiments, less than 8%, 6%, 5%, 3%, or 1%. In some embodiments, the total number of platelets in the cryopreserved platelets in a cryo-vessel within a batch has a coefficient of variance of less than 25%, 20%, in illustrative embodiments, less than 15%, 10%, 8%, 6%%, 4%, 3%, 2%, or 1%. In some embodiments, the total number of platelets in the cryopreserved platelets in a cryo-vessel within a batch has a coefficient of variance in the range of 0.01-25%, 0.01-20%, 0.01-10%, 0.01-8%, 0.01-6%, 0.01-5%, 0.01-3%, 0.01-2%, 0.5-10%, 0.5-8%, 0.5-5%, 0.5-3%, 1-10%, 1-8%, 1-6%, or 1-5%. In some embodiments, the cryopreserved platelets comprise plasma in the range of 50-95%, 50-90%, 50-85%, 50-80%, 50-75%, 60-95%, 65-95%, 70-95%, or 70-85% (v/v). In some embodiments, the cryopreserved platelets comprise DMSO in the range of 1-10%, 1-8%, 2-10%, 2-8%, 3-10%, 3-9%, 3-8%, 4-10%, or 4-8% (v/v). In some embodiments, the cryopreserved platelets comprise sodium chloride in the range of 5-30%, 5-25%, 5-20%, 5-15%, or 7-15% (w/v). In some embodiments, the cryopreserved platelets comprise a buffering agent, a salt, one or more saccharide, and at least one organic solvent. In some embodiments, the saccharide comprises trehalose, and polysucrose. In some embodiments, the cryo-vessel is a cryo-bag.

In some embodiments of any of the aspects or embodiments herein that include a process for preparing cryopreserved platelets or cryopreserved platelet composition that include a transition in freezing and storing temperature, a process for preparing a batch of cryopreserved platelets, or a process for preparing cryopreserved platelets or cryopreserved platelet composition, freezing can comprise freezing a population of platelets in a cryopreservation medium, or freezing a pooled resuspension having a cryoprotectant herein at a temperature of equal to or less than −50° C., −55° C., −60° C., or −65° C., to form an initial frozen platelet composition, and storing can comprise storing the initial frozen platelet composition at a temperature equal to or higher than −45° C., −40° C., −35° C., −30° C., −25° C., or −20° C. for at least 2, 4, 6, 7, 10, 15, 20, or 25 days, 1 month, 2, 4, 6, 8, 10, or 12 months, 1 year, 2, 4, 6, 8, or 10 years, to form the cryopreserved platelet composition. In illustrative embodiments, storing comprises storing the initial frozen platelet composition in a freezer set at a temperature of −20° C. +/−2° C. In some embodiments, the freezing comprises subjecting the population of platelets in the cryopreservation medium or a pooled resuspension having a cryoprotectant as disclosed herein at the temperature for at least 30 minutes, 1 hour, 2 hours, 3 hours, 4 hours, 8 hours, 12 hours, 1 day, 2 days, 7 days, 1 week, 2 weeks, 1 month, 2 months, 3 months, or 6 months to form the initial frozen platelet composition. For example, freezing can be done for a time period of 24 hours to 6 months, 24 hours to 5 months, 24 hours to 4 months, 1 day to 6 months, 1 day to 5 months, 1 day to 4 months. In some embodiments, the process further comprises thawing the cryopreserved platelets to form a liquid platelet composition, and administering an effective amount of the liquid platelet composition to a subject in need thereof. In some embodiments, the storing of an initial frozen platelet composition can be done for a time period in the range of 1 month to 10 years, 1 month to 8 years, 1 month to 7 years, 1 month to 5 years, 1 month to 3 years, or 1 month to 1 year. In some embodiments, the cryopreservation medium comprises dimethyl sulfoxide (DMSO) in a concentration in the range of 5% to 8%, for example, DMSO can be 6%+/−1%, +/0.8%, +/−0.6%, +/−0.5%, +/−0.4%, +/−0.3%, +/−0.2%. +/−0.1%. In some embodiments, the cryopreservation medium comprises DMSO in a concentration range of 0.5% to 8%, 2% to 8%, 3% to 8%, or 5% to 8%. In some embodiments, the initial frozen platelet composition is stored at the temperature of in the range of −10° C. to −30° C. for at least 1 month, 2, 3, 4, 5, or 6 months to form cryopreserved platelets, and the cryopreserved platelets upon thawing exhibits one or more of the following: i) retains the ability to reduce bleeding in a subject in need thereof, ii) exhibit a platelet count recovery of at least 65%, 70%, or 75%; iii) exhibits a pH of equal to or more than 6.2; iv) exhibit an aggregate-free swirling upon a visual inspection; v) exhibits a platelet count of equal to or more than 1.7×10¹¹ platelets/bag or platelets/cryo-vessel, or per 20-35 ml. In some embodiments, the mean DMSO concentration in the cryopreserved platelets across at least 2, 3, 4, 5, 6, 7, 8, 9, 10, 15, 20, 25, 50, 75, or 100 batches has a coefficient of variance of less than 10%, 8%, 5%, 3%, 2%, in illustrative embodiments, less than 1%, 0.9%, 0.8%, 0.75%, 0.7%, 0.6%, 0.5%, 0.4%, 0.3%, 0.2%, or 0.1%. In some embodiments, the mean DMSO concentration in the cryopreserved platelets across at least 2, 3, 4, 5, 6, 7, 8, 9, 10, 15, 20, 25, 50, 75, or 100 batches has a coefficient of variance in the range of 0.01-5%, 0.01-4%, 0.01-3%, 0.01-2%, 0.01-1%, 0.05-1%, 0.05-0.75%, 0.05-0.50%, or 0.05-0.4%. In some embodiments, the concentration of DMSO in the cryopreserved platelets in a cryo-vessel within a batch or across at least 5 batches varies by no more than 5%, 4%, 3%, 2%, 1%, in illustrative embodiments, no more than 0.5%, 0.4%, 0.3%, 0.2%, 0.1%, or 0.05%. In some embodiments, the cryo-vessels in the batch or the collection comprise the cryopreserved platelets with DMSO in the range of 0.001-10%, 0.01-5.5%, 1-6%, 5-7%, 0.001-8%, 1-10%, 1-8%, 1-7%, 2-10%, 2-8%, 2-7%, 3-10%, 3-8%, 3-7%, 4-10%, 4-9%, 4-8%, or 4-7%. In some embodiments, the cryopreserved platelets upon thawing have a diameter in the range of 0.5-5 μm, 1-4 μm, 1-3 μm, or 0.5-2.5 μm, and wherein the composition upon thawing has a CD61-positive-microparticle content in the range of 10-30%, 10-35%, 10-40%, 10-45%, or 10-50%. In some embodiments, a therapeutically effective amount of cryopreserved platelets or cryopreserved platelet derivatives, frozen platelets or frozen platelet derivatives upon storing at a temperature equal to or higher than −45° C., −40° C., −35° C., −30° C., −25° C., or −20° C. for at least 2, 4, 6, 7, 10, 15, 20, or 25 days, 1 month, 2, 4, 6, 8, 10, or 12 months, 1 year, 2, 4, 6, 8, or 10 years, upon thawing retains the ability to reduce bleeding in a subject in need thereof. In some embodiments, a therapeutically effective amount is an amount that is capable of reducing bleeding in a subject as compared to the bleeding before administering the therapeutically effective amount of thawed cryopreserved platelets or cryopreserved platelet derivatives, frozen platelets or frozen platelet derivatives. Accordingly, in some embodiments, a therapeutically effective amount can be at least 0.25 unit, 0.5 unit, 0.75 unit, 1 unit, 2, or 3 units of thawed cryopreserved or frozen platelets. For example, a therapeutically effective amount can be in the range of 0.25 unit to 5 units, in illustrative embodiments, 0.5 unit in a lower end and 3 units, 2 units, or 1 unit in a higher end, or 1 unit in a lower end and 4, 3, or 2 units in a higher end. In some embodiments, 1 unit corresponds to at least 1×10¹¹, 1.5×10¹¹, 2×10¹¹, or 2.5×10¹¹ platelets in a volume of at least about 20 ml, 25 ml, 30 ml, 40 ml, 45 ml, 50 ml, 55 ml, or 60 ml. In some embodiments, 1 unit corresponds to platelets in a range of 1×10¹¹ to 5×10¹¹, 1 ×10¹¹ to 4×10¹¹, 1×10¹¹ to 4×10¹¹, or 2×10¹¹ to 3×10¹¹ platelets in at least 20 ml, 25 ml, 30 ml, 40 or 50 ml. In illustrative embodiments, 1 unit corresponds to 2.5×10¹¹+/−4.2×10¹¹ platelets in 50+/−4 ml. The unit can refer to a unit of frozen platelets, frozen platelet derivatives, cryopreserved platelets or cryopreserved platelet derivatives, as will be understood depending on the context.

In some embodiments of any of the aspects or embodiments herein that include a composition comprising frozen platelets or cryopreserved platelets in a cryopreservation medium in a frozen state as disclosed herein, a collection as disclosed herein, or a process for preparing a cryopreserved platelet composition or for preparing a batch of cryopreserved platelets, the composition upon storage for at least 1 day, 2, 4, 6, 7, 10, 15, 20, 25 days, 1 month, 2, 4, 6, 8, 10, or 12 months, 1 year, 2, 4, 5, 6, 8, or 10 years, in illustrative embodiments, at a temperature in a range of −5° C. to −30° C., or −20° C. +/−5° C. upon thawing exhibits a platelet count of at least 1.0×10¹¹/20-35 ml of the composition. For example, exhibits a platelet count of at least 1.0×10¹¹/20 ml of the composition, 1.0×10¹¹/25 ml of the composition, 1.0×10¹¹/30 ml of the composition, 1.0×10¹¹/35 ml of the composition. In some embodiments, a composition herein upon thawing exhibits a platelet count of at least 1.1×10¹¹/20-35 ml of the composition, 1.2×10¹¹/20-35 ml of the composition, 1.3×10¹¹/20-35 ml of the composition, 1.4×10¹¹/20-35 ml of the composition, 1.5 ×10¹¹/20-35 ml of the composition, 1.6×10¹¹/20-35 ml of the composition, or 1.7×10¹¹/20-35 ml of the composition. In some embodiments, the composition, upon thawing and storing at a temperature in the range of 20° C. to 40° C., or 20° C. to 28° C., for at least 6, 8, 10, 12, or 24 hours, or 6 to 24 hours, exhibits a total platelet count in the range of 1.0×10¹¹ to 4.0×10¹¹, 1.3×10¹¹ to 4.0×10¹¹, 1.5×10¹¹ to 4.0×10¹¹, 1.7×10¹¹ to 4.0×10¹¹, 1.75×10¹¹ to 4.0×10¹¹, or 1.8×10¹¹ to 3.5×10¹¹. In some embodiments, the cryopreserved platelets herein are cryopreserved platelet derivatives. In some embodiments, the frozen platelets herein are frozen platelet derivatives. Typically, a composition as disclosed herein upon thawing is in a liquid state without requiring the addition of a liquid to achieve such a liquid state. In some embodiments, cryopreserved platelets, or frozen platelets herein do not comprise freeze-dried platelet derivatives or lyophilized platelet derivatives. In some embodiments, a composition upon thawing yields a single peak that corresponds to a compromised membrane peak in a membrane integrity assay. In some embodiments, the membrane integrity assay comprises incubating the composition with calcein acetoxymethyl (AM) to form a treated composition and analyzing the treated composition using flow cytometry. In some embodiments, the membrane integrity assay comprises incubating the composition with calcein acetoxymethyl (AM) for 20 minutes before performing the flow cytometry, and wherein the flow cytometry comprises detecting fluorescence produced by metabolized calcein AM and retained by particles in the treated composition. In some embodiments, the in vitro thrombin generation assay comprises generating thrombin in the presence of tissue factor, and phospholipids. In some embodiments, the in vitro thrombin generation assay consists of generating thrombin in the presence of tissue factor and phospholipids. In some embodiments, the frozen platelets or cryopreserved platelets herein upon thawing have a diameter in the range of 0.5-5 μm, 1-4 μm, 1-3 μm, or 0.5-2.5 μm. In some embodiments, a composition comprising frozen platelets herein upon thawing has a CD61-positive-microparticle content in the range of 5-50%, 5-45%, 5-40%, 5-35%, 5-30%, 10-30%, 10-40%, 10-50%, or 10-60%. In some embodiments, a composition as disclosed herein comprise CD61-positive-microparticles having a diameter less than 1 μm, 0.8 μm or 0.5 μm. In some embodiments, a composition herein upon thawing has a pH equal to or more than 6.0, 6.2, or 6.5. In some embodiments, a composition herein upon thawing has a pH in the range of 6.2 to 8.0. In some embodiments, a composition herein have a dispersed property such that after thawing the composition, the frozen platelets therein form no visible aggregates upon a visual inspection. In some embodiments, the composition comprises a dispersed suspension property such that aggregation of platelets in the composition is not observed by visual inspection of the composition after thawing. In some embodiments, the composition has a swirling property, such that swirling of the composition can be observed by visual inspection of the composition after thawing. In some embodiments, a composition comprises a cryopreservation medium comprises dimethyl sulfoxide (DMSO) at a concentration of 0.5% to 10%, 1% to 8%, or 3% to 8%. In some embodiments, in a composition herein, no more than 50% of the frozen platelet derivates upon thawing are positive for CD62, or Annexin V. For example, in a composition herein, particles positive for CD62 are less than 90%, 85%, 80%, 70%, 65%, 60%, or 50%. For example, in a composition herein, particles positive for Annexin V are less than 90%, 85%, 80%, 70%, 65%, 60%, or 50%. For example, in a composition herein, particles positive for Annexin V are in a range of 1-50%, 1-40%, 1-30%, 1-20%, 1-10%, 5-50%, 5-40%, 5-30%, or 5-20%. For example, in a composition herein, particles positive for CD62 are in a range of 1-50%, 1-40%, 1-30%, 1-20%, 1-10%, 5-50%, 5-40%, 5-30%, or 5-20%. In some embodiments, frozen platelets or cryopreserved platelets in a composition herein are less activated as compared to freeze-dried platelet derivatives or lyophilized platelet derivatives. In some embodiments, frozen platelets or cryopreserved platelets in a composition are not lyophilized platelet derivatives, or not freeze-dried platelet derivatives. In some embodiments, a composition herein exhibits a platelet count of at least 1.0×10¹¹/20 ml of the composition. In some embodiments, a composition upon storage for at least 1 day, 2, 4, 6, 7, 10, 15, 20, 25 days, 1 month, 2, 4, 6, 8, 10, or 12 months, 1 year, 2, 4, 5, 6, 8, or 10 years, in illustrative embodiments, at a temperature in a range of −5° C. to −30° C., upon thawing exhibits a CD61-positive-microparticle content of less than 50% of the CD61 positive particles in the composition. In some embodiments, a composition upon storage for at least 1 day, 2, 4, 6, 7, 10, 15, 20, 25 days, 1 month, 2, 4, 6, 8, 10, or 12 months, 1 year, 2, 4, 5, 6, 8, or 10 years, in illustrative embodiments, at a temperature in a range of −5° C. to −30° C., upon thawing yields a single peak that corresponds to a compromised membrane peak in a membrane integrity assay. In some embodiments, a composition upon storage for at least 1 day, 2, 4, 6, 7, 10, 15, 20, 25 days, 1 month, 2, 4, 6, 8, 10, or 12 months, 1 year, 2, 4, 5, 6, 8, or 10 years, in illustrative embodiments, at a temperature in a range of −5° C. to −30° C., upon thawing generates thrombin in an in vitro thrombin generation assay. In illustrative embodiments, a composition herein is capable of yielding one or more, two or more, three or more, or all of the properties after storing for 12 months. In some embodiments, a composition comprising frozen platelets in a cryopreservation medium in a frozen state, after storage for at least 1 month, 2, 3, 4, 5, 6, 8, 10, or 12 months, in an illustrative embodiments at a temperature in a range of −10° C. to −30° C., or −20° C. +/−5° C., upon thawing exhibits a liquid state without the addition of a liquid, exhibits a platelet count of at least 1.0×10¹¹/35 ml of the composition, exhibits a CD61-positive-microparticle content of less than 50% of the CD61 positive particles in the composition, and generates thrombin in an in vitro thrombin generation assay.

In some embodiments of any of the aspects or embodiments herein that comprise a composition, collection, process or method, the population of platelet particles are capable of further activation upon stimulation, in some embodiments, in vitro stimulation. In some embodiments, the stimulation is performed with one or more platelet agonists, as non-limiting examples, TRAP-6, ADP, epinephrine (Epi), and/or any agonist disclosed herein. In some embodiments, the composition collection, process or method comprises, the activation is detected by detecting an activated conformation of GPIIb/IIIa, or in some embodiments, detecting any platelet activation cell surface marker disclosed herein. In some embodiments, detecting of the activated conformation of GPIIb/IIIa is performed using the PAC-1 monoclonal antibody, or using any antibody disclosed herein.

In some embodiments of any of the aspects or embodiments herein that comprise a composition, a collection, process or method, the population of platelet particles has the property, upon thawing and when analyzed using flow cytometry, of forming two detectable sub-populations of platelet particles, a more activated sub-population, and a less activated sub-population upon
gating a population of platelet-sized particles using a fluorescently-tagged protein that recognizes or is specific in binding to at least one platelet-specific marker; and
analyzing the population of platelet-sized particles obtained in the gating using a fluorescently-tagged protein that recognizes or is specific in binding to a platelet-activation marker, and a fluorescently-tagged protein that recognizes or is specific in binding to a platelet-specific marker. In some embodiments, the platelet-specific marker is CD41, or CD42b, and the platelet-activation marker is selected from the group consisting ofphosphatidylserine (PS), P-Selectin, CD62, and activated GPIIb/IIIa complex. IN illustrative embodiments, the platelet-activation marker is PS, and the fluorescently-labeled protein is lactadherin. In some embodiments, the gating includes a forward scatter height (FSC-H). In illustrative embodiments, the platelet particles in the more activated sub-population are PS positive. In illustrative embodiments, the platelet particles in the less activated sub-population are PS negative. In some embodiments, the fluorescently-tagged protein that recognizes or is specific in binding to a platelet-specific marker is a fluorescently-tagged or fluorescently-labeled antibody specific for the platelet-specific marker. In some embodiments, the fluorescently-tagged protein that recognizes or is specific in binding to a platelet-activation marker is a fluorescently-tagged or fluorescently-labeled protein, wherein the protein is lactadherin, such that lactadherin specifically binds to phosphatidylserine on the platelet particles.

In some embodiments of any of the aspects or embodiments herein that comprise a composition, a collection, process or method, the population of platelet particles has the property, upon thawing and when analyzed using flow cytometry, of comprising at least two detectable sub-populations of platelet particles, a more activated sub-population, and a less activated sub-population. In some embodiments, the two detectable sub-populations can be detected upon gating a population of platelet-sized particles using a fluorescently-tagged protein that recognizes a platelet-specific marker CD41 or CD42b, and forward scatter height (FSC-H); and analyzing the population of platelet particles obtained in the gating using a fluorescently-tagged protein specific for a platelet-activation marker selected from the group consisting of phosphatidylserine, P-Selectin, CD62, and activated GPIIb/IIIa complex phosphatidylserine (PS) and a fluorescently-tagged antibody specific for CD41 or CD42b. In illustrative embodiments, the platelet particles in the more activated sub-population are PS positive.

In some embodiments of any of the aspects or embodiments herein that comprise a composition, a collection, process or method, the population of platelet particles, upon thawing, comprises at least two sub-populations based on the level of activation when measured for the presence of and/or quantified for at least one marker selected from the group consisting of phosphatidylserine, P-Selectin, CD62, and activated GPIIb/IIIa complex.

In some embodiments of any of the aspects or embodiments herein that comprise a composition, a collection, process or method, the population of platelet particles has the property, upon thawing of comprising at least two sub-populations based on the level of activation when measured for the presence of and/or quantified for at least one marker selected from the group consisting of phosphatidylserine, P-Selectin, CD62, and activated GPIIb/IIIa complex.

In some embodiments of any of the aspects or embodiments herein that comprise a composition, a collection, process or method, the population of platelet particles has the property, upon thawing and when analyzed using flow cytometry, of forming two detectable sub-populations of platelet particles, a more activated sub-population, and a less activated sub-population. upon gating a population of platelet-sized particles using a fluorescently-tagged protein that recognizes CD41 and forward scatter height (FSC-H); and analyzing the population of platelet particles obtained in the gating using a fluorescently-tagged protein specific for phosphatidylserine (PS) and a fluorescently-tagged protein specific for CD42b. In illustrative embodiments, the platelet particles in the more activated sub-population are PS positive.

In some embodiments of any of the aspects or embodiments herein that comprise a composition, a collection, process or method, the population of platelet particles upon thawing comprise a more activated sub-population, and a less activated sub-population. In illustrative embodiments, the platelet particles in the more activated sub-population are PS positive. In some embodiments, the platelet particles in the less activated sub-population are PS negative. In some embodiments, the less activated sub-population comprise resting platelets. In some embodiments, the less activated sub-population has resting platelets.

In some embodiments of any of the aspects or embodiments herein that comprise a composition, a collection, process or method, the population of platelet particles upon thawing comprise at least two sub-populations based on the level of activation when measured for the presence of and/or quantified for at least one marker selected from the group consisting of phosphatidylserine, P-Selectin, CD62, and activated GPIIb/IIIa complex. In some embodiments, the level of activation is measured based on the presence of at least one marker selected from the group consisting of phosphatidylserine, P-Selectin, CD62, and activated GPIIb/IIIa complex. In some embodiments, the level of activation is measured based on the presence of phosphatidylserine in a flow cytometry assay, such that the population of platelet particles, upon thawing comprise two sub-populations. In some embodiments, the two sub-populations comprise a first sub-population comprising platelet particles positive for phosphatidylserine, and a second sub-population comprising platelet particles negative for phosphatidylserine. In some embodiments, the first sub-population comprising platelet particles positive for phosphatidylserine is a more activated sub-population, and the second sub-population comprising platelet particles negative for phosphatidylserine is a less activated sub-population. In some embodiments, the level of activation is measured based on the quantification of at least one marker selected from the group consisting of phosphatidylserine, P-Selectin, CD62, and activated GPIIb/IIIa complex. In some embodiments, the level of activation is measured based on the presence of at least one marker selected from the group consisting of phosphatidylserine, P-Selectin, CD62, and activated GPIIb/IIIa complex, and is based on the quantification of at least one marker selected from the group consisting of phosphatidylserine, P-Selectin, CD62, and activated GPIIb/IIIa complex. In some embodiments, the level of activation is measured based on the quantification of phosphatidylserine in a flow cytometry assay, such that the population of platelet particles, upon thawing comprise two sub-populations, and wherein the phosphatidylserine is quantified by measuring the binding of lactadherin to the platelet particles. In some embodiments, the two sub-populations comprise a first sub-population comprising platelet particles exhibiting a mean fluorescence intensity (MFI) of lactadherin of at least 50,000, 60,000, 70,000, or 80,000 when measured using an antibody capable of binding to lactadherin, and a second sub-population comprising platelet particles exhibiting a mean fluorescence intensity (MFI) of phosphatidylserine of less than 30,000, 25,000, 20,000, 15,000, or 10,000 when measured using an antibody against or otherwise capable of binding to lactadherin, such that the first sub-population is a more activated sub-population and the second sub-population is a less activated sub-population. In some embodiments, the median height of the less activated sub-population in a flow cytometry assay is higher than that of the more activated sub-population, for example, at least 2-, 3-, or 4-fold higher median height. In some embodiments, the MFI of CD42b in less activated sub-population is higher than that of the more activated sub-population, for example, at least 2-, 3-, 4-, 5-, or 6-fold higher MFI as compared to that of the more activated sub-population. In some embodiments, the MFI of PS in more activated sub-population is higher than that of the less activated sub-population, for example, at least 5-, 6-, 10-, 15-, 20-, 30-, 40-, 50-, 60-, or 70-fold higher MFI as compared to that of the less activated sub-population.

In one aspect, provided herein is a method for reducing bleeding/controlling blood loss in a subject, the method comprising: thawing the frozen activated platelets herein, or the cryopreserved platelet composition obtained from the process herein, to obtain a thawed composition comprising thawed platelet particles, and administering the thawed composition comprising a dose, a first dose, or an effective amount of the thawed platelet particles to the subject, wherein the administering leads to the subject having reduced bleeding such that after the administering the bleeding in the subject is reduced as compared to a bleeding in the subject before the administering. In some embodiments of the method, the subject is undergoing a surgery and the administering is performed in connection with the surgery. In some embodiments, the subject underwent a surgery, and the administering is performed in connection with reducing bleeding or controlling blood loss in the subject. In some embodiments of the method, wherein the surgery is cardiopulmonary bypass surgery, the administering is done intraoperatively, or post-surgery.

In some embodiments of any of the aspects and embodiments that include method for controlling blood loss or for reducing bleeding in a subject who is undergoing or underwent cardiopulmonary bypass surgery, the administering is done intraoperatively, or post-surgery. In some embodiments of the method, the frozen activated platelets or the cryopreserved platelet composition is non-inferior to liquid stored platelets with respect to reducing the bleeding. In some embodiments of the method, the reduced bleeding is measured and/or detected by measuring blood loss, said method being a method for controlling blood loss. In some embodiments of the method, the blood loss is determined by measuring total volume of chest tube drainage. In some embodiments of the method, the non-inferior is determined based on a difference in LS Means of volume of chest tube drainage and a non-inferiority margin of less than 400 ml. In some embodiments, the margin is less than 380 ml, 350 ml, 325 ml, or 300 ml. In some embodiments of the method, the non-inferior is determined based on a difference in LS Means of volume of chest tube drainage and a non-inferiority margin of less than 350 ml. In some embodiments, the difference in LS Means of volume of chest tube drainage is determined by subtracting the LS Means for LSP from the LS Means for cryopreserved platelets herein. In some embodiments, the non-inferiority margin is determined such that at least 90% statistical powder for the upper limit of the CI around the mean difference in total chest tube drainage excludes 400 ml, for example, less than 400 ml. In some embodiments, the mean difference in total chest tube drainage is determined by subtracting the LS Means for LSP from the LS Means for cryopreserved platelets herein. In some embodiments, the at least 90% statistical power is determined by calculating/analyzing the 95.576% CI of the mean difference in total chest tube drainage. In some embodiments of the method, the total volume of chest tube drainage is measured by measuring the volume of blood collected from the mediastinal and pleural drains from the time of chest closure or equivalent for up to 24 hours or chest tube removal, whichever is earlier. In some embodiments of the method, the total volume of chest tube drainage is measured by measuring the volume of blood collected from the mediastinal and pleural drains with suction determined in ml/kg every hour during the first 12 hours and at 6-hour intervals thereafter, for up to 24 hours or chest tube removal, whichever is earlier. In some embodiments of the method, the method leads to a balanced risk benefit adverse effects between the method and treatment with liquid stored platelets.

In one aspect, provided herein is a method for reducing bleeding in a subject, comprising thawing frozen activated platelets or a cryopreserved platelet composition obtained from a cryopreservation process to obtain a thawed composition comprising thawed platelet particles, and administering the thawed composition comprising a dose, a first dose, or an effective amount of the thawed platelet particles to the subject, wherein the administering leads to the subject having reduced bleeding such that after the administering the bleeding in the subject is reduced as compared to a bleeding in the subject before the administering. In some embodiments of any of the aspects and embodiments herein that include a method, the administering of the thawed composition to the subject does not lead to an increase in the platelet count in the subject.

In some embodiments of any of the aspects and embodiments herein that include a method, the administering of the thawed composition to the subject leads to an increase in a mean platelet count in the subject such that the increase in the mean platelet count is less than or equal to 25%, 22%, 20%, 18%, 17%, 16%, 15%, 14%, 12%, 10%, 8%, 6%, 5%, 4%, 3%, 2%, 1%, or 0.5% as compared to a mean platelet count in the subject before the administering of the thawed composition. For example, in the range of 0.01-15%, 0.01-12%, 0.01-10%, 0.01-8%, 0.01-5%, 1-5%, or 1-4%.

In some embodiments of any of the aspects and embodiments herein that include a method, the administering of the thawed composition to the subject leads to a change in the mean platelet count in the subject such that the change in the mean platelet count is in the range of ±10%, ±8%, ±6%, or ±5% as compared to a mean platelet count in the subject before the administering of the thawed composition.

In some embodiments of any of the aspects and embodiments herein that include a method, the platelet count in the subject is measured from a post-heparin reversal time point to 24 hours post time zero, or when chest tubes are removed, whichever is earlier.

In some embodiments of any of the aspects and embodiments herein that include a method, the time zero is the time of chest closure or equivalent, chest tubes or equivalent are attached to a graduated post-drainage system, or chest tubes or equivalent are attached to a graduated post-drainage system with a suction.

In some embodiments of any of the aspects and embodiments herein that include a method, the administering of the thawed composition to the subject does not lead to an increase in the platelet count in the subject.

In some embodiments of any of the aspects and embodiments herein that include a method, the administering of the thawed composition to the subject leads to an increase in the platelet count in the subject, such that the increase is lower as compared to the increase upon administering liquid stored platelets (LSP) or room temperature platelets (RTP) to the subject.

In some embodiments of any of the aspects and embodiments herein that include a method, the administering of the thawed composition comprises administering 1 to 5, 1 to 4, 1 to 3, or 2 to 3 units of the thawed composition.

In some embodiments of any of the aspects and embodiments herein that include a method, each unit of the thawed composition comprises at least 1.0×10¹¹ platelets, 1.2×10¹¹ platelets, 1.5×10¹¹ platelets, 1.7×10¹¹ platelets, or 1.9×10¹¹ platelets.

In some embodiments of any of the aspects and embodiments herein that include a method, each unit of the thawed composition comprises platelets in the range of 1.5×10¹¹ to 1.9×10¹¹ platelets.

In some embodiments of any of the aspects and embodiments herein that include a method, each unit of the thawed composition comprises a volume in the range of 35 mL to 70 mL, 40 mL to 70 mL, or 45 mL to 60 mL.

In some embodiments of any of the aspects and embodiments herein that include a method, the frozen activated platelets or the cryopreserved platelet composition have the property of being non-inferior to liquid stored platelets with respect to reducing bleeding in a controlled clinical trial, wherein 1-3 units each of the frozen activated platelets or the cryopreserved platelet composition, and the liquid stored platelets are administered to the subject independently.

In some embodiments of any of the aspects and embodiments herein that include a method, 1-3 units of a thawed composition obtained from the frozen activated platelets or the cryopreserved platelet composition are administered to the subject, wherein each unit comprises at least 1.7×10¹¹ platelets.

In some embodiments of any of the aspects and embodiments herein that include a method, 1-3 units of liquid stored platelets are administered to another subject as a part of the clinical trial, wherein each unit of the liquid stored platelets comprises at least 3×10¹¹ platelets.

In some embodiments of any of the aspects and embodiments herein that include a method, the frozen activated platelets or the cryopreserved platelet composition have the property of being non-inferior to liquid stored platelets with respect to reducing the bleeding in a controlled clinical trial, wherein the frozen activated platelets or the cryopreserved platelet composition is administered in an amount/dose/unit that is lower than the amount/dose/unit of the liquid stored platelets. In some embodiments, the frozen activated platelets or the cryopreserved platelet composition is administered in an amount/dose/unit that is at least 5%, 10%, 20%, 25%, 30%, 35%, or 40% lower than the amount/dose/unit of the liquid stored platelets. For example, in some embodiments, the frozen activated platelets or the cryopreserved platelet composition is administered in an amount/dose/unit is 2-50%, 5-50%, 5-47%, or 5-50%, 10-50%, or 20-50% lower than the amount/dose/unit of the liquid stored platelets.

Also provided herein are compositions produced by any of the methods described herein. In some embodiments, any of the compositions provided herein can be made by the methods described herein. Specific embodiments disclosed herein may be further limited in the claims using “consisting of” or “consisting essentially of” language.

The following non-limiting examples are provided purely by way of illustration of exemplary embodiments, and in no way limit the scope and spirit of the present disclosure.

### Examples

### Example 1. Single Donor Method

A single donor CPP unit is composed of one transfusable dose of apheresis platelets that has been concentrated by centrifugation and plasma expression, and is cryopreserved in ˜6% DMSO at colder than or equal to −65° C. (inside a −80° C. freezer). The following is a description of the single donor process described in the art, such as in Valeri, C. Robert et al. Transfusion 45.12 (2005): 1890-1898, also referred to as the Vitalant method.

### Step 1—Initial Inspection of APU Upon Receipt

Following receipt of the APU, it is visually inspected for swirling, aggregates, RBC contamination, and intact ports. The unit is checked for an indication that it has been irradiated. If no irradiation indicator is present, then Vitalant irradiates the unit later in the process (step 4). The time elapsed from end of collection to receipt of the APU is confirmed to be less than or equal to 48 hours.

### Step 2—Determination of Initial APC Volume

The initial APC volume is determined. Throughout the single donor process, volume of a component is determined using a weight/volume conversion method where the total weight of the component minus the tare weight of the empty bag is divided by the specific gravity of the component. For the purposes of this process the units for various measurements are as follows: weights are in grams (g), volumes are in milliliters (mL), and specific gravities are in grams per milliliter (g/mL). Equation 1 is used to determine APC weight and volume.

$$Determination of APC Weight and Volume$$

$$\begin{matrix}APC Weight = APU Weight - Empty Bag Weight \\ APC Volume = \frac{APC Weight}{APC Specific Gravity}\end{matrix} \qquad ( Equation 1 )$$

APU weight contains the APC weight and the weight of the empty apheresis collection bag. The terms APC weight and APC volume describe the weight and volume of the apheresis platelet concentrate (APC) that is contained inside of an APU (apheresis platelet unit). APU volume and APC volume can be used interchangeably, as an APU contains the APC volume inside of it. The following steps are used in the manufacturing process to determine APC volume. The APU weight is determined with a scale. The weight of the empty collection bag is subtracted from the APU weight to find the APC weight. Vitalant used the empty collection bag weights that are described in TMRL.801, Common Tare Weights Job Aid. The APC weight is converted to volume, using a specific gravity of 1.027 g/mL. This specific gravity is from TMRL.301 V.3, “Cryopreservation (Closed System) of Apheresis Platelet Units Procedure” and is considered the known specific gravity of APC, according to literature and standard blood banking practices. This APC specific gravity is used throughout herein.

### Step 3—APC Volume Reduction (if Required)

If the APC volume is above 375 mL then typically a sample is removed to reduce the APC volume until it is inside of the processing range. A 5 mL transfer set (Charter Medical, 6″ Tubing with Piercing Pin and Removeable 5 mL BD™ Syringe, product number 03-220-XL, or equivalent) is connected to the APU using a sterile connecting device (SCD, Terumo TSCD® Sterile Tubing Welder, SC-201A). This 5 mL transfer set is then used to remove the sample. This method of sample removal is used throughout the single donor process to remove samples. After sample removal, the APC volume is redetermined.

### Step 4—APU Irradiation (if Required)

If the APU was not previously irradiated, then it is irradiated at this point in the process.

### Step 5—APU Sampling and Confirmation of Pre-Manufacturing Specifications

The APU is then placed on a platelet incubator/agitator for at least 30 minutes. The APU is inspected for swirling and aggregates again. The APU is then sampled for pH/blood-gas and platelet concentration analysis. A copy of the pH/blood-gas printout is placed in the batch record. After sampling, the APC volume is redetermined. Equation 2 is used to determine the APU total platelet count, using the platelet concentration and APC volume.

$$Determination of APU Total Platelet Count$$

$$Total Platelet Count = APC Volume * Platelet Concentration \qquad ( Equation 2 )$$

The cryopreservation process can continue once it is confirmed that the APU meets all pre-manufacturing specifications. The following excerpt from TMRL.301 V.3 describes the handling of APUs that do not pass the pre-manufacturing specifications, “If the APU does not meet criteria (has a negative swirl, aggregates are present, has a platelet yield <3.0×10¹¹, or a volume <165 or >375 mL), place the APU back in the platelet incubator/agitator and discuss with TMRL Director (or designee) how to proceed. Complete a deviation report as necessary per the Occurrence/Deviation Reporting Procedure: TMRL 113.”

### Step 6-27% DMSO Addition

Sterile, injectable grade 27% DMSO and 0.66% sodium chloride in water (Bio Life Solutions, BloodStor® 27 NaCl Biopreservation Media, part number 327207, or equivalent) is used to formulate the APC to a final DMSO percentage (% DMSO) of 5.65%-6.52%. Throughout this report the APC and DMSO solution is referred to as APC/DMSO. The APU is formulated with 27% DMSO using Table 1:

TABLE 1
TMRL.806 V.1, “Weight of 27% DMSO
to Add for Freezing Platelet Job Aid”

| APC Volume | | | | | | |
|---|---|---|---|---|---|---|
| (mL) | 165-189 | 190-219 | 220-259 | 260-299 | 300-339 | 340-375 |
| 27% DMSO | 50 | 60 | 71 | 81 | 92 | 102 |
| Weight to | | | | | | |
| add (g) | | | | | | |

The 27% DMSO bag is welded onto the APU using an SCD. The APU is placed on a scale to measure the weight of the 27% DMSO that is added to the APU. The 27% DMSO bag is suspended above the APU to allow the 27% DMSO to flow gravimetrically for the 27% DMSO addition step. The 27% DMSO is then slowly added to the APU, and the APU is lightly massaged to promote mixing. The 27% DMSO is added at a rate of 10-15 g/min until the 27% DMSO target weight is reached. The start and end times of the 27% DMSO addition are recorded. If the rate of 27% DMSO addition falls outside of the desired range of 10-15 g/min a deviation report is completed, and manufacturing continues.

### Step 7—% DMSO Calculation

The APU is then weighed to find the APC/DMSO Volume and the % DMSO, using Equations 3 and 4.

$$Determination of APC / DMSO Volume$$

$$APC / DMSO Volume = \frac{APU / DMSO Weight - Tare Weight}{APC / DMSO Specific Gravity} \qquad ( Equation 3 )$$

Vitalant lists the APC/DMSO specific gravity as 1.03 g/mL in TMRL.301 V.3, Cryopreservation (Closed System) of Apheresis Platelet Units Procedure.

$$Determination of \% DMSO$$

$$\begin{matrix}27 \% DMSO Dispensed ( mL ) = APC / DMSO Volume - APC Volume \\ DMSO \% = \frac{27 \% DMSO Dispensed ( mL )}{APC / DMSO Volume}\end{matrix} \qquad ( Equation 4 )$$

The % DMSO is recorded and it is determined if the % DMSO is in range for the product specification (5.65%-6.52%).

### Step 8—Sampling for Culture of APU Post-DMSO Addition

A sample of the APC/DMSO is removed for the purposes of aerobic and anaerobic culturing using BacT/ALERT.

### Step 9—Platelet Material Transfer

A tubing extension set (such as a plasma transfer set, Charter Medical, 24″ Tubing, Roller Clamp and Two Piercing Pins, product number 03-220-00, or equivalent) is welded between a cryobag (500 mL EVA CryoStore freezing bag, Origen Reference CS500, or equivalent) and the APU to connect the two bags and to extend the working length of the tubing for centrifugation and expression. The APC/DMSO solution is then transferred from the APU bag into the cryo-bag. Any excess air in the cryobag is expressed into the empty APU bag, and the tubing is clamped.

### Step 10—Centrifugation

The cryo-bag and empty APU bag then undergo centrifugation at 1250 G for 10 minutes with maximum acceleration, no brake, and slow stop setting set to 3 (Thermo Scientific, Sorvall RC3BP+centrifuge).

### Step 11—Expression

The cryo-bag is carefully removed from the centrifuge cup, as not to disturb the platelet pellet, and is placed in a plasma expressor (such as Fenwal Inc., manual plasma extractor, product code 4R4414, or equivalent). If leaks are noticed, then typically the unit is not used for patient infusion. The plasma expressor is used to remove the supernatant without losing platelets. After expression is complete, the cryobag is weighed. The cryobag should weigh between 50 g-55 g to meet the volume specification needed and to account for additional volume removed for pre-freezing sampling. If the weight is below 50 g, release the tubing clamp and slowly flow material from the plasma/DMSO waste bag back into cryobag to achieve a weight of 50 g-55 g. This range is equivalent to 25.5 mL-30.4 mL. The pre-sampling volume is then determined to ensure that the post-sampling volume will be 20-35 mL.

### Step 12—Platelet Pellet Rest and Resuspension

A 30-minute (±5 minutes) resting period at 20-24° C. is needed prior to resuspension of the platelet pellet. The pellet is resuspended by gentle manual agitation for several minutes. The cryobag is inspected for visible aggregates and it is recorded if aggregates are present.

### Step 13—Post-Resuspension Sampling

If the resulting volume after sampling will be less than 20 mL then the sampling is skipped. If the volume is enough to proceed then a pre-freezing sample of ˜2 mL is removed. The sample is removed by gently filling a 5 mL syringe (part of the 5 mL transfer set) and expelling back several times to mix the platelet suspension to obtain a well-mixed sample.

### Step 14—Pre-Freeze Volume Determination

The pre-freeze volume is determined using a standard empty cryobag tare weight of 22.8 g and a weight/volume conversion that uses the APC/DMSO specific gravity of 1.03 g/mL. This post-freeze volume determination is to ensure that the volume is within range for freezing. The pre-freeze volume range is 20 mL-35 mL.

### Step 15—Freezing

The cryobag is labeled and a final visual inspection of the cryobag is performed to ensure the bag is intact and not leaking. The cryobag's excess tubing is sealed off using a tube sealer (SEBRA® Hand-Held RF Tube Sealing System, model number 2380, or equivalent) and safety seals are added to the remaining tubing. The cryobag is placed into a thawing bag and freezer carton for storage in a −80° C. freezer. The unit is placed in the freezer and the start time of freezing is recorded.

### Step 16—Timing Specifications

The time elapsed from the end of 27% DMSO addition to the start time of freezing typically be kept less than or equal 2 hours and 45 minutes. Time elapsed from the end of APU collection to placement in the freezer typically be less than or equal to 57 hours. If these time specifications are not met, a deviation report is completed.

### Post Manufacturing Specifications:

TMRL.805 V.2 lists the following post-manufacturing specifications for a CPP unit:
Freeze Volume: 20 mL-35 mL
Time from addition of 27% DMSO to freezer: less than or equal to 2 hours and 45 minutes
Age at end of processing: less than or equal to 57 hours.
Visible aggregates: none
Sterility testing: no growth
DMSO: 5.65%- 6.52%
Maximum DMSO in a CPP unit: 2.53 g (the mass corresponding to the maximum freeze volume with the maximum % DMSO)
   Any product not meeting final acceptable criteria (non-conforming products) shall be, at the discretion of the TMRL Director, designated as a miscellaneous product or will be disposed of.

Analysis of certain important steps in single donor process

27% DMSO addition

DMSO at 5% to 10% is commonly used as a cryoprotectant in biological applications and is added to the APC for its cryoprotective properties.

Weaknesses in the Previous 27% DMSO Addition Method

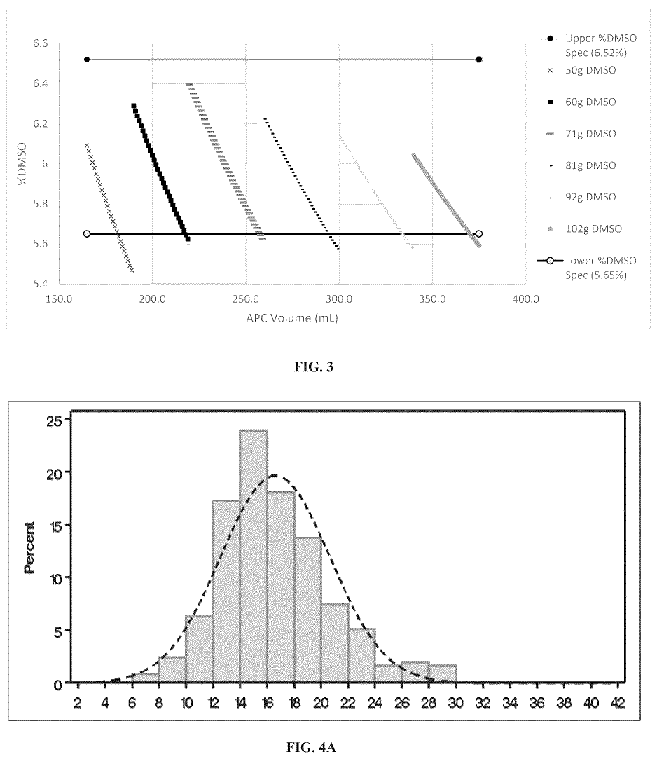

The Vitalant method of adding 27% DMSO based on Table 1 is a very simplistic method of formulating the product, however it has several shortcomings. Dosing the 27% DMSO in this manner creates a process that targets a variable % DMSO. This leads to a product that has an inherently variable % DMSO. Additionally, Table 1 can lead to units that are outside of the % DMSO specification. FIG. 3 illustrates these issues. FIG. 3 displays the target % DMSO that will be achieved using Table 1. Equations 1, 3, and 4 were used to determine the target % DMSO. The variability of % DMSO displayed in FIG. 3 equates to an average percent difference of 10.6%, from the mean of 27% DMSO dosing range. Analysis A explains the math that was used in determining all data points in FIG. 3.

### Analysis A: Analysis of Table 1

A 189.0 mL APU falls in the 50 g of 27% DMSO dosing range according to Table 1. Using a volume/weight conversion like Equation 1, the APC weight of this APU will be 194.1 g. After the addition of the 50 g of 27% DMSO required by Table 1, the APC/DMSO weight will be 244.1 g, or 237.0 mL. Using Equation 4 to determine the percent DMSO indicates that a 189 mL APU dosed with 50 g of 27% DMSO will be formulated to 5.47% DMSO, which is below the stated specifications.

### Freezing Volume

The fundamental purpose of the centrifugation and expression step is to concentrate the platelets into a lower volume so less DMSO is infused when the CPP transfusion takes place. Centrifugation accomplishes this concentration and reduction in overall volume by pelleting the platelets from the plasma/DMSO during the spin, then excess plasma/DMSO can be aspirated using a plasma expressor. The expression step (Step 11) is the determining factor in the freezing volume. The overall variability that is present in Vitalant's expression step and freezing volume is demonstrated in FIG. 4A (FIG. 2A from Vitalant's Long Term Stability Characteristics of Cryopreserved Platelets at −80° C., Protocol 11-5-J). A large range of volumes is observed, suggesting little control over this processing step. In Vitalant's Long Term Stability Characteristics of Cryopreserved Platelets at −80° C., Protocol 11-5-J, aggregates were observed in CPP units that had low freeze volumes. This demonstrates the importance of controlling the expression and fill volume of the process. FIG. 4B (FIG. 2B from the stability study) gives the distribution of freezing volumes for units in which aggregation was observed after thawing; aggregation was observed only in the lower freeze volumes.

The previous expression step instructed manufacturing staff to, “remove as much supernatant as possible without losing platelets.” This method of using visual confirmation is uncontrolled and variable, as shown by FIG. 4A.

### Freezing Protocol

The freezing protocol ensures that the freezing is uniform, from unit to unit. All CPP units are placed in a thawing bag and then placed flat in a cardboard box. The flat orientation of the CPP unit ensures uniformity in the heat transfer from the cryobag to the freezer shelf. The same product enclosure system is used for all units, and all units are placed on an empty shelf in a freezer that is at −80° C., to minimize unit-unit variability.

### Example 2. Exemplary Improved Process of Preparing Cryopreserved Platelets

A non-limiting, exemplary improved processes for preparing cryopreserved platelets using a pool of platelet units as a starting material as disclosed in the Examples, is referred to in these Examples as the pooled CPP process or the exemplary pooled CPP process. Accordingly, a cryopreserved platelet composition comprising frozen activated platelets prepared by using a pool of platelet units from a plurality of donors, or a composition comprising cryopreserved platelets that have a biomolecular profile indicative of a plurality of donors, is referred as a pooled CPP, multi-donor CPP, or cryopreserved platelets disclosed or described herein. Wherever, cryopreserved platelets prepared from a single donor platelet product is referred, it is referred as single-donor cryopreserved platelet composition, or single-donor CPP. The exemplary pooled CPP process accommodates the inclusion and pooling of platelets from 12 apheresis platelet units (APUs), with the goal of improving product control, decreasing variability of the final product, and increasing scalability. The exemplary pooled CPP process incorporates all major process steps that are present in the single donor CPP process of Example 1, however the process has been improved to streamline the process and to create batches of a more homogeneous final product, while limiting batch-to-batch variability of the final product. FIG. 1C is a non-limiting flowchart of the exemplary pooled CPP process used in this example. A summary comparing steps between the exemplary pooled CPP process and the single donor process is shown below.

TABLE 2

| | Single Donor Process | Exemplary Pooled Process |
|---|---|---|
| Step # | (1 APU) | (up to 12 APUs) |
| 1 | Formulate APU | Pool 2 APUs Together |
| | to~6% DMSO | |
| 2 | Centrifuge | Centrifuge |
| 3 | Express | Express Plasma |
| | Plasma/DMSO | |
| 4 | Resuspend Platelets | Resuspend Platelets to a |
| | | target weight |
| 5 | Freeze | Pool Resuspended |
| | | Platelets |
| 6 | N/A | Formulate to ~6% DMSO |
| 7 | N/A | Fill/Freeze |

The exemplary pooled CPP process creates multiple doses of a homogeneous final product. This allows one to perform representative product characterization testing for all pooled CPP units sent for infusion and allows for a product archive of all batches produced. The single donor process does not allow for the same kind of product quality assurance. Multiple doses from the same final product will also allow one to initiate stability studies that will directly compare the same product at different time points, allowing for a true representation of product stability. This protocol maintains the same product formulation of the cryopreserved platelets (ratios of platelets, plasma, saline, and DMSO) and a similar overall process.

An illustrative, non-limiting process of preparing the cryopreserved platelets as disclosed herein is shown below.

The exemplary pooled CPP process has been designed to incorporate up to 12 apheresis platelet units (APUs) from 5 to 10 donors into a single pool to produce up to 12 pooled CPP units. The process can be completed with less than 12 APUs, however a minimum of 5 APUs from 5 donors are needed. The number of initial APUs equals the number of pooled CPP units produced. The pooled CPP product has the same composition as the single donor product (77.5% residual plasma from the APUs, 16.5% of a 0.66% NaCl saline solution, and ˜6% DMSO). Like a single donor CPP unit, a pooled CPP unit is composed of one transfusable dose of apheresis platelets that has been concentrated, by centrifugation and plasma expression, and is cryopreserved in ˜6% DMSO at colder than or equal to −65° C. (inside a −80° C. freezer).

### Step 1—Initial Quality Control (QC) of APUs Upon Receipt

Prior to the APUs being released for manufacturing, the QC department confirms that all APUs meet the following pre-manufacturing specifications, according to QCT-001.
Leukoreduced: Less than 5×10⁶ WBCs—responsibility of collecting facility.
Platelet Age: less than or equal to day 2.
Gamma irradiated at 25 Gy, responsibility of the collecting facility.
Ports of APU are intact.
No indication of red blood cell (RBC) contamination.
Aggregate free swirling of APC.
pH: greater than or equal to pH 6.2.
Average APU total platelet count: greater than or equal to 2.5×10¹¹ platelets.

### Step 2—Quality Assurance (QA) Line Clearance

The QA Department completes line clearance of the processing before manufacturing begins.

### Step 3—Transfer of APUs to Manufacturing and Manufacturing Inspection of APUs

Following the QA and QC duties, the APUs are released for manufacturing (101 of FIG. 1C). The start time of production is recorded when the APUs are removed from the QC platelet incubator and physically given to Manufacturing Staff Upon receipt of the released APUs, the Manufacturing Staff completes several quality checks to verify the APUs are acceptable. The APUs are visually inspected for swirling, aggregates, red cell contamination, and it is confirmed that the APUs have been irradiated. The Donor ID and expiration date of the APUs are recorded in the batch record for traceability and tracking purposes.

### Step 4—Creating 2-Unit Pools of APUs

After all units are determined to be acceptable, an SCD is used to weld a plasma transfer set onto an APU and then a second APU is welded onto the other end of the plasma transfer set. The plasma transfer set is added to extend the working length of the tubing. The two APUs are then pooled together into a single APU bag (121 of FIG. 1C). This is done 6 times to create 6 pools of 2 APUs from the initial 12 APUs. The sterile connecting device used is a Terumo, TSCD II Sterile Tubing Welder, model number 3me-SC203a (or equivalent). The plasma transfer sets used are Charter Medical, 24″ Tubing, Roller Clamp and Two Piercing Pins, product number 03-220-00 (or equivalent).

### Step 5—Determination of Pooled APC Weight

The pooled APC weights are determined and recorded for later use in the expression step. For each of the 6 pooled APUs, the pooled APC weight is determined using the following equation:

$$Pooled APC Weight = Pooled APU Weight - Empty Bag Weight$$

The pooled APU weights are determined with a scale (Ohaus Adventurer Precision Balance, product number AX8201/E, or equivalent). The empty bag weight is a known value that corresponds to the type of bag that was used for the apheresis platelet collection.

### Step 6—Centrifugation

Each pooled APU is placed into a centrifuge cup. The cups are balanced by weight (if needed) and then loaded into the centrifuge. The pooled APUs undergo a 1250 G centrifugation (131 of FIG. 1C) for 10 minutes at maximum acceleration, with a 10-minute deceleration (Beckman-Coulter Avanti J-HC Centrifuge).

### Step 7—Expression

The plasma removal target weight for expression is determined using Equation 5 by subtracting 46.5 g from the pooled APC weight. This is done to leave behind approximately 46.5 g of platelet pellet and plasma after the pooled APU is expressed.

$$Determination of Expression Endpoint$$

$$Plasma Removal Target = Pooled APC Weight - 46.5 g \qquad ( Equation 5 )$$

Each pooled APU is taken out of the centrifuge and expressed one-by-one. The pooled APU is carefully removed from the centrifuge cup, as not to disturb the platelet pellet, and then placed in the plasma expressor (Fenwal Inc., manual plasma extractor, product code 4R4414, or equivalent). The empty APU bag is placed on a scale and tared to weigh the expressed plasma. The pooled APU is then expressed (135 of FIG. 1C). Once the plasma removal target is reached (±1.0 g) the expression is stopped.

### Step 8—Post-Expression Weight Check

The post-expression pellet weights are determined to ensure that the weight of the platelet pellet and supernatant is within a target range for further processing (31.8 g-55.7 g). If the post-expression weight is outside of the range, the supernatant will be added or removed accordingly, until the post-expression weight is within range.

### Step 9—Resuspension

Once the post-expression weight is within range, the pellet is resuspended (141 of FIG. 1C) by gently rocking and massaging the APU bag until the pellet is no longer visible. After the pellet is no longer visible there is a 5-minute ambient temperature resting of the resuspended platelet pellet. The resuspended pellet is then visually inspected for aggregates. If aggregates are observed at this point in the process, and they do not disappear after further resting and agitation, manufacturing management is informed, processing continues, and a 30-minute ambient temperature rest with gentle agitation is added to the process after the addition of 27% DMSO.

### Step 10—Pooling

Once the 6 pellets are resuspended, the 6 APU bags are welded onto a sterile tubing tree (Optimum Processing, Inc., part number 02817, or equivalent) to create a “pooling tree system” and the resuspended platelet pellet material is pooled into a single APU bag (151 of FIG. 1C). FIG. 1D is a picture of the tubing tree that was used to create batches or lots of cryo-vessels using the method herein.

### Step 11—Calculation of 27% DMSO Target Weight

The sum of the previously measured post-expression weights is used to determine the total post-expression weight, using Equation 6. The total post-expression weight is used in Equation 7 to calculate the 27% DMSO Target Weight needed to formulate the pooled, resuspended platelet material to ˜6% DMSO. Equation 7 uses a “DMSO Constant” of 0.2946, derived in the section titled “Development of 27% DMSO Addition Method for Pooled CPP.”

$$Determination of Total Post - Expression Weight$$

$$Total Post Expression Weight = Sumation of Platelet Pellet Weights \qquad ( Equation 6 )$$

$$Determination of 27 \% DMSO Target Weight$$

$$27 \% DMSO Target Weight = APC Weight * 0.2946 \qquad ( Equation 7 )$$

### Step 12—Addition of 27% DMSO

A bag containing sterile, injectable grade 27% DMSO and 0.66% sodium chloride in water (Bio Life Solutions, BloodStor® 27 NaCl Biopreservation Media, part number 327207, or equivalent) is then welded onto the pooling tree system. The 27% DMSO bag is placed on a scale to weigh how much 27% DMSO is leaving the 27% DMSO bag and entering the pooling tree system. The 27% DMSO bag is placed higher than the pooling tree system to allow the 27% DMSO to flow gravimetrically. The 27% DMSO Target Weight ±1.0 g is added (161 of FIG. 1C) to the pooling tree system. The tubing to the 27% DMSO bag is clamped to prevent additional 27% DMSO from entering the system.

### Step 13—Rinsing

The 27% DMSO that entered the pooling tree system is used to rinse the system (165 of FIG. 1C) to recoup any residual platelet material that remains in the APU bags and in the tubing of the pooling tree system. The solution is then added to the APU that contains the pooled, resuspended platelet pellets. Tube strippers are used to strip any solution that may remain in the pooling tree system, to ensure that all 27% DMSO is added to the platelets.

### Step 14—Fill Volume Determination of Cryobags

The APU bag containing the final product is welded onto a new tubing tree to fill 12 cryobags. This creates a “filling tree system.” The final product weight is then determined by weighing the APU bag and subtracting the empty bag weight (empty bag weight determination is explained in Step 5 “Determination of Pooled APC Weight”). The final product weight is divided by 12 to determine the maximum fill weight of the cryobags. The minimum fill weight is determined by subtracting 2.0 g from the maximum fill weight. This determines the fill weight range of the cryobags.

### Step 15—Cryobag Filling Procedure

The 12 cryobags (250 mL EVA CryoStore freezing bag, Origen Reference CS250, or equivalent) are then welded onto the dosing tree system. The filling procedure for a cryobag takes place by placing the cryobag on the scale, priming the lines of the cryobag until just before the product enters the cryobag, and then taring the cryobag. The cryobag is then filled (171 of FIG. 1C) with the final product until it is within range. The fill weight of each cryobag is recorded and their volume is determined by dividing the weight by 1.03 g/mL.

### Step 16—Freezing

Each cryobag is then placed into a thawing bag and freezer carton (175 of FIG. 1C) for storage in a −80° C. freezer. The units are then placed in the freezer (181 of FIG. 1C) and the start time of freezing is recorded. Whenever required, after a certain time-period of freezing at −80° C. freezer, certain number of cryobags were transferred to a freezer set at a temperature of −20° C., and stored at the temperature to form transition temperature cryopreserved-product. The time elapsed from the end of 27% DMSO addition to the start time of freezing typically be equal to or less than 3 hours.

### Post-Manufacturing Specifications:

All CPP units produced using the exemplary pooled CPP process, typically, meet the following specifications prior to freezing:
Freeze Volume: 20 mL-35 mL
Time from addition of 27% DMSO to freezer: less than or equal to 3 hours
Frozen by the end of day 2 of platelet age
Visible aggregates in cryobag: none
% DMSO: 5.65%- 6.52%
Maximum DMSO in a CPP unit: 2.53 g (the mass corresponding to the maximum freeze volume with the maximum % DMSO).
   Summary for Batches with an Odd Number of Initial APUs

If there is an odd number of initial APUs then the following steps are altered to include additions that will allow for the pooled process to accommodate the single APU. All other steps, calculations, and specifications for the single APU, and the pooled process, remain unchanged.

### Step 4 (Creating 2-Unit Pools of APUs):

A plasma transfer set is welded onto the odd APU and a 600 mL transfer bag (Terumo, TeruFlex Transfer Bag, catalog number: 1BB*T060CB71, or equivalent) is welded onto the other end of the plasma transfer set. The APC of the odd APU remains in the APU bag.

### Step 7 (Expression):

Equation 5, which calculates the plasma removal target to determine the expression endpoint, is altered to account for the odd APU being a single APU and not a pooled APU:

$$Plasma Removal Target ( for single APU ) = Odd APC Weight = 23.3 g$$

### Step 8 (Post-Expression Weight Check):

The post-expression pellet target weight range is changed to 15.9 g-27.9 g, to account for the odd APU being a single APU and not a pooled APU.

### Example 3. Process Development for Pooled CPP as Disclosed Herein

### Standardization of Weighing Practices:

To increase 1precision and accuracy, a standard orientation of weighing all bags has been implemented for the purposes of weight determination and weight/volume conversion. APU bags are weighed by folding one third of the bag underneath itself and then it is centered and placed to the back edge of the scale, while ensuring all of the bag is on the scale and that no unnecessary tubing is on the scale. Cryobags are placed on the scale in a similar fashion, however their smaller profile does not require the bag to be folded.

### Elimination of Incoming APC Volume Specifications:

The single donor process had a pre-processing APC volume specification of 165 mL-375 mL. This volume specification was included to ensure that the APUs were acceptable for their process. APUs outside of this specification would not adhere to the 27% DMSO addition table (Table 1). Due to changes in centrifugation practices and 27% DMSO addition methods this volume specification is no longer relevant for the pooled process. The upper end of this volume specification was included due to a processing constraint stemming from the cryobag size that was previously used and the centrifugation practices of the single donor method. The single donor method requires formulating the APC to ˜6% DMSO prior to centrifugation and transferring the APC/DMSO to a cryobag, then centrifuging the APC/DMSO in the cryobag. An incoming APU with a volume of 375 mL will become 473 mL after the 27% DMSO addition. Since a 500 mL cryobag was used for centrifugation, anything above ˜473 mL will either not fit in the cryobag or will result in significant “pillowing” of the cryobag. The pooled process does not add the 27% DMSO prior to centrifugation, so there is no increase in the working volume of the platelet material prior to centrifugation. The pooled process also uses an APU bag for centrifugation rather than a cryobag. An apheresis platelet bag can hold a maximum volume of ˜1600 mL and can comfortably hold ˜800 mL without pillowing. Therefore, a processing constraint for volume is not necessary with the pooled process. Eliminating this volume specification allows more APUs to be acceptable for processing. Additionally, unnecessary stress on the cryobag has been removed from the process, since the cryobag is not undergoing centrifugation. This practice of centrifuging in the cryobag allows for the possibility of a cryobag bursting in the centrifuge and induces unnecessary stress on the cryobag. This unnecessary stress can cause loss of integrity and increases the chance for potential breakage further downstream in the freezing and thawing steps of product handling.

### Derivation of DMSO Constant:

The DMSO constant in Equation 7 is derived from the C1V1=C2V2 calculation that is used in Equation 4 to calculate % DMSO. The derivation of the DMSO constant is shown below:

$$\begin{matrix}C 1 * V 1 = C 2 * V 2 \\ C 1 = 27 \% DMSO \\ V 1 = 27 \% DMSO Volume = X ( the value of interest ) \\ C 2 = 6.085 \% DMSO ( middle of \% DMSO specificiation ) \\ V 2 = APC / DMSO Volume = APC Volume + 27 \% DMSO Volume = APC Volume + X\end{matrix}$$

Substitute V1 and V2 into the C1*V1=C2*V2 equation:

$$C 1 * X = C 2 * ( APC Volume + X )$$

Substitute in the known DMSO percentages (C1 and C2):

$$27 \% * X = 6.085 \% * ( APC Volume + X )$$

Rearranging the equation to solve for X gives the following:

$$\begin{matrix}X = \frac{6.085 \%}{27 \% - 6.085 \%} * APC Volume \\ X = 0.2909 * APC Volume = 27 \% DMSO Volume\end{matrix}$$

This constant of 0.2909 when multiplied by the APC volume will calculate the 27% DMSO volume needed to achieve 6.085% DMSO. Since the in-process measurements are weights the constant is then converted to account for this, to eliminate unnecessary weight/volume conversions and to simplify the calculations that the operator must perform. This conversion was done by using the 27% DMSO specific gravity and the APC specific gravity.

$$27 \% DMSO Volume = \frac{27 \% DMSO Weight}{1.04 \frac{g}{mL}}$$

This specific gravity of 1.04 g/mL is from the excipient SDS.

$$APC Volume = \frac{APC Weight}{1.0 \; 27 \frac{g}{mL}}$$

1.027 g/mL is the known specific gravity of APC.

Substitute in these formulas for APC volume and 27% DMSO volume into the previous equation that solved for 27% DMSO volume:

$$\frac{27 \% DMSO Weight}{1.04} = 0.2909 * \frac{APC Weight}{1.027}$$

Rearrange equation to solve for 27% DMSO weight:

$$27 \% DMSO Weight = ( \frac{0.2909 * 1.04}{1.027} ) * APC Weight$$

$$DMSO Constant = \frac{0.2909 * 1.04}{1.027} = 0.2946$$

This DMSO constant (0.2946) is a unitless value that when multiplied by the APC weight, will determine the weight 27% DMSO needed to formulate the APC/DMSO to 6.085% DMSO (6.09% DMSO when rounded).

### Implementation of 27% DMSO Addition Calculation

For the exemplary improved pooled process or exemplary pooled CPP process, a method of formulating the CPP product to a fixed % DMSO was developed to eliminate the problems identified in the analysis of the previous 27% DMSO addition method of Example 1 (Table 1 and Analysis A). This exemplary pooled CPP process is intended to reduce batch-to-batch variability of the product formulation and ensure that the APC/DMSO is consistently inside the percent DMSO (% DMSO) specification. Using the DMSO constant produces a method where every or virtually every pooled CPP batch will be formulated to achieve the same target percent DMSO. A tolerance of 1.0 g of 27% DMSO has been included to ensure that the percent DMSO is always in-specifications. To demonstrate the theory of this, Analysis B applies the DMSO constant method of formulating a 189 mL APU as performed for the improved exemplary pooled CPP process. A comparison of Analysis B with Analysis A demonstrates how the DMSO constant can eliminate the issues in the prior single-donor method of Example 1.

### (Analysis B): Case Study of 27% DMSO Addition Methods for Pooled CPP

$$Weight of 27 \% DMSO needed = APC Weight * DMSO Constant$$

$$\begin{matrix}Weight of 27 \% DMSO needed = 194.1 g * 0.2946 \\ = 57.2 g of 27 \% DMSO\end{matrix}$$

Using the same mathematical approach as Analysis A, when a 189.0 mL APU is dosed with 57.2 g of 27% DMSO, the APU in Analysis B is formulated to 6.09% DMSO.

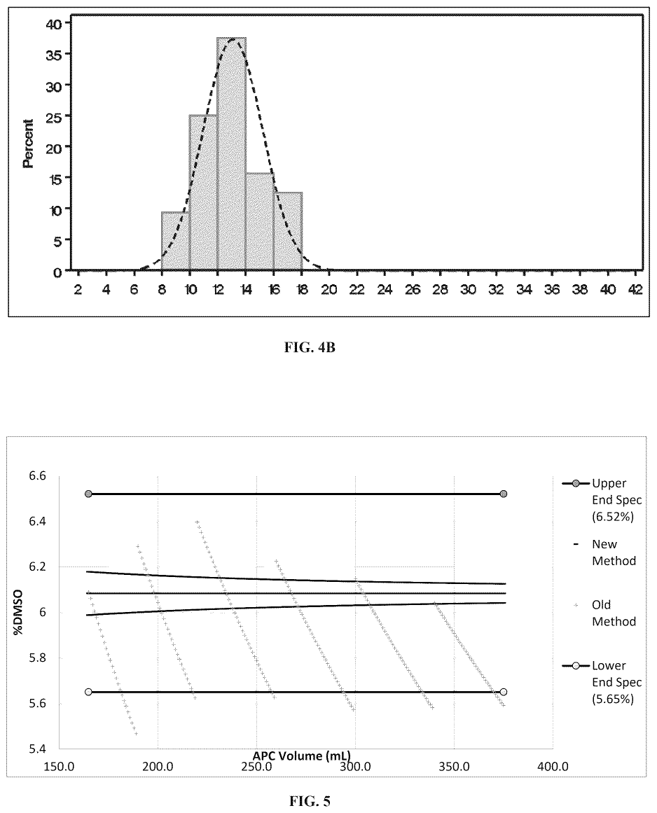

FIG. 5 is like FIG. 5 and compares the correlation of APC volume to % DMSO of the two 27% DMSO addition methods. FIG. 5 illustrates the increased precision and accuracy of the exemplary pooled CPP process. The old method refers to the use of Table 1 that is used in the single donor CPP process and Analysis A. The exemplary pooled CPP process refers to the use of Equation 7 that is used in the exemplary pooled CPP process and Analysis B. The plot for the exemplary pooled CPP process also displays lines for the tolerances of ±1 g from the 27% DMSO target weight.

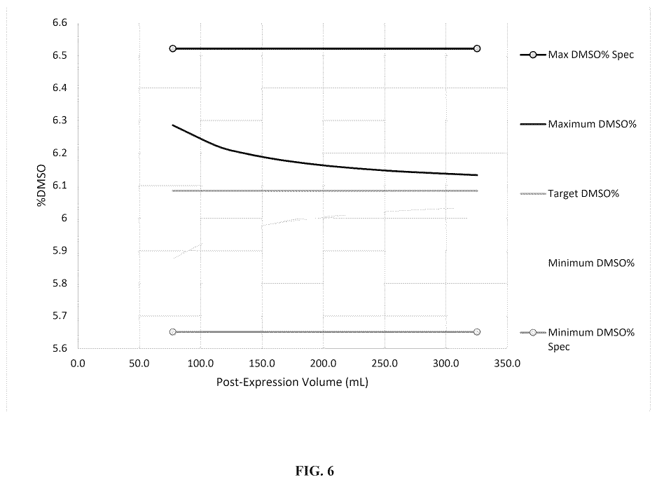

FIG. 6 illustrates the correlation between post-expression volume and percent DMSO for the exemplary pooled CPP process.

FIG. 6 encompasses the full range of post-expression volumes that are capable with the pooled process. The lowest achievable post-expression volume is from a 5-unit batch where all APUs are expressed to the lower end of the post-expression range. The highest achievable post-expression volume is from a 12-unit batch where all APUs are expressed to the upper end of the post-expression range. Lines for the ±1 g tolerances of the 27% DMSO target weight are also included. FIG. 6 demonstrates that percent DMSO of the exemplary pooled CPP process will always be in specification, for all batch sizes (including the minimum batch size of 5 APUs), this eliminates the need for a confirmatory percent DMSO calculation. Table 3 is composed of in-process percent DMSO data from all cGMP-like pooled CPP batches that have been produced (12 batches, encompassing 136 pooled CPP units).

TABLE 3

| Batch# | 28 | 30 | 31 | 35 | 36 | 37 | 38 | 41 | 42 | 44 | 46 | 47 | Mean | CV |
|---|---|---|---|---|---|---|---|---|---|---|---|---|---|---|
| % DMSO | 6.14 | 6.05 | 6.09 | 6.06 | 6.09 | 6.10 | 6.09 | 6.10 | 6.12 | 6.09 | 6.09 | 6.08 | 6.09 | 0.39% |

Table 3 demonstrates the precision (low CV) and accuracy (target % DMSO is 6.09%) that was obtained with the 27% DMSO addition method (a step of exemplary pooled CPP process) embodiment provided in Example 2.

### Optimization of Excipient Usage:

The single donor process of Example 1 requires adding 27% DMSO prior to the platelets being concentrated by centrifugation and plasma expression. Adding 27% DMSO prior to centrifugation increases the working volume of the solution by ˜30%, which limits scalability for the single donor process. An additional hinderance to scalability of the single donor process comes from the fact that this method also requires most of the 27% DMSO to be disposed of as waste, as the majority of the added 27% DMSO is removed during plasma expression. For instance, in Analysis B, a 189 mL APU will have 57.2 g of 27% DMSO added to the whole APU. After centrifugation, if the plasma/DMSO is expressed to retain 28.0 mL of residual plasma/DMSO and platelet pellet, then 216.0 mL of the plasma/DMSO is removed. This equates to 48.7 mL of the 27% DMSO being removed from the CPP unit, while 6.3 mL of the 27% DMSO is retained in the cryobag with the platelets. This is an inefficient use of the product's only excipient as ˜90% of the excipient is removed from the final product and disposed of as waste. The pooled process adds the 27% DMSO after centrifugation and plasma removal, therefore the excipient usage is optimized, none is disposed of as waste, and only the amount of 27% DMSO that is required to formulate the product to the target % DMSO is consumed.

### Addition of Plasma Removal Target In-Process Control:

Equation 5 was created to determine the endpoint of expression to standardize the expression step. This equation uses the pre-centrifugation weight to calculate an endpoint weight for expression. The plasma is weighed as it is expressed to determine when the endpoint is reached. Expression is stopped once the plasma removal target weight (±1.0 g) is reached. The tolerances on the plasma removal target are included to ensure that the expression step doesn't lead to a post-expression weight that is outside of the range needed to continue processing. These additions to the expression step are included to increase control over the process and decrease batch-to-batch variation. The plasma removal target is calculated using the calculation below.

$$Determination of Expression Endpoint$$

$$Plasma Removal Target = Pooled APC Weight - 46.5 g \qquad ( Equation 5 )$$

This method typically ensures that approximately 46.5 g of platelet pellet and plasma are left behind after expression. After expression, there is typically a weight check to ensure that the weight of the platelet pellet and supernatant is within range for further processing (31.8 g-55.7 g). If the post-expression weight is outside of the range, the supernatant will be added/removed accordingly until within range.

### Determination of Post-Expression Ranges:

The single donor plasma expression takes place after the addition of 27% DMSO, and the pooled process centrifugation and plasma expression step take place prior to the addition of the 27% DMSO. This had to be accounted for when determining the post-expression weight ranges. The calculations for determining the post-expression weight ranges are below:

First, the contribution of the APC in the final APC/DMSO solution typically be determined. The APC volume fraction of the APC/DMSO solution typically be calculated to determine this:

$$Volume Fraction = \frac{Solute Volume}{Solution Volume}$$

$$APC Volume Fraction = \frac{APC Volume}{APC / DMSO Volume}$$

$$APC / DMSO Volume = APC Volume + 27 \% DMSO Volume$$

Substitute the formula for APC/DMSO Volume into the APC Volume Fraction equation:

$$APC Volume Fraction = \frac{APC Volume}{APC Volume + 27 \% DMSO Volume}$$

As previously discussed in the “Determination of DMSO Constant” section, if formulating the APC/DMSO to 6.09% DMSO a formula for 27% DMSO volume is:

$$27 \% DMSO Volume = 0.2909 * APC Volume$$

Substitute this equation for 27% DMSO Volume into the APC Volume Fraction formula:

$$\begin{matrix}APC Volume Fraction = \frac{APC Volume}{APC Volume + 0.2909 * APC Volume} \\ = \frac{APC volume}{( 1 + 0.2909 ) * APC Volume}\end{matrix}$$

Since APC Volume is in the numerator and denominator of the equation, this value cancels out of the equation and the APC Volume Fraction can be solved:

$$APC Volume Fraction = \frac{1}{( 1 + 0.2909 )} = \frac{1}{1.2909} = 0.775$$

This means that the APC makes up 77.5% of the CPP product after being formulated to 6.09% DMSO.

The single donor freeze volume ranges are for the APC/DMSO product, so to convert them to an equivalent pre-DMSO range, this APU Volume Fraction of 0.775 typically be used:

| Low End Volume: | High End Volume: |
|---|---|
| 20 mL * 0.775 = 15.5 mL | 35 mL * 0.775 = 27.1 mL |

The pooled process involves pooling two APUs for centrifugation and expression, so these values are doubled, then they are converted weights for ease of use in the process, using the APC specific gravity of 1.027 g/mL:

| Low End Weight Specification: | High End Weight Specification: |
|---|---|
| $15.5 mL * 2 * 1.027 \frac{g}{mL} = 31.8 g$ | $27.1 mL * 2 * 1.027 \frac{g}{mL} = 55.7 g$ |

### Determination of Post-Expression Target Weight of 46.5 g:

Vitalant's single donor process had a post-expression volume range of 25.5 mL-31.3 mL. The middle of this range is 28 mL, or 28.8 g. When rounded to a 1 g interval, this becomes 29 g. This APC/DMSO weight of 29 g was chosen as a starting point for determining the post-expression target weight of the exemplary pooled CPP process. To account for potential dead-space losses introduced by the pooled process, this value of 29 g was increased by 1 g, to become 30 g (or 29.1 mL). This was then converted to an equivalent amount of pre-DMSO product using the same conversions in the previous section:

$$\begin{matrix}29.1 mL of APC / DMSO * APC Volume Fraction = 29.1 mL * 0.775 \\ = 22.6 mL of APC\end{matrix}$$

29.1 mL of APC/DMSO is composed of 22.6 mL of APC. The pooled process involves pooling two APUs for centrifugation and expression, so this value of 22.6 mL of APC was then doubled and then converted to a weight for ease of use in the process, using the APC specific gravity of 1.027 g/mL:

$$22.6 mL * 2 * 1.0 \; 27 \frac{g}{mL} = 46.4 g$$

This value of 46.4 g was then rounded to the nearest half-gram interval to become the post-expression target weight value of 46.5 g.

### Implementation of New Expression and Filling Methods

Table 4 is composed of in-process post-expression and fill/freeze volume data from all cGMP-like pooled batches that have been produced to date using the improved exemplary pooled CPP method of Example 2. These 12 pooled CPP batches encompassed 69 expression steps and 136 manufactured pooled CPP units. The single donor post-expression and freeze volume specification ranges are listed, along with the middle of these ranges. The pooled CPP post-expression volumes have been converted to the equivalent post-DMSO values, to allow for adequate comparisons, using the previously discussed APC/DMSO volume to APC volume conversion. The minimum and maximum batch-averages from the pooled CPP batches are listed, along with the mean intra-batch CV.

TABLE 4
Summary of New Expression and Filling Methods

| | | Middle/Mean | Spec. Range | Batch-Average | Mean Intra- |
|---|---|---|---|---|---|
| Process | Step | (mL) | (mL) | Range (mL) | Batch CV |
| Single | Post-Expression | 28.0 | 25.5-31.3 | N/A | N/A |
| Donor | Freeze | 27.5 | 20.0-35.0 | N/A | N/A |
| Pool | Post-Expression (n = 69) | 27.8 | 20.0-35.0 | 26.5-28.8 | 7.7% |
| | Freeze (n = 136) | 26.4 | | 25.0-28.1 | 1.6% |

As discussed in the analysis of the single donor process, the expression step and freeze volume are important for the product, and therefore in illustrative examples, they should be as controlled as possible. The additions to the expression step have created an accurate and precise method, as the pooled CPP post-expression mean is 0.2 mL away from the single donor post-expression volume of 28.0 mL (a 0.7% difference), and the CV is below 10%. Additionally, the pooled process post-expression range is inside of the single donor post-expression range of the process of Example 1. Furthermore, all pooled CPP batches manufactured to-date using the improved exemplary pooled CPP process of Example 2 have been inside the pooled CPP post-expression range and have not required additional handling, manipulation, and weight readjustment. This demonstrates the reliability of the method improvements. The in-process calculations and controls in the expression and filling operations ensure that every cryobag will be filled inside of the 20 mL-35 mL freeze volume range. The pooled process allows for greater control over the freeze volume as each bag is individually filled from a common pool of product. The freeze volume CV demonstrates that the pooled process has a very controlled filling procedure, which decreases variability in freeze volume. The pooled CPP freeze volume data demonstrates that the improvements in the expression step coupled with the filling procedure will lead to acceptable freeze volumes. As per the improved exemplary processes provided herein in Example 2, 12 batches of CPP as obtained has a mean intra-batch CV of post-expression volume (resuspension volume) of 7.7%, and a mean intra-batch CV of freeze volume (pooled resuspension with cryoprotectant in each cryo-bag) of 1.6%.

### Example 4. Homogeneity of Intra-batch Pooled CPP

The intra-batch homogeneity was tested across 28 units or cryo-vessels from 6 cGMP-like pooled CPP batches. The pooled CPP batches were manufactured according to the method of Example 2. The platelet count per bag, platelets/μL, TGA measurements in IU/10⁶ platelets, percent positivity of CD61 microparticles, and pH were determined across the 28 units or cryo-vessels from the 6 batches. The platelet count per bag and count/μL was obtained using either the Beckman Coulter AcT Diff 2 Hematology Particle Analyzer or the Beckman Coulter D×H Hematology Analyzer (Beckman Coulter, beckmancoulter.com).

Thrombin Generation Assay was performed according to the following steps. Thrombinoscope CAT software was opened, and instrument was set-up according to manufacturer's guideline. PRP reagent containing tissue factor and phospholipids, fluobuffer, and fluo-substrate were prepared according to manufacturer's guidelines (Stago, stago.com). Thawed Octaplas® (Octaphama, octaphramausa.com) thawed TGA dilution buffer were combined to create a buffer containing 30% Octaplas. The combined buffer was used to dilute cephalin 1:50 to be used as a positive control. Octaplas was used to dilute CPP to 1584×10³/μL based on the platelet count data.

1584×10³/μL CPP dilution was used to make 325×10³/L, 160×10³/L, and 80×10³/L serial dilutions of CPP using Octaplas. A multichannel pipette was used to add 20 μL of PRP reagent to each test well and 20 μL of calibrator to each calibration well. Then 15 μL of the 325×10³/μL, 160×10³/L, and 80×10³/μL samples were added to each of their test and calibration wells. 65 μL of Octaplas was added to every test and calibration well. 80 μL of 1:50 cephalin was added to the positive control wells. Each plate was inserted into the tray and incubated for 10 minutes at 41° C. After incubations fluo-buffer and fluo-substrate were dispensed into the active wells. The plate was read for 75 minutes at 20 second intervals to capture the full thrombin generation profile. Thrombin generation profile measurements were reported as IU/10⁶particles reading.

The flow cytometry result for CD61 microparticle positivity was obtained using the Novocyte Flow Cytometry (Agilent, agilent.com) according to the following method. The Novocyte Flow Cytometry was prepared according to manufacturer's guidelines. CPP sample was diluted 100-fold in saline. 10 μL of anti-human CD61-APC (BD Biosciences, bdbiosciences.com) was diluted into 70 μL. A gated isotope control stain mix was created by combining 20 μL of mouse IgG1-APC (BD Biosciences, bdbiosciences.com) with 60 μL of saline. A test stain mix was created by combining 20 μL of the diluted Cd61-APC and 60 μL of saline. The control samples were stained in triplicate by adding 5 μL of 1:100 CPP to 20 μL of the gated isotope control stain mix. The test samples were then stained in triplicate by adding 5 μL of 1:100 CPP to 20 μL of test stain mix. All samples were incubated away from light at room temperature for 20-30 minutes. Once incubation was complete, 400 μL od saline was added to each sample, then 100 μL of each sample was added to each well, beginning with the gating control samples and then the test samples. Well plate was then inserted into tray and plate was run. After sample acquisition was complete, gates were adjusted according to controls and used to determine CD61 positive microparticle count positivity on the test samples. The table below shows the results of the 28 units across the 6 batches.

TABLE 5

| | | | | | | | | Mean |
|---|---|---|---|---|---|---|---|---|
| | | | | | | | | Intra- |
| | | 1 | 2 | 3 | 4 | 5 | 6 | Batch |
| | Batch# | (n = 8) | (n = 2) | (n = 6) | (n = 3) | (n = 3) | (n = 6) | CV |
| Total Plts | Batch Average | 2.3E+11 | 2.3E+11 | 2.1E+11 | 2.1E+11 | 2.0E+11 | 2.4E+11 | 4.2% |
| | Intra-Batch CV | 5.1% | 4.2% | 6.4% | 1.6% | 2.2% | 5.7% | |
| Plts/μL | Batch Average | 4.7E+06 | 4.6E+06 | 4.1E+06 | 4.1E+06 | 3.9E+06 | 4.6E+06 | 3.7% |
| | Intra-Batch CV | 4.2% | 4.8% | 5.8% | 1.5% | 0.4% | 5.8% | |
| IU/10⁶ Plts | Batch Average | 1.6 | 1.6 | 1.5 | 1.5 | 1.6 | 1.6 | 2.0% |
| | Intra-Batch CV | 2.4% | 1.5% | 0.8% | 1.8% | 3.0% | 2.7% | |
| CD61+ | Batch Average | 7.4E+06 | 5.2E+06 | 3.7E+06 | 4.4E+06 | 3.6E+06 | 6.0E+06 | 6.9% |
| MP/μL | Intra-Batch CV | 10.0% | 10.6% | 7.2% | 1.9% | 7.3% | 4.7% | |
| pH | Batch Average | 6.45 | 6.29 | 6.64 | 6.46 | 6.47 | 6.55 | 0.8% |
| | Intra-Batch CV | 1.1% | 1.0% | 0.9% | 0.4% | 0.5% | 0.8% | |

The mean intra-batch coefficient of variation are substantially less than 10% demonstrating that the pooled process has very low variability between units with a batch. The low CV's clearly indicate that the pooled process creates batches of homogeneous units. This is not the case with CPP units created from single donor APU that are known to have donor-to-donor variability. The improved exemplary pooled CPP process of Example 2 averages out the donor variability, which decreases batch to batch variability of the product. The mean intra-batch CV achieved using the improved exemplary pooled CPP process of Example 2 was less than 5% (4.2%), that of concentration of platelets (/ul) is less than 4% (3.7%), that of thrombin generation ability (IU/10⁶ platelets) is less than 3% (2%), that of CD61-positive microparticles is less than 7% (6.9%), and that of pH is less than 1% (0.8%).

### Example 5. One Year Stability Study of Pooled CPP Product Stored at −20° C. (Transition Temperature Cryopreserved-Product)

Pooled CPP product was manufactured according to the method of Example 2. The stability of the pooled product was tested at different timepoints for a storage period of 1 year. 7 Apheresis Platelet Units (APU) were pooled resulting in 7 units or cryo-vessels of Pooled CPP product. Initially the 7 units were stored at ≤−65° C. freezer for a minimum of 24 hours to form an initial frozen platelet composition. After the initial storage, the cryo-vessels were transferred to a −20° C. freezer for storage to form cryopreserved platelets (transition temperature cryopreserved-product). One unit of the Pooled CPP product (transition temperature cryopreserved-product) was removed at different time points and tested according to the criteria below.

Visual Inspection of cracks, tears, breaks of the unit bag.

Visual Inspection of aggregate free swirling of the product in the bag.

Platelet count per bag.

pH of the product.

Before initiating each test, the product was removed from the freezer, thawed in a 37° C. water bath for 8 minutes, then rehydrated by adding 25 mL of 0.9% saline. Visual inspection of breaks, visual inspection of aggregate free swirling, platelet count per bag, and pH of product are indicators for the release criteria of the product. The platelet count per bag was obtained using either the Beckman Coulter AcT Diff 2 Hematology Particle Analyzer or the Beckman Coulter D×H Hematology Analyzer (Beckman Coulter, beckmancoulter.com). The table below details the results of the tests time points.

TABLE 6

| | Release Criteria | | | |
|---|---|---|---|---|
| | | Aggregate- | | |
| | Visual | free | | Platelet |
| Timepoint | Inspection | Swirling | pH | Count/Bag |
| (Months) | (Pass/Fail) | (Pass/Fail) | (≥6.2) | (≥1.7 × 10¹¹) |
| 1 | Pass | Pass | 7.3 | 2.20E+11 |
| 3 | Pass | Pass | 7.6 | 2.20E+11 |
| 6 | Pass | Pass | 7.7 | 2.20E+11 |
| 9 | Pass | Pass | 7.8 | 2.10E+11 |
| 12 | Pass | Pass | 7.7 | 2.10E+11 |

The results of the stabilization study demonstrate product stability of a transition temperature cryopreserved-product after 12 months of storage at −20° C. The product at all time points passed all release criteria. There were no cracks, breaks, or leaks by visual inspection. Aggregate-free swirl test was confirmed at all time points. The minimum pH and minimum platelet count/bag criteria were achieved at all time points. Although a gradual increase in pH was observed for the 12 months, it did not affect the viability of the product. The platelet count/bag was consistent at the different time points. The findings here confirm the stability of the CPP pooled product (transition temperature cryopreserved-product) at the −20° C. storage temperature for 1 year.

### Example 6: Exemplary CPP Manufacturing Method

The present Example exemplifies one of the methods to produce cryopreserved platelets, such as a single-donor CPP which were then used to compare the cryopreserved platelets obtained by a process comprising transition in freezing temperature as exemplified in this method, also referred to as transition temperature cryopreserved-product with the cryopreserved platelets stored only at −80° C., also referred to as single temperature cryopreserved-product.

Cryopreserved platelets were produced using a single APU per batch of mix ABO and Rh types for all three baches created. The APU was acidified to reach a pH level range of 6.6-6.8 using an Anticoagulant Citrate Dextrose (ACD) solution. The pH level was tested using the Fisher brand Accument XL 250 pH meter (Oakton Instruments). Once the APU was acidified to a pH of 6.6-6.8 a solution of 27% DMSO was used to achieve a final DMSO concentration of 6%. This was done by first determining the platelet count of the APU and then calculating the volume needed to produce the desired number of aliquots. The volume obtained from the APU was then multiplied by a constant of 0.2909 in order to obtain the correct volume of DMSO solution to add. The AcT diff 2 complete blood count (CBC) analyzer (Beckman Coulter, beckmancoulter.com) was used to determine platelet counts for the purpose of this research. Once a 6% DMSO concentration was obtained, the solution of 6% DMSO and platelet rich plasma (PRP) derived from the APU was centrifuged at 1250 g for 20 minutes. Post centrifugation the supernatant plasma/6% DMSO solution was aspirated and reserved for re-suspension. Platelets were then resuspended in approximately half to one mL of plasma/6% DMSO solution, and a concentration test was performed at a 1:20 dilution (50 μL of product+950 μL of PBS) to determine platelet count. The platelet count was then used to calculate the needed volume of plasma/6% DMSO supernatant that was previously aspirated to achieve a platelet concentration of 8,000×10³ platelets/μL. The count was then confirmed before aliquots of the platelets in 6% DMSO (standard formulation) were made. The final product was aliquoted at a volume of 1.5 mL into 5 mL cryogenic vials. The aliquots were transferred to the −80° C. freezer for 48 hours. After the 48-hour period elapsed half of the aliquots produced were transferred to the −20° C. freezer, thereby forming transition temperature cryopreserved-products while the other aliquots remained at −80° C. to form single temperature cryopreserved-products.

### Example 7: Comparing Transition Temperature Cryopreserved-product Stored at −20° C., and Single Temperature Cryopreserved-product Stored at −80° C.

Batch 1, 2, and 3 of each transition temperature cryopreserved-product and single temperature cryopreserved-product prepared by the methods of Example 6 were thawed by transferring the cryotubes to a 37° C. water bath for a period of 8-10 minutes. After thawing 1.5 mL of saline was added to each cryotube. Products were then allowed to rest for a period of 30 minutes with agitation at room temperature. Once the products were allowed to rest, stability and aggregation testing was performed.

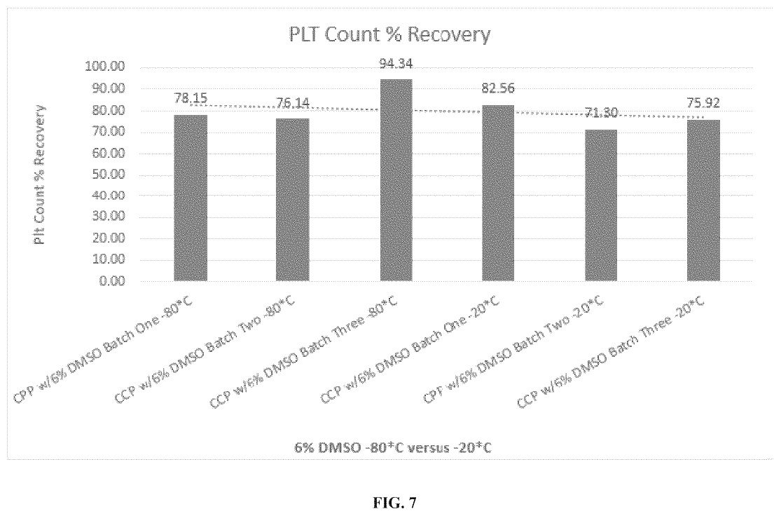

For stability testing the percent platelet recovery was determined. Percent platelet recovery was done using the AcT diff 2 and determining the percent yield. FIG. 7 shows the % recovery of platelets for the batches stored at −80° C. (single temperature cryopreserved-product) and −20° C. (transition temperature cryopreserved-product). The average percent recovery amongst the −80° C. batches (single temperature cryopreserved-product) was 83% versus 77% when stored at −20° C. (transition temperature cryopreserved-product). The goal of the experiment was to obtain a % recovery of greater than 70%, which was obtained by all batches in both storage temperatures. Therefore, the experiment shows similar percent recovery of both storage temperatures, thereby showing that the batches having transition temperature cryopreserved-product (stored at −20° C.) are stable even when stored at a temperature higher than the single temperature cryopreserved-product, which is stored at −80° C.

Aggregation testing was performed using the PAP8 Aggregometer (Bio Data Corporation, biodatacorp.com). Batches 1, 2, and 3 of each product was diluted to 250 k/μl in Hexamethylenetetramine (HMTA) based on the AcT count. In each cuvette 225 μl of diluted sample was added. The cuvettes were then transferred to the incubation wells of the PAP8 Aggregometer. Either 25 μl of collagen (final concentration 10 μg/ml; stock is 100 μg/ml), or 25 μl of TRAP-6 (final concentration 20 μM), or 25 μl of arachidonic acid (AA) (final concentration 500 μg/ml; stock is 5 mg/ml) was added and measurements were taken at 2 minutes. FIG. 8 shows the % aggregation of platelets for the batches stored at −80° C. (single temperature cryopreserved-product) and −20° C. (transition temperature cryopreserved-product), and comparison with apheresis platelets with AA, collagen, and TRAP-6. Included in the aggregation data collection was an apheresis platelet for comparison. A two-tailed t-test was conducted for comparison between the batches of single temperature cryopreserved-product stored at −80° C. against the transition temperature cryopreserved-product stored at −20° C. The AA agonist produced a p value of 0.80, there is no significant difference within the groups. The collagen agonist produced a p value of 0.59, no significant difference. The TRAP-6 produced a p value of 0.45, also with no significant difference. Since there was no statistical difference observed between the batches of both the products, this experiment demonstrated that cryopreserved platelets of transition temperature cryopreserved-product stored at −20° C. exhibits stability and hemostatic properties, such as aggregation, similar to the single temperature cryopreserved-product stored at −80° C., thereby addressing a long-felt need of storing cryopreserved platelets at a temperature higher than −65° C.

### Example 8: Calcein Acetoxymethyl (AM) Cell Membrane Testing

Batch 1, 2, and 3 of each transition temperature cryopreserved-product and single temperature cryopreserved-product prepared by the methods of Example 6 were thawed by transferring the cryotubes to a 37° C. water bath for a period of 8-10 minutes. After thawing 1.5 mL of saline was added to each cryotube. Products were then allowed to rest for a period of 30 minutes with agitation at room temperature. Once the products were allowed to rest calcein AM assay was performed. Calcein AM is a substance that is able to cross the cell membrane and reach the cytosol where Calcein AM gets hydrolyzed by the enzyme esterase to produce fluorescence. Platelets that are intact are able to retain this fluorescence while non-intact platelets do not. Based off the AcT count a 400 μL sample at a concentration of 1,000,000/μL was created. 1 mM Calcein AM stock solution was diluted in 10 μM in HMTA (1:100). Duplicate samples of 1 μL of 10 μM Calcein and 9 μL diluted cryopreserved platelets of each transition temperature cryopreserved-product and single temperature cryopreserved-product were prepared. All samples were incubated away from open light at room temperature for 20 minutes. 990 μL PBS was added to each sample to dilute. 100 μL of each sample was transferred to an individual well in a 96-well plate. Each sample was acquired on the flow cytometer with the following conditions: parameters: FSC, SSC, FB530 (FITC), stop conditions: 30 μL or 30,000 events, FSC-H threshold larger than 1,000, and flow rate: medium. Included in the assay was an aphesis platelet for comparison. FIG. 9 shows the findings performed for the Calcein AM testing. As expected, the apheresis platelets, indicated by “+” depicts a single peak, and were able to retain more of the Calcein AM as compared to transition temperature cryopreserved-product and single temperature cryopreserved-product. It was observed that the transition temperature cryopreserved-product stored at −20° C., depicted by “*” in batches 1, 2, and 3 show a single peak depicting a single population of cells based on the membrane integrity while the single temperature cryopreserved-product stored at −80° C., depicted by “#” show two peaks depicting two populations of cells based on the membrane integrity. Furthermore. it was observed that all the three batches of the transition temperature cryopreserved-product stored at −20° C. had similar medium sized peaks, possibly demonstrating similar levels of membrane integrity across the batches of the transition temperature cryopreserved-product. The observation possibly suggests that there are two kinds of populations in the single temperature cryopreserved-product, a first population that is not able to retain the fluorescence of Calcein AM (See first peak from left of “#” in FIG. 9) possibly because of compromised membrane and a second population (See second peak from left of “#” in FIG. 9) showing higher retention of the fluorescence possibly because they have intact membranes. It was further observed that the single peak of the transition temperature cryopreserved-product of all the three batches (See “*” in FIG. 9) corresponded to the first population of the single temperature cryopreserved-product that shows less retention of Calcein AM. Therefore, not to be limited by theory, it is believed that the single population of the transition temperature cryopreserved-product observed in the Calcein AM assay has platelets with compromised membranes. However, surprisingly, as shown in Example 7, all the batches 1, 2, and 3 of the transition temperature cryopreserved-product showed a platelet recovery of more than 70%. Similarly, as shown in Example 5, Table 6, transition temperature cryopreserved-products prepared by a process as disclosed in Example 2 after being stored at −20° C. even for 12 months were able to pass all the criteria related to aggregate-free swirling, pH, and platelet count.

### Example 9: Characterization of Transition Temperature Cryopreserved-product Stored at −20° C. for Various Time Points

Three batches, W4464-22-000010, W4464-22-000013, and W4464-22-000016 of cryopreserved platelets, such as pooled CPP, were prepared by the process disclosed in Example 2 with freezing done in a transition manner by freezing the obtained cryo-vessels at a temperature of ≤−65° C. for 141 days, 85 days, and 43 days, respectively, to form an initial frozen platelet composition, and storing the initial frozen platelet composition at −20° C. for timepoints of 1 month, 3 months, 6 months, and 12 months to form cryopreserved platelets (transition temperature cryopreserved-product). The data included in this Example is only for one of the batches, for the batch W4464-22-000010 until 12 months. Each cryo-vessel consisted of a volume of 20-35 ml in 6% DMSO. Once the time-point was reached, cryopreserved platelets from a cryo-vessel was thawed and tested for the following parameters:

### Visual Inspection: Pass/Fail

Aggregate-Free: Pass/Fail
pH (≥6.2)
Platelet Count per Bag (≥1.7E+11)
Thrombin Generation Assay (TGA) (20K/μL IU/10⁶Particles)
CD61 Positive Microparticles ×10⁶/μL
lactadherin Positive Particles (%)

### Platelet Count

The platelet count per bag was obtained using either the Beckman Coulter AcT Diff 2 Hematology Particle Analyzer or the Beckman Coulter D×H Hematology Analyzer (Beckman Coulter, beckmancoulter.com).

### TGA Protocol

Fluoroskan Ascent instrument was used to assess the thrombin generation of the products herein.

The concentration of cryopreserved platelets, or platelet derivatives from one of the cryo-vessels of a batch of this Example that needs to be tested was determined by using Beckman Coulter® AcT Diff and AcT Diff 2 Hematology Analyzer (EQU-045) or D×H 520 Hematology Analyzer. After the concentration was determined, the platelets were diluted with Octaplas (solvent detergent treated pooled plasma manufactured by Octapharma) to create serial dilution tubes having platelet concentrations of 352×10³, 160×10³, 80×10³ platelets/μl. For obtaining output for the samples, in-plate dilutions of the platelets of 44×10³ platelets/μl, appropriate volumes of PRP reagent (tissue factor and phospholipids), and Octaplas was added along with the platelets, and a fluorogenic thrombin substrate, FluCa (Stago), into the wells of a 96-well plate. The raw fluorescence intensity of FluCa was measured over time in the Fluoroskan Ascent instrument. The parameters of TGA Count check and TGA particles were generated from the Fluoroskan Ascent instrument. A detailed protocol is provided below.

Frozen platelets were thawed in a water-bath or plasma thawer set to 37° C. for 8 minutes.

After thawing, the platelets were diluted by adding 25 mL of 0.9% saline.

The manufacturer's guidelines were followed in CAT software and the instrument was set up according to manufacturers' guidelines.

PRP reagent containing tissue factor and phospholipids, calibrator, and fluoro-buffer and fluoro-substrate was prepared according to manufacturer's guidelines.

Octaplas and TGA dilution buffer were thawed in 37° C. water bath for 10 minutes.

Thawed Octaplas was added to TGA dilution buffer to create a buffer containing 30% Octaplas.

The 30% Octaplas solution was used to dilute reconstituted cephalin 1:50 to be used as a positive control.

Octaplas was used to dilute thawed platelets to 1584×10³/μL based on the platelet count data from the D×H hematology analyzer.

The 1584×10³/μL dilution of thawed platelets was used to make 325×10³/μL, 160×10³/L, and 80×10³/μL serial dilutions of platelets using Octaplas.

20 μL of PRP reagent was added to each test well, and 20 μL of calibrator was added to each calibration well.

15 μL of the 325×10³/μL, 160×10³/L, and 80×10³/L samples was added to each of their test and calibration wells.

65 μL of Octaplas was added to every test and calibration well.

80 μL of 1:50 cephalin was added to the positive control wells.

The plate was inserted into tray and incubated for 10 minutes at 41° C. After incubation, fluoro-buffer was dispensed and fluoro-substrate mixture (including a fluorescent-labeled peptide, that when cleaved by thrombin, generates a fluorescent signal) was added into active wells.

The plate was read for 75 minutes at 20 second intervals to capture full thrombin generation profile.

### Calculation of CD61 Positive Microparticles and Lactadherin Positive Particles

The method employed in this Example measures the platelet surface CD61 marker and particle size using a flow cytometer (NovoCyte® flow cytometer, ACEA Biosciences, Inc.). Using the flow cytometry method, purity of the composition comprising cryopreserved platelets herein is determined by the concentration of platelet-derived microparticles. The principle of this method is that platelet-derived particles are identified by antibody binding to the CD61 surface marker (Glycoprotein IIIa, a subunit of the GPIIb/IIIa fibrinogen receptor), which is an abundant platelet marker. Within the CD61 positive population, microparticles are distinguished by CD61 mean fluorescence intensity, and particle size (measured using forward scatter). Microparticle concentration (per μL) is calculated by the instrument, which uses a volumetric syringe pump for event acquisition to determine events per unit volume. One of the protocols that was followed to determine the concentration of microparticles, and lactadherin positive particles is provided below. Approximately, 1 ml of cryopreserved platelets after thawing was diluted with 2-fold saline, and was further diluted 100-fold by serial dilutions.

CD61, also known as integrin ˜3 (GPllla) is a glycoprotein subunit of the fibrinogen receptor. GPllb/llla (and other integrin complexes like the vitronectin receptor av ˜3) are found on the surface of platelets. They are found in high levels on normal platelets, CD61 is used in this protocol to identify platelet-derived particles. Allophycocyanin (APC) is a fluorochrome with an excitation maximum at 650 nm and an emission maximum at 660 nm. This fluorochrome is detected in channel R660 on the NovoCyte instrument. Appropriate dilutions of CD61-APC were prepared.

Lactadherin is a milk fat globule-epidermal growth factor-factor 8 protein, binds to phosphatidylserine in a calcium independent manner. Fluorescein isothiocyanate (FITC) is a fluorochrome with an excitation maximum at 495 nm and an emission maximum at 519 nm. This fluorochrome is detected in channel B530 on the NovoCyte instrument. Appropriate dilutions of lactadherin-FITC were prepared.

For test staining, the platelets were stained with CD61-APC, and lactadherin-FITC in tubes in a manner where stain mix was added first to the tubes followed by diluted platelets. All the samples were incubated away from light for about 20 minutes. Each sample was acquired on the flow cytometer with the following conditions: parameters: FSC, SSC, FB530 (for FITC), R660 (for APC) stop conditions: 50 μL or 30,000 events, FSC-H threshold larger than 500, and flow rate: medium (35 μl/min).

For measuring the mean fluorescence intensity of CD61, the gating control specimen was analyzed, and the “CD61+” range gate was adjusted on the APC-H histogram such that it includes between 0.8% and 1.0% of the most fluorescent events. The CD61+gate was copied and pasted from the APC-H histogram of the Gating Control specimen directly to the APC-H histogram of the corresponding Test Sample specimen (composition comprising cryopreserved platelets upon thawing). As a next step, the specimens were analyzed for the size of the population gated with the CD61+range gate. Each Test Specimen was examined and replicated to confirm the “CD61+Microparticles” polygon gate on the APC-H vs FSC-H bivariate plot included the microparticle population, but not the larger platelet population, among CD61+events for each replicate. One of the representative drawings depicting the distinct populations based on the size in APC-H vs FSC-H bivariate plot is shown in FIG. 11. It can be observed that the microparticles having a smaller size can be distinctively separated out from the larger platelet population by applying a polygon gate on the population of particles having a smaller size as compared to the larger platelet population. The population of CD61+microparticles was determined based on the above steps, and was retrieved as an output from the flow cytometer instrument.

Regarding quantification of the lactadherin positive particles, the “Lact+” range gate was adjusted on the FITC-H histogram of the Gating Control specimen such that it includes between 0.8% and 1.0% of the most fluorescent events. The Lact+gate was copied and pasted from the FITC-H histogram of the Gating Control specimen directly to the FITC-H histogram of the corresponding Test specimen (composition comprising cryopreserved platelets upon thawing), to quantify the overall particles positive for lactadherin binding.

Results for the specifications relevant for quality criteria are shown in Table 7

TABLE 7

| Parameter | Specification | Initial | 1 Month | 3 Month | 6 Month | 12 Month |
|---|---|---|---|---|---|---|
| Visual | No Visible | PASS | PASS | PASS | PASS | PASS |
| Inspection | Aggregates | | | | | |
| Swirl Test | Swirling | PASS | PASS | PASS | PASS | PASS |
| | Observed | | | | | |
| Platelet | ≥1.7E+11 | 2.00E+11 | 2.30E+11 | 2.20E+11 | 2.10E+11 | 1.90E+11 |
| Count/Bag | | | | | | |
| pH | ≥6.2 | 6.8 | 7.32 | 7.55 | 7.6 | 7.51 |

Results for the rest of the specifications are shown in Table 8.

TABLE 8

| | | | TGA | | Lactadherin | |
|---|---|---|---|---|---|---|
| | | TGA | (20K/μL, | | Positive | Percentage of |
| W4464-22- | | Count | IU/10⁶ | CD61 Positive | Particles | CD61 Positive |
| 000010 | Count/μL | Check | Particles) | Microparticles/μL | (%) | Microparticles |
| Initial | 3.80E+06 | 557.9 | 1.5 | 3.85E+06 | 81.47% | N/A |
| 1 Month | 4.40E+06 | 465 | N/A* | 9.10E+05 | 99.37% | 21% |
| 3 Months | 4.20E+06 | 465 | N/A* | 7.35E+05 | 99.11% | 18% |
| 6 Months | 4.00E+06 | 537.1 | 0.96 | 6.62E+05 | 88.90% | 17% |
| 12 Months | 3.60E+06 | 569.7 | 0.72 | 4.98E+05 | 96.10% | 14% |
| Min | 3.65E+06 | 465 | 0.7 | 4.98E+05 | 81.47% | N/A |
| Max | 4.42E+06 | 569.7 | 1.5 | 3.85E+06 | 99.37% | N/A |
| Average | 4.01E+06 | 518.94 | 1.1 | 1.33E+06 | 92.99% | N/A |
| SD | 3.14E+05 | 50.6 | 0.4 | 1.42E+06 | 7.70% | N/A |

Results from Table 7 show that the transition temperature cryopreserved-product, i.e. cryopreserved platelets when stored at a temperature of −20° C. for 1 month, 3, 6, and 12 months upon thawing do not show any visible aggregates, and pass the visual inspection of a swirl test, and satisfies the criteria of platelet counts and pH. Therefore, these results demonstrate that the transition temperature cryopreserved platelets herein when stored at a temperature of −20° C. for at least 12 months exhibit the properties shown in Table 7.

Further, results shown in Table 8 demonstrate that in spite of storing at a temperature of −20° C. for at least 12 months, cryopreserved platelets upon thawing retain thrombin generation ability. Also, it can be observed that the cryopreserved platelets upon thawing display a CD 61 positive microparticle content in the range of 21% (1 month) to 14% (12 months). Accordingly, it is observed that the percentage of CD 61 positive microparticles gradually decreases over the storage time. Also, cryopreserved platelets herein demonstrate lactadherin positivity in the range of 80-99.5%.

### Example 10: Characterization of the Stability of Pooled CPP Product at Different Post-thaw Timepoints

The post-thaw stability of a cryopreserved platelet composition comprising frozen activated platelets, prepared in accordance with the process disclosed in Example 2 herein, also referred herein as “pooled CPP product”, or “pooled CPP” in this and foregoing examples was tested at various time points. Three batches—Batch 1, Batch 2, and Batch 3 of the cryopreserved platelet composition comprising frozen activate platelets in cryo-vessels were prepared by the process disclosed in Example 2, and were stored at ≤−65° C. for approximately 9 months before being tested. Each batch underwent quality control characterization and was determined to be suitable for use prior to initiation of the tests. Three cryo-vessels from each of the three batches were randomly selected for inclusion. Each cryo-vessel was tested in triplicates. The composition of cryo-vessels that were randomly selected from each of the three batches were thawed according to the below process:
The cryo-vessels were removed from the freezer and placed into a plasma thawer overwrap;
The cryo-vessels were then placed into a 37° C. plasma thawer with agitation for 8 minutes; and
The cryo-vessels were removed from plasma thawer and rechecked for cracks, breaks, or leakage.
The cryopreserved platelet composition of each of the cryo-vessels was resuspended with 25 mL of 0.9% saline (previously stored at room temperature) to obtain the thawed-resuspended composition comprising thawed platelet particles and visual inspection was performed to confirm that no aggregates were present before testing the composition. At specified timepoints, 0 hours (TO), 4 hours (T4), 8 hours (T8), and 24 hours (T24), a 10-15 mL aliquot was prepared by slowly drawing the thawed-resuspended composition comprising thawed platelet particles into a syringe, then the aliquot was deposited into a clean conical tube for testing. The timepoints are the post-thaw time durations at which the composition was analyzed for its stability by assessing the parameters as disclosed below. At the specified timepoints the samples comprising the compositions were tested for the following parameters according to the process disclosed in Example 9. After thawing and resuspending the cryopreserved platelet composition, the thawed-resuspended compositions were stored at room temperature for different timepoints of 0 hours (TO), 4 hours (T4), 8 hours (T8), and 24 hours (T24). The sample for TO analysis was taken for testing immediately after resuspending with the saline without any storing period.

Platelet count per cryo-vessel, such as a cryo-bag (>1.7E+11)

pH of product (>6.2)

Thrombin Generation (IU/10⁶particles)

CD61 positive Microparticles×10⁶/μL

Lactadherin positive particles (%)

The results of platelet count are shown in Table 9.

TABLE 9
Total platelet counts

| Time | Batch 1 | | Batch 2 | | Batch 3 | | Overall | |
|---|---|---|---|---|---|---|---|---|
| Point | Mean | SD | Mean | SD | Mean | SD | Mean | SD |
| T0 | 2.21 × 10¹¹ | 1.02 × 10¹⁰ | 2.52 × 10¹¹ | 4.75 × 10¹⁰ | 2.26 × 10¹¹ | 7.37 × 10⁹ | 2.33 × 10¹¹ | 1.64 × 10¹⁰ |
| T4 | 2.24 × 10¹¹ | 1.55 × 10¹⁰ | 2.27 × 10¹¹ | 8.06 × 10⁹ | 2.19 × 10¹¹ | 9.00 × 10⁹ | 2.24 × 10¹¹ | 3.98 × 10⁹ |
| T8 | 2.05 × 10¹¹ | 1.06 × 10¹⁰ | 2.25 × 10¹¹ | 1.24 × 10¹⁰ | 2.12 × 10¹¹ | 7.12 × 10⁹ | 2.14 × 10¹¹ | 1.02 × 10¹⁰ |
| T24 | 2.01 × 10¹¹ | 6.5 × 10⁹ | 1.98 × 10¹¹ | 1.06 × 10¹⁰ | 1.90 × 10¹¹ | 1.13 × 10¹⁰ | 1.96 × 10¹¹ | 5.60 × 10⁹ |

It can be observed that at all timepoints the platelet count was above the minimum criteria as shown in Table 9. Therefore, in terms of the platelet count, the cryopreserved platelet composition as disclosed herein remains stable at each timepoint, and at even 24 hours after thawing.

Further, for assessing the pH of the composition at different post-thaw timepoints, the pH of the samples was as taken at every timepoint as well. The lower limit for pH stability was set to 6.2 for all cryo-vessels. The results of pH at different timepoints are shown in Table 11.

TABLE 10
pH

| Time | Batch 1 | | Batch 2 | | Batch 3 | | Overall | |
|---|---|---|---|---|---|---|---|---|
| Point | Mean | SD | Mean | SD | Mean | SD | Mean | SD |
| T0 | 6.75 | 0.067 | 6.79 | 0.035 | 6.84 | 0.038 | 6.79 | 0.042 |
| T4 | 6.61 | 0.006 | 6.61 | 0.006 | 6.73 | 0.010 | 6.65 | 0.067 |
| T8 | 6.85 | 0.061 | 6.76 | 0.036 | 6.88 | 0.020 | 6.83 | 0.062 |
| T24 | 6.68 | 0.093 | 6.85 | 0.025 | 6.91 | 0.032 | 6.82 | 0.120 |

It can be observed that for all the compositions, the pH at each timepoint remained well above the lower limit (pH 6.2) at all timepoints. Therefore, it can be observed that the cryopreserved platelet composition comprising frozen activated platelets as disclosed herein maintained stability in terms of pH even at 24 hours upon thawing. Further, referring to Table 10, it can be observed that the mean pH of the compositions from cryo-vessels across the three batches was maintained within +/−0.05 pH units after 24 hours post-thawing as compared to the timepoint immediately after thawing (TO). Referring to the change in pH at each timepoints, it can be observed that the mean pH of the compositions from cryo-vessels across the three batches was maintained within +/−0.1 pH unit at 4 hours (T4), and 8 hours (T8) after thawing as compared to T0. Therefore, the cryopreserved platelet composition comprising frozen activated platelets disclosed herein, maintains stability in terms of pH at timepoints within and at 24 hours after thawing.

Further, thrombin generation ability (TGA), CD61 positive microparticles concentration, and phosphatidylserine positivity, in terms of lactadherin binding were determined at TO and T8 for the compositions from the cryo-vessels across the three batches.

Thrombin generation ability (TGA) was represented as international units per one million particles (IU/10⁶). Tables 11, 12, and 13 show the thrombin generation ability (TGA), CD61 microparticle concentration, and phosphatidylserine positivity, in terms of lactadherin binding, respectfully at TO and T8.

TABLE 11
TGA

| | Batch 1 | | Batch 2 | | Batch 3 | | Overall | |
|---|---|---|---|---|---|---|---|---|
| Time | (IU/10⁶ particles) | | (IU/10⁶ particles) | | (IU/10⁶ particles) | | (IU/10⁶ particles) | |
| Point | Mean | SD | Mean | SD | Mean | SD | Mean | SD |
| T0 | 1.76 | 0.02 | 1.83 | 0.09 | 1.84 | 0.08 | 1.81 | 0.04 |
| T8 | 1.63 | 0.03 | 1.74 | 0.08 | 1.75 | 0.01 | 1.70 | 0.07 |

It can be observed that the thrombin generation ability slightly decreased from T0 to T8, but the decrease was not significant. Further, it can be observed that the composition comprising frozen activated platelets has the ability to generate thrombin 8 hours post-thaw.

Table 12 shows the amount of CD61 positive microparticles per microliter at T0 and T8.

TABLE 12
Microparticles

| | Batch 1 | | Batch 2 | | Batch 3 | | Overall | |
|---|---|---|---|---|---|---|---|---|
| Time | (CD61⁺/μl) | | (CD61⁺/μl) | | (CD61⁺/μl) | | (CD61⁺/μl) | |
| Point | Mean | SD | Mean | SD | Mean | SD | Mean | SD |
| T0 | 5.85 × 10⁶ | 4.87 × 10⁵ | 1.07 × 10⁷ | 3.54 × 10⁵ | 6.96 × 10⁶ | 2.05 × 10⁵ | 7.82 × 10⁶ | 2.52 × 10⁶ |
| T8 | 6.26 × 10⁶ | 4.08 × 10⁵ | 8.86 × 10⁶ | 3.45 × 10⁵ | 7.51 × 10⁶ | 4.26 × 10⁵ | 7.54 × 10⁶ | 1.30 × 10⁶ |

As shown on Table 12, the mean microparticle concentration at T8 (7.54×10⁶) is similar to T0 (7.82×10⁶). Further, the concentration of microparticles is below 10×10⁶/μl. Therefore, it is demonstrated that the composition comprising frozen activated platelets as disclosed herein maintains stability in terms of microparticle concentration over time after thawing.

Table 13 shows the percent positivity for lactadherin binding at T0 and T8.

TABLE 13
Lactadherin

| Time | Batch 1 (%) | | Batch 2 (%) | | Batch 3 (%) | | Overall (%) | |
|---|---|---|---|---|---|---|---|---|
| Point | Mean | SD | Mean | SD | Mean | SD | Mean | SD |
| T0 | 85.93 | 1.32 | 92.19 | 0.77 | 88.88 | 0.91 | 89.00 | 3.13 |
| T8 | 86.53 | 0.99 | 92.42 | 0.64 | 88.48 | 0.94 | 89.14 | 3.00 |

It can be observed that the mean positivity for lactadherin binding at T0 and T8 post-thawing are similar to each other. Therefore, it is demonstrated that the composition comprising frozen activated platelets as disclosed herein maintains stability in terms of phosphatidylserine positivity as measured using lactadherin binding over time after thawing.

According to the post-thaw stability experiments on the different batches, the composition comprising frozen activated platelets, or the CPP product herein passes the release criteria metrics of platelet counts (>1.7 ×10¹¹) and pH (>6.2) at all timepoints with similar values for T0 and T8. Furthermore, thrombin generation ability (TGA), CD61 positive-microparticle concentrations, and lactadherin binding positivity demonstrated minimal changes from T0 and T8. Thus, the results support the use of the cryopreserved platelet compositions comprising frozen activated platelets herein stored at ambient temperatures after thawing for up to 24 hours.

### Example 11: Pooled CPP Product is Superior to Single Donor Cryopreserved Platelet Product

A single donor cryopreserved platelet composition was performed according to the process disclosed in Example 1 herein. A pooled cryopreserved platelet composition comprising frozen activated platelets, in cryo-vessels, (referred herein as “pooled CPP product”, or “pooled CPP”) was produced according to the process disclosed in Example 2 herein. The required units of the single donor cryopreserved platelet composition (single-donor CPP), and one cryo-vessel each from 88 different batches of the cryo-vessels comprising pooled CPP were removed from the freezer and placed into a plasma thawer overwrap. The units and the cryo-vessels were then placed into a 37° C. plasma thawer with agitation for 8 minutes. The units and the cryo-vessels were removed from the plasma thawer and rechecked for cracks, breaks, or leakage. The composition of each of the units and the composition of each of the cryo-vessels were diluted with 25 mL of 0.9% saline and visual inspection was performed to confirm that no aggregates were present before testing the compositions of the units and the cryo-vessels. The composition of each unit and the composition in each cryo-vessel were tested for the following parameters according to the process disclosed in Example 9 herein.

Platelet count per cryo-vessel, or bag such as a cryobag (>1.7E+11)

### Platelet Concentration

pH of composition (>6.2)

Thrombin Generation (IU/10⁶particles)

CD61 positive Microparticles×10⁶/μL

Lactadherin positive particles (%)

The results of platelet count, platelet concentration, thrombin generation, CD61-positive microparticle concentration, lactadherin positivity, and pH are shown in table 14.

TABLE 14

| | | | | Pooled CPP Product | | |
|---|---|---|---|---|---|---|
| | | | | n = 88 batches | | |
| | | | | (1 cryo-vessel | | |
| | Single-Donor CPP | | | tested from | | |
| Product/Test | N = 20 units | | | each batch) | | |
| Sample Size | Mean | Range | % CV | Mean | Range | % CV |
| Total Platelets | 3.1 | 2.2-3.6 | 12% | 2.2 | 1.8-2.7 | 8% |
| in a | | | | | | |
| Unit/cryo- | | | | | | |
| vessel | | | | | | |
| (×10\hat{ }11) | | | | | | |
| Platelet Conc. | 5.9 | 3.7-6.6 | 14% | 4.3 | 3.5-5.1 | 8% |
| (×10\hat{ }6 | | | | | | |
| plts/μL) | | | | | | |
| Thrombin | 1.3 | 1.1-1.6 | 12% | 1.4 | 0.9-2.2 | 19% |
| Generation | | | | | | |
| (IU/10\hat{ }6 | | | | | | |
| plts) | | | | | | |
| CD61 + | 6.3 | 2.7-13.0 | 36% | 4.0 | 0.9-6.9 | 24% |
| Microparticle | | | | | | |
| Conc. | | | | | | |
| (10\hat{ }6 | | | | | | |
| MP/μL) | | | | | | |
| Lactadherin | 77 | 64-85 | 7% | 81 | 66-88 | 5% |
| Positivity (%) | | | | | | |
| pH | 6.65 | 6.40-6.86 | 2% | 6.58 | 6.2-6.8 | 2% |

It can be observed from Table 14 that the pooled CPP product, or the composition comprising frozen activated platelets, prepared as per the process of Example 2 herein displays a superior homogeneity in terms of total platelets in a cryo-vessel, platelet concentration, CD61-positive microparticle concentration, and lactadherin positivity where the % CV across 88 batches are lower than the % CV of the single-donor CPP product prepared as per the process of Example 1 herein across 20 units. This indicates less variability of the pooled CPP product in comparison to the single donor CPP product. It can also be observed that the mean concentration of CD61-positive microparticles across 88 batches of pooled CPP prepared as per the process of Example 2 is 4×10⁶/μl, that is lower as compared to the mean concentration of CD61-positive microparticles across 20 units of the single-donor CPP. Further, the mean lactadherin positivity and the mean thrombin generation ability of the pooled CPP prepared as per the process of Example 2 is higher as compared to the single-donor CPP, thereby showing that the cryopreserved platelet composition comprising frozen activated platelets herein is activated to a higher level as compared to the single-donor CPP. Also, it can be observed that the better activation state of the pooled CPP is when the mean platelet concentration, and the total platelets are lower in the pooled CPP as compared to the single-donor product.

Furthermore, statistical analysis of the two products was performed for CD61+microparticle content as well as the ratio of CD61+microparticle to platelet concentration. Table 15 shows the statistical analysis results of CD61+microparticle (particles/μl) for the single donor CPP product and the pooled CPP product.

TABLE 15

CD 61+ Microparticles / μl

| | Single Donor | Pooled |
|---|---|---|
| | CPP Product | CPP Product |
| Count | 20 | 88 |
| Min | 2.70 × 10⁶ | 9.27 × 10⁵ |
| Mean | 6.30 × 10⁶ | 4.00 × 10⁶ |
| Max | 1.30 × 10⁷ | 6.93 × 10⁶ |
| SD | 2.3 × 10⁶ | 9.62 × 10⁵ |
| CV (%) | 36.5 | 24.0 |
| Range (max-min) | 9.9 × 10⁶ | 6.00 × 10⁶ |
| F-test for CV | 1.43 × 10⁻⁸ | Significant |
| | | (p < 0.05) |
| t-test for means | 2.79 × 10⁻⁴ | Significant |
| | | (p < 0.05) |

From table 15, firstly it can be observed that there are significantly less CD61+microparticles/μl in the pooled CPP product (mean value of 4.00×10⁶) relative to the single donor CPP product (mean value of 6.30×10⁶). A t-test was performed for the means of the CD61+microparticle concentration of the two products with a p=2.8 ×10⁻⁴. This p value indicates a significant difference between the two mean values. Secondly, it can be observed that the pooled CPP product was statistically less variable than the single donor CPP product as evidenced by a CV of 24.0% versus 36.5% and an F-test for variance of 1.43 ×10⁻⁸. Also, the maximum concentration of CD61+microparticles in the pooled CPP product herein observed across 88 batches is 6.93×10⁶/μl, which is much less than that for the single donor CPP product across 20 units (1.30×10⁷/μl).

Table 16 shows statistical analysis of the two products for the ratio of CD61+microparticle concentration to platelet concentration.

TABLE 16

CD 61+ Microparticle / Platelet Cell
(thawed platelet particles) Ratio

| | Single Donor | Pooled CPP |
|---|---|---|
| | CPP Product | Product |
| Count | 20 | 88 |
| Min | 0.40 | 0.23 |
| Mean | 1.1 | 0.93 |
| Max | 2.4 | 1.5 |
| SD | 0.43 | 0.20 |
| CV (%) | 39.6 | 21.9 |
| Range (max-min) | 1.9 | 1.3 |
| % > 1.0 (Ratio) | 55.0 | 33.6 |
| F-test for | 1.00 × 10⁻⁵ | Significant |
| variance | | (p < 0.05) |
| t-test for | 0.11 | Not Significant |
| means | | |
| Chi-Square for | 0.039 | Significant |
| Ratio > 1.0 | | (p < 0.05) |

While the means of the ratios between the groups were not significant, 1.1 versus 0.93, P >0.1, there was a statistically significant difference between the variance of the ratios for the two groups favoring the pooled CPP product as evidenced by a CV of 39.6% for the single donor CPP product versus 2190 for the pooled CPP product (p<0.0001). Moreover, there was a statistically significant greater percentage of units having a microparticle to thawed platelet particle ratio that exceeds 1.0, favoring the pooled CPP product (33.6% versus 55.0% o) that was statistically significant as judged by Chi-squared analysis (p<0.05).

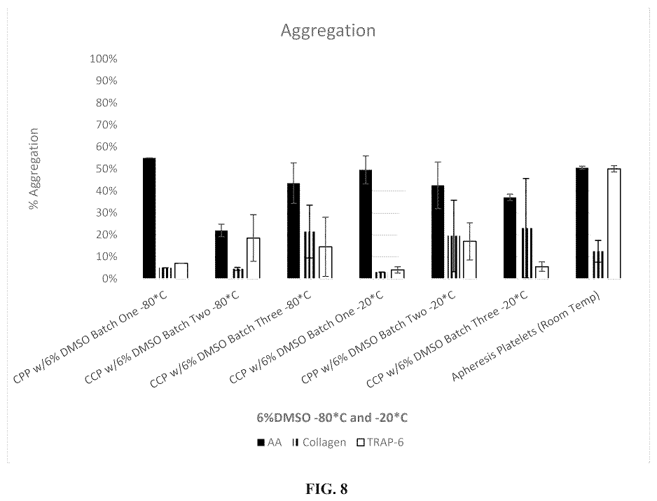

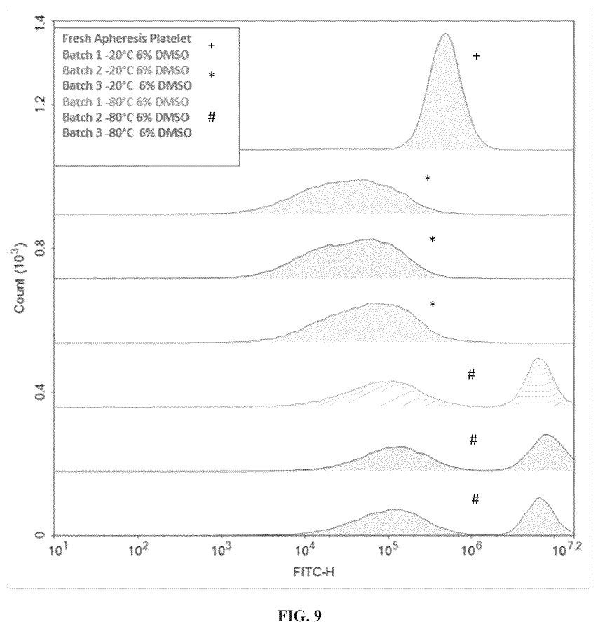

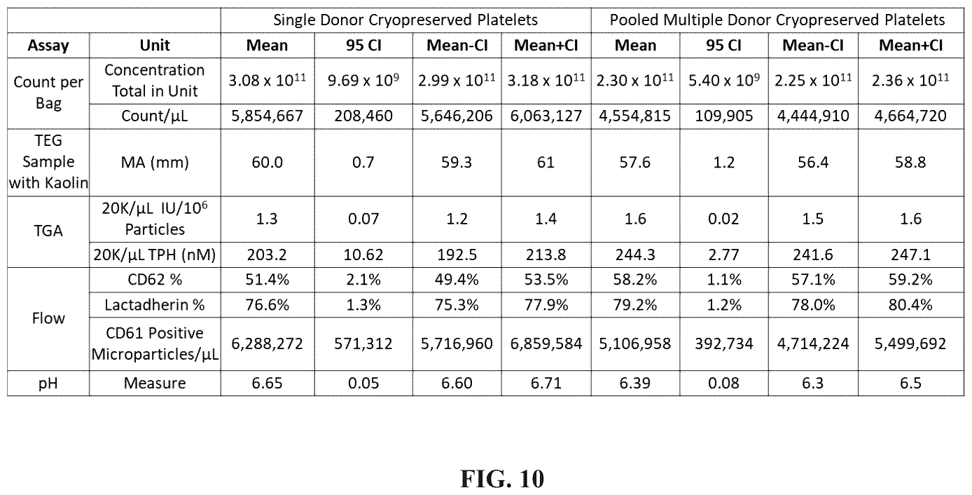

Another analysis was performed using 20 units of single-donor CPP, and one cryo-vessel each from 27 different batches of the cryo-vessel comprising the cryopreserved pooled platelet composition prepared in accordance with Example 2 herein, also referred herein as pooled CPP product. In addition to the attributes demonstrated in Table 14, this analysis demonstrated flow cytometric data for CD62 percent positivity and lactadherin percent positivity in the products. Table 17 shows the differences between single-donor CPP and pooled CPP products for the various attributes. An expanded version of the data is shown in FIG. 10 where the statistical analysis of 95% confidence interval (95CI) of mean values is included.

TABLE 17

| | | Single Donor CPP Product (n = 20) | | | Pooled CPP Product (n = 27*) | | |
|---|---|---|---|---|---|---|---|
| Assay | Unit | Mean | SD | Range | Mean | SD | Range |
| Count per | Concentration | 3.1E+11 | 3.7E+10 | 2.2E+11-3.6E+11 | 2.3E+11 | 2.4E+10 | 1.9E+11-2.7E+11 |
| Bag | Total in Unit | | | | | | |
| | Count/μL | 5.9E+06 | 8.1E+05 | 3.7E+06-6.6E+06 | 4.6E+06 | 4.9E+05 | 3.9E+06-5.4E+06 |
| TGA | 20K/μL IU/10⁶ | 1.3 | 0.2 | 1.1-1.6 | 1.6 | 0.0 | 1.5-1.6 |
| | Particles | | | | | | |
| | 20K/μL TPH | 203.2 | 24.2 | 165.9-246.5 | 244.3 | 7.2 | 230.2-254.3 |
| | (nM) | | | | | | |
| Flow | CD62% | 51.4% | 8.2% | 38.2%-69.0% | 58.2% | 4.9% | 51.4%-65.9% |
| | Lactadherin % | 76.6% | 5.1% | 63.6%-85.0% | 79.2% | 5.4% | 67.0%-87.2% |
| | CD61 Positive | 6.3E+06 | 2.3E+06 | 2.7E+06-1.3E+07 | 5.1E+06 | 1.8E+06 | 2.9E+06-8.5E+06 |
| | Microparticles/μL | | | | | | |
| pH | Measure | 6.65 | 0.13 | 6.40-6.86 | 6.39 | 0.22 | 5.94-6.73 |

Table 17 above shows the data for three replicates taken for the count per bag and flow markers, and single measurements for all other attributes (denoted by “*”).

It can be observed from Table 17 that the pooled CPP product herein is more activated in terms of higher mean percentage positivity for CD62, and phosphatidylserine, when measured using lactadherin binding, of 58.2%, and 79.2%, respectively, as compared to 51.4%, and 76.6%, respectively for single donor CPP product. Referring to CD61 positive microparticle concentration, it can be observed that the pooled CPP product herein has less microparticles in terms of the mean of 5.1×10⁶/μl, as compared to the mean concentration of 6.3×10⁶/μl in the single donor CPP product. Further, regarding thrombin generation activity, it can be observed that the pooled CPP product has a higher mean of thrombin generation activity in terms of TPH, and IU/10⁶/particles as compared to the single donor CPP product. Furthermore, it can be observed that in spite of having a lower mean concentration, and mean total platelet particles as compared to the single donor CPP product, the pooled CPP product exhibits a higher mean of thrombin generation activity.

An expanded version of Table 17 is shown in FIG. 10 where the statistical analysis of 95% confidence interval (95CI) of mean values is included. The 95CI data demonstrate that the differences in mean values discussed in the above paragraph, are statistically significant at a 95% confidence interval, since there is no overlap in the values of 95CI of mean values for all the attributes shown in Table 17 and FIG. 10. For example, considering the 95CI of the mean for the concentration of CD61-positive microparticles, the pooled CPP product across 27 batches herein has a lower mean concentration that does not overlap with that of single-donor CPP. In terms of the activation markers, the mean values for CD62% positivity, and phosphatidylserine positivity, when measured using lactadherin binding across 27 pooled CPP batches herein, are higher and have 95CI intervals that do not overlap with those of the single-donor CPP, thereby demonstrating that more particles in the cryo-vessels of pooled CPP product are in an activated state (i.e., are frozen activated platelets), as compared to cryo-vessels of the single-donor CPP.

### Example 12: Thrombin Generation Assay for Pooled CPP Product and Room Temperature Platelets (RTP)

Single donor room temperature platelets, also referred to herein as room temperature platelets (RTP) were purchased from LifeShare Blood Center. The RTP can also be referred to as apheresis platelet units (APU). Cryopreserved platelet composition comprising frozen activated platelets, in cryo-vessels, was developed according to the process disclosed in Example 2 herein (referred herein as “pooled CPP product” or “pooled CPP”). Cryo-vessels comprising pooled CPP were removed from freezer and placed into a plasma thawer overwrap. The cryo-vessels comprising pooled CPP were then placed into a 37° C. plasma thawer with agitation for 8 minutes. The cryo-vessels comprising pooled CPP were removed from plasma thawer and rechecked for cracks, breaks, or leakage. Each cryopreserved platelet composition of each cryo-vessel was diluted with 25 mL of 0.9% saline and visual inspection was performed to confirm that no aggregates were present before testing the product.

A concentration of 60 mM GPRP (BACHEM, bachem.com), thrombin (Sigma Aldrich, sigmaaldrich.com) and Octaplas® (Octophrama, octopharma.com) were thawed according to manufacturer's guidelines. CLARIOstar^{Plus} was used to perform the Thrombin generation assay (TGA). A master mix of Octaplas, PRP reagent (Diagnostica Stago, Cat. No. 86196), and GPRP was prepared in a ratio of 14:3:1, respectively. Flu substrate and Fluo-buffer were combined according to manufacturer's guidelines to create FluCa (Diagnostica Stago, stago.com). Master mix and FluCa solution were placed in the 37° C. air incubator with agitation for 45±5 minutes. The following dilutions of pooled CPP product and RTP were prepared in duplicates in TGA dilution buffer: 1:256, 1:512, and 1:1024. A volume of 10 μl of each sample was dispensed into selected wells of a 96-well plate (Corning, coming.com) by reverse pipetting. TGA Dilution Buffer for thrombin was used in the thrombin standard ladder. CLARIOstar^{Plus} injectors 1 and 2 were primed with master mix and FluCa solution with 250 μl, then the injector was placed in the active position. The CLARIOstar^{Plus} TGA program was loaded with parameters for TGA assay with injectors set to add 90 μl of master mix and 40 μl of FluCa solution to each well. Plate was inserted to be read. This protocol was repeated for 5 different lots of pooled CPP product and 7 units of RTPs.

FIG. 12 shows the peak thrombin of the pooled CPP product and RTP. It can be observed that the peak thrombin of the pooled CPP product is substantially higher than that of RTP. For example, as per FIG. 12, the peak thrombin of RTP was less than 5 NIH units/10⁶ particles, whereas the peak thrombin of the pooled CPP product was about 15 NIH units/10⁶ particles. This example demonstrates that the thrombin generation ability of the frozen activated platelets in the cryopreserved platelet compositions (pooled CPP product) as disclosed herein is about 3-fold higher than that of RTP.

### Example 13: Flow Cytometry Surface Marker Characterization of Pooled CPP product Versus Room Temperature Platelets (RTP)

Single donor room temperature platelets, also referred to herein as room temperature platelets (RTP) were purchased from LifeShare Blood Center. The RTP can also be referred to as apheresis platelet units (APU). Cryopreserved platelet composition comprising frozen activated platelets, in cryo-vessels, was developed according to the process disclosed in Example 2 herein, referred herein as “pooled CPP product” or “pooled CPP”. Cryo-vessels comprising the pooled CPP product were removed from freezer and placed into a plasma thawer overwrap. The cryo-vessels comprising pooled CPP product were then placed into a 37° C. plasma thawer with agitation for 8 minutes. The cryo-vessels comprising pooled CPP were removed from plasma thawer and rechecked for cracks, breaks, or leakage. Each cryopreserved platelet composition of each cryo-vessel was diluted with 25 mL of 0.9% saline and visual inspection was performed to confirm that no aggregates were present before testing the product.

RTP and pooled CPP product counts were determined by diluting RTP and pooled CPP product samples 1:500 in phosphate-buffered saline (PBS) and then acquiring events on the Quanteon flow cytometer (Agilent, agilent.com). Counts were determined using a bivariant logarithmic size density plot of forward scatter (FSC-Hvs (SSC-H) in a known volume of sample. Based on counts, pooled CPP product and RTP were normalized to 100 k cells/μl in HEPES modified Tyrode's buffer with albumin (HMTA) before staining. 1 million total cells (10 μL) of either pooled CPP product or RTP were stained and incubated for 20 minutes in final sample volumes of 30 μL containing the antibody of interest and HMTA to fill the remaining volume. Staining was performed on pooled CPP product and RTP across 10 samples measured in duplicate for each group.

All antibodies volumes/concentrations used in this characterization work was first obtained through antibody titration testing using isotype controls on pooled CPP product and RTP in similarly prepared sample staining volumes. Final concentrations used was as follows: 17.7 μg/mL of vWF antibody (Novus Biologicals, novusbio.com), 267 μg/mL at 1:3 mix of bovine lactadherin-FITC labeled (Prolytix, goprolytix.com) and bovine lactadherin (Prolytix, goprolytix.com), 16.7 μg/mL APC anti-human CD63 antibody (Biolegend, biolegend.com), 0.45 μg/mL PE mouse anti-human CD62P (BD Biosciences, bdbiosciences.com), 6 μL of FITC mouse anti-human CD42b (BD Biosciences, bdbiosciences.com), 2 μg/mL of PE mouse anti-human platelet GPVI (BD Biosciences, bdbiosciences.com), 6 μL PE mouse anti-human CD49b (BD Biosciences, bdbiosciences.com), 33.3 μg/mL of Alexa Fluor 546 anti-thrombospondin 1 antibody (Santa Cruz, scbt.com), 5 μL FITC anti-fibrinogen antibody (Abcam, abcam.com) prediluted 1:7 in HMTA, 5 μL anti-fibrinogen antibody, receptor-induced binding site (RIBS) clone 9F9 (BioCytex, biocytex.com). All samples were then diluted 3:50 (v/v) in PBS and mean fluorescence intensity (MFI) was acquired on the Quanteon Flow Cytometer (Agilent, agilent.com).

FIG. 13 shows the MFI of the different surface markers of pooled CPP product and RTP. It can be observed that the MFI for surface markers GPVI, CD49, and CD42b is lower for pooled CPP product. On the other hand, the MFI for activation markers vWF, phosphatidylserine (lactadherin), and fibrinogen is significantly higher (p<0.05), and MFI for activation markers Thrombospondin, CD62P, CD63, and CD61 is equal to or higher, for pooled CPP product as compared to RTP. This Example demonstrates that the frozen activated platelets in the cryopreserved platelet compositions disclosed herein are more activated as compared to the starting material, such as RTP as shown here.

### Example 14: Aggregation Response of Pooled CPP Product and Fresh Platelets

Cryopreserved platelet composition comprising frozen activated platelets, in cryo-vessels was developed according to the process disclosed in Example 2 herein, referred herein as “pooled CPP product” or “pooled CPP”. The cryo-vessels comprising pooled CPP were removed from freezer and placed into a plasma thawer overwrap. The cryo-vessels comprising pooled CPP were then placed into a 37° C. plasma thawer with agitation for 8 minutes. The cryo-vessels comprising pooled CPP were removed from plasma thawer and rechecked for cracks, breaks, or leakage. Each cryopreserved platelet composition of each cryo-vessel was diluted with 25 mL of 0.9% saline and visual inspection was performed to confirm that no aggregates were present before testing the product.

Whole blood was collected from healthy donors in vacutainer ACD blood collection tubes (Becton Dickinson, bdbiosciences.com) centrifugated at 180×g for 20 minutes to collect platelet rich plasma (PRP). To obtain washed pooled CPP and washed PRP, 6 μl of 1M citric acid was added to 1 ml of PRP and 1 ml of pooled CPP product, then centrifuged at 1000×g for 10 minutes and resuspended in HMTA. This step was repeated 5 times. The aggregation of pooled CPP product and fresh platelets was screened in the presence of different agonists using the PAP-8 Platelet Aggregometer, by using the washed pooled CPP, and washed PRP prepared as disclosed herein. Washed pooled CPP and washed platelets count was 300,000/μl using the Beckman Coulter AcT Diff 2 Hematology Analyzer in duplicate to reach 7 ml and 5 ml, respectively. These samples were prepared immediately prior to running on the PAP-8E Platelet Aggregometer.

The PAP-8E Platelet Aggregometer was turned on and samples were incubated with stirring for 10 minutes at 37° C. with the different agonists for all aggregation experiments. The samples with HMTA were used as a control for agonists used in aggregation. The following agonists were used to induce aggregation in samples: 10 μg/ml collagen (Helena Biosciences, Helena-biosciences.com), 10 U/ml thrombin (Helena Biosciences, Helena-biosciences.com), 10 μg/ml arachidonic acid (Helena Biosciences, Helena-biosciences.com), and 25 μM TRAP-6 (Helena Biosciences, Helena-biosciences.com). A volume of 225 μl of the samples were added to cuvettes by reverse pipetting and inserted in the channels. Cuvettes were transferred from the warming wells to the testing wells. The test was started and 25 μl of each agonist was added to the appropriate cuvettes when the timer for the channel reached 8:00 minutes. HMTA was added in place of the agonist to the first channel in each module. Once measurements were completed for each agonist, the samples were transferred to a microcentrifuge tube and AcT count was taken in the presence of different agonists.

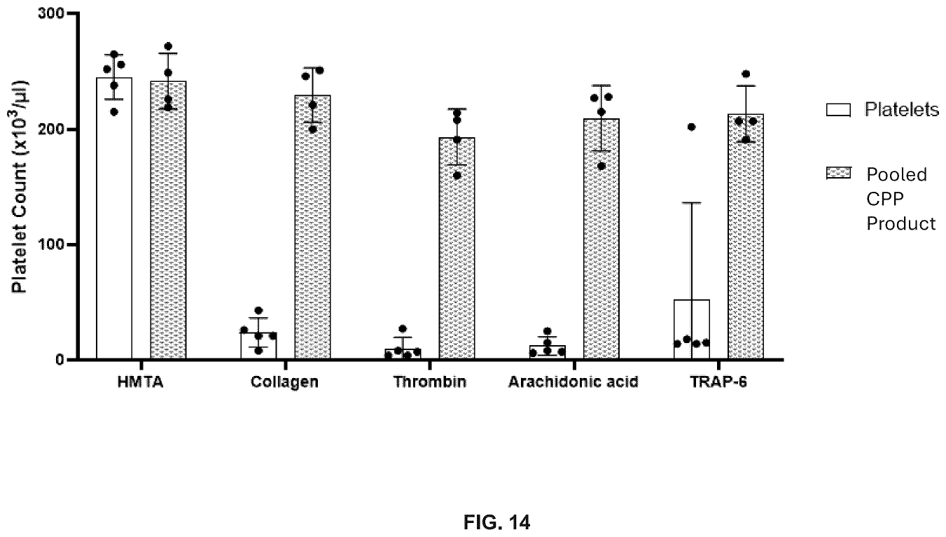

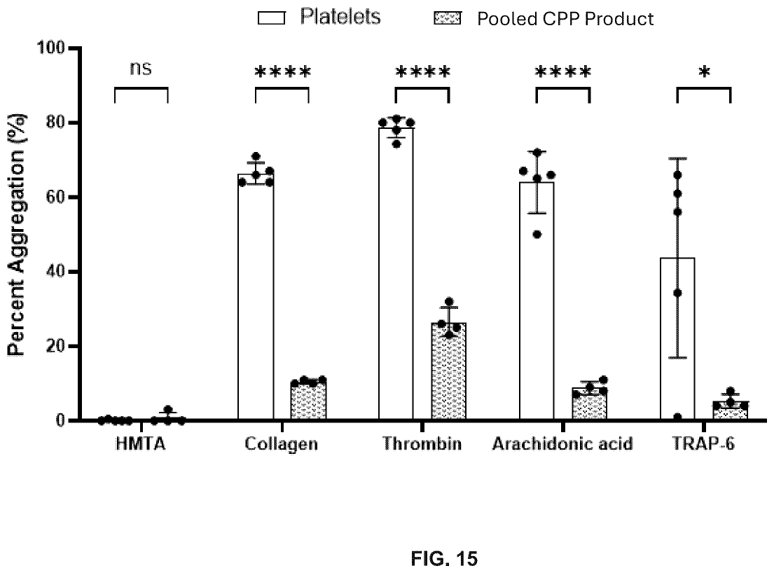

FIG. 14 and FIG. 15 show the response of pooled CPP product, and fresh platelets (denoted as “platelets”) to the different agonists. As can be observed, the pooled CPP product does not significantly aggregate in response to any of the agonist based on the percent aggregation (shown in FIG. 15) and the high platelet count (shown in FIG. 14) that is similar to the HMTA control as compared to the aggregation response by fresh platelets. The pooled CPP product herein demonstrates, as shown by platelet count in FIG. 14, less than 5-fold aggregation for collagen, thrombin, and arachidonic acid as compared to the aggregation shown by fresh platelets, and less than 2-fold aggregation for TRAP-6 as compared to that of fresh platelets. The pooled CPP product herein demonstrates, as shown by percent aggregation in FIG. 15, less than 20% aggregation for collagen, arachidonic acid, and TRAP-6, and about 25-35% aggregation for thrombin, as compared to more than 50% aggregation shown by fresh platelets for all of the tested agonists.

### Example 15: Characterization of Transitional Temperature Pooled CPP Product at Different Post-thaw Benchtop Stability Timepoints

This Example demonstrated the post-thaw stability of batches comprising transition temperature cryopreserved products. The post-thaw stability of cryopreserved platelet compositions having a biomolecule profile indicative of more than 1 platelet donor, in cryo-vessels, (referred to herein as “pooled CPP product” or “pooled CPP”), that was stored at −20° C. after it had been frozen at −80° C. was tested at various time points post-thaw. Three batches, W4464-21-000001, W4464-21-000018, and W4464-22-000011 of the cryopreserved platelet compositions in cryo-vessels were prepared by the process disclosed in Example 2 herein. Batch W4464-21-000001 was frozen at −80° C. and stored at −80° C. for 43 months and 5 days. Batch W4464-21-000018 was frozen at −80° C. and stored at −80° C. for 41 months and 29 days. Batch W4464-22-000011 was frozen at −80° C. and stored at −80° C. for 27 months and 10 days. The cryo-vessels from each of the three batches were transferred from a −80° C. freezer to a −20° C. freezer, and stored at −20° C. for 1 month and 21 days before being tested for the parameters as shown below. The cryopreserved platelet compositions in cryo-vessels from each of the three batches were thawed according to the below process:

The cryo-vessels were removed from a freezer and placed into a plasma thawer overwrap.

The cryo-vessels were then placed into a 37° C. plasma thawer with agitation for 8 minutes,

The cryo-vessels were removed from plasma thawer and rechecked for cracks, breaks, or leakage.

Each cryo-vessel was diluted/resuspended with 25 mL of 0.9% saline and visual inspection was performed to confirm that no aggregates were present before storing the pooled CPP product obtained after thawing and diluting/resuspending at the timepoints. At specified timepoints, 0 hours (T0), 4 hours (T4), 8 hours (T8), and 24 hours (T24), 5 mL aliquot was prepared by slowly drawing pooled CPP product into a syringe, then the aliquot was deposited into a clean conical tube for testing. The time-points are the post-thaw time durations at which point the composition was analyzed for its stability by assessing the parameters as disclosed below. After thawing and diluting/resuspending the cryopreserved platelet composition, the thawed-resuspended compositions were stored at room temperature for the specified timepoints above of T0, T4, T8, and T24. At the specified timepoints above, the samples were tested for the following parameters according to the process disclosed in Example 9 herein.

Platelet count per cryo-vessel

pH of pooled CPP product (>6.2)

Thrombin Generation (IU/10⁶particles)

CD61 positive Microparticles×10⁶/Ma

Lactadherin positive particles (%)

Tables 18-20 show the average of the parameters for each timepoint for the pooled CPP product from the different batches.

TABLE 18
W4464-21-000001

| | Platelet | | TGA | | Lactadherin |
|---|---|---|---|---|---|
| | Count/ | | (20K/μL, | CD61 | Positive |
| | Cryo- | | IU/10\hat{ }6 | Positive | Particles |
| Parameter | vessel | pH | Particles) | MP/μL | (%) |
| 0 hours | 2.2E+11 | 7.46 | 1.06 | 4.78E+05 | 99.0% |
| 4 hours | 1.7E+11 | 7.31 | 1.19 | 2.87E+05 | 99.0% |
| 8 hours | 1.5E+11 | 7.32 | 0.97 | 3.33E+05 | 98.0% |
| 24 hours | 9.4E+10 | 7.34 | 1.18 | 2.63E+05 | 97.8% |

TABLE 19
W4464-21-000018

| | Platelet | | TGA | | Lactadherin |
|---|---|---|---|---|---|
| | Count/ | | (20K/μL, | CD61 | Positive |
| | Cryo- | | IU/10\hat{ }6 | Positive | Particles |
| Parameter | vessel | pH | Particles) | MP/μL | (%) |
| 0 hours | 2.5E+11 | 7.28 | 0.91 | 3.76E+05 | 99.4% |
| 4 hours | 2.1E+11 | 7.2 | 1.02 | 2.36E+05 | 99.6% |
| 8 hours | 1.5E+11 | 7.05 | 0.89 | 2.38E+05 | 99.5% |
| 24 hours | 3.5E+10 | 7.23 | 0.69 | 5.53E+04 | 99.6% |

TABLE 20
W4464-22-000011

| | Platelet | | TGA | | Lactadherin |
|---|---|---|---|---|---|
| | Count/ | | (20K/μL, | CD61 | Positive |
| | Cryo- | | IU/10\hat{ }6 | Positive | Particles |
| Parameter | vessel | pH | Particles) | MP/μL | (%) |
| 0 hours | 2.3E+11 | 7.4 | 1.01 | 7.33E+05 | 98.1% |
| 4 hours | 2.0E+11 | 7.13 | 1.1 | 4.67E+05 | 98.4% |
| 8 hours | 1.8E+11 | 7.26 | 0.9 | 5.59E+05 | 98.0% |
| 24 hours | 1.7E+11 | 7.37 | 0.96 | 4.64E+05 | 97.1% |

As shown in tables 18-20 above, it can be observed that the pH of the compositions in each batch remained well above the acceptance criteria (>6.2) for all the timepoints. Thus, the results support that the cryopreserved platelet compositions, such as the transition temperature cryopreserved product disclosed herein (pooled CPP product) remain stable in terms of pH for up to 24 hours post-thaw. It can be observed that the pooled CPP product across the three batches at all timepoints had the ability to generate thrombin. The thrombin generation between the pooled CPP of the three batches differed by 0.1, 0.2, and 0.1 IU/10⁶ particles from 0 hours post-thaw (T0) and 24 hours post-thaw (T24), for the batches W4464-21-000001, W4464-21-000018, and W4464-22-000011, respectively. As shown in the tables above, the CD61 positive microparticle concentration of the CPP product across the three batches decreased over the course of the benchtop stability testing from T0 to T24. The CD61 positive microparticle counts of the three batches remained below 10×10⁶/μL at all timepoints from post-thaw (T0) to 24 hours post-thaw (T24). Therefore, it is demonstrated that the transition temperature cryopreserved product disclosed herein remains stable in terms of CD61 microparticle concentration up to 24 hours post-thaw. It can be observed that the phosphatidylserine positivity, when measured using lactadherin positivity values of the pooled CPP product across the three batches were stable over the course of the benchtop stability testing, values at 24 hours post-that (T24) differed from 0 hours post-thaw (T0) values by 1%, 0%, and 1% for the batches W4464-21-000001, W4464-21-000018, and W4464-22-000011, respectively. Further, it can be observed that the phosphatidylserine positivity, when measured using lactadherin positivity, is in the range of 97% to 99.7% upon storing the product at different timepoints demonstrated herein. This Example demonstrates that the pooled CPP product herein that is stored at −20° C. for 1 month and 21 days after initially being frozen at −80° C., therefore, also referred to as a transition temperature cryopreserved product shows stability over 24 hours at room temperature post-thaw when assessing parameters of pH, CD61 positive microparticle concentration, and lactadherin positivity.

### Example 16: Clinical Trial for Investigating the Role of Cryopreserved Platelets in Controlling Blood-Loss in Subjects Undergoing Cardiopulmonary Bypass Surgery

Cryopreserved platelet composition comprising frozen activated platelets, prepared according to the process disclosed in Example 2 herein, referred herein as “cryopreserved platelets”, or “pooled CPP product”, or “pooled CPP” were compared to liquid stored platelets (LSP) in their non-inferiority or superiority to control blood loss in subjects undergoing cardiopulmonary bypass surgery (CPB). For preparing the cryopreserved platelets, or the pooled CPP product for the clinical trial investigation, platelets from a minimum of 3 donors to a maximum of 8 donors were used, and a minimum total of 7 platelet units to a maximum total of 12 platelet units were used.

As a part of the clinical trial investigation, patients received up to 3 units of cryopreserved platelets or liquid stored platelets but could have received more than 3 units if required. Cryopreserved platelets have an extended shelf-life compared to liquid stored platelets, and as such, the control of blood loss in patients undergoing cardiopulmonary bypass surgery was used to evaluate non-inferiority or superiority of the efficacy and safety of the cryopreserved platelets compared to liquid stored platelets. After over 150 subjects were randomized and treated, an interim analysis was conducted. Such analysis determined that the cryopreserved platelets demonstrated statistical non-inferiority to the liquid stored platelets. Such determination was based on analysis of efficacy and safety based on the primary and secondary outcome measures detailed below.

### The Study

A randomized, parallel group, active comparator-controlled trial to evaluate the non-inferiority or superiority of cryopreserved platelets with liquid stored platelets (LSP) in controlling blood loss in patients undergoing Cardiopulmonary Bypass Surgery was performed. Patients who were planning to undergo Cardiopulmonary Bypass Surgery with risk factors for significant bleeding post-surgery were approached. Subjects were randomized in a 1:1 ratio to receive either cryopreserved platelets or liquid stored platelets. Eligible subjects underwent Cardiopulmonary Bypass Surgery and at the completion of bypass and heparin reversal, the subjects were assessed for eligibility before coming off bypass. Study platelets, either cryopreserved platelets or LSPs, were given either intraoperatively after heparin reversal and return of active clotting time (ACT) to <140 see or post operatively (after chest closure). In some subjects, the cryopreserved platelets herein or LSPs were administered up to 3 doses intraoperatively and post operatively, i.e., up to 3 doses in total considering intraoperatively and post operatively. One unit (1 unit) of the cryopreserved platelets used in the clinical study comprised at least 1.7×10¹¹ platelets. Whereas 1 unit of the LSP used in the clinical study comprised at least 3×10¹¹ platelets.

The cryopreserved platelets herein comprises at least 1.7×10¹¹ platelets and approximately 6% DMSO in a final frozen volume of ≥20 mL to ≤35 mL. Prior to clinical administration, the cryopreserved platelets were thawed and resuspended with 25 mL sterile 0.9% sodium chloride (NaCl) for injection. After thawing and resuspension, the thawed platelet units are stable at room temperature (20° C. to 24° C.) for up to 8 hours, and were administered intravenously. After thawing and resuspension, the thawed and resuspended product comprises at least 1.7×10¹¹ irradiated platelets in sterile 0.9% NaCl solution in a volume of ≥45 mL to ≤60 mL and 1250 to 2530 mg residual DMSO.

The subjects, over 150 selected in total, met the following inclusion criteria:
1. Male or female, at least 18 years of age
2. Undergoing CPB surgery with at least one risk factor for post-surgical bleeding including:
   i. All re-operative cardiac procedures.
   ii. Expected bypass >120 minutes.
   iii. Any combined cardiac surgery procedures (e.g. multiple valve, valve/CABG).
   iv. Any procedure that in the estimation of the surgical attending, has a high likelihood of receiving platelets
3. All patients had the ability to comprehend, and signed the informed consent.
4. If was a female of childbearing potential, had a negative pregnancy test on the day of the surgery and prior to the surgery agreed to use a method of highly effective birth control from the time of consent through the end of the safety follow-up period (Day 6 or discharge from hospital, whichever was earlier). Note: women were either surgically sterilized [bilateral tubal ligation, bilateral oophorectomy, total hysterectomy) or were at a postmenopausal (≥50 years of age and continuous amenorrhea for 24 months) stage to have been considered non-childbearing potential.

The study evaluated the non-inferiority or superiority of up to 3 units of cryopreserved platelets compared to up to 3 units of control (LSP). However, more than 3 units of study platelets could have been administered if required. Cryopreserved platelets and control dosage are listed in Table 21.

TABLE 21

| Trial-Arm | |
|---|---|
| Description | Dosage Description |
| Cryopreserved | Cryopreserved platelets were given intraoperatively or |
| platelets | post operatively up to 3 units post-heparin reversal |
| Control | Liquid stored platelets were given intraoperatively or |
| | post operatively up to 3 units post-heparin reversal |

The specific outcomes analyzed are listed in Table 22.

TABLE 22

| | |
|---|---|
| Efficacy Endpoint | Time Frame |

Primary Outcome Measures

| | |
|---|---|
| Total volume of chest tube drainage assessed by | From “time zero” until the drain tubes |
| measurement of the volume of blood collected from the | were removed or 24 hours post time |
| mediastinal and pleural drains from “time zero”, the time of | zero, whichever was earlier |
| 1) chest closure or equivalent, 2) chest tubes or equivalent are | |
| attached to a graduated post drainage system, and 3) with | |
| suction (defined as time zero for analytical purposes) until | |
| the drain tubes are removed or 24 hours post time zero, | |
| whichever is earlier. | |

Secondary Outcome Measures

| | |
|---|---|
| Total volume of chest tube drainage, given in mL/kg, | From “time zero” until the drain tubes |
| assessed by measurement of the volume of blood collected | were removed or 24 hours post time |
| from the mediastinal and pleural drains from “time zero”, the | zero, whichever was earlier |
| time of 1) chest closure or equivalent, 2) chest tubes or | |
| equivalent are attached to a graduated post drainage system, | |
| and 3) with suction (defined as time zero for analytical | |
| purposes) until the drain tubes are removed or 24 hours post | |
| time zero, whichever is earlier. | |
| Chest tube drainage volume (mL) collected at 6 hours | 6 hours intervals through 24 hours post |
| intervals through 24 hours post time zero or tube removal, | time zero or when chest tubes were |
| whichever was earlier. | removed, whichever was earlier. |
| Drainage rate (mL/hr) collected at 6 hours intervals through | 6 hours intervals through 24 hours post |
| 24 hours post time zero or when chest tube was removed, | time zero or when chest tubes were |
| whichever was earlier. | removed, whichever was earlier |
| Total units by type of other post-operative blood products | Infused after the end of the first study |
| (pRBC, non-study platelets, CRYO, plasma, clotting factor | platelet transfusion through 24-hour |
| concentrates) that were infused after the end of the first study | post heparin reversal (Efficacy follow- |
| platelet transfusion until end of the efficacy follow-up period | up period) |
| Incidence of surgical re-exploration and incidence of verified | Within the 24 hour period after heparin |
| surgical or other causes for bleeding within the 24 hour | reversal |
| period after heparin reversal | |
| Time to hemostasis (defined as the time from first protamine | Time from first protamine |
| administration to the time when the surgeon initiates the first | administration to the time when the |
| suture for incision closure) | surgeon initiates the first suture for |
| | incision closure, Day 1 (Day of |
| | operation) |
| Treatment failure (defined as requiring more than three units | Time of first study platelet transfusion |
| of study treatment (cryopreserved platelets or LSP)) | through 24-hour post heparin reversal |
| | (Efficacy follow-up period) |

Statistical design of primary efficacy endpoint is as follows

Non-inferiority margin: A margin of 400 mL was used as the non-inferiority margin for this study.

Model used: To test the null hypotheses, the least squares (LS) mean, SE, and CI was obtained from an analysis of covariance (ANCOVA) that included treatment as an independent variable and adjusts for the following covariates: clinical site (the randomization strata) and receipt of anti-platelet agent (during the 5 days prior to the administration of the first study treatment).

Primary efficacy endpoint: The difference between the least squares (LS) mean total chest tube drainage (CTD) volume for subjects receiving LSP compared to those receiving cryopreserved platelets herein was analyzed with an overall two-sided 95% CI across the interim and final analysis. For the interim analysis, the two-sided CI was analyzed at 98.07%, and for the final analysis, the two-sided CI was analyzed at 95.576%.

This study is designed with at least 90% statistical power for the upper limit of the CI around the mean difference in total CTD volume (least squares mean for cryopreserved platelets—least squares mean for LSP) to exclude 400 ml. If the upper limit of the CI of the mean difference excludes 400 ml then the cryopreserved platelets herein will be considered non-inferior to LSP.

Subjects with the following criteria were excluded from the study:
1. Underwent any of the following surgical procedures:
2. Coronary artery bypass surgery alone
3. Implantation of ventricular assist device
4. Thoracoabdominal aortic aneurysm repair
5. Were known or suspected pregnancy or breastfeeding
6. Had history of any major unprovoked thrombotic events
7. Had history of heparin-inducted thrombocytopenia
8. Had active infection treated with antibiotics
9. Refused transfusion of blood products for religious or other reasons
10. Previous enrollment in this study
11. Immune thrombocytopenic purpura
12. Known allergy to DMSO
13. In the judgement of the investigator, were not a good candidate for the study

### Summary of Interim Analysis:

The clinical study as disclosed herein met the primary efficacy endpoint of 24-hour chest tube drainage to demonstrate statistical non-inferiority of the cryopreserved platelets disclosed herein to the standard room temperature control platelet product (LSP) with a one-sided significance level less than p-value 0.00965.

### Summary of Final Topline Results:

The Least Square (LS) Means of primary efficacy endpoint measure of 24-hour chest tube drainage was 142.0 ml. Further, the 95.576% confidence interval of volume difference was lower than the non-inferiority margin (400 ml). The sensitivity analyses of the primary endpoint were consistent and supportive. Comprehensive review of the study efficacy data based on clinically relevant secondary endpoints further supports noninferiority of cryopreserved platelets disclosed herein to the standard room temperature control platelet product (LSP). Adverse events were well balanced between the two treatment groups.

The clinical trial (CRYPTICS) was a prospective, randomized, parallel group, active comparator-controlled double-blinded Phase 2/3 adaptive trial that evaluated noninferiority of cryopreserved platelets herein (pooled CPP) to LSP (also called as RTP). The subjects were randomized to receive ≤3 units of the cryopreserved platelets (pooled CPP) or RTP for perioperative bleeding. A standardized algorithm was used to guide platelet administration. The primary efficacy endpoint was chest tube drainage (CTD) volume from the time of chest closure until chest tube removal or 24 hours after chest closure. Missing CTD data were imputed using a monotone linear regression multiple imputation approach. Safety outcomes included the proportion of patients who experienced adverse events (AEs) and 30-day mortality.

### Results

Overall, 161 CPB patients from 18 sites were enrolled (modified intention-to-treat population: pooled CPP, n=70; RTP, n=91; safety population: pooled CPP, n=68; RTP, n=93) (Refer Table A). Demographics of the two treatment groups were mostly similar (Refer Table B), as were the surgical procedures performed (p=0.80) (Refer Table C). The least-squares mean difference in the primary efficacy endpoint (ANCOVA) between groups was 142 mL (−48.8, 332.9) (Refer Table E), which was below the prespecified non-inferiority margin of 400 mL. The mean (SD) number of pooled CPP units used was 1.9 (0.83) compared to 1.7 (0.75) units for RTP (p=0.11). The proportion of patients who experienced AEs was similar between treatment groups (p=0.38) (Table D). There was also a similar proportion of deaths in the pooled CPP and RTP groups (4.4% [3/68]vs 5.4% [5/93], respectively, p=1.00); no deaths were considered definitely related to pooled CPP or RTP. There was no difference in the incidence of surgical re-exploration for bleeding in the pooled CPP and RTP groups (2.9% [2/70]vs 2.2% [2/91]; p=1.00) and transfusion requirements (p=0.19) did not differ between groups.

TABLE A

| | | |
|---|---|---|
| Pooled CPP | LSP | Total |
| (N = 151) | (N = 161) | (N = 338) |
| n (%) | n (%) | n (%) |

All Screened Population

| Screened Subjects | | | 338 |
|---|---|---|---|
| Screen Failed | | | 6 (1.8) |
| Rescreened | | | 18 (5.3) |
| Enrolled but not | | | 20 (5.9) |
| Randomized | | | |
| Randomized | 151 (100) | 161 (100) | 312 (92.3) |
| No Surgery | 8 (5.3) | 5 (3.1) | 13 (3.8) |
| Surgery, No Platelets | 58 (38.4) | 62 (38.5) | 120 (35.5) |
| Required | | | |
| Treated | 70 (46.4) | 91 (56.5) | 161 (47.6) |

Study Disposition

| | | | |
|---|---|---|---|
| Ongoing in Study | 0 | 0 | 0 |
| Completed Study | 66 (43.7) | 79 (49.1) | 145 (4.9) |
| Follow-up | | | |
| Exited Study | 85 (56.3) | 82 (50.9) | 162 (49.4) |
| Enrolled Population | | | 332 |
| ITT Population | 150 | 159 | 309 |
| mITT Population | 70 (46.7) | 91 (57.2) | 161 (52.1) |
| Safety Population | 68 (45.3) | 93 (58.5) | 161 (52.1) |
| PP Population | 58 (38.7) | 86 (54.1) | 144 (46.6) |

The per-protocol (PP) population includes all mITT subjects without major protocol deviations, including errors in study treatment and transfusion.

TABLE B
Subject Demographics

| | | |
|---|---|---|
| Pooled CPP | LSP | Total |
| (N = 70) | (N = 91) | (N = 161) |
| n (%) | n (%) | n (%) |

Age at Study Entry (years)

| | | | |
|---|---|---|---|
| N | 70 | 91 | 161 |
| Mean (SD) | 67.2 (9.86) | 63.2 (12.41) | 64.9 (11.51) |
| Median | 69.0 | 64.0 | 67.0 |
| Min, Max | 32, 85 | 19, 82 | 19, 85 |

Age Category (n(%))

| | | | |
|---|---|---|---|
| <Median Age | 25 (35.7) | 48 (52.7) | 73 (45.3) |
| ≥Median Age | 45 (64.3) | 43 (47.3) | 88 (54.7) |

Sex (n(%))

| | | | |
|---|---|---|---|
| Female | 13 (18.6) | 27 (29.7) | 40 (24.8) |
| Male | 57 (81.4) | 64 (70.3) | 121 (75.2) |

Race (n(%))

| | | | |
|---|---|---|---|
| White | 61 (87.1) | 76 (86.5) | 137 (85.1) |
| Black or African | 4 (5.7) | 10 (11.0) | 14 (8.7) |
| American | | | |
| Asian | 2 (2.9) | 3 (3.3) | 5 (3.1) |
| American Indian or | 0 | 0 | 0 |
| Alaska Native | | | |
| Native Hawaiian or | 0 | 1 (1.1) | 1 (0.6) |
| Other Pacific Islander | | | |
| Multiple | 0 | 1 (1.1) | 1 (0.6) |
| Other | 3 (4.3) | 0 | 3 (1.9) |
| Not Reported | 0 | 0 | 0 |

TABLE C
Safety Population

| | | | |
|---|---|---|---|
| | Pooled CPP | LSP | Total |
| | (N = 68) | (N = 33) | (N = 161) |
| Surgery Category | n (%) | n (%) | n (%) |

Primary Surgery

| | | | |
|---|---|---|---|
| Total Primary Surgery | 51 (75.0) | 76 (81.7) | 127 (78.9) |
| CABG | 8 (11.8) | 11 (11.8) | 19 (11.8) |
| Valve-any valve | 6 (8.8) | 13 (14.0) | 19 (11.8) |
| replacement or repair | | | |
| CABG and Valve | 13 (19.1) | 17 (18.3) | 30 (18.6) |
| combination (any | | | |
| valve) | | | |
| Other | 24 (35.3) | 35 (37.6) | 59 (36.6) |

Redo Surgery

| | | | |
|---|---|---|---|
| Total Redo Surgery | 17 (25.0) | 17 (18.3) | 34 (21.1) |
| CABG | 1 (1.5) | 0 | 1 (0.6) |
| Valve-any valve | 7 (10.3) | 6 (6.5) | 13 (8.1) |
| replacement or repair | | | |
| CABG and Valve | 2 (2.9) | 1 (1.1) | 3 (1.9) |
| combination (any | | | |
| valve) | | | |
| Other | 7 (10.3) | 10 (10.8) | 17 (10.6) |

TABLE D

| | Pooled CPP | RTP | Total |
|---|---|---|---|
| Number of | (N = 68) | (N = 93) | (N = 161) |
| subjects with | n (%) [E] | n (%) [E] | n (%) [E] |
| Any AE | 60 (88.2) [330] | 78 (83.9) [441] | 138 (85.7) [771] |
| Serious AE | 26 (38.2) [74] | 39 (41.9) [107] | 65 (40.4) [181] |
| Any | 4 (5.9) [5] | 9 (9.7) [10] | 13 (8.1) [15] |
| Thromboembolic | | | |
| Complications | | | |
| Any AE of Special | 35 (51.5) [52] | 47 (50.5) [84] | 82 (50.9) [136] |
| Interest | | | |
| Any AE requiring | 19 (27.9) [36] | 24 (25.8) [55] | 43 (26.7) [91] |
| or prolonging | | | |
| hospitalization | | | |
| Any AE leading | 3 (4.4) [3] | 5 (5.4) [6] | 8 (5.0) [9] |
| to Death* | | | |

Multiple imputation: The primary efficacy endpoint for determination of noninferiority was the total CTD volume as assessed by measurement of the blood volume collected from the mediastinal and pleural drains from the time of 1) chest closure with chest tubes and/or equivalent (such as when JP drains are added in an Ioban® sealed, open sternum chest wound), and 2) when chest tubes or equivalent as noted in (1) are attached to the graduated post drainage system, and 3) when attached to suction until the time the chest tubes are removed or 24 hours after time zero, whichever is earlier, between subjects who received CLPH-511 and those who received LSP. The CTD volume was recorded hourly for the first 12 hours and at 6-hour intervals thereafter, through 24 hours post time zero (or chest tube removal if earlier); the cumulative CTD volume was used for the primary efficacy endpoint analysis. To test the null hypotheses, the least squares mean, SE, and CI will be obtained from an analysis of covariance (ANCOVA) model that includes treatment as an independent variable and adjusts for the following covariates: clinical site (the randomization strata) and receipt of anti-platelet agent (during the 5 days prior to the administration of the first study treatment). The primary efficacy analysis will use multiple imputation methods to account for subjects with missing CTD volume measurements in the mITT population. Subjects who died before insertion of the drainage tube will be analyzed under the treatment to which they were randomized, with any missing data imputed in the same manner as discussed below for subjects with other types of missing data. Missing CTD volumes were imputed using a monotone linear regression MI approach for continuous outcome data.

This study is designed with at least 90% statistical power for the upper limit of the interim or final CI around the mean difference in total CTD volume (least squares mean CLPH-511—least squares mean LSP) to exclude 400 mL. If the upper limit of the interim or final CI of the mean difference excludes 400 mL then CLPH-511 will be considered non-inferior to LSP. Overall, the difference in LS Means between CLPH-511 and LSP was 142.0 with 95.576% CI of −48.8 to 332.9, indicating that CLPH-511 meets the criteria of noninferiority to LSP (if the upper limit of the interim or final analysis CI of the mean difference excludes 400 mL then CLPH-511 will be considered noninferior to LSP. The noninferiority margin of 400 mL has been determined for the CRYPTICS study based on existing clinical evidence from published literature (Stafford-Smith et al., 2005)

miTT sensitivity analysis: Multiple sensitivity analyses were performed to verify the noninferiority result, all of which confirmed noninferiority of the cryopreserved platelets (pooled CPP) to LSP. A sensitivity analysis was performed using only available CTD volumes with no imputation demonstrated noninferiority by the difference in LS means (Refer to Table F).

Table E shows the primary efficacy endpoint (ANCOVA analysis with multiple imputation)

TABLE E

| | Pooled CPP | | RTP | |
|---|---|---|---|---|
| ANCOVA of MI Datasets | | | | |
| LS Means (SE) | 1156.5 | (86.55) | 1014.5 | (80.45) |
| 95% CI | 986.9, | 1326.2 | 856.8, | 1172.2 |
| Difference in LS Means | 142.0 | | | |
| 95.576% CI | −48.8, | 332.9 | | |

| | |
|---|---|
| One-sided p value | <0.02212 |

Table F shows the primary efficacy endpoint (ANCOVA) sensitivity analysis imputation

TABLE F

| | |
|---|---|
| Pooled CPP | RTP |

| ANCOVA | | | | |
|---|---|---|---|---|
| LS Means (SE) | 1142.5 | (88.9) | 1005.6 | (80.89) |
| 95% CI | 966.8, | 1318.3 | 675.0, | 1246.0 |
| Difference in LS Means | 136.9 | | | |
| 95.576% CI | −55.8, | 329.7 | | |

| | |
|---|---|
| One-sided p value | <0.02212 |

Platelet Counts in Subjects that were Administered Pooled CPP Herein Versus the Subjects that were Administered RTP:

With respect to monitoring platelet count, there was an overall significant correlation between platelet count and 24 hour CTD volume for both pooled CPP (Correlation coefficient=−0.31 [p=0.0113]) and LSP (Correlation coefficient=−0.33 [p=0.0020]), indicating that increasing platelet count is associated with decreased bleeding. Platelet count sampling performed prior to dosing, after each dose, and through the postoperative period revealed a more robust post-dose platelet count increase in the LSP group compared to the pooled CPP group. There was an overall increase in mean platelet count from the post-heparin reversal time point in the LSP group (109.96×10⁹/L, range=24-326.0 10⁹/L) to the end of efficacy period 129.05×10⁹/L, range 21-256.0×10⁹/L). Whereas the pooled CPP group remained relatively the same with transient platelet increases (post-heparin reversal=102.10×10⁹/L, range=30-194.0×10⁹/L; end of efficacy=100.67×10⁹/L, range=31-189.0×10⁹/L). The end of the efficacy period is 24 hours post time zero or when chest tubes are removed, whichever is earlier. The time zero is the time of 1) chest closure or equivalent, or 2) chest tubes or equivalent are attached to a graduated post drainage system, or 3) with suction (defined as time zero for analytical purposes).

Conclusions: Cryopreserved platelets herein (pooled CPP) was non-inferior to RTP (also referred to as LSP) for the treatment of acute hemorrhage in patients undergoing CPB, with a similar incidence of AEs. A similar number of platelets were used in both groups, and the transfusion requirements were not significantly different. These outcomes support the use of the cryopreserved platelets herein (pooled CPP) as an alternative to RTP (or LSP) in bleeding patients in whom a platelet transfusion is required.

### Example 17: Production of Pooled CPP Product, Room Temperature Platelets (RTP), and Cold Stored Platelets (CSP)

This example provides details regarding the preparation of 3 different platelet products: Pooled CPP product, RTP, and CSP.

### Pooled CPP Product

Pooled CPP product was manufactured from group O apheresis platelet units (from up to 10 unique donors). For this production 8-12 units of apheresis platelets were used to manufacture the pooled CPP product. Six GMP batches of pooled CPP product were manufactured, 3 units of each batch. The pooled CPP product was manufactured according to the method of Example 2 and stored at ≤−65° C. For the purposes of the below Examples, the pooled CPP product was stored at ≤−65° C. for either 6 months or 9 months before thawing for further testing. Wherever indicated in the below experiments, data demonstrating the testing of pooled CPP product is a collection of individual data with the pooled CPP products stored for 6 months and the pooled CPP product stored for 9 months. For the comparison experiments hereinafter, the following steps were performed for testing the pooled CPP product at different timepoints of storage. The pooled CPP product stored in cryo-vessels was removed from the −80° C. freezer and transferred to the lab on −80° C. ice packs. The required number of cryo-vessels were than placed into a plasma thawer set at 37° C. for eight (8) minutes. The cryo-vessels comprising thawed platelet particles were removed from the plasma thawer and checked for cracks, breaks, or leakage. The thawed platelet particles in each cryo-vessel were diluted with 25 mL of 0.9% saline. The cryo-vessel was gently massaged to mix, unit was fitted with a clean syringe via the leur port, and approximately 10 mL aliquot was prepared by slowly drawing the thawed platelet particles into a syringe and then was deposited into a clean conical tube for testing.

### Room Temperature Platelets (RTP)

Apheresis platelet units (APU) were purchased from a licensed, qualified provider with overnight delivery. The apheresis units (APU) were received within 24 hours of collection. APUs were sampled to produce cold stored platelet (CSP) units. After the sampling, a sterile 32 mL FEP bag was welded to the APU and the connecting tubing was clamped shut. The platelets in the APU were stored in Helmer platelet incubator at 18-22° C. with agitation and were termed as room temperature platelets (RTP). These RTP were tested at different timepoints; at each timepoint tested the RTP unit was removed from the platelet incubator and a 10 mL sample was taken from the luer lock port of the connected 32 mL FEP bag.

### Cold Stored Platelets (CSP)

CSP units were produced from the RTP units. Upon receipt of an APU, a sterile 32 mL FEP bag was welded to the APU and approximately 30 mL of the apheresis platelets was transferred to the FEP bag. The FEP bag was welded off and this aliquot was transferred to a 2-8° C. refrigerator without agitation.

Each FEP bag was labelled with the corresponding APU identification number. CSP units were tested at different timepoints; at each timepoint the CSP unit was removed from the refrigerator and a 10 mL sample was taken from the luer lock port of the 32 mL FEP bag. CSP units and APU were matched, coming from the same donor and source.

### Example 18: Pooled CPP product is a more concentrated platelet product with a lower pH as compared to RTP and CSP

Samples of the platelet products—RTP, CSP, and Pooled CPP product were taken at different timepoints as disclosed in Example 17 for testing.

The platelet products were tested for total platelet count, platelet concentration and pH according to the following methods.

The total platelet count and platelet concentration (per μl) for pooled CPP product, RPT, and CSP was obtained using the Beckman Coulter D×H 520 Hematology Analyzer (Beckman Coulter, beckmancoulter.com) according to the following steps.

Prior to testing samples of the D×H 520 Hematology Analyzer, the controls were run on the instrument and verified.

The thawed platelet particles from the pooled CPP product (as disclosed in Example 17) were diluted 1:20 by 0.9% saline. A 50 μl of the thawed platelet particles were added to 950 μl of saline. RTP and CSP sample tubes were diluted 1:10 by 0.9% saline. A 100 μl of sample of RTP or CSP was added to 900 μl of saline.

Sample tubes were inverted several times to mix.

Each tube was run on the D×H 520 Hematology Analyzer for platelet count and platelet concentration (per μl).

The volume from which the samples of different platelet products were obtained for testing was documented and calculations were performed to obtain total platelet count and platelet concentration for each sample.

Samples were Run in Triplicate

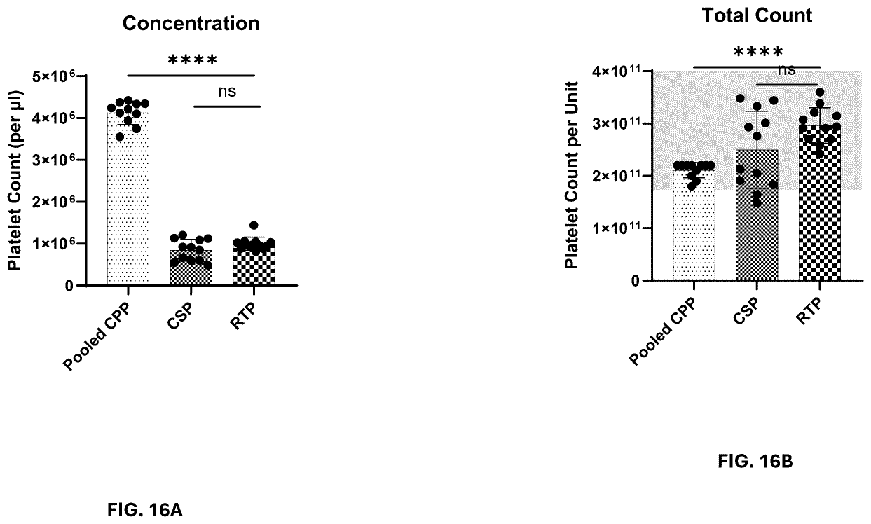

FIG. 16A and FIG. 16B show the platelet concentration/μl and total platelet count for the three samples—thawed platelet particles obtained from pooled CPP product (shown as “pooled CPP product”), RTP, CSP.

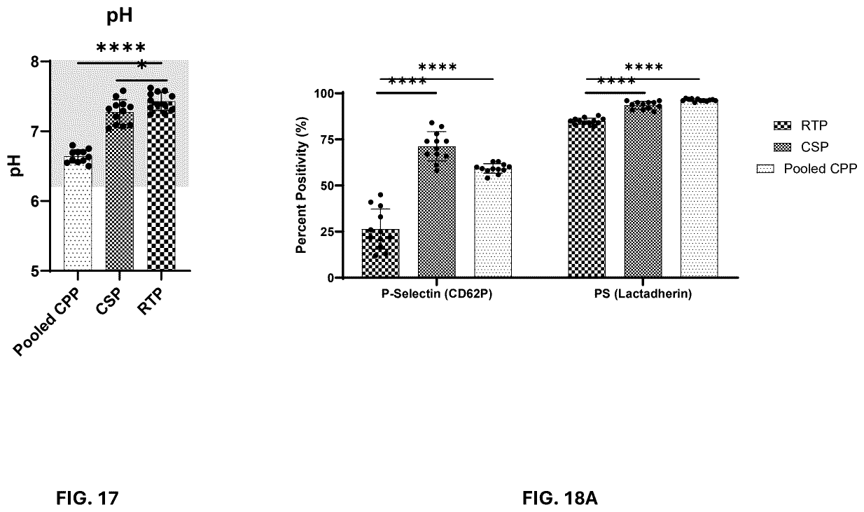

The pH for each sample of the platelet products was obtained using a pH meter. The pH standardization was verified prior to testing samples on pH meter. FIG. 17 shows the pH value for all three samples. The shaded area on FIG. 16B and FIG. 17 represents the release criteria for pooled CPP product.

### Example 19: Pooled CPP Product has an Activated Phenotype Based on Flow Cytometry of Platelet Surface Mechanistic Markers

The platelet products were taken at different timepoints as disclosed in Example 17 and tested for percent positivity and mean florescence intensity (MFI).

The Novocyte Quanteon Flow Cytometer (Agilent, agilent.com) was started up for testing to initiate a new experiment file.

The three samples of platelet products were prepared according to the following steps.

Sample from each platelet product was diluted to 1:500 in triplicate by adding 2 μl of the platelet product to 998 μl of PBS.

The 100 μl of the combined PBS and the platelet product were transferred from dilution to an individual well on a 96 well U-bottom plate.

The platelet products were tested for the surface marker positivity in terms of % positivity, and MFI for the surface markers—Phosphatidylserine (PS) when measured using binding of lactadherin, Ps-Selectin (CD62P), GPVI, GPIba (CD42b), vWF, TSP, and fibrinogen.

The count of the size gated platelet product population was determined on a bivariate plot of FSH-H/SSC-H in the diluted platelet samples by acquiring the samples on the Quanteon with the following steps.
Parameters: FSC, SSC
Stop conditions: 25 μL
FSC-H Threshold: 1,000
Flow Rate: Medium
Absolute count dilution: 1:500

Based on the flow count of each sample, a 400 μl sample was created at a concentration of 100,000/μL diluted in HMTA.

Matched isotype controls were prepared with their respective antibodies in the steps that follow

The following antibodies/reagents were prediluted in the following step.

FITC labeled Lactadherin (Haematologic Technologies, goprolytix.com) was diluted to 1:3 by combining 34 μL labeled Lactadherin with 68 μL of unlabeled Lactadherin

Anti-GPVI antibody (BD Biosciences, biosciences.com) was diluted 3:10 by combining 3 μL of antibody with 7 μL of HMTA.

Anti-VWF antibody stock (Novus Biologicals, novusbio.com) was diluted 1:10 by combining 4 μL

Polyclonal anti-fibrinogen antibody (Abcam, abcam.com) was diluted 1:7 by combining 4 μL antibody with 24 μL of HMTA

The following stains were generated in duplicate with the 100,000/μL platelet products (thawed platelet particles from pooled CPP product, RTP, CSP) using the prediluted antibodies/reagents prepared above where applicable:
Unstained: 10 μL cells +20 μL HMTA
Lactadherin (Haematologic Technologies, goprolytix.com): 5 μL cells +20 μL Lactadherin+5 μL HMTA
Anti-GPVI: 10 μL cells +2 μL antibody+18 μL HMTA
Anti-VWF: 10 μL cells +9 μL antibody+11 μL HMTA
Anti-CD62 (BD Biosciences, biosciences.com): 10 μL cells +9 μL antibody+11 μL HMTA
Anti-CD42 (BD Pharmingen, biosciences.com): 10 μL cells +6 μL antibody+14 μL HMTA
Anti-fibrinogen: 10 μL cells +5 μL antibody+15 μL HMTA
Anti-thrombospondin (Santa Cruz Biotechnology, scbt.com): 10 μL+5 μL antibody+15 μL HMTA

All stain samples were incubated away from light for 20 minutes.

After incubation, 500 μL of PBS was added to each platelet sample.

100 μL of each diluted sample was transferred to an individual well in a 96 well U-bottom plate

The Samples were Acquired on the Quanteon with the Following Settings
Parameters: FSC, SSC, fluorescence was collected for each stain appropriately
   FITC (B530) for Lactadherin and anti-fibrinogen

PE (Y586) for anti-GPVI, anti-VWF, anti-CD62P, and anti-thrombospondin

The stop conditions were 30 μL or 30,000 events
FSC-H Threshold: larger than 1,000 (5,000 for Lactadherin samples)
Flow Rate: Medium
Absolute count dilution: 1:500

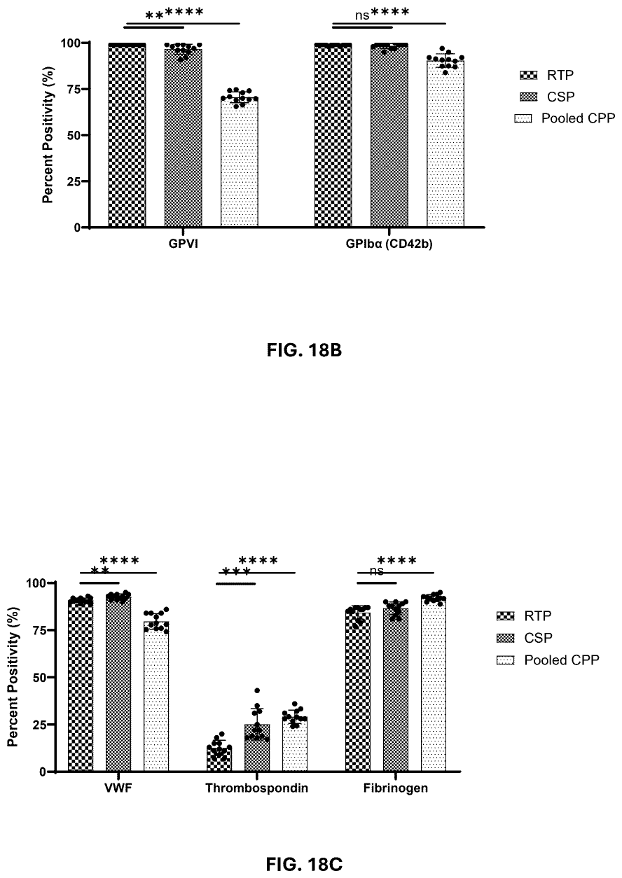

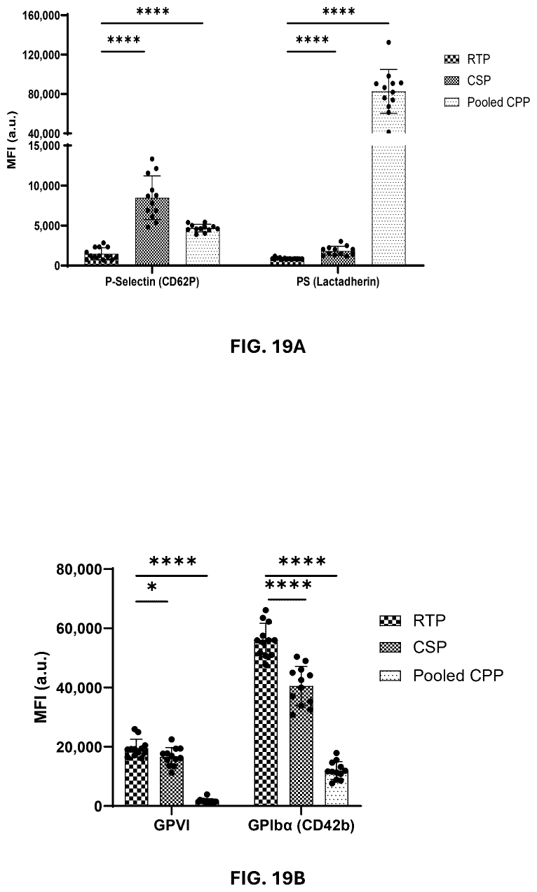

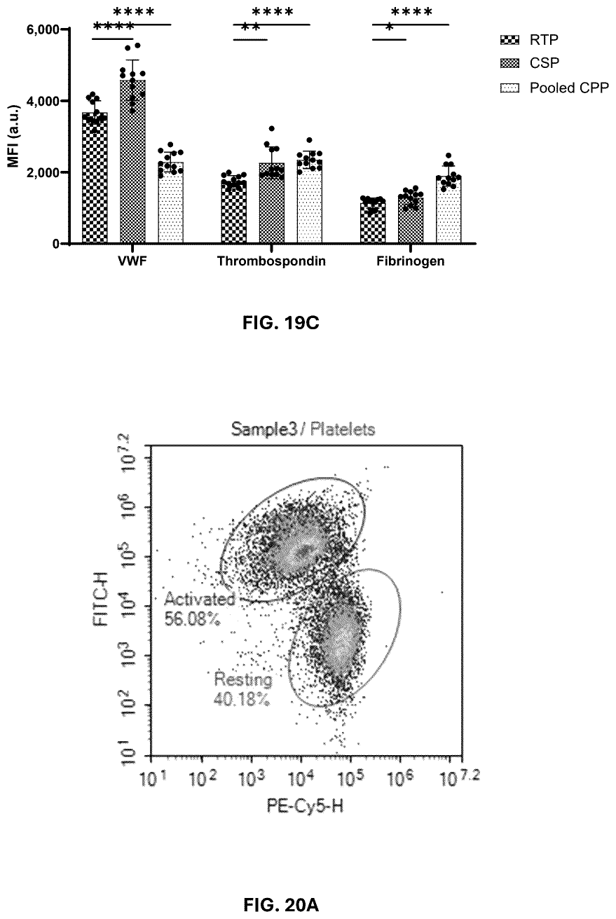

FIG. 18A, FIG. 18B, and FIG. 18C show the surface marker percent positivity for the three platelet products after different timepoints of storage. FIG. 19A, FIG. 19B, and FIG. 19C show the mean fluorescence intensity (MFI) of the surface markers for all three platelet products. The pooled CPP product was tested after 6 and 9 months of storage. The data shown for the pooled CPP product in the figures above is a collection of individual data points obtained separately from the two different storage timepoints of 6 and 9 months of the CPP product. For example, in case a bar demonstrates ten data points, it consists of individual data points, such as 3, 4, or 5 from the pooled CPP product that is stored for 6 months and individual data points, such as 7, 6, or 5 from pooled CPP product that is stored for 9 months before testing. RTP samples were tested after 4 and 7 days of storage. CSP samples were tested after 7 and 14 days of storage.

The results show that pooled CPP product upon thawing shows at least 75% positivity for the presence of PS when measured using lactadherin binding (FIG. 18A), and displays greater than 60K MFI of Lactadherin (FIG. 19A), thereby demonstrating the presence of phosphatidylserine exposed on the surface of the thawed platelet particles obtained from the pooled CPP product. Further, it was observed that the pooled CPP product upon thawing shows a reduced surface positivity for GPVI (about 70-74%) (FIG. 18B), and less than 4K MFI (FIG. 19B) as compared to the percent positivity and MFI data for CSP and RTP. Similarly, the pooled CPP product upon thawing shows a reduced surface positivity of CD42b (FIG. 18B), and less than 16K MFI for CD42b (FIG. 19B) as compared to the percent positivity and MFI data for CSP and RTP. In contrast, the pooled CPP product upon thawing show increased surface positivity of fibrinogen (about 90%) (FIG. 18C), and at least 1.5K MFI (FIG. 19C). The presence of higher % positivity and higher MFI in the pooled CPP product upon thawing as compared to CSP and RTP for phosphatidylserine, and fibrinogen are indicative that the pooled CPP product upon thawing comprises a population of platelet particles that are activated.

### Example 20: Pooled CPP Product Comprises Two Sub-Populations of Platelet Particles, One More Activated than the Other

Three batches of pooled CPP product was manufactured according to the process disclosed in Example 2 and stored at ≤−65° C. The samples from each batch were tested for mean florescence intensity (MFI) for multiple surface marker stains in combination with PS positive (+) and PS negative (−) platelet population obtained according to the method of Example 19. The method further includes the below steps:

The samples were acquired on the Quanteon with the following settings:
Parameters: FSC, SSC, fluorescence was collected for each stain appropriately.
FITC (B530) for Lactadherin (phosphatidylserine (PS)).
PE (Cy5) for anti-CD42.
PE: TSP-1
Pacific blue for anti-GPVI
Data and analyzed plots were generated.

FIG. 20A, and FIG. 20B show scattergram identifying the two sub- populations of platelet particles (a more activated subpopulation and a less activated subpopulation) in the pooled CPP product upon thawing, and median height for the FSC and SSC of the two sub-populations, respectively. FIG. 20C shows the MFI of the PS, CD41, TSP-1, GPVI, and CD42 for platelet sized particles in the two sub-populations: negative for phosphatidylserine, also termed as less activated sub-population (shown in FIG. 20C as “Less Activated”) and those that are positive for phosphatidylserine, also termed as more activated sub-population (shown in FIG. 20C as “More Activated”). This Example when read with Example 19 demonstrates that the pooled CPP product as disclosed herein upon thawing comprises two sub-populations of platelet particles, a first sub-population comprising platelet particles positive for PS, and a second sub-population comprising platelet particles negative for PS. Further, a population of platelet-sized particles was obtained by using fluorescently-tagged antibody specific for CD41 (Alexa Fluor 700-H) and FSC-H analysis using flow cytometry of the pooled CPP product upon thawing. Referring to FIG. 20A, the population thus obtained when analyzed using fluorescently-tagged protein specific for PS (FITC-H), using the protein: lactadherin, and fluorescently-tagged antibody specific for CD42b (PE-Cy5-H) distinctively shows two sub-populations of platelet particles, a more activated sub-population (56.48%), and a less activated sub-population (40.18%). FIG. 20B shows that both FSC and SSS gave higher median height for the less activated sub-population as compared to the more activated sub-population. FIG. 20C shows an MFI of about 140,000 for the more activated sub-population, whereas only about 2,000 for the less activated sub-population. Regarding CD41, CD42b and GPVI, the less activated sub-population had the higher MFI as compared to the more activated sub-population. The MFI of TSP did not change significantly but was marginally higher for the less activated sub-population as compared to the more activated sub-population.

### Example 21: Pooled CPP product shows 3X higher thrombin activity than RTP and CSP in TGA performed using the CLARIOstar^{Plus} machine

Samples of the three platelet products were taken at different timepoints as disclosed in Example 17 for testing.

Necessary reagents and platelet samples were prepared according to the following steps.

TGA dilution buffer was removed from freezer and thawed in 37±2° C. water bath for approximately 10 minutes.

Thrombin standards were removed from −80° C. freezer and the required amount of aliquots were thawed at room temperature for approximately 10 minutes.

The Master Mix was removed from −80° C. freezer and thawed in a water bath at 37° C. for approximately 5 minutes. The tubes were inverted several times, making sure to not exceed 10 minutes in the water bath.

The counts of Pooled CPP product, RTP, and CSP were acquired using the Beckman Coulter D×H 520 Hematology Analyzer (Beckman Coulter, beckmancoulter.com) according to the following steps.

Prior to testing samples of the D×H 520 Hematology Analyzer, the controls were run on the instrument and verified.

The pooled CPP product were thawed as per a protocol disclosed in Example 17 to obtain thawed platelet particles, and the thawed platelet particles were diluted 1:20 by 0.9% saline. A 50 μl of thawed platelet particles was added to 950 μl of saline. RTP and CSP sample tubes were diluted 1:10 by 0.9% saline. A 100 μl of sample of RTP or CSP was added to 900 μl of saline.

Sample tubes were inverted several times to mix.

Each tube was run on the D×H 520 Hematology Analyzer for platelet count.

The volume for each bag that samples were obtained from was documented and calculations were performed to obtain total platelet count for each sample.

Samples were run in triplicate.

A vial of the current Potency Standard (PTSD) preparation was reconstituted according to the following steps.

The TGA potency standard was removed from −80° C. storage.

Plasma thawer or water bath was verified to be set to 30-37° C.

PTSD was placed into plasma thawer or water bath and allowed to than for 3 minutes.

1 mL of 0.9% saline was added directly to the tube and mixed gently via a pipette.

Three distinctly labeled microcentrifuge tubes were prepared as follows:

960 μL of 0.9% saline was added followed by 50 μL of TGA potency standard, making sure to invert several times to mix.

Prior to testing samples on D×H Hematology Analyzer, the controls were verified and had met passing criteria.

Each diluted tube was run on the D×H Hematology Analyzer.

FluCa solution was prepared by combining two 1.6 mL tubes of Fluo-Buffer and 80 μL of Fluo-Substrate (Stago, stago.com) in a 10 mL cryovial.

The Master mix and FluCa solution were then immediately placed in a 37±2° C. air incubator with agitation for 45±5 minutes, protected from light.

The ClarioStar was turned on according to manufacture's instructions.

The following dilutions were prepared in triplicate for each sample of the platelet products being tested.

Each sample of the platelet products was diluted 1:10 by adding 100 μl of platelet sample to 900 μl of TGA Dilution Buffer.

The 1:10 diluted sample was further diluted to 1:32 by adding 100 μl of the respective platelet product to 220 μl of TGA Dilution Buffer

The 1:32 diluted platelet sample was further diluted to 1:64 by adding 100 μl of the respective platelet product to TGA Dilution Buffer

The 1:64 diluted samples were further diluted to 1:128 by adding 100 μl of the respective platelet product to 100 μl of TGA Dilution Buffer.

The ClarioStar Plus was prepared according to manufacture's instructions. Prior to inserting samples, it was confirmed that there were no abnormalities on the bottom of the assay plate. 10 μl of a platelet product sample and thrombin standards were dispensed into appropriate wells corresponding to the ClarioStar protocol template by reverse pipetting. The samples were run to determine the thrombin activity. The thawed platelet particles obtained upon thawing the pooled CPP product was run for 1 hour. CSP was run for 1-2 hours. RTP was run for 2 hours.

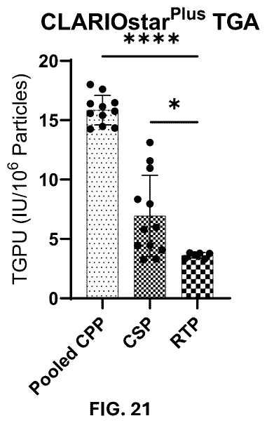

FIG. 21 shows the TGPU (IU/10⁶ particles for each platelet particle. The data shown for pooled CPP product corresponds to the collection of data from the pooled CPP product that was tested after storage in ≤−65° C. freezer for 6 months and the pooled CPP product that was tested after storage in ≤−65° C. freezer for 9 months. RTP (n=6) was tested after storage of 4 days. CSP (n=6) was tested after storage of 7 days and 14 days. Thrombin generation assay measures the potential of platelets and/or platelet particles to catalyze the formation of thrombin, which creates the hemostatic fibrin clot.

FIG. 21 shows that pooled CPP product upon thawing promotes thrombin generation to a greater extent, mean value of TGPU about 15 as compared to CSP (approx. 7 TGPU), and RTP (less than 5 TGPU).

### Example 22: Pooled CPP Product Exhibits Greater Thrombin Generation Achieving Higher Peak Height in Less Time to Peak in a TGA Assay Using Thrombinoscope

Samples from the three platelet products were taken from respective platelet units at different timepoints as disclosed in Example 17 for testing.

The Thrombinoscope was prepared according to manufacturer's guideline verifying the calibrator activity, plate layout, and settings.

Necessary reagents and samples from platelet products were prepared according to the following steps.

30 mL of deionized water from Millipore filter was placed into a new conical tube and tube was placed into a water bath.

14 mL of TGA Dilution buffer was removed from −20° C. freezer and placed in a water bath for approximately 10 minutes.

24 mL of Octaplas was removed from the −80° C. freezer and placed in a water bath for approximately 15 minutes, inverting tubes several times after 15 minute water bath period to ensure uniform plasma concentration.

1 vial of PRP reagent, 1 vial of Thrombin Calibrator, 2 vials of Fluo-Buffer, and 1 vial of Fluro-substrate (Stago, stage.com) were removed from a 4° C. freezer and stored at room temperature. The Fluo-substrate (Stago, stage.com) was thawed in 37 water bath for approximately 2 minutes; once thawed it was kept at room temperature in the dark.

A 1:50 dilution of UPTT Cephalin control was created to be plated with the platelet products being tested. First a 1:5 Cephalin was created by combining 200 μL rehydrated UPTT Cephalin with 500 μL TGA dilution buffer and 300 μL Octaplas (Octapharma, octapharmausa.com). Then a 1:50 Cephalin was created by combining 1:5 Cephalin with 900 μL of 30% Octaplas (300 μL Octaplas+700 μL TGA Dilution buffer).

All above reagents were rehydrated with sterile water according to label instructions. Rehydrated reagents were mixed by inverting the capped vials five times, 10 minutes after rehydration, without vortexing.

A 3.2 mL volume of Fluo-Buffer was pipetted into a conical tube, capped, and placed in a water bath.

A vial of the current Potency Standard (PTSD) preparation was reconstituted according to the following steps.

The TGA potency standard was removed from −80° C. storage.

Plasma thawer or water bath was verified to be set to 30-37° C.

PTSD was placed into plasma thawer or water bath and allowed to than for 3 minutes.

1 mL of 0.9% saline was added directly to the tube and mixed gently via a pipette.

Three distinctly labeled microcentrifuge tubes were prepared as follows:
960 μL of 0.9% saline followed by 50 μL of TGA potency standard, making sure to invert several times to mix.

Prior to testing samples on D×H Hematology Analyzer, the controls were verified and had met passing criteria.

Each diluted tube was run on the D×H Hematology Analyzer

Thrombinoscope plate was then prepared for testing according to the following steps.

For PTSD and pooled CPP product upon thawing, a 1584×10³/μL dilution was used to make 325×10³/μL, 160×10³/L, and 80×10³/L serial dilutions using Octaplas.

Samples with 1584×10³ platelet particles/μL of pooled CPP product upon thawing and PTSD was tested for platelet particle concentration using either the Beckman Coulter® Act Diff and Act Diff 2 Hematology Analyzer or D×H 520 Hematology Analyzer.

For RTP and CSP, samples with 352×10³/μL were directly prepared by diluting the respective platelet product in Octaplas according to the below formula.

C1V1=352×1000, where C1 is the measured CSP/RTP concentration and V1 is the calculated volume (in μL) needed to obtain 1000 μL of CSP/RTP at the desired concentration of 352×10³/μL.

The 352×10³/μL platelet tubes of RTP and CSP were tested for platelet concentration using either the Beckman Coulter® Act Diff and Act Diff 2 Hematology Analyzer or D×H 520 Hematology Analyzer.

20 μL of PRP reagent was added to each test well. 20 μL of calibration was added to each calibration well.

For pooled CPP product upon thawing and PTSD, a 15 μL of each 325×10³/μL, 160×10³/μL, and 80×10³/L samples were added to each test well of their test and calibration well.

For RTP and CSP, a 15 μL of 325×10³/L sample was added to each test well of their test and calibration well.

65 μL of Octaplas was added to every test and calibration wells.

80 μL of 1:50 Cephalin was added to the positice control wells.

The plate was inserted into tray and incubated for 10 minutes at 40. After incubation Fluo-Buffer was dispersed and Fluo-Substrate mixture was added into the active wells.

The plate was read for 75 minutes at 20 second intervals to capture full thrombin generation profile.

Analysis of raw data was performed.

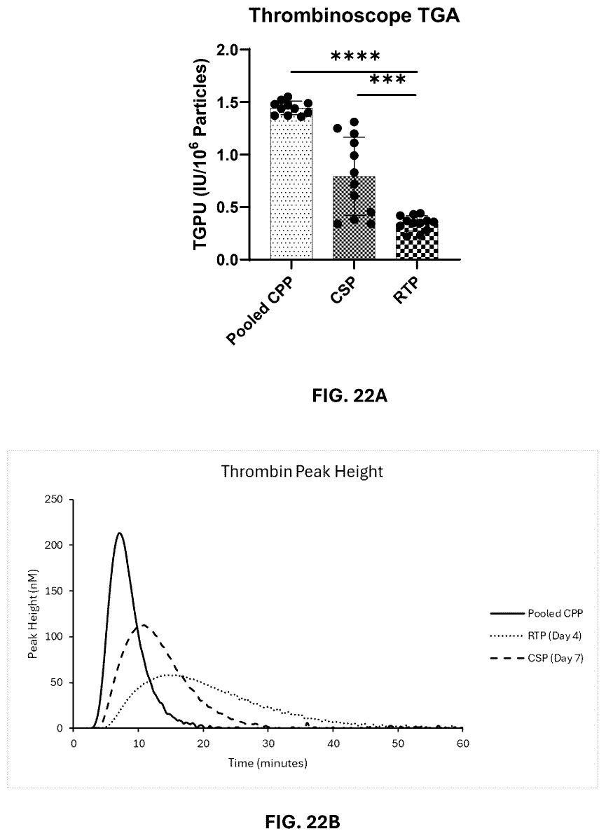

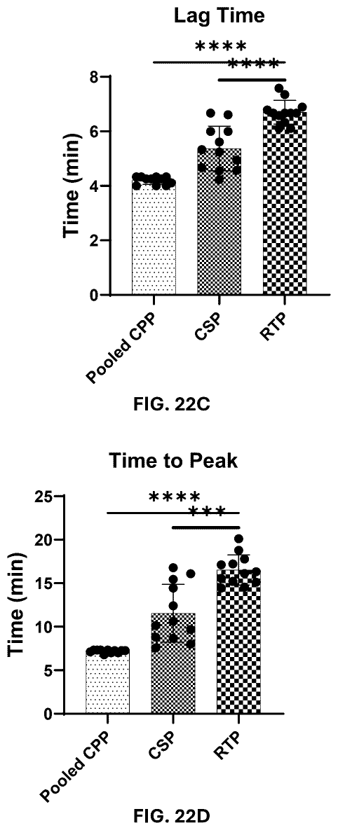

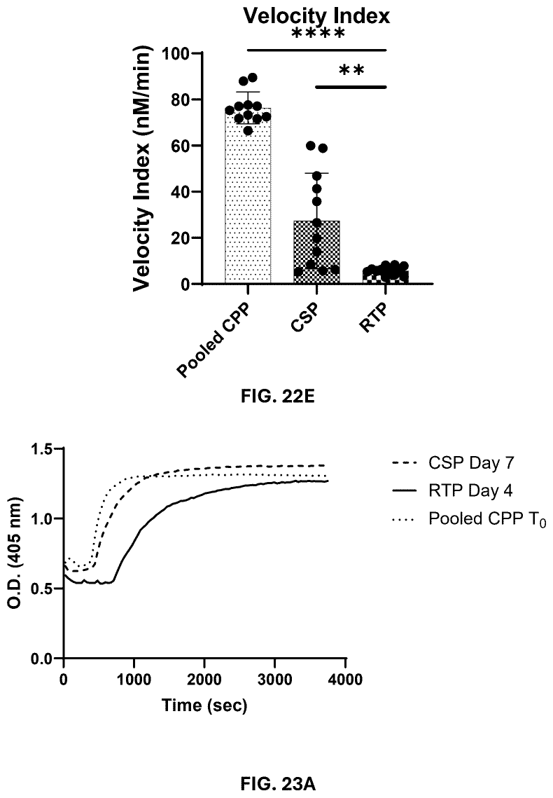

FIG. 22A, FIG. 22B, FIG. 22C, FIG. 22D, and FIG. 22E show the TGPU (IU/10⁶ particles), thrombin peak height, lag time, time to peak, and velocity index for the three platelet products, respectively. The data shown for pooled CPP product corresponds to the collection of data from the pooled CPP product that was tested after storage in ≤−65° C. freezer for 6 months and the pooled CPP product that was tested after storage in ≤−65° C. freezer for 9 months. RTP (n=6) was tested after storage of 4 days and 7 days. CSP (n=6) was tested after storage of 7 days and 14 days.

The results clearly show enhanced thrombin generation activity for the pooled CPP product. FIG. 22A shows that pooled CPP product upon thawing promotes thrombin generation to a greater extent, mean value of TGPU about 15 as compared to CSP (approx. 7 TGPU), and RTP (less than 5 TGPU). In fact, it was observed that the pooled CPP product reached a thrombin peak height of at least 150 nM in less than 10 minutes in an in vitro TGA assay performed using Thrombinoscope, whereas, CSP and RTP were not able to attain the thrombin peak height of 150 nM (FIG. 22B). It was further observed that the pooled CPP product initiates thrombin generation in a lesser amount of time as compared to CSP and RTP (FIG. 22C and FIG. 22D). Further the standard deviation of the various TGA parameters is a lot tighter for the pooled CPP product as compared to CSP and RTP, thereby demonstrating that the pooled CPP product has less variability and maintains better consistency in terms of functional parameters like thrombin generation as compared to CSP and RTP.

### Example 23: Pooled CPP product has a higher speed of clot formation in a clot formation assay done using Clariostar^{Plus}

Samples from the three platelet products were taken at different timepoints as disclosed in Example 17 for testing. ClarioStar^{Plus} was turned on and settings were established for testing clot formation of the platelet products. The clot formation assay measures formation of the hemostatic fibrin clot. This occurs as a consequence of the thrombin generation activity of each platelet product as shown in the TGA assays of Examples 21 and 22. The clot formation assay for the three platelet products was run according to the following methods.

Each platelet product sample was counted in duplicate on the ActDiff2 using 1:20 dilution in PBS for pooled CPP product upon thawing and a 1:10 dilution for RTP and CSP in PBS. The average of the counts was used to calculate the volume of a 1:1000 dilution needed to create a 2,500 particles/μL sample in 1.5 mL PBS. 50 μL of the sample was added in triplicate to a 96 well U-bottom plate.

A serial dilution was performed with 2,500 particles/μL sample on the plate, resulting in 50 μL of sample in triplicate in the following concentrations, plus a blank (PBS only): 2,500 particles/μL, 1,250 particles/μL, 625 particles/μL, 312.5 particles/μL.

Octaplas was thawed at 37° C. for five minutes and filtered using a 0.22 μm syringe filter.

2 vials of PRP reagent are each rehydrated with 928 μL water plus 72 μL 1M calcium chloride and periodically mixed for 10 minutes.

The ClarioStar^{Plus} incubator was set to 37° C., and the plate was placed inside the instrument.

The ClarioStar^{Plus} was primed using Octaplas in pump 1 and PRP reagent/CaCl₂ in pump 2, using a rate of 100 μL/sec to prevent bubbles.

The run was started using the following conditions:

### Plate Mode

Spiral read, 4 mm diameter, 10 flashes

Absorbance reading at 405 nm wavelength

Time between reads: 38 seconds

Total time: 1 hour

Reading direction: top to bottom for each column

Shaking: 5 seconds, orbital, 400 rpm, after injections Octaplas: 50 μL each well, trigger: 20 μL each well, injected at same time on cycle 1, both at speed 100 μL/see

For each unit tested, steps 2 through 7 were performed immediately before the start of the assay.

Assay was run and data was analyzed for all three platelet products. The data shown for pooled CPP product corresponds to the collection of data from the pooled CPP product that was tested after storage in ≤−65° C. freezer for 6 months. RTP (n=6) was tested after storage of 4 days. CSP (n=6) was tested after storage of 7 days.

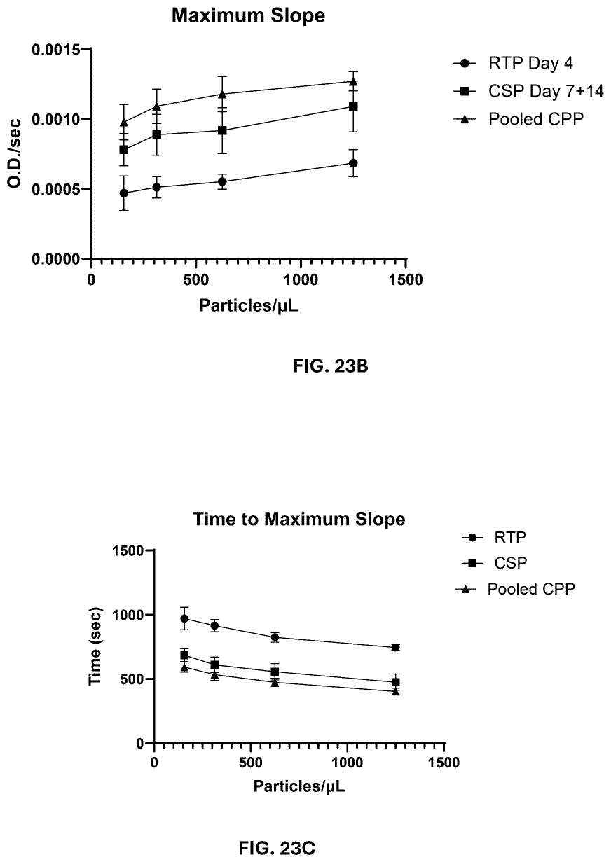

FIG. 23A, FIG. 23B, and FIG. 23C show the optical density over time, maximum slope per platelet particles/μL, and time to maximum slope per platelet particles/μL for the three platelet products, respectively. It was observed that the pooled CPP product upon thawing exhibited greater clot formation strength (FIG. 23B) and reduced reaction time, i.e., the time taken to produce a clot (FIG. 23C).

### Example 24: Pooled CPP Product Triggers Faster Clot Formation in Clot Formation Assay Using TEG 5000 Instrument as Compared to RTP and CSP

Samples of the three platelet products were taken at different timepoints as disclosed in Example 17 for testing. Thromboelastographic (TEG) 5000 was prepared according to manufacturer's guidelines. TEG is a viscoelastic testing platform, and it assesses hemostasis and measures the ability of whole blood sample to form a clot. This test is widely used in the clinical setting to assess hemostatic imbalance and guide appropriate blood component therapy when managing bleeding in patients. The TEG clot formation assay was performed according to the method steps below.

Fresh donor whole blood was collected in sodium citrate vacutainer tubes. Whole blood was used 3 hours within collection.

Pooled CPP product upon thawing was diluted 1:1 with saline. RTP and CSP were not diluted.

Platelet counts for all three platelet products was obtained using ActDiff.

The three platelet products were prepared for spiking at a final concentration of 50K platelets or platelet particles/μL.

1 mL of whole donor blood was transferred to 1.7 mL tube and 50K platelets or platelet particles/μL of the three platelet products or saline was spiked in.

1 mL of the mixture was transferred to a Kaolin vial and mixed well by inversion.

340 μL of the previous sample was transferred to a cup that was preloaded with 20 μL of 0.2 CaCl₂

The samples were then analyzed using the TEG 5000. Samples were run in triplicates simultaneously.

The data shown for pooled CPP product corresponds to the collection of data from the pooled CPP product that was tested after storage in ≤−65° C. freezer for 6 months and the pooled CPP product that was tested after storage in ≤−65° C. freezer for 9 months. RTP (n=6) was tested after storage of 4 days. CSP (n=6) was tested after storage of 7 days and 14 days.

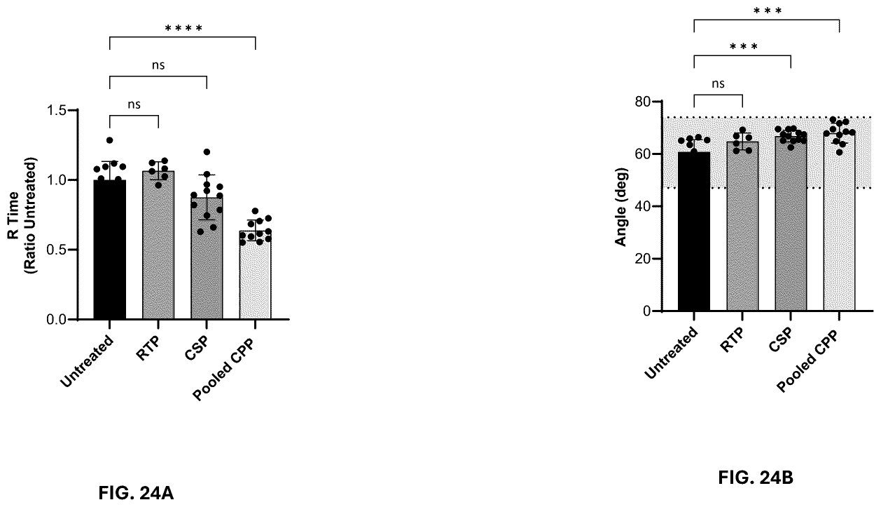

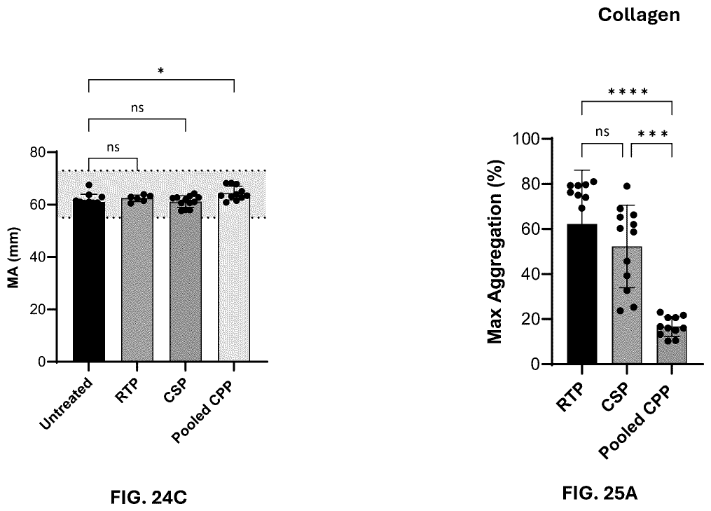

FIG. 24A, FIG. 24B, and FIG. 24C show the R time, a angle, and maximum amplitude (MA) for the blood spiked with different platelet products and untreated (saline addition), respectively.

The Reaction time (R time) is the time measure of clot initiation, and initial fibrin formation. The a angle measures the rate of clot formation by analyzing the angle formed between the end of R time and clotting time, the angle represents clot kinetics during clot build up and crosslinking between fibrin and platelets. The maximum amplitude (MA) measures the maximum displacement, indicative of maximum clot strength of fibrin and platelet cross linking.

FIG. 24A shows a significantly shortened R-time in the presence of pooled CPP product upon thawing (mean R time of about 0.6), suggesting a faster clot initiation as compared to RTP (mean R time of more than 1.0), and CSP (mean R time of about 0.9). FIG. 24B shows a steeper angle for the pooled CPP product upon thawing suggesting a faster clot formation as compared to RTP and CSP. FIG. 24C shows a slight but significant increase of MA in the presence of the pooled CPP product upon thawing. The two dashed lines in FIG. 24B and FIG. 24C are representative of the reference values, the normal range expected in a healthy human. All four assays described in Examples 21-24 demonstrate an improved clot formation dynamics for the pooled CPP product upon thawing relative to the comparative platelet products of RTP and CSP.

### Example 25: Pooled CPP Product has Reduced Activation Potential in a Light Transmission Aggregometry (LTA) Assay Using Agonists

Samples of RTP, CSP, and pooled CPP product were taken from respective platelet units at different timepoints as described in Example 17 for testing. The PAP-8® model 8E platelet aggregometer (Bio/Data, biodatacorp.com) was prepared according to manufacturer's guideline. The LTA assay was performed according to the following method steps.
1. 1 mL of platelet sample (pooled CPP product, RTP, or CSP) was transferred into a 1.7 mL Eppendorf tube. To the same tube, 6 μL of citric acid was added.
2. The platelet samples were pelleted by centrifugation at 1000×g for 10 minutes. The supernatant was removed and the pellets were resuspended at a target concentration of 300K platelets/μL in Octaplas (octapharmausa.com).
3. The platelet counts were measured using the ActDiff2 hematology analyzer.
4. 225 μL of the platelet sample in Octaplas was transferred to a cuvette and placed on the PAP-8®.
5. After two minutes of incubation, aggregation of the samples were triggered by adding 25 μL of the agonist stock to the corresponding cuvette. Agonists used were 10 μg/mL of collagen, 20 μM of ADP, 1.63 μg/mL of arachidonic acid (AA), 10 U/mL of thrombin, 25 μM of TRAP-6, and untreated. The samples were run in triplicates.

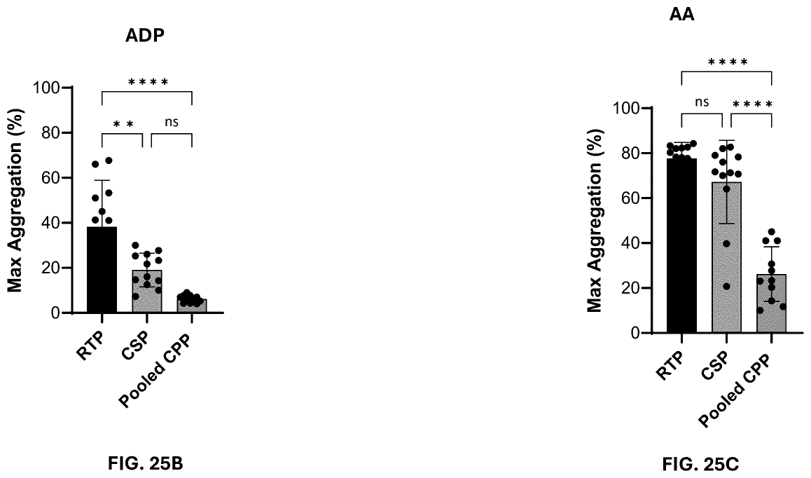

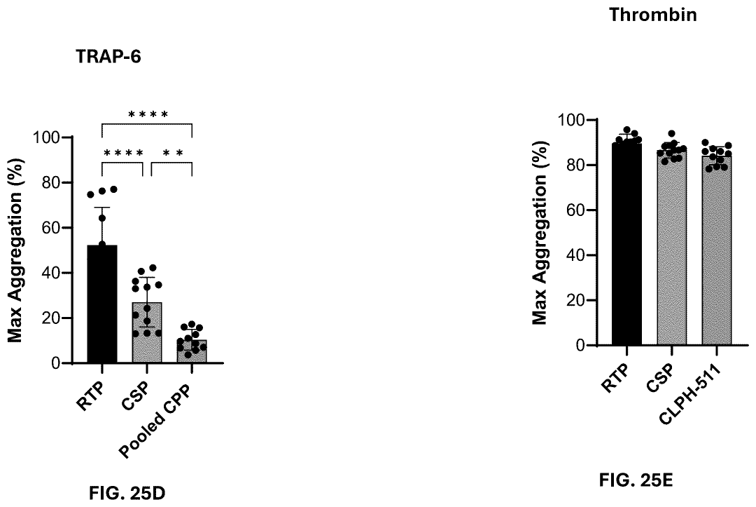

The data shown for pooled CPP product corresponds to the collection of data from the pooled CPP product that was tested after storage in ≤−65° C. freezer for 6 months and the pooled CPP product that was tested after storage in ≤−65° C. freezer for 9 months. RTP (n=6) was tested after storage of 4 days and 7 days. CSP (n=6) was tested after storage of 7 days and 14 days. FIG. 25A, FIG. 25B, FIG. 25C, FIG. 25D, and FIG. 25E show the maximum aggregation (%) response of the different platelet products with the addition of different agonists. The figures shown here illustrate the reduced activation potential of pooled CPP product in response to 4 out of the 5 agonists (collagen, ADP, AA, and TRAP-6). There is a significant difference between the aggregation response of pooled CPP product to RTP in those 4 agonists. Pooled CPP product only responded to thrombin as shown in FIG. 25E. Furthermore, weaker agonists, ADP and TRAP-6, show a minimal aggregation response from pooled CPP product.

### Example 26: Pooled CPP Product Maintains Reduced Aggregation Response to Combined Agonist Stimulation and has Reduced ATP Release in a Lumi-Aggregometry Assay

Chronolog aggregometers can measure platelet aggregation and dense granule release at the same time. Samples of RTP, CSP, and pooled CPP product were taken from respective platelet units at different timepoints as disclosed in Example 17 for testing. The Chrono-log corporation Model 700 (chronolog.com) was prepared according to manufacturer's instructions. The lumi-aggregometry assay was performed according to the following method steps.

1. 1 mL of the platelet sample was placed in a 1.7 mL tube, 6 μL of 1M citric acid was added. This mixture was centrifugated at 1,000×g for 5 minutes.

2. The supernatant was removed and discarded. 1 ml of platelet wash buffer was added and pellet resuspended. The centrifuge step was repeated.
3. The supernatant was discarded, and the pellet was resuspended in 1 mL Octaplas (octapharmausa.com).
4. The samples were diluted 1:20 in PBS and the platelet count was taken using the ActDiff2 hematology analyzer.
5. Samples of 300K particles/μL in 8 mL of Octaplas were created from each unit based on the counts.
6. Since ATP release was measured, the ATP standard was performed using a representative sample in each channel and Octaplas as the blank, according to manufacturer's instructions.
7. Lumi-aggregation tested was initiated according to manufacturer's instructions using the below agonists/agonist combinations for each sample. Samples were run in duplicate/
a. TRAP-6 (10 μM)+ADP (20 μM)+Epinephrine(Epi) (5 μM)
b. Collagen (10 μg/mL)+Epi (10 μM)
c. AA (1.0 mM)+ADP (2 μM)
d. TRAP-6 (25 μM)
e. Collagen (10 μg/mL)

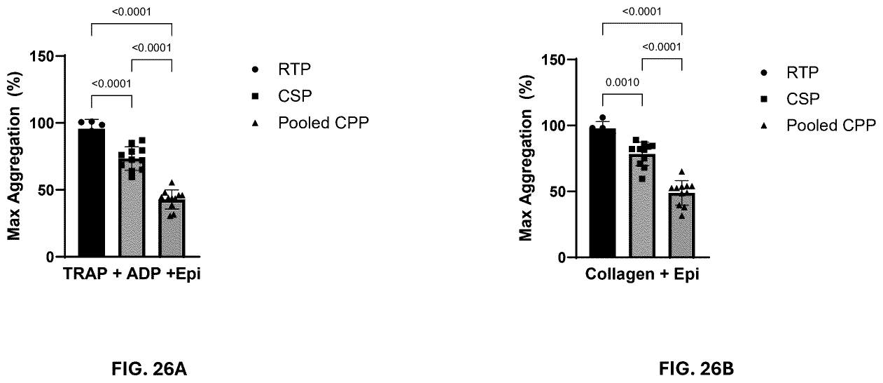

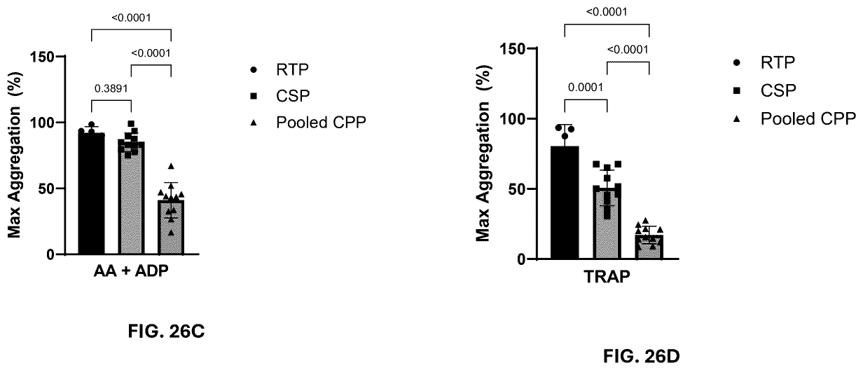

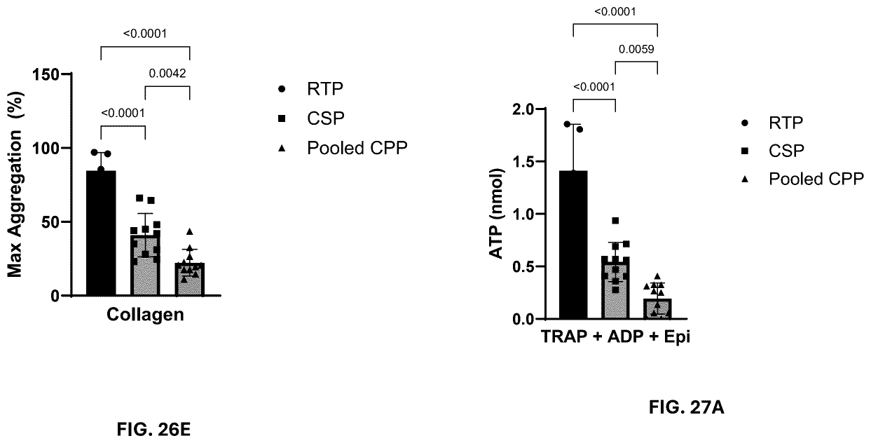

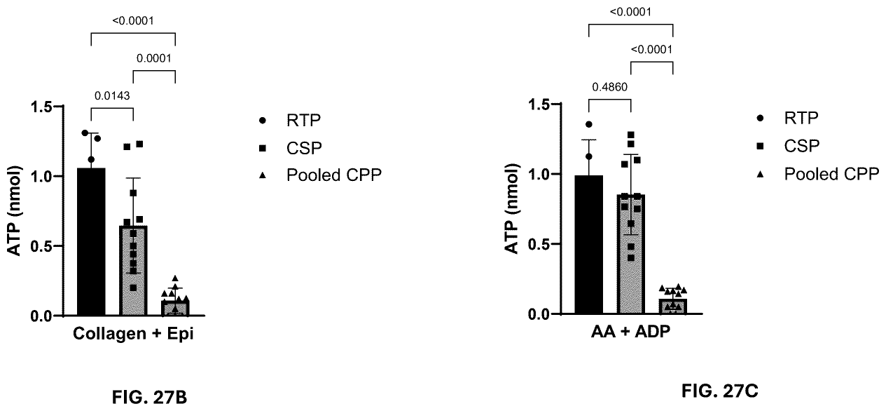

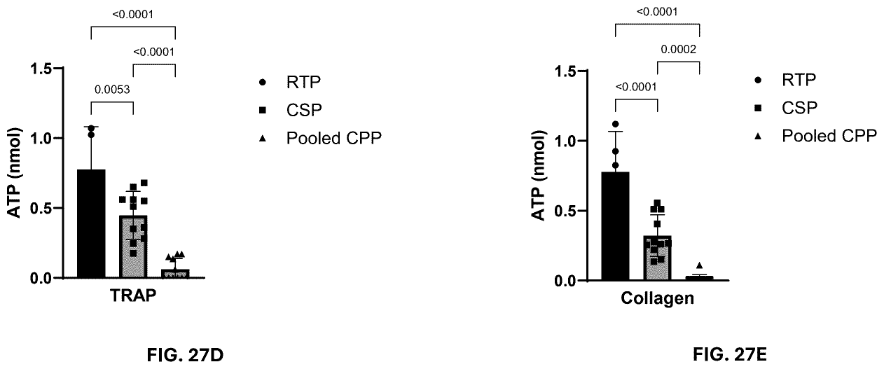

The data shown for pooled CPP product corresponds to the collection of data from the pooled CPP product that was tested after storage in ≤−65° C. freezer for 6 months and the pooled CPP product that was tested after storage in ≤−65° C. freezer for 9 months. RTP (n=6) was tested after storage of 4 days. CSP (n=6) was tested after storage of 7 days and 14 days. FIG. 26A, FIG. 26B, FIG. 26C, FIG. 26D, and FIG. 26E show the maximum aggregation (%) response to the single or combined agonists for each platelet product (pooled CPP product, RTP, CSP). FIG. 27A, FIG. 27B, FIG. 27C, FIG. 27D, and FIG. 27E show the ATP release from the maximum aggregation (%) response to the single or combined agonists for each platelet product. The maximum aggregation response from pooled CPP product improves with combination agonists but still remains relatively reduced with respect to comparative platelet products (RTP and CSP). The reduced aggregation response of pooled CPP product is consistent with the results from Example 25 (PAP-8 aggregation assay). There is significant difference between the aggregation response of pooled CPP product and comparative platelet products shown in FIGS. 26A-26E. Pooled CPP product shows some dense granule retention and ability for release in response to stimulation. But the amount of ATP release is minimal. In fact, there is significant difference in ATP release from comparative platelet products from all the stimulation assays as shown in FIGS. 27A-27E. There was a high amount of ATP in the supernatant of pooled CPP product (not shown); many of these dense granules containing ATP are released during processing or thawing. This feature further demonstrates that pooled CPP product is an activated product.

### Example 27: Inducing activated GPllb/llla in pooled CPP product

Samples of RTP, CSP, and pooled CPP product were taken from respective platelet units at different timepoints as described in Example 17 for testing. The ability to stimulate GPllb/llla in its active conformation was tested for the different platelet products using a NovoCyte Quanteon Flow Cytometer (Agilent, agilent.com). The PAC-1 monoclonal antibody only binds to GPllb/llla in its active conformation, before fibrinogen is bound. This is useful to monitor the baseline activation status, and to monitor the active process of the platelet inducing this adhesion/clot growth receptor conformation change after stimulation. The Quanteon Flow Cytometer was prepared according to manufacturer's instructions. The necessary reagents for the assay were prepared as follows. An agonist mixture containing TRAP-6, ADP, and epinephrine (Epi) was used to stimulate the platelet products and was prepared by mixing 50 μL 1 mM TRAP-6, 100 μL 1M ADP, and 25 μL 1M Epi. A 3.5 μL of the agonist mixture was added to 100 μL of the platelet product to obtain the same concentration of the agonists as in the Lumi-aggregometry assay of Example 26 (10 μM TRAP-6, 20 μM ADP and 5 μM Epi). The three platelet products (pooled CPP product, RTP, and CSP) were stained with PAC-1 and run on the flow cytometer according to the following method steps.
1. A 200K platelet particles/μL stock in PBS was made for each platelet product (pooled CPP product, RTP, CSP) by taking the platelet particle count using the ActDiff.
2. HMTA was thawed at 37° C. and filtered through a 0.22 syringe filter. MgCl₂ was added to achieve a final concentration of 2 mM MgCl₂ in HTMA.
3. HMTA+MgCl₂, PAC-1 antibody or isotype control, and agonist mixture were added to a 96 well U bottom plate for each platelet product tested as follows.
   a. PAC-1 stained, unstimulated
      i. 90 μL HMTA+5 μL PAC-1 antibody
   b. PAC-1 stained, stimulated
      i. 86.5 μL HMTA+5 μL PAC-1 antibody+3.5 μL agonist mixture
   c. IgM isotype stained, unstimulated
      i. 93.5 μL HMTA+1.5 μL IgM (concentration matched to PAC-1 antibody)
   d. IgM isotype stained, stimulated
      i. 90 μL HMTA+1.5 μL IgM+3.5 μL agonist mixture
4. 5 μL of the 200K platelet particles/μL stock for each platelet product stock were added to one set of wells. Samples were gently mixed with pipette after addition
5. The plate was incubated at room temperature for 20 minutes in the dark.
6. The wells were mixed gently with a pipette and 25 μL of each was transferred to 75 μL of HMTA/MgCl₂. This sample was further mixed and 10 μL was transferred to 90 μL HMTA/MgCl₂
7. The final dilution was acquired on the Novocyte Quanteon flow cytometer using the following conditions:
   a. 50 μL or 10,000 events in platelet size gate
   b. Medium flow rate
   c. Mix speed of 800 rpm, 5 seconds
   d. FSC threshold, 1,000
   e. FSC, SSC, APC area and height

The data shown for pooled CPP product corresponds to the collection of data from the pooled CPP product that was tested after storage in ≤−65° C. freezer for 16 hours and the pooled CPP product that was tested after storage in ≤−65° C. freezer for 1 month. RTP (n=6) was tested after storage of 4 days. CSP (n=6) was tested after storage of 7 days and 14 days.

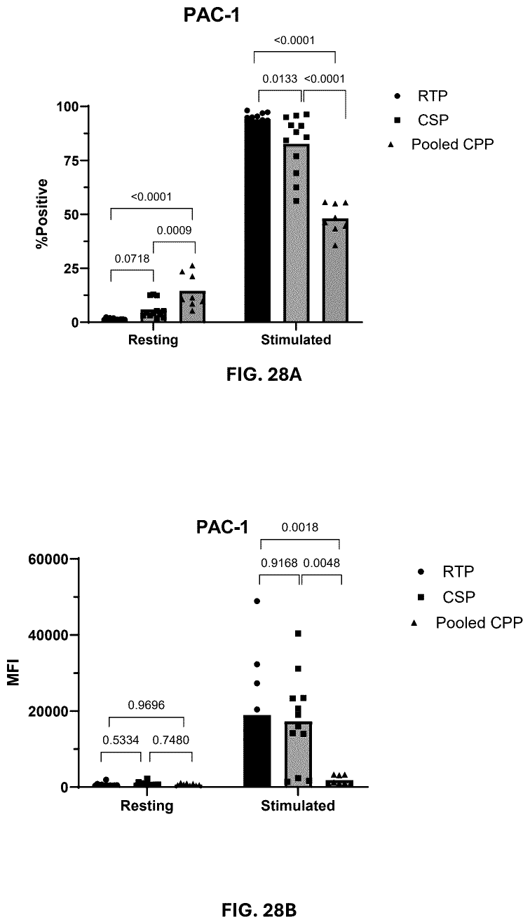

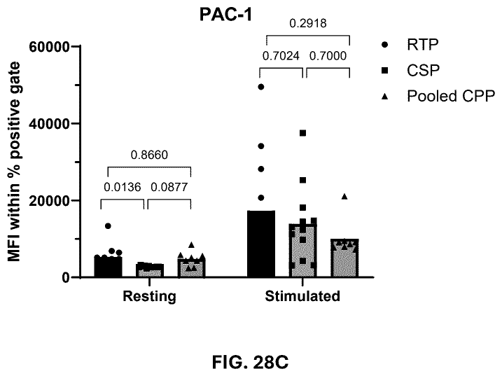

Analysis of the results was performed. FIG. 28A, FIG. 28B, and FIG. 28C show the percent positivity, MFI, and MFI within the percent positive gate for PAC-1 for all three platelet products (pooled CPP product, RTP, and CSP) resting and stimulated with agonist mixture. A large increase in positivity was observed after stimulation for each of the 3 platelet products tested that was apparent when assessing the data as percent positivity (FIG. 28A). Specifically with respect to the pooled CPP product, the resting data in FIG. 28A is consistent with the observation that the pooled CPP product comprises frozen activated platelets. Furthermore, on comparing the readings for “resting” and “stimulated” pooled CPP product in each of the figures (FIG. 28A, FIG. 28B, and FIG. 28C), it was observed that the population in the pooled CPP product as a whole becomes further activated as measured by GPIIb/IIIa confirmation, upon stimulation with an agonist in vitro. Further, it can be observed that even upon stimulation, the pooled CPP product samples did not reach the levels of GPIIb/IIIa activation as shown by CSP and RTP.

### Example 28: Pooled CPP Product is a More Homogeneous Platelet Product as Compared to RTP and CSP

Additional statistical analysis of variability was performed on the data observed from Examples 18, 19, and 22 disclosed herein. These analysis parameters include:
1. Range: the difference between the highest and lowest values
2. Interquartile range: the range of the middle half of a distribution (25% to 75% range)
3. Standard deviation: average distance from mean
4. Coefficient of Variation (CoV): the ratio of the standard deviation to the mean
5. Mean normalized: the mean of a set of values after normalization
   a. Mean normalized=individual value ÷ group mean

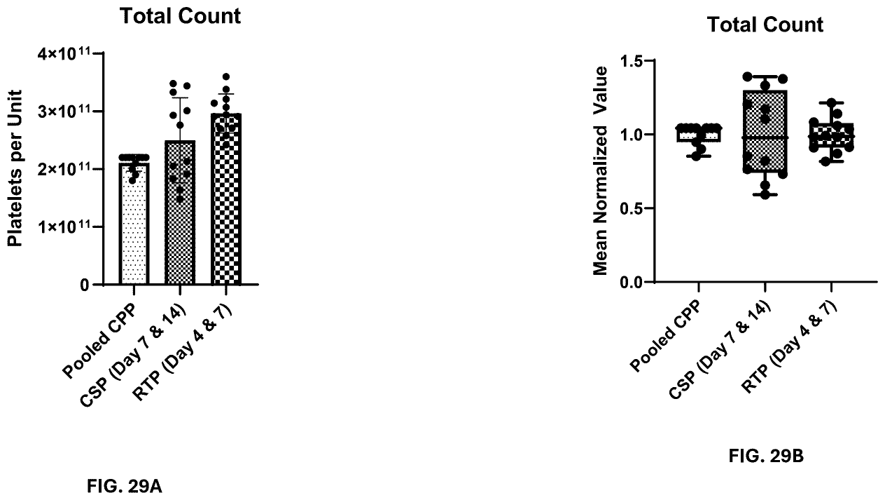

The data shown for pooled CPP product (n=6) corresponds to the collection of data from the pooled CPP product that was tested after storage in ≤−65° C. freezer for 6 months and the pooled CPP product that was tested after storage in ≤−65° C. freezer for 9 months. RTP (n=6) tested after storage of 4 days and 7 days, and CSP (n=6) tested after storage of 7 days and 14 days. FIG. 29A and FIG. 29B shows the total platelet count and mean normalized value, respectively.

Table 23 and table 24 below show the coefficient of variation (CoV) and absolute range for the platelet count, respectively, for the three products.

TABLE 23
Coefficient of Variation

| Pooled CPP Product | CSP | RTP |
|---|---|---|
| 7% | 29% | 11% |

TABLE 24
Absolute Range: Min-Max (Range)

| Pooled CPP Product | CSP | RTP |
|---|---|---|
| 1.8-2.2e11 (0.4) | 1.5-3.5e11 (2.0) | 2.4-3.6e11 (1.2) |

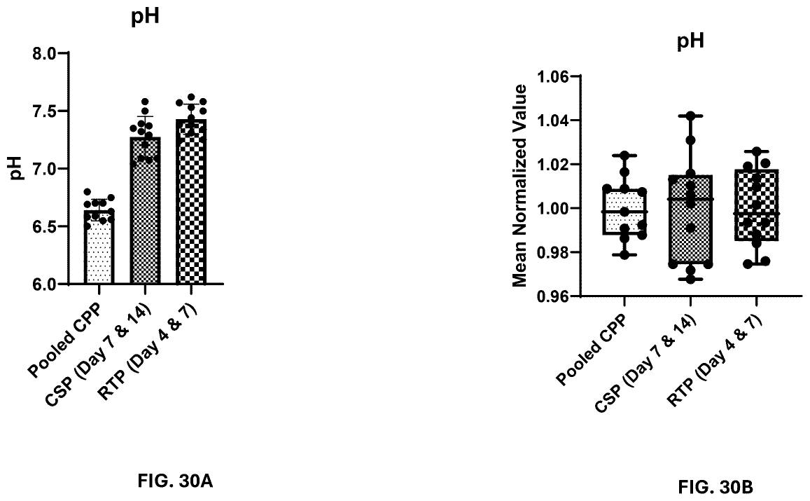

The mean normalized value for pooled CPP product is the tightest of the comparative platelet products with the lowest CoV. FIG. 30A and FIG. 30B show the pH and the mean normalized value of the pH, respectively. Table 25 and table 26 below show the CoV and absolute range for the pH, respectively, for the three products.

TABLE 25
Coefficient of Variation

| Pooled CPP Product | CSP | RTP |
|---|---|---|
| 1% | 2% | 2% |

TABLE 26
Absolute Range: Min-Max (Range)

| Pooled CPP Product | CSP | RTP |
|---|---|---|
| 6.5-6.8 (0.3) | 7.0-7.6 (0.6) | 7.2-7.6 (0.4) |

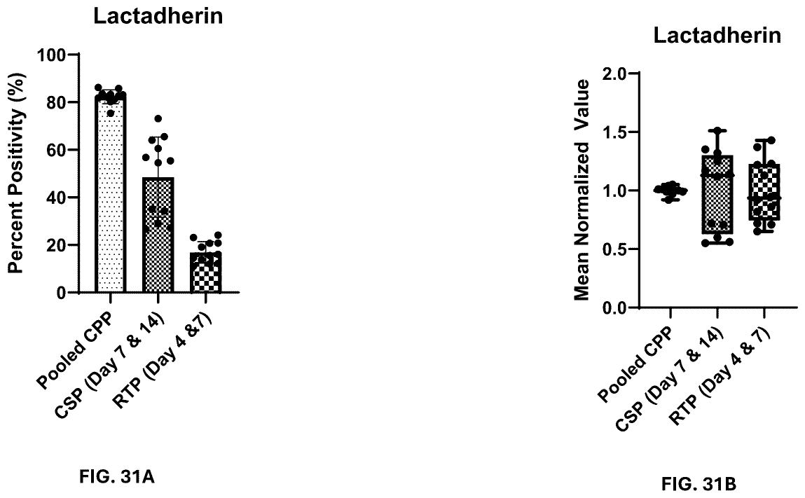

The mean normalized value for the pH remains to be the tightest of the group and CoV is the lowest as well for pooled CPP product. FIG. 31A and FIG. 31B show the percent positivity and mean normalized value, respectively, for Lactadherin. Table 27 and table 28 below show the coefficient of variation and absolute range for Lactadherin positivity, respectively, for the three products.

TABLE 27
Coefficient of Variation

| Pooled CPP Product | CSP | RTP |
|---|---|---|
| 4% | 35% | 27% |

TABLE 28
Absolute Range: Min-Max (Range)

| Pooled CPP Product | CSP | RTP |
|---|---|---|
| 75-86 (11) | 27-73 (47) | 11-24 (13) |

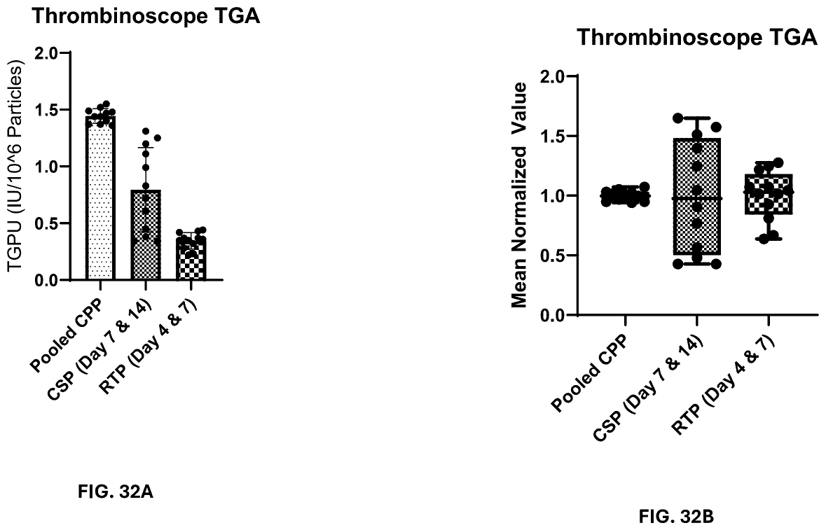

Pooled CPP product consistently has high phosphatidylserine. The mean normalized value for Lactadherin positivity is very tight and the coefficient of variation is less than 5%. Pooled CPP product is demonstrated herein to be the most homogeneous platelet product when measured at different timepoints of storage as compared to CSP and RTP. FIG. 32A and FIG. 32B show the thrombin activity-TGPU (IU/10⁶ particles) and the mean normalized value of the thrombin activity TGPU (IU/10⁶ particles). Table 29 and table 30 below show the coefficient of variation and absolute range for the thrombin activity, respectively, for the three products.

TABLE 29
Coefficient of Variation

| Pooled CPP Product | CSP | RTP |
|---|---|---|
| 4% | 47% | 21% |

TABLE 30
Absolute Range: Min-Max (Range)

| Pooled CPP Product | CSP | RTP |
|---|---|---|
| 1.4-1.6 (0.2) | 0.3-1.3 (1.0) | 0.2-0.4 (0.2) |

Pooled CPP product has the lowest absolute range and highest thrombin activity values. The mean normalized value and coefficient of variation is extremely lower for pooled CPP product. Pooled CPP product has lower unit to unit variability than the other compared platelet products (RTP and CSP).

### Example 29: Pooled CPP Product is Compatible with Different Thawing Machines/Processes to Achieve a Reduced Thawing Time

Pooled CPP product is presently thawed using a wet-thaw process using a circulating water bath. Frozen units are placed in plastic bags to protect unit. Mechanical arms agitate units up and down to help facilitate thawing. Cellphire routinely uses a Helmer QuickThaw DH8 for thawing pooled CPP product. The objective of this study was to determine alternative mechanisms/machines for thawing to optimize the procedure. Helmer QuickThaw is a water-bath based plasma thawer with a mechanical arm for agitation, it is considered a “wet” thawer. Thawers are considered “dry” if they do not use open water or use water that cannot contact the product. Dry thawers tend to utilize circulating air and/or heating plates or a water-filled protective bladder to facilitate heat transfer and product thawing. The two additional thawers that will be compared to the QuickThaw are the Fremon Scientific ZipThaw and the Sarstedt Sahara III. ZipThaw uses heated, liquid filled cushion with a ZipSleeve to facilitate heat transfer and mechanical massaging to agitate the product during thawing. The Sahara III uses a heating plate and heated circulating air to thaw. Mechanical rocking of the heating plate is used to agitate the product. All three thawers (QuickThaw, ZipThaw, and Sahara III) are FDA exempt or cleared medical devices for warming/thawing blood components. ZipThaw has an internal temperature sensor that continuously monitors and logs the pooled CPP product unit's temperature during thawing to ensure required temperature has been reached and thawing cycle is complete. The Sahara III also has a temperature sensor to monitor and log product temperature during thawing. For utilizing the Sahara III machine, the internal temperature sensor was not used and instead a time-based thawing method was used to evaluate the time needed to reach at least 30° C. using an external infrared (IR) thermometer. An initial experiment was conducted to determine if the QuickThaw process of the current pooled CPP product thawing protocol can be optimized before moving forward with the comparison study with dry thawers. In addition, QuickThaw serves as a control group for wet plasma thawers in this study. The initial preliminary evaluation was conducted according to the following method steps.
1. 6 units of non-GMP pooled CPP product were frozen with temperature probes inside of the units to track pooled CPP product thawing profile using a 37° C. water bath and manual agitation, which is comparable to QuickThaw method.
   a. The pooled CPP product units were manufactured and stored according to the method of Example 2 disclosed herein.
2. By 4 minutes all pooled CPP product units had reached at least 30° C.

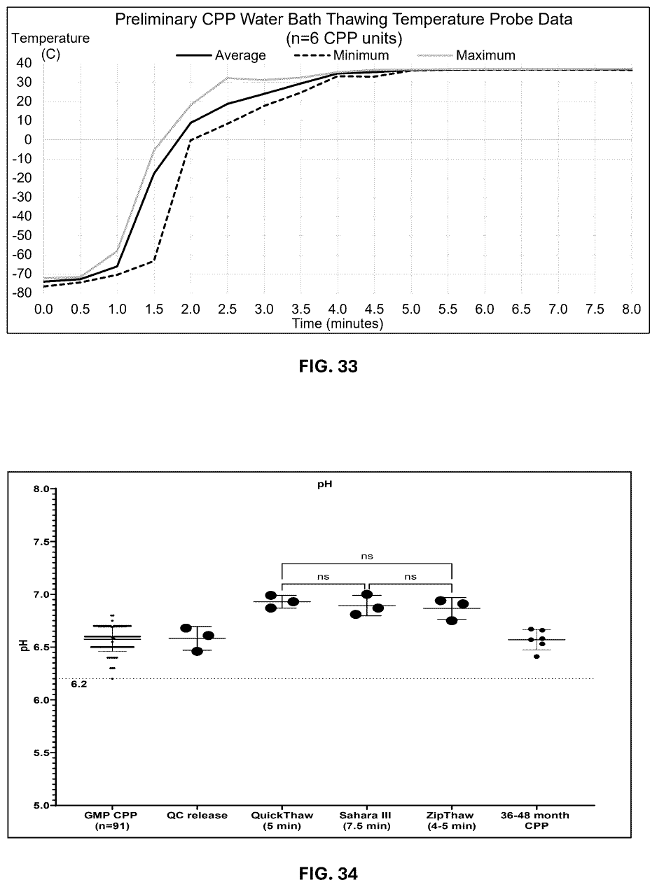

FIG. 33 shows the temperature probe data from the preliminary experiment that tracked the thawing profile of 6 pooled CPP product units in water bath and manual agitation. FIG. 33 contains traces of minimum, maximum, and average temperatures of the units at 30 second intervals. As shown, by 4 minutes all units exceeded 30° C. Given this preliminary data, a thaw time of 5 minutes was preset for QuickThaw and accordingly, the study reports on the stability of a 5 minute Quickthaw cycle. The method of determining the thaw time/process to reach at least 30° C. for each thawer (QuickThaw, Zipthaw, Sahara III) was performed according to the following steps.
1. 9 non-GMP pooled CPP product units were manufactured according to the method of Example 2 as disclosed herein, and stored at ≤65° C. for approximately 3.5 years before testing.
   a. 3 units of W4464 21 000031
   b. 3 units of W4464 21 000041
   c. 3 units of W4464 21 000049
2. Each unit of pooled CPP product was thawed according to the specific instructions for the thawer.
   a. ZipThaw—removed frozen pooled CPP product units were removed from supplied overwrap and placed into ZipSleeve, as indicated by labeling, with the pooled CPP product's ports going into the bottom of the ZipSleeve. The “THAW” function was initiated and cycle was completed once pooled CPP product units reached at least 30° C. IR thermometer was used to detect that the pooled CPP product temperature had reached at least 30° C., additional thawing time was added if 30° C. was not reached.
   b. Sahara III—the overwrap bag was not used. The pooled CPP product unit was placed onto the heating plate with the label facing upward and the ports facing into Sahara III. The unit was thawed using the “37° C. function” for 7 minutes. An initial time of 7 minutes was chosen since this was similar to the maximum amount of time required during the ZipThaw. IR thermometer was used to detect that the pooled CPP product temperature had reached at least 30° C., additional thawing time was added if 30° C. was not reached.
   c. QuickThaw—the overwrap bag was used with the pooled CPP product unit. Thawing was performed in a 5 minute cycle. IR thermometer was used to detect that the pooled CPP product temperature had reached at least 30° C., additional thawing time was added if 30° C. was not reached.
3. After thawing, each pooled CPP product unit was resuspended with 25 mL of 0.9 saline making the total volume of 45-60 mL as is disclosed in pooled CPP product thawing processes disclosed herein.

Table 31 below contains the thaw time and post-thaw temperature results from the experiment. All units reached required minimum temperature in the 5 minute QuickThaw method. ZipThaw performed well too with a temperature range from 5.08 to 7.14 minutes. Sahara III had 1 pooled CPP product unit that required an additional 30 seconds (total time 7.5 minutes) to reach the required minimum temperature, the remaining two pooled CPP product units satisfied minimum temperature at 7 minute thaw cycle.

TABLE 31

| | | Pooled | | | | | Mean |
|---|---|---|---|---|---|---|---|
| | Unit ID | CPP | Initial | Initial | Final | Final | Post- |
| | (W4464- | Product | Thaw | Thaw | Thaw | Thaw | Thaw |
| | 21- | Volume | Time | Temp. | Time | Temp. | Temp. |
| Thawer | 0000) | (mL) | (min.) | (° C.) | (min.) | (° C.) | (° C.) |
| Quick | 41-L | 27.2 | 5:00 | 34.4 | n/a | | 35.1 |
| Thaw | 31-A | 28.3 | 5:00 | 35.1 | | | |
| (5 min.) | 49-H | 27.2 | 5:00 | 35.7 | | | |
| Zip | 41-G | 27.4 | 5:08 | 31.6 | n/a | | 32.1 |
| Thaw | 31-F | 28.9 | 7:14 | 33.1 | | | |
| | 49-C | 27.0 | 5:38 | 31.6 | | | |
| Sahara | 41-F | 27.3 | 7:00 | 29.8 | 7:30 | 33.2 | 32.9 |
| III | 31-E | 28.3 | 7:00 | 32.2 | n/a | | |
| | 49-G | 27.0 | 7:00 | 33.3 | | | |

This experiment clearly shows the use of alternative thawers and a shortened QuickThaw cycle to thaw pooled CPP product units to at least 30° C. The compatibility of a dry thaw machine process with pooled CPP product units allows for a more accessible thawing process in platelet transfusion environments. Furthermore, the 5 minutes thaw cycle of Quickthaw reduces the standard time (8 minutes) by 3 minutes.

### Example 30: Pooled CPP Product Meets all Release Criteria Requirement, TGA, and Percent Positivity for Lactadherin and Microparticles with No Significant Differences Among the Three Thawing Machines/Mechanisms (QuickThaw, ZipThaw, Sahara)

This experiment was a confirmatory study of the thaw time and method with release criteria, TGA, and percent positivity of MP and Lactadherin tested. The study was performed according to the following method steps.
1. 9 clinical pooled CPP product were manufactured and stored for 3.5 year prior to testing according to the process of Example 2 disclosed herein.
   a. 3 units from W4464 21 000013
   b. 3 units from W4464 21 000019
   c. 3 units from W4464 21 000021
2. Each unit of pooled CPP product was thawed according to the instructions defined in Example 30 disclosed herein. The only deviation was that a 7.5 minute thaw cycle was used for Sahara III, per results from Example 29.
3. Each pooled CPP product was tested for the following parameters according to the procedures of Example 9 disclosed herein.
   a. Platelet count per cryo vessel (>1.7×10¹¹)
   b. pH (>6.2)
   c. Thrombin generation assay (TGA-IU/10⁶particles)
   d. Flow cytometry characterization for CD61+microparticles (×10⁶/4μL)
   e. Lactadherin positive particles (%)
4. Prior to moving forward with any of the analytical testing, visual inspections for bag integrity and aggregate-free swirling were performed.

Table 32 below contains the thaw time and post-thaw temperature results for the units.

TABLE 32

| | Unit ID | CPP | Thaw | Post-Thaw | Mean Post- |
|---|---|---|---|---|---|
| | (W4464- | Volume | Duration | Temp. | Thaw Temp. |
| Thawer | 21-0000) | (mL) | (min:sec) | (C.) | (° C.) |
| Quick | 13-C | 27.8 | 5:00 | 35.4 | 35.1 |
| Thaw | 19-F | 27.3 | 5:00 | 34.7 | |
| (5 min.) | 21-A | 26.8 | 5:00 | 35.1 | |
| Zip Thaw | 13-D | 28.2 | 4:47 | 32.3 | 32.4 |
| | 19-H | 27.3 | 4:41 | 32.6 | |
| | 21-C | 26.9 | 4:12 | 32.2 | |
| Sahara | 13-E | 27.9 | 7:30 | 33.6 | A34.3 |
| (7.5 min.) | 19-G | 27.3 | 7:30 | 34.1 | |
| | 21-B | 26.8 | 7:30 | 35.2 | |

All units reached at least 30° C. following initial thaw cycle and passed visual inspections for aggregate-free swirling and bag integrity. In addition to the analytical results obtained from the 9 units, data was compared to GMP pooled CPP (also referred to as GMP CPP) units tested from 91 clinical/stability batches (8 minute thaw using Quickthaw) majority falling within the range of 2 weeks to 2 months post storage, and stability data from 36-48 month stored pooled CPP product (stability study data (n=3) for 36 and 48 month storage timepoints and 8 minute thaw using Quickthaw). In addition, all data points were compared to QC release that incorporates the QC release data of the batches used in this experiment. The totality of these batches were thawed using standard 8 minute QuickThaw and tested after 3-15 days of storage. FIG. 34 and Table 33 show the results of the pH testing using the three thawers and the GMP CPP, 36-48 month pooled CPP product, and QC release. The dotted line on the graph identifies the release criteria for that parameter. The pH values for all three thawing machines were within specification and no significant statistical difference were observed between the three thawers via ANOVA Tukey's test. Although the pH values for the three thawers are slightly higher that historical pooled CPP datasets (GMP CPP, QC release, 36-48 month pooled CPP product), these differences are small in magnitude (˜5% different) as shown in Table 33. The higher pH is possibly due to the lower thaw time (5 minutes versus 8 minutes).

TABLE 33

| | mean | Difference from |
|---|---|---|
| Dataset | pH | GMP CPP |
| GMP CPP | 6.57 | n/a |
| QC release | 6.58 | 0.01 |
| QuickThaw (5 min) | 6.93 | 0.36 |
| Sahara III (7.5 min) | 6.89 | 0.32 |
| ZipThaw (4-5 min) | 6.87 | 0.30 |
| 36-48-month pooled CPP product | 6.57 | 0 |

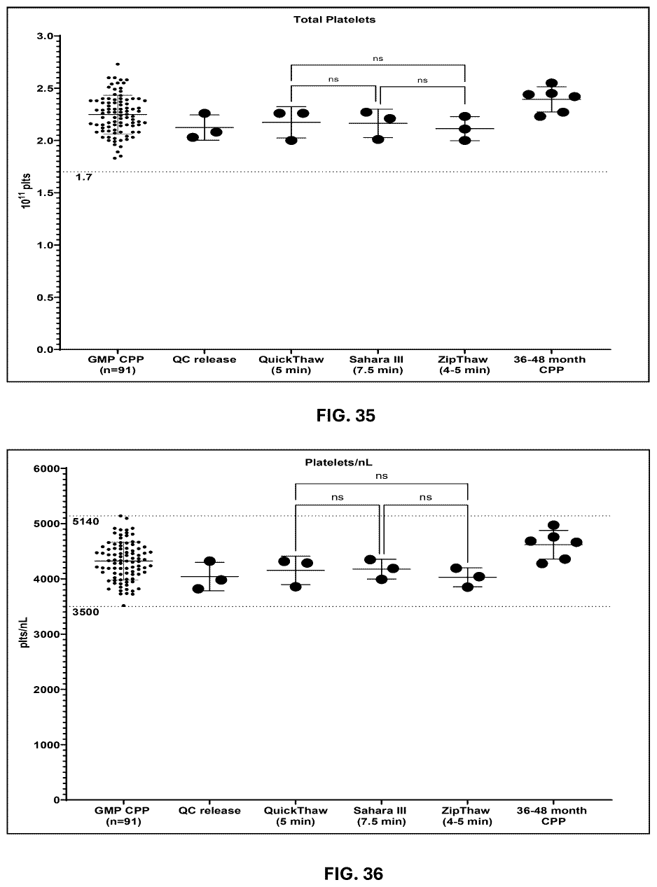

FIG. 35 shows the results of the total platelet count using the three thawers and the GMP CPP, 36-48 month pooled CPP product, and QC release. The dotted line on the graph identifies the release criteria for that parameter. Total platelet count values were all within specifications and there was no statistical different between values from the three thawers via ANOVA Tukey test (p>0.05). Furthermore, the total platelet counts from using the three thawers was consistent with historical pooled CPP data (GMP CPP, QC release, and 36-48 month pooled CPP product).

FIG. 36 shows the results of the platelet concentration (platelets/nL) using the three thawers and the GMP CPP, 36-48 month pooled CPP product, and QC release. The dotted lines on the graph identifies the release criteria for that parameter. The platelet concentration values from the units using the three thawers were within range of the GMP CPP and QC release historical data and were not statistically significant amongst each other using ANOVA Tukey test (p>0.05).

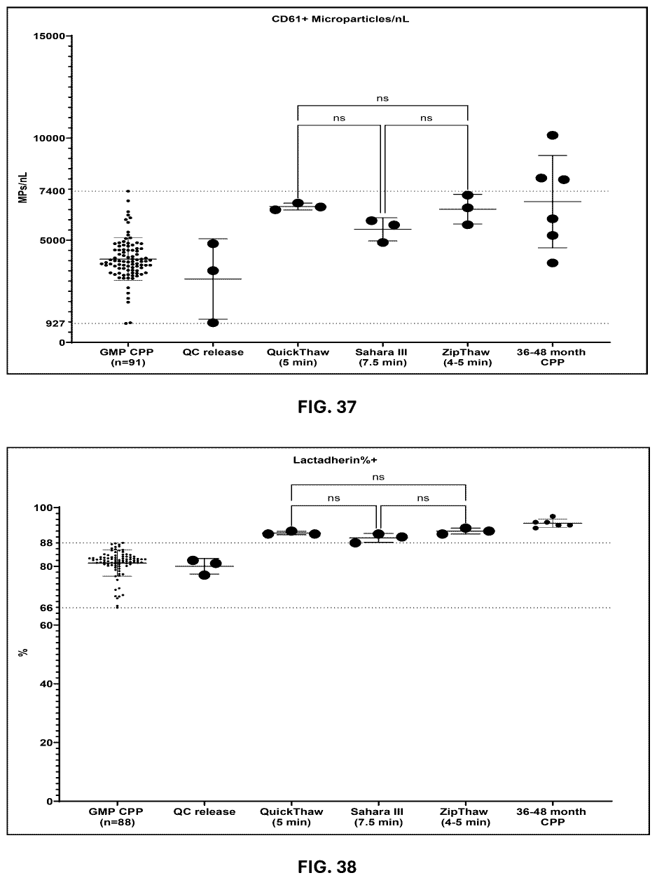

FIG. 37 shows the CD61+ microparticle (MP) concentration using the three thawers and the GMP CPP, 36-48 month pooled CPP product, and QC release. The dotted lines on the graph identifies the release criteria for that parameter. The CD61+ MP concentration values using the three thawers are within the range of the value for GMP CPP historical database. Furthermore there is no statistical significance observed between the CD61+ MP concentration values from the three thawers using ANOVA Tukey test (p>0.05). Although the values from the three thawers appear to be increased compared to QC release dataset, this is consistent with the 36-48 month pooled CPP product dataset. Note there is no release specifications for this attribute and its impact on clinical outcomes is unknown. The CD61+ MP concentration is used as an information only purity test (FIO).

FIG. 38 shows the percentage of CD61+ particles that are also positive for Lactadherin (Lactadherin %+) using the three thawers and the GMP CPP, 36-48 month pooled CPP product, and QC release. The dotted lines on the graph identifies the release criteria for that parameter. The Lactadherin %+ values from the three types of thawers are higher than the range of the GMP CPP dataset, but consistent with 36-48 month pooled CPP product dataset. This is expected of CPP product from a stability profile since the products thawed with the different thawers are approximately 43 months old (3.5 years). Finally no statistical significance was observed between the %+Lactadherin values from the different thawers based on ANOVA Tukey test (p>0.05).

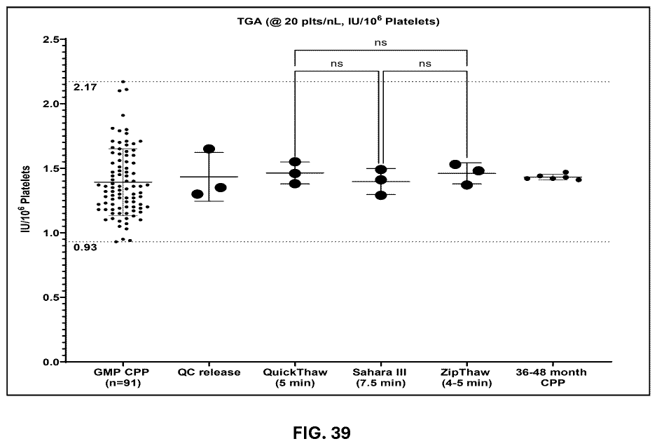

FIG. 39 shows the TGA data using the three thawers and the GMP CPP, 36-48 month pooled CPP product, and QC release. The dotted lines on the graph identifies the release criteria for that parameter. The TGA values from the three thawing processes were within range of the values from the GMP CPP and QC release datasets. Like the CD61+MP concentration, the TGA is used as a FIO potency test. There is no release specifications for this attribute and its impact on clinical outcomes is unknown. Similar to all tests in the example, there is no statistical significance between the TGA values obtained from using the different thawers using ANOVA Tukey test (p>0.05). Table 34 below captures the entirety of the data collected in this experiment.

TABLE 34

| | | 8 min. Quick Thaw Mean & Range | | | | | |
|---|---|---|---|---|---|---|---|
| | | | | 36-48 mo. | Experiment #2 Mean & Range | | |
| | | | | Pooled | (n = 3 batches/thawer, 1 batch/unit) | | |
| | Release | GMP | QC | CPP | Quick Thaw | Sahara | ZipThaw |
| QC Test | Criteria | CPP | Release | Prod. | (5 min.) | (7.5 min.) | (4-5 min.) |
| Aggregate-free | PASS | PASS | PASS | PASS | PASS | PASS | PASS |
| Swirling | | | | | | | |
| Bag | PASS | PASS | PASS | PASS | PASS | PASS | PASS |
| Intact | | | | | | | |
| pH | ≥6.2 | 6.6 | 6.6 | 6.6 | 6.9 | 6.9 | 6.9 |
| | | (6.2-6.8) | (6.5-6.7) | (6.4-6.7) | (6.9-7.0) | (6.8-7.0) | (6.8-7.0) |
| Total Platelets | ≥1.7 | 2.2 | 2.1 | 2.4 | 2.2 | 2.2 | 2.1 |
| (×10¹¹) | | (1.8-2.7) | (2.0-2.3) | (2.2-2.6) | (2.0-2.3) | (2.0-2.3) | (2.0-2.2) |
| Platelet Conc. | FIO (no | 4.3 | 4.0 | 4.6 | 4.2 | 4.2 | 4.0 |
| (×10³ plts/nL) | release | (3.5-5.1) | (3.8-4.3) | (4.3-5.0) | (3.9-4.3) | (3.9-4.3) | (3.9-4.2) |
| CD61 + MP | criteria, | 4.1 | 3.1 | 6.9 | 6.6 | 5.5 | 6.5 |
| Conc. (×10³ | unknown | (0.9-7.4) | (1.0-4.8) | (3.9-10.) | (6.5-6.8) | (4.9-6.0) | (5.8-7.2) |
| MP/nL) | clinical | | | | | | |
| Lactadherin | impact) | 81% | 80% | 95% | 91% | 90% | 92% |
| %+ | | (66-88%) | (77-82%) | (93-97%) | (91-92%) | (88-91%) | (91-93%) |
| TGA (IU/10⁶ | | 1.4 | 1.4 | 1.4 | 1.5 | 1.4 | 1.5 |
| plts) | | (0.93-2.2) | (1.3-1.7) | (1.4-1.5) | (1.4-1.6) | (1.3-1.5) | (1.4-1.5) |

In conclusion, no statistical differences were observed between the QuickThaw (5 minutes), Sahara III (7.5 minutes), and ZipThaw groups. The thawing methods explored in this example produced pooled CPP product with acceptable final product characteristics. Overall, the data is acceptable and pooled CPP product is considered compatible with new thawing methods (dry thawers).

### Example 31: Pooled CPP Product Maintains its Homogeneity after Extended Storage Conditions

Samples of pooled CPP product were taken from product stored in ≤−65° C. freezer for 12 months. Each sample was tested for platelet count, pH, lactadherin binding positivity, thrombin generation, and clot time analysis.

Lactadherin binding positivity was determined according to the method of Example 9. Platelet count and pH were determined according to the methods of Example 18. The thrombin generation was determined according to the method of Example 22. The clot time analysis was determined according to the method of Example 24. The data for pooled CPP product at 12-month storage timepoint for each of the parameters was compilated with the data taken for pooled CPP product at 6-month and 9-month storage timepoints that were measured according to the previous examples. The compiled pooled CPP data for each parameter was compared to previously compiled data for RTP and CSP. The previously compiled data for RTP and CSP is captured in earlier examples. Table 35 shows the minimum, maximum, range, and coefficient of variation (CV) for the parameters for the three different platelet products (pooled CPP, RTP, and CSP). Overall, the pooled CPP product maintains its homogeneity after being stored at ≤−65° C. freezer for 12 months, the CV remains low even when the pooled CPP product is stored for a longer period of time, up to 12 months of storage. For example, referring to Table 35, in case of pooled CPP, the % CV for platelet count, pH, PS expression, thrombin generation, and maximum amplitude (MA) is less than 10% after 12 months storage. In case of pooled CPP, the % CV for R time is less than 15% after 12 months storage.

Additional statistical analysis of variability and stability were performed on the data. These analysis parameters include:
1. Mean normalized: the mean of a set of values after normalization
   a. Mean normalized=individual value % group mean
2. Percent difference from initial testing.
   a. Each product was evaluated for percent difference from results on the initial timepoint of testing.
   i. Pooled CPP was initially tested after 6 months of storage in ≤−65° C. freezer
   ii. RTP was initially tested at day 1.
   iii. CSP was initially tested at day 7

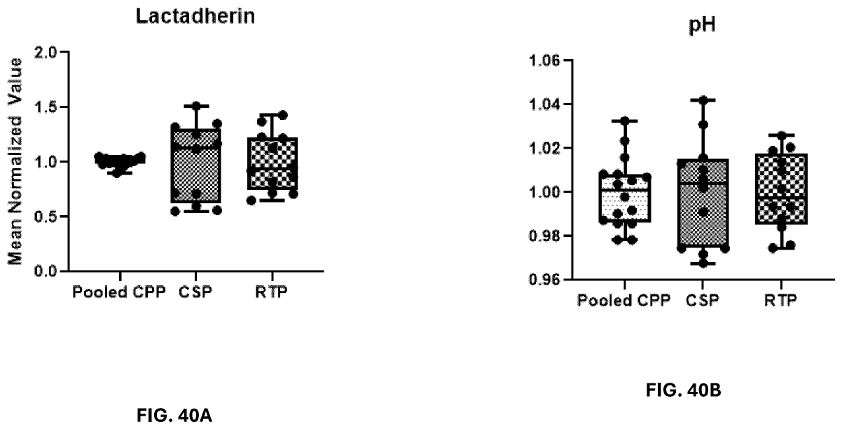

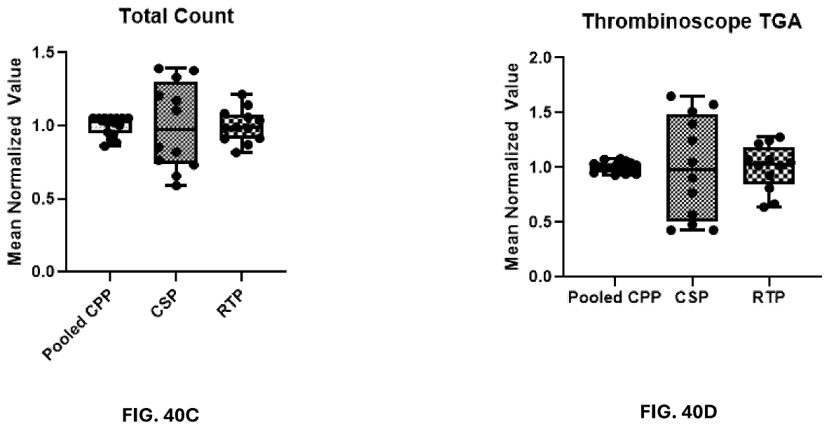

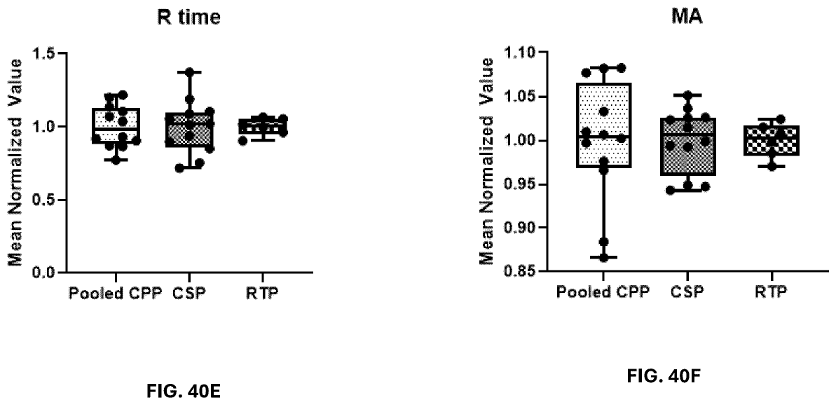

The FIG. 40 A to FIG. 40 F show the different attributes and the comparison of those attributes at 6-month, 9-month, and 12-month storage in case of pooled CPP, and in case of CSP the data is from initial test at day-7 and then at day-14, and in case of RTP the data is from initial test at day-1, and day-4 and day-7. FIG. 40A shows the mean normalized value for Lactadherin binding positivity. FIG. 40B and FIG. 40C show the mean normalized value for pH and platelet count, respectively. FIG. 40D shows the mean normalized value for thrombin generation. FIG. 40E and FIG. 40F show the mean normalized value for the R-time and maximum amplitude (MA). Pooled CPP has the tightest mean normalized values for lactadherin binding, pH, platelet count, thrombin and generation. The data presented herewith shows that the pooled CPP product herein is less variable, therefore homogenous even with extended storage times.

TABLE 35

| | | |
|---|---|---|
| Pooled CPP | CSP | RTP |

Platelet Count

| | | | |
|---|---|---|---|
| min | 1.8E+11 | 1.48E+11 | 2.42E+11 |
| max | 2.2E+11 | 3.48E+11 | 3.6E+11 |
| range | 4.00E+10 | 2E+11 | 1.18E+11 |
| % CV | 7% | 29% | 11% |

pH

| | | | |
|---|---|---|---|
| min | 6.5 | 7.04 | 7.24 |
| max | 6.86 | 7.58 | 7.62 |
| range | 0.36 | 0.54 | 0.38 |
| % CV | 2% | 2% | 2% |

PS Expression

| | | | |
|---|---|---|---|
| min | 75.4 | 26.6 | 11 |
| max | 87.9 | 73.1 | 24.1 |
| range | 12.5 | 46.5 | 13.1 |
| % CV | 4% | 35% | 27% |

Thrombin Generation

| | | | |
|---|---|---|---|
| min | 1.36 | 0.34 | 0.22 |
| max | 1.58 | 1.31 | 0.44 |
| range | 0.22 | 0.97 | 0.22 |
| % CV | 5% | 47% | 21% |

R time

| | | | |
|---|---|---|---|
| min | 0.49 | 0.63 | 0.96 |
| max | 0.78 | 1.20 | 1.14 |
| range | 0.28 | 0.57 | 0.17 |
| % CV | 13% | 18% | 6% |

MA

| | | | |
|---|---|---|---|
| min | 54.57 | 57.63 | 60.53 |
| max | 68.2 | 64.23 | 63.9 |
| range | 13.63 | 6.6 | 3.37 |
| % CV | 6% | 4% | 2% |

Table 36 shows the percent difference of the three products from the initial timepoint of testing.

TABLE 36

| | % Difference from Initial Testing | | | | |
|---|---|---|---|---|---|
| | Pooled CPP | Pooled CPP | CSP | RTP | RTP |
| | (9 month) | (12 month) | (Day 14) | (Day 4) | (Day 7) |
| Platelet Count | 1% | 2% | 53% | 4% | 3% |
| pH | 0% | 0% | 1% | 1% | 1% |
| PS Expression | 3% | 5% | 58% | 89% | 111% |
| Thrombin Generation | 7% | 7% | 81% | 8% | 25% |

Table 36 shows that the percent difference for all the tested metrics in case of pooled CPP even after 9 and 12 months of storage is less than 10% from initial testing of product. For example, the change in platelet count from the initial testing for pooled CPP is 1% and 2% at 9-month and 12-month storage periods, respectively. And, the change in PS expression from the initial testing for pooled CPP is 3% and 5% at 9-month and 12-month storage periods, respectively. Further, the change in thrombin generation from the initial testing for pooled CPP is 7% at both 9-month and 12-month storage periods. In contrast, CSP and RTP show a significant change in PS expression, and thrombin generation at day 14, for CSP, and at days 4 and 7 for RTP from the initial testing. The data further supports that the pooled CPP as disclosed herein maintains its attributes and homogeneity even after storing the pooled CPP product for 12 months at ≤−65° C.

### Example 32: Another Exemplary Pooled CPP Manufacturing Method

A non-limiting exemplary process for preparing cryopreserved platelets using a pool of platelet units as a starting material is as disclosed in this Example. Accordingly, a cryopreserved platelet composition comprising frozen activated platelets prepared by using a pool of platelet units from a plurality of donors, or a composition comprising cryopreserved platelets that have a biomolecular profile indicative of a plurality of donors, is referred as a pooled CPP, multi-donor CPP, or cryopreserved platelets disclosed or described herein. The exemplary pooled CPP process accommodates the inclusion and pooling of platelets from up to 30 apheresis platelet units (APUs), with the goal of improving product control, decreasing variability of the final product, and increasing scalability. The exemplary pooled CPP process incorporates all major process steps that are present in the exemplary CPP process of Example 2 with the addition of pre-spin equilibrium step and rinsing with plasma step.

The exemplary pooled CPP process creates multiple doses of a homogeneous final product. An illustrative, non-limiting process of preparing the cryopreserved platelets as disclosed herein is shown below. The exemplary pooled CPP process has been designed to incorporate up to 30 apheresis platelet units (APUs) from up to 10 donors into a single pool to produce up to 30 pooled CPP units. The process can be completed with less than 30 APUs, from 4-30 APU units from up to 10 donors as needed. The number of initial APUs equals the number of pooled CPP units produced.

### Step 1—Initial Quality Control (QC) of APUs and Weight Recording

The APUs were first received by QC before being released to manufacturing. The QC team visually inspected each unit for physical appearance and damage and then transferred the units to manufacturing.

At initial receipt of APUs, the manufacturing team inspected all units again to confirm acceptability. The weight of each unit was then measured and recorded and organized according to highest to lowest. This initial step is part of the pre-spin equilibrium step implemented in this exemplary pooled CPP manufacturing process. The manufacturing process will be herein described for receipt of 15 APUs.

### Step 2—Initial 2—Unit Pooling and Balancing Prior to Centrifugation

According to the weight of each APU, it is paired with another one such that the weight of all pooled units will be balanced. Each 2-unit pair is welded together using an Origen transfer set (S-F50) and pooled together making 1-7 pools and 1 single unit. The weight of each of the pooled bags (1-7) was taken and recorded.

The 7 pooled bags and 1 single unit were then welded onto an Origen manifold (4S-4M60) and redistributed into six pooled bags (1-6 pooled bags). The weight of 1-6 pooled bags were taken, and average weight was calculated. Using the heavier pooled bags, each bag was adjusted to be within +/−10 g of the average weight of the 1-6 pooled bags. The pre-spin equilibrium step was added to ensure all cups in the centrifugation machine were properly balanced. The step of taking the weight of 1-6 pooled bags, recalculating the average weight and readjusting the weights to be within +/−10 g of the average weight can be repeated until all bags are within the range.

### Step 3—Centrifugation

Each pooled bag (1-6) was sterile disconnected from the Origen manifold and sterile welded to a corresponding empty bag for plasma expression. The pre-spin weights of each bag were determined, and the plasma expression weights were calculated based on target pellet weight of 43.8 g.
Plasma expression weight=pre-spin weight−43.8 g

Prior to running the centrifugation, the rotor was ensured to be balanced (+/−5 g or each other). The configuration was run with the following settings: 1,380 g for 12 minutes with 10 minute slowdown.

After centrifugation, plasma expression was performed for each pellet to achieve the calculated plasma expression (+/−1 g). The weights of each pellet was recorded and ensured to be within the range of 31.8-55.7 g. After that the pellets were resuspended in the remaining plasma.

Step 4—Pool Platelet Pellets and Rinse with Plasma

The bags of resuspended platelet pellets were attached to an Origen manifold to be pooled into a 1 L bag (CS1000N). A 2 L bag can be used as well. The combined pooled platelet pellets in the 1 L bag were weighed and recorded. This weight is identified as pellet pool.

An amount of plasma rinse was calculated according to the following equation.

$$Plasma rinse target ( g ) [ + / - 1 g ] = pre - DMSO target ( g ) - pellet pool ( g )$$

The pre-DMSO target is evaluated based on the pellet pool estimate (on a per APU basis) from Example 2 plus any small aliquot(s) that were removed for in-process testing.

To initiate the plasma rinse target is added to an empty APU bag and welded to the Origen manifold. The plasma rinse is transferred to each of the 1-6 empty platelet pellet bags for rinsing and then finally dispensed into 1 L pellet pool bag. The weight of the pellet pool after adding the plasma rinse is taken and recorded (pre-DMSO pool) to ensure it's within an indicated range for the number of APUs.

### Step 5 Adding DMSO

To add the DMSO to the pre-DMSO pool, the amount of DMSO addition target weight was calculated according to the below equation.

$$DMSO addition target ( g ) [ + / - 1 g ] = pre - DMSO pool ( g ) * 0.2946$$

Once the DMSO addition target was calculated that amount was dispensed into an empty CS250N bag. To ensure accuracy, the weight of the CS250N bag was taken to confirm the correct DMSO addition target and the bag was attached to the pre DMSO pool bag such that the DMSO flowed gravimetrically into the pre DMSO pool bag. The weight of the combined DMSO and pellet pool bag is taken (total weight) and recorded

### Step 6—Filling CPP Bag and Storing

The maximum and minimum cryobag fill was determined based on the following equations.

$$Maximum cryobag fill ( g ) = Total weight ( g ) / number of units$$

$$Minimum cryobag fill ( g ) Maximum cryobag fill ( g ) - 2 g$$

If after DMSO addition small aliquots were removed from the bag for in-process testing, the total weight will account for the removal of the weight of the small aliquots. Since the process started with 15 units of apheresis platelets, the number of units to be filled were 15. 15 cryobags were filled according to the minimum and maximum cryobag fill (g) and labeled accordingly. Each cryobag was then placed into a thawing bag and a freezer carton for storage in a≤−65° C. freezer.

### Example 33: Impact of DMSO Exposure Time

The Pooled CPP product was manufactured according to the method of Example 32. Several cryobags of the pooled CPP product were held for different timepoints (0, 3, 4, 5 hrs) before transferring to ≤−65C freezer for storage to test for the impact of DMSO exposure prior to freezing. After being stored for ˜1 month at ≤−65° C. freezer, 2 cryobags of pooled CPP for each timepoint were removed for testing.

The pooled CPP product stored in cryobags was removed from the −80° C. freezer and transferred to the lab on −80° C. ice packs. The required number of cryobags were then placed into a plasma thawer set at 37° C. for eight (8) minutes. The cryobags comprising thawed platelet particles were removed from the plasma thawer and checked for cracks, breaks, or leakage. The thawed platelet particles in each cryobag were diluted with 25 mL of 0.9% saline. The cryobag was gently massaged to mix, unit was fitted with a clean syringe via the leur port, and approximately 10 mL aliquot was prepared by slowly drawing the thawed platelet particles into a syringe and then was deposited into a clean conical tube for testing.

To determine the DMSO impact, the pooled CPP product was visually inspected for aggregate-free swirling and tested for platelet count, pH, lactadherin positivity, and TGA. The platelet count, pH, lactadherin percent positivity, and TGA (TGPU) was determined according to the methods of Example 9. Table 37 shows the results of the measurements taken to test the impact of DMSO exposure prior to freezing at the different timepoints. The minimum platelet count requirement is met at all timepoints. The pH parameter fails at 5 hours post DMSO exposure.

TABLE 37

| | | Aggregate- | Platelet | | | |
|---|---|---|---|---|---|---|
| | Load | free | count | | Lactadherin | |
| Cryobag | time | swirling | (≥1.7E+ | pH | percent | TGA |
| Sublot | (hrs) | (pass/fail) | 11) | (≥6.2) | positivity | (TGPU) |
| Sublot A | 0 | Pass | 2.1E+11 | 6.7 | 76% | 1.6 |
| Sublot B | | Pass | 2.1E+11 | 6.4 | 78% | 1.5 |
| Sublot C | 3 | Pass | 2.1E+11 | 6.6 | 77% | 1.5 |
| Sublot D | | Pass | 2.2E+11 | 6.5 | 77% | 1.5 |
| Sublot E | 4 | Pass | 2.3E+11 | 6.6 | 78% | 1.5 |
| Sublot F | | Pass | 2.3E+11 | 6.5 | 76% | 1.5 |
| Sublot G | 5 | Pass | 2.2E+11 | 6.1 | 78% | 1.7 |
| Sublot H | | Pass | 2.1E+11 | 6.1 | 78% | 1.7 |

| | | | | |
|---|---|---|---|---|
| Mean | 2.2E+11 | 6.4 | 77% | 1.5 |
| CV | 4% | 2% | 1% | 2% |

The table shows that the pooled CPP product passes minimum pH requirements up to 4 hours of post DMSO exposure prior to being frozen. The Lactadherin percent positivity and TGA were obtained for informational purposes only.

### Example 34: Pooled CPP Product Demonstrated Adhesion to Collagen Coated Channel Using the BioFlux Platelet Adhesion In Vitro Assay

This Example demonstrates the specific adhesion of pooled CPP cryopreserved platelet compositions herein, and platelet rich plasma (PRP) from healthy donors onto collagen-coated channels. The adhesion was assessed using the BioFlux system under shear flow. The BioFlux assay was performed according to manufacturer's operating procedure for platelet adhesion under shear flow. The BioFlux system is a microfluidic flow platform designed to replicate physiological relevant shear conditions for studying cell behavior under controlled flow. It integrates microchannel plates, pneumatic flow control, and real time imaging capabilities to enable high-throughput assays that mimic vascular, inflammatory, and tissue-interface environments.

Coating the BioFlux Plates with Collagen or BSA In order to prepare the BioFlux system, the plates were first either coated with collagen or bovine serum albumin (BSA). The BSA is used as a negative control to confirm that any adhesion identified is specific to collagen. Albumin is a generic protein in the blood and is not part of the injury site. The BioFlux channels were primed according to standard procedure. Collagen was diluted to 80 μg/mL in 20 mM acetic acid and BSA was diluted to 0.5% in Dulbecco's Phosphate Buffered Saline (+Ca/+Mg) forming DPBS-BSA. Collagen or DPBS-BSA were perfused into the BioFlux plate channels at 5 dyn/cm² for 10 minutes and incubated in room temperature for 1 hour to coat the channel. After coating, the channels were rinsed with 0.5% BSA in DPBS at 30 dyn/cm2 for 6 minutes and then incubated overnight at 4° C. to block.

### Preparation and Staining of Platelet Products

Each platelet product was prepared and stained according to the following method steps:

Platelet rich plasma (PRP)
1. Whole blood was collected in sodium citrate tubes
2. The whole blood was centrifuged at 180×g for 20 minutes and top layer containing PRP was collected and transferred to a new tube.
3. The PRP collected in the new tube is centrifuged again at 20×g for 5 minutes to pellet any remaining leukocytes and top layer of PRP was collected
4. A 1 mL sample of PRP was transferred to a fresh tube and Calcein AM was added to a final concentration of 10 μM. The sample was incubated for 20 minutes at room temperature in the dark to stain the platelets.
5. As the stain was incubated, the platelets were counted using the CASY cell counter by obtaining a small aliquot.
6. Platelets were diluted in CASYton buffer to an appropriate measuring range of the instrument and a sample volume of 200 μL of the diluted platelets were counted on CASY.
7. The CASY cell counter enumerates particles between 1.56 and 5 μm in diameter size.
8. The stained PRP particles were then diluted in Octaplas based on the CASY counts to achieve 2.5×10⁵ platelets/μL to a final volume of 1 mL.

### Pooled CPP Product

1. Three different batches of CPP (Lot 13, Lot 21, and Lot 36) were manufactured according to the method of example 2 and frozen at −80° C.
2. 1 pooled CPP unit from each batch was tested on the BioFlux for adhesion
3. Each pooled CPP unit was thawed in a 37° C. water bath for 8 minutes before use.
4. Each pooled CPP unit was diluted with 25 mL of 0.9% saline and a 2 mL sample was transferred to a fresh tube.
5. Calcein AM was then added to the 2 mL sample to achieve a final concentration of 10 μM and incubated at room temperature for 20 minutes in the dark to stain cryopreserved platelets in each sample.
6. As the stain was incubated, the pooled CPP samples were counted using the CASY cell counter by obtaining a small aliquot.
7. Pooled CPP was diluted in CASYton buffer to an appropriate measuring range of the instrument and a sample volume of 200 μL of the diluted pooled CPP was counted on CSAY.
8. The CASY cell counter enumerates particles between 1.56 and 5 μm in diameter size.
9. The stained pooled CPP samples were then diluted in Octoplas based on the CASY counts to achieve a 2.5×10⁵ platelets/μL to a final volume of 3 mL and a 5×10⁵ platelets/μL to a final volume of 3 mL

### Platelet Adhesion Assay

Prior to initiating the platelet adhesion assay, the BioFlux heater plate adapter was pre-warmed to 37° C. The coated BioFlux microfluidic plate stored at 4° C. was transferred onto the heated adapter within a 37° C., 5% CO₂ incubated environment and allowed to equilibrate for 20 minutes. The assay was performed one column of flow cells at a time, each column containing six channels corresponding to three viewing windows. Excess PBS-BSA blocking solution was aspirated from the inlet and outlet wells associated with the first two columns of channels. A volume of 300 μL of fluorescently stained platelet sample was dispensed into each inlet well of the BioFlux plate. The microfluidic plate was positioned on an inverted microscope using a 10×objective. A shear stress of 2 dyn/cm² was applied to all channels until stained platelet samples entered the viewing-window region. The Z-position for each viewing window was then set and recorded under the FITC fluorescence channel, and the coordinates were saved to an automated stage list. Following Z-positioning, platelet samples were perfused through the channels at 30 dyn/cm² for 7 minutes, and time-lapse images were captured at 30-second intervals. Upon completion of imaging for the first column of flow cells, the procedure described above was repeated for each subsequent column until all flow cells on the plate had been processed. The platelet samples tested on the BioFlux were as follows:
a) 3 channels: PRP on collagen at 2.5×10⁵ platelets/μL (positive control)
b) 9 channels: pooled CPP on collagen at 2.5×10⁵ platelets/μL
c) 9 channels: pooled CPP on collagen at 5×10⁵ platelets/μL
d) 3 channels: pooled CPP on albumin at 5×10⁵ platelets/μL (negative control)

### Image Analysis

Image analysis was conducted using the Montage Plus software platform in order to quantify fluorescent area coverage as a function of time. For each field of view, background fluorescence intensity was first measured and subtracted from the corresponding image set to yield background-corrected images. A threshold value was then determined such that the resulting binary mask selectively identified fluorescent platelet signal while excluding non-specific background. The same threshold mask was applied uniformly to all background-subtracted images within the time-lapse sequence. The total fluorescent area (μm²) for each time point was quantified, thereby generating an area-coverage-over-time dataset.

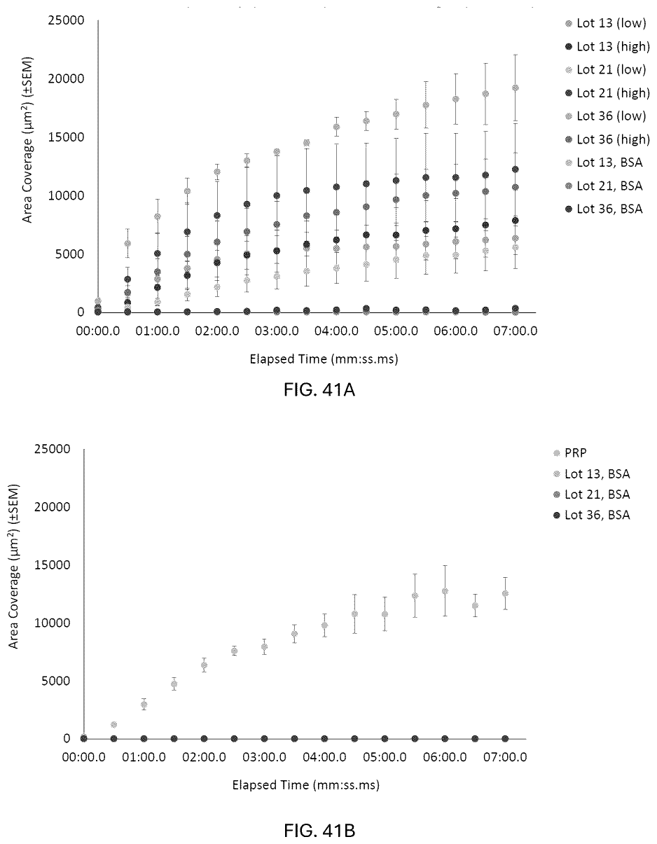

FIG. 41A shows the output from the image analysis for determining the adhesion of thawed activated platelets herein to a collagen-coated channel. FIG. 41B shows the output from image analysis for determining the adhesion of platelets from platelet rich plasma to a collagen-coated channel. Referring to FIG. 41A, the BioFlux assay confirmed that the pooled CPP product from all the three different batches (lot 13, lot 21, and lot 36) adheres to collagen-coated channels independent of external platelets, like endogenous platelets. The adhesion further indicates that the pooled CPP product as disclosed herein is capable of initiating a hemostatic response at the site of injury.

Further, no adhesion to albumin-coated channels was observed with any of the batches (lot 13, lot 21, and lot 36) of pooled CPP product tested in this Example. No adhesion to albumin-coated channel was detected even at the longest contact time (or elapsed time) of 7 minutes and highest concentrations of pooled CPP tested. The ability of thawed activated platelets in pooled CPP produced using methods herein to adhere to collagen-coated channels, but not albumin-coated channels demonstrates the property of frozen activated platelets in these pooled CPP cryopreserved platelet compositions to specifically adhere to collagen-coated channels.

Furthermore, based on the area under the curve (represented by the fluorescence signal from the thawed frozen-activated platelets or platelets from PRP bound to collagen-coated channel), a dose dependent adhesion to the collagen-coated channels was observed for all of the lots tested for the pooled CPP product. It was observed that the frozen-activated platelets in pooled CPP from lot 36 at a concentration of 2.5×10⁵ thawed active platelets/μL demonstrated a mean area coverage starting at less than 1,000 μm² at time 0 to 11,000 μm² to 14,000 μm² when observed after 2 minutes, and increased to 15,000 μm² to 22,000 μm² at 7 minutes (FIG. 41A) of contacting the collagen-coated channel. On the other hand the PRP at the 2 minute time-point and also starting with a mean area coverage of less than 1,000 μm² at time 0, exhibited 5,000 μm² to 7,000 μm² area coverage (FIG. 41B). At the 7 minute time point, the PRP control sample exhibited between 11,000 μm² and 14,000 μm² area coverage, It was further observed that the pooled CPP from lot 13 at a concentration of 2.5×10′ thawed activated platelets/μL at the 2 minute time point demonstrated a mean area coverage of between 3,500 μm² and 5,000 μm², and increased to between 5,000 μm² and 8,000 μm² at 7 minutes of contacting. It was noted that the lot 36 at the high concentration (5.0×10⁵ thawed activated platelets/μL) clogged the channel and negatively affected the assay performance for this sample.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects, exemplary aspects and optional features, modification and variation of the concepts herein disclosed may be resorted to by those skilled in the art, and that such modifications and variations are considered to be within the scope of this invention as defined by the appended claims. The specific aspects provided herein are examples of useful aspects of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific aspects that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents of any such materials and methods are intended to be included in this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects and optional features, modification and variation of the concepts herein disclosed may be resorted to by those skilled in the art, and that such modifications and variations are considered to be within the scope of this invention as defined by the appended claims.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

1-7. (canceled)

8. A collection of cryo-vessels, comprising
a plurality of cryo-vessels, each cryo-vessel in the collection comprising 1 unit equivalent of frozen activated platelets comprising a population of platelet particles in a cryopreservation medium in a frozen state,
wherein the population of platelet particles in the frozen activated platelets in each cryo-vessel have a set of biomolecule profiles indicative of more than 1 platelet donor, and
wherein the frozen activated platelets in each cryo-vessel in the collection, exhibit the following properties, upon thawing:
form a thawed activated platelet composition weighing between 24.50 and 30.50 grams when diluted with 25 ml of saline;
have a capacity to generate thrombin in an in vitro thrombin generation assay, and
have less than 10×10⁶ CD61-positive microparticles/μl of the thawed activated platelet composition, and
wherein the thawing comprises placing the cryo-vessel in a water bath set at a temperature of 37° C.+/−2° C. until the frozen activated platelets are thawed to form the thawed activated platelet composition.

9. The collection of claim 8, wherein the frozen activated platelets in each cryo-vessel in the collection, have the property of having less than 9.0×10⁶ CD61-positive microparticles/μl of frozen activated platelets, upon thawing and when diluted with 25 ml of saline.

10. The collection of claim 8, wherein the frozen activated platelets in each cryo-vessel in the collection, have the property of having CD61-positive microparticles in the range of 2.0×10⁶ to 9.0×10⁶ CD61-positive microparticles/μl of frozen activated platelets, upon thawing and when diluted with 25 ml of saline.

11. The collection of claim 10, wherein the frozen activated platelets in each cryo-vessel in the collection have the property of exhibiting a pH of greater than 6.0 upon thawing, and when diluted with 25 ml saline, and storing at room temperature for 6 hours to 24 hours.

12. (canceled)

13. The collection of claim 10, wherein the collection comprises at least 10 cryo-vessels from at least 3 batches of cryo-vessels comprising frozen activated platelets.

14. (canceled)

15. A process for preparing a batch of a cryopreserved platelet composition comprising a population of platelet particles,
comprising:
a) forming a concentrated pooled platelet resuspension (CPR) by removing some plasma from pooled platelet units to achieve a weight or volume based on the number of platelet units (PU) to form the CPR;
b) adding dimethyl sulfoxide (DMSO) to the CPR to obtain a CPR having DMSO
c) distributing the CPR having DMSO among more than 1 cryo-vessel from a collection of cryo-vessels; and
d) freezing the collection of cryo-vessels to prepare the batch of the cryopreserved platelet composition comprising the population of platelet particles,
wherein the distributing is done to obtain 1 PU equivalent weight of the CPR having DMSO in each cryo-vessel, and
wherein the freezing is initiated within 2 hours after adding the DMSO.

16. (canceled)

17. The process of claim 15, wherein the freezing is initiated less than or equal to 1 hour after adding the DMSO.

18-19. (canceled)

20. The process of claim 15, wherein the forming the CPR comprises pooling 2 or 3 platelet units in one vessel, and pooling 1 to 3 platelet units in one or more additional vessels, to form a plurality of vessels, wherein at least 5 platelet units are provided, wherein the concentration of DMSO in each vessel is in the range of 4% to 8%, and wherein the platelet units are from two or more donors.

21-23. (canceled)

24. The process of claim 20, wherein the process further comprises:
introducing the platelet units from the plurality of vessels to a tangential flow filtration (TFF) system, and
concentrating the platelet units to form the CPR having a target weight based on the number of platelet units pooled or provided in the plurality of vessels.

25. (canceled)

26. The process of claim 15, wherein the process is repeated at least 3 times to produce at least 3 batches of the cryopreserved platelet composition, and wherein each batch comprises 3 to 30 cryo-vessels.

27. (canceled)

28. The collection of claim 13, wherein the platelet particles in the population exhibit phosphatidylserine positivity of between 60% to 95% when measured using lactadherin binding.

29-34. (canceled)

35. The collection of claim 10, wherein each cryo-vessel has the equivalent of 1 unit of the frozen activated platelets, and has the following property: having CD61-positive microparticles in the range of 2.0×10⁶ to 9×10⁶ CD61-positive microparticles/μl of the frozen activated platelets upon thawing, and storing at room temperature for 6 to 24 hours, wherein the thawing comprises placing the cryo-vessel in a water bath set at a temperature of 37° C.+/−2° C. until the frozen activated platelets are thawed to form a thawed activated platelet composition, and diluting the thawed activated platelet composition with 25 ml of saline.

36. (canceled)

37. The collection of claim 10, wherein each cryo-vessel has the equivalent of 1 unit of frozen activated platelets, and has the following property: exhibiting a total count of thawed platelet particles in the range of 1.8×10¹¹ to 3.5×10¹¹, upon thawing and when diluted with 25 ml saline, and storing at room temperature in for 6 to 24 hours, wherein the thawing comprises placing the cryo-vessel in a water bath set at a temperature of 37° C.+/−2° C. until the frozen activated platelets are thawed to form a thawed activated platelet composition.

38-41. (canceled)

42. The collection of claim 10, wherein each cryo-vessel has the equivalent of 1 unit of frozen activated platelets, and has the following property: exhibiting a coefficient of variance of the ratio of concentration of CD 61-positive microparticles to concentration of thawed platelet particles in the frozen activated platelets in the cryo-vessels, across at least 5 batches, of less than 35%, 30%, or 25% within the batch of the cryo-vessels, upon thawing and when diluted with 25 ml saline, wherein the thawing comprises placing the cryo-vessel in a water bath set at a temperature of 37° C.+/−2° C. until the frozen activated platelets are thawed to form a thawed activated platelet composition.

43-58. (canceled)

59. The collection of claim 10, wherein the concentration of DMSO in the frozen activated platelets in a cryo-vessel within a batch or across at least 5 batches varies by no more than 0.5%, and wherein the DMSO is in the range of 6%+/−0.5%.

60. (canceled)

61. The process of claim 26, wherein each cryo-vessel across at least 2 batches has the equivalent of 1 unit of frozen activated platelets, and has the following property: having less than 10×10⁶/μl CD61 positive microparticle concentration of the cryopreserved platelet composition, upon thawing platelets-er-the cryopreserved platelet composition, and when diluted with 25 ml saline.

62-63. (canceled)

64. The collection of claim 13, wherein the collection has at least 20 cryo-vessels and has the following property: having less than 35.0% of the cryo-vessels in the collection with a CD61 positive microparticles to thawed platelet particles ratio of greater than 1.0.

65. The process of claim 26, wherein the collection has at least 20 cryo-vessels, and wherein the collection has the following property having less than 35% of the cryo-vessels in the collection with a CD61 positive microparticles to thawed platelet particles ratio of greater than 1.0.

66. A method for reducing bleeding in a subject, comprising:
thawing the frozen activated platelets in a cryo-vessel from the collection of claim 8, to obtain a thawed composition comprising thawed platelet particles, and
administering the thawed composition comprising a dose of the thawed platelet particles to the subject,
wherein the administering leads to the subject having reduced bleeding such that after the administering the bleeding in the subject is reduced as compared to a bleeding in the subject before the administering,
wherein the method comprises resuspending the thawed composition with saline to obtain a thawed-resuspended composition comprising thawed platelet particles
wherein the subject is undergoing, or has undergone surgery, and
wherein the surgery is cardiopulmonary bypass surgery, and the administering is done intraoperatively, or post-surgery.

67-69. (canceled)

70. The method of claim 66, wherein the post-surgery administration comprises administering the composition to the subject wherein the subject has an active clotting time (ACT) of less than 250 seconds.

71-145. (canceled)

146. A method for reducing bleeding in a subject, comprising:
thawing the frozen activated platelets in a cryo-vessel from the collection of claim 8, to obtain a thawed composition comprising thawed platelet particles, and
administering the thawed composition comprising a dose of the thawed platelet particles to the subject,
wherein the administering leads to the subject having reduced bleeding such that after the administering the bleeding in the subject is reduced as compared to a bleeding in the subject before the administering, and
wherein the subject has traumatic brain injury (TBI) and/or intracranial hemorrhage (ICH).
